SEQUENCE LISTING (46 sequences)

SEQ ID NO: 1 - protein, 424 aa, Glycine max
MLQDVVHPSTPAEQLPIDEISGPISARIFELCDPDFFPHTLQNSEVTSSSNCCHEEKSSYATTISPPLDVVDNNKFNINSNSSNIVTTTSSSTTTTSTTTNNNNNATNGNNLSIFFDTQDEIDNDISASIDFSSSPSFVVPPLLPISTQQDQFDFPSAQPQVQLSTAAGSILTGLSHYPTDPVIAPLIGAPLPSVFDDDCISSIPSYVPLNPSSPSCSYLSPGIGVYMPPPGSLNTALSADSSGLFGGNILLGSELQAHELDYQGENGGMYCTDSIQRVFNSPDLQALGNESQKLVAGAGSSATLAPEISHLEDSTLKVGKLSVEQRKEKIHRYMKKRNERNFSKKIKYACRKTLADSRPRVRGRFAKNDDFGESHKQGSSNHEDDDEEIIVKEDDDMVDSSDIFAHISGVNSFKCNYSIQSLI

SEQ ID NO: 2 - DNA, 1275 bp, Glycine max
  source 1..1275: mol_type = genomic DNA
atgttgcaggatgttgtccacccctcaacaccggctgagcaactccccattgatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaagtatgcttgccgcaaaactttggcggatagccggccccgggttagaggaaggtttgcaaagaatgatgactttggagagagccataaacaaggaagtagcaatcatgaagatgatgatgaagagataattgtgaaagaagatgatgatatggttgattcctcagatatctttgcacatatcagcggagtgaactctttcaaatgcaactattccatccagtccttgatttga

SEQ ID NO: 3 - DNA, 5035 bp, Glycine max
  source 1..5035: mol_type = genomic DNA
catcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccatgttgcaggatgttgtccacccctcaacaccggctgagcaactccccattgtaatcacctcaagcttgttacatttcatatctcgatcatgtatatttagtgttaatgcatgtattggaactaagctatatatgtatgtgtgtatgtatctttgtgtcatgcaatatttatgaccacagctaaatggtttccttctgggttttgtgatcgtgtgtaacttgacaagtttgtttggatcaagggcttgtttttttctttcaaaataatgatttcacactccaagagtgtgtaaaccttgggagggaagagaaagacaggaaagagaccccagaaaagaacaaaagaagtctaaaattgatgtacggattaatagcgtgtttgaaaacccattgagaagtaagaaaatctcatctgagatatgaaagttatgactattaacttcctaataaactcaagaaatcacatctgagatgtgaaaccaaacatgctcttacgaattgtagaaattttgatggtttttctctggggtactctttcttatattttgaggccaattattactaatttctctaggaattattcgatcaattgctccattcctgcattttaattctttccatgcatgaatcacgaatatggttttgttaatgtttgttggcatgttaattaatttcatcctaattagttctctgagaaacctgaacaatatttcccttatttggttatattcctggagctaagtggaagctgctagtgctattagccatcttagaaaacccacaagccatgctcactatttgtgggacggaacgttttctttcctctatttatatggttggagagcataacgtaattttattgaccgaacaataggaaggaatcaaagactccgtgaccgtgctgtcaactcttttgatatttattatcatccaatgggccctaccaccaaaatgtttaatgtcattttccaaggggcaaagttgtaaaaatgatgtaacatttccgtgatagtaatttagacatagctttcggttcttgtagaatatatatatctctttgggggcttgttgtgtaagctttgctaatttttgatattctttattcagttagatagggagaagaggtcttagtgacaaacaattcatatggatagcatatatagcattatggggttctcttcaagtggctttcaattcccatttaagttcaaattttccacttaaaatgaacaaaaataacatagcatgaggttccttgtttgtttttgcatacttgtatttcatactaacattggctaccttggctcctacctgtcacatgaaggcatagatgcacatcactttcaccaataaataacccaacaatgcaaaccctttaggaaaatttctcttcagtctattaccaaatatggacaaaactgactagactatgcaaccaaccacagtcatatgtagaattcttcgttggggcctttgcacatcgatttatattgttttcctcttgataaagaaggaagggggtgttgtggcagatactattatatcatttggcatgtctttcacttttgaatgcccactacatctacaagccaaagcttcaattgattagagaatattagcttttgaatttcttttctgttctaagtgatcagatcagactcatgtctatcaacaaaaagaggaattggattcagatttcaaacccatcataatgaagaaaaaaataaaaaaaataaactatagagaatcacgtttcctagattgtttataaagaagaggctttgttaagactattctcaggctgtgttttagtagtttatggtaaacatgttgttcacaatgcttatatgtcaccactcattgcaggatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggtatgtgcaatttcgcaagccaattagagtttaatagacattcattgtctggtataaaagtttttacattatcaatcaatcagataccattgttgatataaattttaaaataattgttataataattaataatttaatgtacttgataatttgtgatttgataataatataaaaaaaatttacactgcattattatttatttttcctgtcgactgtcaataaactaaatgaaaattttcagttccaatgttcaatgtgttcagaataaggaaaaagaagtttaataatgctgcaaaggttactataccttgcagcagtgaagtttttattttaaaatagaagaggctttatcagaggtggacttttgggggaaagctcagggtccacaaatctctaaactataaactcataggtgccccatgaccatcaaatagtaggtagcacaagatatgagtccatttataaagtcacatgcattaaaaaatactataaatttggcctagcaagaaggaagaaccactttcatccaaaagaaaaatagaaaaaaggataataaactgtagcatcattagatagaaagacccacttcaagggtggcagtgttatatctctttctacagtctataaagttaatgtgcagtttttattgaataagtaagaaattgatctttaattataatttctctctcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaaggtactacatctgaacaccaacattaacaaacaaatttcaaatcttatactgttttacatgatttccaatctactgcatcaaccaagccttatgcatattttcaaaattcaactaatgatgcaattttttttatataaaaaaaatgcagtatgcttgccgcaaaattagagagagggttggagtagcgcctattgtagagaagatggtggaaaatagacttaggtggtttgggcatgtagagagaagaccggtagactctgtagtgaggagagtagaccagatggagagaagacaaacaattcgaggcagaggaagacccaaaaagactataagagaggttataaaaaaggatctcgaaattaatggtttggatagaagtatggtacttgatagaacattatggcggaagttgatccatgtagccgaccccacctagtgggataaggcgttgttgttgttgttgttgttgtatgcttgccgcaaaactttggcggatagccggccccgggttagaggaaggtttgcaaagaatgatgactttggagagagccataaacaaggaagtagcaatcatgaagatgatgatgaagaggtaagattcccttaatcggatactgttgttcaacttgccttagtctaaaaattaaaatacaaaaaaattcccgatcacttttaccttttcaattatttgatggcataattccttgatgttatattccttccattttttgtacttgcagataattgtgaaagaagatgatgatatggttgattcctcagatatctttgcacatatcagcggagtgaactctttcaaatgcaactattccatccagtccttgatttgaattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttat

SEQ ID NO: 4 - DNA, 12557 bp, synthetic construct
  source 1..12557: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgttcctaacacctggagaaccttttatgtacttcacaaccctcaatgctgcttccaggtgtgactgtttgggttgctgcatgaattgactcagcacctgcactgcaaagctgatatctggccttgtgattgtcaagtagagaagctttcccaccagtctctgataggatcccacatcctctaactctgcatcatcagtctttcctaagtgcttgtcatattctacagtagtcagcctttggttctgttccattggggttgacactggtctgcagcctcccagaccaacacctgatatcagttccagtgcatacttcctctggttcagtaggattcccttttctgatctcagcacctcaatgcctaagaagtattttagttctcccaaatctttcatcttgaaatgctgatgcagggttgcctttgcttctgaaatcaaaacattgctgctgcctgttattaacagatcatccacataaatcaggattatgacaaggtcagtcccctctcttttggtaaacaaggagtgatcataagcactttgcataaaaccagcctgcataaggacagtggtaagcttgatgttccactgccttgatgcttgctttaaaccatagagggattcaacagcctgcacactttggtttccccttggctgtgaaaaccctgaggcagagacatataaacttcttccatgaggtcaccttgtagaaaagcattgttgacatccatctggaaaaggaaccagcccttggaagcagcaacagatatgacagcttttacagtgaccattttggccactggagaaaaagtttcatggtagtcaaggccttcttgctgagtgtatcccttggccactagccttgccttaaacctgtcaacttcaccattagctttgtatttaattttgtacacccatttggaccctataggctgtttaccagggggtaaagggacaatctcccaggtgttattatcctcaagagcctgtatctcaagggacatggcctccatccatttctcatcttgagctgcttctttgaaggatttaggttcagtataagtggagaaagcactcaaataagcttgatagtgaacaggcaagtgatcataggaaacatagttggctatagggtatggaacatccctagagcctttgttcagtgtcacaaagtccttgagccagatgggaggacctgcattgcgtttaggtctagtgtgcaggtttgctggaactagagagggatcagctacagcagtatggtgctcaaattcagcatttgctaggtcaggctcagctgactcaactgactctgcaggagcatgcaggtggcctgaaggtgcagcatcagctgaagtgatatgattttgggtagaatgagtccccaaagtgatatcctcattggcctctacaatgtcaggaataagaactggagtatcatcatcatcagtatgaaagctggaaggaaaaatatcattatacactagctgcaaagctgtatcttcagagctaagtgaacctgctcgagtgaacatatcaggttcatgggagatggactctttgaaagggaactgaaactctctgaaaactacatccctgctcactatgatcaccttattatccaagtcatacaacctgtaacccttttgagtctcagaataaccaatgaagatggttcgcttggctctaggtgcaagtttgtcacctttgggcagtgttgctgcaaaagcaagacacccaaacactctcaagtgatcaagcttggcatgtttctgatagagtagttcatatggacatttgccttgtaagattggagtagggagtctattgatcaggtatacaacagtcttgacacagtctccccaaaacctggtaggtacaccactctgaaacttaagtgcccttgccatctcaaggatgtgtctgtgctttctctccacaacaccattctgttgtggtgtgtagggacagctactttgatgaacaatcccaagagaggccagcaactcattacaacttctccactagaattcgagctcggagctgtaggccggcctagcttattctcacccctaattaagacaagctgtaggtcggcccctgttcatgttgttcatcaattattgtttaaccgagttaaaggagatggtcaattttacagtagtaaatatgacaagaaatacacgaaatttgtgtttaagtattgaaggacttccatttttttgaatccaccatcctaaaactaatagtgtctaattttcccgtaacaatttcttttgtataacattcaaataaatgtggttgacgatatatatttagcttgcaaatttcctaaaaattattgctccaaacttagtatgcacttaggattggattgtgtcattattgtacttggcaagggtacatgtgttaattatatgcacttaagcgtgtcattgtacttggctacttaaaattgttttttgttcctcggatgatgttgatgagttataaaaaagaaatatatgccttgaaggaaagaaagtttatttttacggtttatgttaaaatacaatactttcaactatgtagattgtggagcaaacaagctttcagtttctctatcgtgttccgatgacacgttaataatgaacttttcctataaacatcaattagaagatgttgattttgttggtcaattggtgttattgttgtgactactttatattttcgttagattccaagcattatttccgtctatagggtttgctcttaactgattgttttgagttattaattattcaagttgatttgattggaatagtaaatctggatctattatgtaatatattagggacgaaatcttgcacattgagaatgatgtagtctcctcaaaattttgataaccattgttttcgagtgatagatgttgtctatgtcaaggttgtcgatgaaattagcaatgatggacagactttgtcgaacaagttttgtaatgccatcacagaaaagcatctagaagtactgaaaaacattttttttaacatatacttttacaaaatcaagagaatgaataattaaatatgaaataagaattacaaaaattttatatttttttaataaatttcaaatcaataataagaaagaatgggataaaattaaagaatatgttggaagatgtgttattagtatttctgtgcataagaatgcttccaaaattaaactctttccccaaaattgtatgagtaaaatttcaaatgtctcgttttaaatataacataagctaaattaaacccagcttagtaaaaaaaatgcatttccgtaaatttcgatgtccaaacaattgtaatgacttaaactcagaacaaaacctatttttataccatgtaaattcttacacattataatcgtatgtttatccatccatgcaccccaacaaatgaagaaaaggaataaaaaaaaaggtgaatttgccaaaaacaaattaatgggatgctaacattaacataaggcggcatagggcatcatttaattgtgaataaaaaggcttggtcaacccaatttcttctcctagttactagttattaaccatgtcatttggcatatatataataatccatatccatataaaaatacatgtcccaaaaattgaattccactacaccccaccaaagtggtgaattaatggccaaacgccactctcgcgttgacacccgcgagtgccaggcttgacgactctacgtacaacctcatcaattcctctatatatgcacataactaactttctctgtttttgacaccctcatacaccccatcatcttagctaacacaacacagcatacacaacttctctctctctactactttctttttatgtcaccttcttgatgtctttaatttgttttcttttttagaaaaaaattaatcctttttaaggctttaatttcctaagcatgcaatattattgtttttagtcaccttcaagttgaggaacatatacacgtttgtgaccacaccaaattccacttctttctaagtgtgtgtacatagattttgttttatatatatttgaggttcacccttcattgcctccttaattctgtgcaaaagaggattcatcaccaccccatgtacccatacgatgttcctgactatgcgggctatccctatgacgtcccggactatgcaggattgtatccatatgacgttccagattacgccactagagctgcttacccatacgatgttcctgactatgcgggctatccctatgacgtcccggactatgcaggattgtatccatatgacgttccagattacgcaatgttgcaggatgttgtccacccctcaacaccggctgagcaactccccattgatgagatttcaggcccgattagtgctcgaattttcgaactttgcgaccccgatttcttcccacacacactgcaaaattctgaggttacctccagctcaaattgttgccatgaagagaagtcctcatatgccacaaccatatctccacctttagatgtagtagacaacaataagttcaatatcaatagcaatagcagcaacatagtcaccactacctcatctagcactaccacaaccagcaccacaaccaacaacaacaacaacgcaacgaacggcaataatctttctatcttctttgacactcaagatgaaattgacaatgacatctcagcctccatagacttctcatcatctccatcttttgtcgttccaccacttctcccaatctcaactcagcaggatcagtttgatttcccttcagctcagccacaggtgcaactatcaacagcagcaggttcaattttgacgggcctctctcactaccctacagatcctgtgattgcaccccttattggagctccgttaccatctgtttttgatgatgattgcatatcttccatcccttcttatgtgcctctcaacccttcatcaccctcttgctcttatctcagtcctggcataggagtgtacatgccacctcctggttcccttaacactgccttatctgctgacagttctggattgtttggtgggaacattctactggggtctgaactgcaggcacatgaattggactaccagggagaaaatggtggaatgtattgtacagattcaattcaaagggtgtttaactccccagaccttcaggcacttggtaatgagagtcagaaacttgtagctggggctggaagctctgccactttggcaccagaaatctcacacttggaggactctaccttgaaggttggaaaactctctgttgagcagaggaaggaaaagattcatagatacatgaagaagagaaacgaaagaaatttcagcaagaaaatcaagtatgcttgccgcaaaactttggcggatagccggccccgggttagaggaaggtttgcaaagaatgatgactttggagagagccataaacaaggaagtagcaatcatgaagatgatgatgaagagataattgtgaaagaagatgatgatatggttgattcctcagatatctttgcacatatcagcggagtgaactctttcaaatgcaactattccatccagtccttgatttgagcttattaaattattagtttgactagtgaaagcttatttatataattagcttctgtagattaattttggtaggacacttttcccatcccggttctctaaaatccgggtttagtggtttgagtaaactgaataaatggggtcaaaataaatataccaataagttaagtgagttagaaacgtacagaaattggaaactgtatacatttttgcagatatatattatctttttcattaagttgtaccagaacatggagttgtgttaaccaagaaaatttccagttacccccatccaagactgatgtaaccaattgatgtagcttcttttatggtagtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgacgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 5 - protein, 423 aa, Glycine max
MLQDVIHPSTPAEQLPIDEISSPISARIFELCEPDFFPDTLQNSDVTSSSNCCHEEKSSYATTISPPLDLVDNKININNNSNIVTTTSSSTTTTSTTTNNNNNNTTNSNNLSILFDTQDEIDNDISASIDFSSCRSLVVPPLLSISTQQDQFDFSSAQPQVQLSAAAGSVLKGLSHYPTDHVIAPLIGSPLPSVFDEDCISSIPSYVPLNPSSPSCSYLSPGIGVYMPPPGSLNTALSADSSGLFGGNILLGSELQAHELDYQGENGGIFCTDSIQRVFNPPDLQALGTETQKLVAGAGSSATLTPEISHLEDSNLKVGKLSVEQRKEKIHRYMKKRNERNFSKKIKYACRKTLADSRPRVRGRFAKNDEFGESHRQGSSNHEEDDEEIIVKEDDDMVDSSDIFAHISGVNSFKCNYSIQSLI

SEQ ID NO: 6 - DNA, 1272 bp, Glycine max
  source 1..1272: mol_type = genomic DNA
atgttgcaggatgttatccacccctcaacaccggctgagcaactccccattgatgagatttcaagcccgattagtgctcgaattttcgaactttgcgagcctgatttcttcccagacacactgcaaaattcagatgttacttccagctcaaattgttgccatgaagagaagtcctcatatgccacaaccatatctccacctttagatttagtagacaacaagatcaatatcaataacaatagcaacatagtcactactacctcatctagcactaccacaaccagcaccacaaccaacaacaacaacaacaacacaacgaacagcaataacctgtccatcctctttgacactcaagatgaaattgacaatgacatctcagcctccatagacttctcatcatgtcgatctttagttgttccaccacttctctcaatctcaactcagcaggatcagtttgatttctcttcagctcagccacaggtgcaactatcagcagcagcaggttcagttttgaagggcctctctcactaccctacagatcatgtgattgcaccccttattggatctccgttaccatctgtttttgatgaagattgcatatcttccatcccttcttatgtgcctctcaacccatcatcaccctcttgctcttatctcagtcctggcataggagtgtacatgcctcctcctggttcccttaacactgccttatctgctgacagttctggattgtttggtgggaacattctactggggtctgaactgcaggcacatgaattggactatcagggagaaaatggtggaatattttgcacagattcaattcagagggtgtttaaccccccagatcttcaggcacttggtactgagactcagaaacttgtagctggggctggaagttctgccactttgacaccagaaatctcacacttggaggactctaacttgaaagttggaaaactctctgttgagcagaggaaggaaaagattcatagatacatgaagaagagaaatgaaagaaatttcagcaagaaaatcaagtatgcttgccgcaaaactttagcagatagccggccccgggttagaggaaggtttgcaaagaatgatgagtttggagagagccatagacaaggaagtagcaatcatgaagaagatgatgaagaaataattgtgaaagaagatgatgatatggttgattcctcagatatctttgcacatatcagtggagtgaactctttcaaatgcaactattccatccagtccttgatttga

SEQ ID NO: 7 - DNA, 19178 bp, synthetic construct
  source 1..19178: mol_type = other DNA
gtgccgaattcggatccagcgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagagaagctcactttccaacaaccttagtaatttctcccttaacataatgaacaaattcttcaagttgatcagctctagaagcaagtctatcttcttcttgtccaagataagcttgtctagcttcaagaataactggttgatattgagctggaagtctttccatagcccaatcagcagcaacatcctttggagcaatctttccagtaacagcagaataccaaattctagaaagagtaagaacaacatttctttcatctccagcccaatctggtggagaattccaaagagtaagagtttcattaagagcttcaaaaagatcttgttctggaactggatcaaaaagttcttcagcagctggtccaacaagagcaacagaatgttctctagccttagtaagaagaatagcaagatcaatatcaatagtagctggttcaaaaattccagcaagaatatcatttctttgccattctccaaattgaagttctctcttagctggatatctccatggaataatatcatcatgaacaacaatagtaacttcaacagctctaagaatttcagattctcctggagaagcagaagtttcaagaagatcattaataagagctcttctagtagtttcatcaagtctaacagtaacagtaacaagaagatcaatatcagaatgtggcttaagtcctccatcaacagcagatccataaagatgaacagcaagaagagtaggttcaagatgtctttcaataactccaacaacttcagaaagttgagtagaaacttcagcaataacagcttctctcatgcaagcagtagcaacagaagcagaaattctgaaagaacagaacttttgcatgaaaatagaatccttcttaagaacaagcatagaattagcagagttcttaagcttcttagatccgaaaacaagaaaagaagatgacttaggaacttgtggcttatggaaattagagttaggattaagagtttgaattccttgagccatattgttaatttgagccattgtatcgataattgtaaatgtaattgtaatgttgtttgttgtttgttgttgttggtaattgttgtaaaaatgagctcttatactcgagcgtgtcctctccaaatgaaatgaacttccttatatagaggaagggtcttgcgaaggatagtgggattgtgcgtcatcccttacgtcagtggagatgtcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttatcacatcaatccacttgctttgtagacgtggttggaacctcttctttttccacgatgctcctcgtgggtgggggtccatctttgggaccactgtcggcagagagatcttgaatgatagcctttcctttatcgcaatgatggcatttgtaggagccaccttccttttctactgtcctttcgatgaagtgacagatagctgggcaatggaatccgaggaggtttcccgaaattatcctttgttgaaaagtctcaatagccctttgatcttctgagactgtatctttgacatttttggagtagaccagagtgtcgtgctccaccatgttgacctccgcaagaattcaagcttagcgcctgtgtggatcatctaaatgtgcattccgcaagccaattaatgttccaactgaagcagattgcttacataatttatatacgataccaaatatgcttcacgagcataatttttaagttgctacctcttggtaaggggcagctaattgaaactagggatcatgatctccgtttgttcaaaaatcctttggtgtgttttcaaaatatcactttagaatctaatccaagagttctccctacctaataaatatctttaacctataataccaaaatctaataattcgtgggatgatcaccatccttcacacaactcatccctgcttttcctccgtgataagcatgaagaataaaaaacccactaatatacgccaattcactggattgagaacaaaattcaaacacaatatgccagcatacagttgataattatatatcagctccacaaattgataacaaacagtagtaacattaatgggacaatagagttctacacaaaccaaacttattttcaaaggtcattacagacccattacacatcacatttcttcttaaaatgtttgggaaaatcctacatcgaacatcacacagaaatcagctccagcaagctcatcctcccacctttctcttcttctttggttgcagatctcctcccagttgggacagatcgattctagtctcgtacagtccagtgatggattggtggatcagagtagcatccagcacctccttagtggaagtgtatctctttctatcgatagtagtatcgaagtacttgaaagcagctggagctcccaggttagtcagagtgaacaggtggatgatgttctcagcttgctctctgattggcttatctctgtgcttgttgtaagcggacagcaccttatccaggttagcatcagccaggatcactctcttggagaactcggagatttgctcgatgatctcatccaggtagtgcttgtgttgctccacgaacagttgcttttgctcgttatcctctggggatcccttcagcttctcgtagtgggaagccaggtacaggaagttcacgtacttggatggcagagccagctcgtttcccttttgcagctctccagcggaagccagcattctctttcttccgttctccagctcgaacagggagtactttggcagcttgatgatcagatccttcttcacctccttgtatcccttagcctccaggaaatcgattgggttcttctcgaaggaggatctctccatgatagtgattcccagcagctccttcacggacttcagcttcttggactttcccttctccaccttagccaccaccagcacggagtaagccacagttggggaatcgaatcctccgtacttctttggatcccaatccttctttctagcgatcagcttatcggagtttctctttggcaggatggactccttggagaatcctccagtttgcacctcagtctttttcacgatgttcacttgtggcatggacagcacctttctcacagtagcgaaatctcttcccttatcccacacgatctctccagtctctccgttagtctcgatcagtggtctttttctgatctctccgttagccagagtgatctcagtcttgaagaagttcatgatgttggagtagaagaagtacttagcagtagcctttccgatctcttgctcggacttagcgatcatctttctcacatcgtacaccttgtaatctccgtacacgaactcggactccagctttgggtacttcttgatcagagcagttcccaccacagcgttcaggtaagcatcgtgagcgtggtggtagttgttgatctctctcaccttgtagaattggaaatcctttctgaaatcggacaccagcttggacttcagagtgatcaccttcacctctctgatcagcttatcgttctcatcgtacttagtgttcattctggaatccaggatttgagccacgtgcttagtgatttgtctagtctccaccagttgtctcttgatgaatccagccttatccagctcggacagtcctcctctctcagccttagtcaggttatcgaactttctttgagtgatcagcttagcgttcagcagttgtctccagtagttcttcatcttcttcaccacctcctcggatggcacgttatcggactttcctctgttcttatcggatctagtcagcaccttgttatcgatggaatcatccttcaggaaggattgtggcacgatgtgatccacatcgtaatcggacagtctgttgatatccagctcttgatccacgtacatatctcttccgttttgcaggtagtacaggtacagcttctcgttttgcagttgagtgttctccactgggtgctccttcaggatttgggatcccagctccttgattccctcctcgattctcttcattctctctctggagttcttttgtcccttttgagtagtttggttctctctagccatctcgatcacgatgttctctggcttgtgtctacccatcaccttcaccagctcatccaccaccttcacagtttgcaggattcccttcttgatagctggggatccagccaggttagcgatgtgctcgtgcagggaatctccttgtccggacacttgagccttttggatatcctccttgaaagtcagggaatcatcgtggatcagttgcatgaagtttctgttagcgaatccatcggacttcaggaaatccaggatagtctttccggattgcttatctctgattccgttgatcagctttctggacagtctaccccatccagtgtatcttcttctcttcagttgcttcatcaccttatcatcgaacaggtgagcgtaagtcttaagtctctcctcgatcatctctctatcctcgaacagagtcagagtcagcacgatatcctccaggatatcctcgttctcctcgttatccaggaaatccttatccttgatgatcttcagcagatcgtggtaagttcccagggaagcgttgaatctatcctccactccggagatctccacggaatcgaagcactcgatcttcttgaagtaatcctccttcagttgcttcacagtcacctttctgttagtcttgaacagcagatccacgatagccttcttttgctctccggacaggaaagctggctttctcattccctcagtcacgtacttcaccttagtcagctcgttgtacacagtgaagtactcgtacagcagggagtgctttggcagcaccttctcgtttggcaggttcttatcgaagttagtcattctctcgatgaaggattgagcggaagctcccttatccaccacctgcacatcaacaaattttggtcatatattagaaaagttataaattaaaatatacacacttataaactacagaaaagcaattgctatatactacattcttttattttgaaaaaaatatttgaaatattatattactactaattaatgataattattatatatatatcaaaggtagaagcagaaacttacctcctcgaagttccatggagtgatagtctcctcggactttctagtcatccaagcgaatctggagtttcctctagccagtggtcccacgtagtatgggattctgaaagtcaggatcttctcgatcttctctctgttatccttcaggaatgggtagaaatcctcttgtctcctcaggatagcgtgcagctctcccaggtggatttggtgtgggatggatccgttatcgaaagttctttgctttctcagcagatcctctctgttcagcttcaccagcagctcctcagttccatccatcttctccaggattggcttgatgaacttgtagaactcctcttgggaagctcctccatcgatgtatccagcgtatccgttcttggattgatcgaagaagatctccttgtacttctctggcagttgttgtctcaccagagccttcagcagagtcagatcttggtggtgctcatcgtatctcttgatcatggaagcggacagtggagccttagtgatctcagtgttcactctcaggatatcggacagcaggatagcatcggacaggttcttagcagccaggaacagatcagcgtattgatctccgatttgagccagcaggttatccagatcatcatcgtaagtatccttggacagttgcagcttagcatcctcagccagatcgaagttggacttgaagtttggagtcagtcccagggacagagcgatcaggtttccgaacagtccgttcttcttctctcctggcagttgagcgatcaggttctccagtctcctggacttggacagtctagcggacaggatagccttagcatccactccggaagcgttgattgggttctcctcgaacagttggttgtaagtttgcaccagttggatgaacagcttatccacatcggagttatctgggttcagatctccctcgatcaggaagtgtcctctgaacttgatcatgtgagccagagccaggtagatcagtctcagatcagccttatcagtggaatccaccagcttctttctcaggtggtagatagttgggtacttctcgtggtaagccacctcatccacgatgtttccgaagattgggtgtctctcgtgcttcttatcctcctccaccaggaaggactcctccagtctgtggaagaaggaatcatccaccttagccatctcgttggagaagatctcttgcaggtagcagattctgttctttcttctagtgtatcttcttctagcagttctcttcagtctagtagcctcagcagtctctccggaatcgaacagcagagctccgatcaggttcttcttgatggagtgtctatcagtgtttcccagcaccttgaacttcttggatggcaccttgtactcatcagtgatcacagcccatcccacggagttagttccgatatccagtccgatggagtacttcttatccattctgtcgagtcaacaatcacagataaatcagaatcaaaatccattacgtaatcttaagtacggatctgaagcaatctcgtgaaaaaaaaccctaatccacggcaatagaatcaacaaacaaggcagttgctaacagatctgagcaatatctctcgaaaaacgatatagaatcaacgaataaggctttttgtcaacggatctgaacaatttcttgaaaaaccacagaagtataaaaaataaggctgttgtttaacggatctgaagtaattcatgaaaaagccctgatctatggttacgtttaacggatctgaagtaatcaatgaaaaaagccctaatctacgattaatgaatcaacaattaaggcttgttgtaaagggatctgacatgaaaaagctctaattagagcaaatctacgattaaagaatcatcaaattaaggacgttgttgtaaagataataaaagctgaatctgattatgaaaattgaagatcgatgaaaccaaacaaaacacaatcaaatcaacatcgtattgatcaaagaacatacacaattcatggaaagaacgaagaagaaaaacagaagaaatcgactaaccttgaagggagaggtagggaaatacacagagagaatggaatatattggaattgaactgcgaaggaaggaggaggggtgtggggcgttttatagcataaggtgtggaacccttctccagtcatattgtgacgcgtgttcatgtatggtgtcacttggtgcctatgacacgggtaatatttggtattggatgcattttcggagagtccttctaggaaattgagaattgcaatttgcgtggtagttggcggtttcgtaatcttgacggagtcacatgcaccttcaagcttcgctttatctgttacgacgtcgttgttatattgggcccaccccgttgcaagtggagcgtccgaagttgatacgggccccactatccattgcctgaagtcggctgcttttacattattaaaaatctatggaaaagctgtcatgctagattttttttattatggaccagaaaagaataattgaaaagttttcttattatggagcaaaaaaatctttaataattattttttaaataaaatcataattattccactaaattctttcttatttaatttcaccttgtaagatatattttcagtagatttttcaagtatttttttaacatacattacttgtatacatttagttacagtttatataaaattattcatttaaaaaatatttagatattttttaaaaaataattcaaaatattttttttaatgaactttcctttttcctaatttttctttcttatttcttattagatgtttttactaccaatcgtttagggaagaatttttttctctaatgaggagatgtgttaaatgatgcaacttaaggacagattcaccaataacataagactaaaagatagctcattgacaaaaacaataatgatgttaaagaaaacgaatccatttccattcggataagccacaccatttacgaacaagttgcctactaataatatatggtgtcagttttttttttttttgggctgaattggaggggaaattatccccaaagtttgattaaaaatcacgcgacaaagtaacaaaacttttattccgctaaaaaaattagtcaaagtcaattaaatctggtattagaattacttttccgctaatgatatatccacaataacaatgtattaatattgaaaaaaataaagtttatagagtgtgactttttctttcatttttacgtaaattgaaaaaaaaatcaagcctaatatataaactttcattgatcctcacctaaatcctaaagttcttgagcccaattaaaaaacttactggaggataaaggcccaaacttctttttttttttaggaacagagcatgtaatgggtaatgaggcagcctaagcccataaccatgttagcccgtatatatttttgttaattagatttccggtttcttatttttttttagatttcagtttttgtttctcaaaaaaataaaataacttttagtgctgtattaactttctgtaaataattttagtctcaataaagattaattctgaataataaaaataaacgaggacaatttttttcacaacttttaattctttatttattttttacacttaattttgggaaacatggacctagtgtcaaagaactcaaactcaacattacaaaaacttactacatattcaaggatcatggtctgaactaatctccacataacatataatcacatttttcaattaaaaagaagaattaataatgtattaaatgatgatcgacaagatcacatgatttgagatttttttttatgatgggaaaaatatgatttgagattaggtaacaatctttccattttagatagacttatgtctccactagaattcgagctcggaggacgtcgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttaaggaaattgtggttgtcggacgaagtccagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgtagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagatgcagatcttaatgacaaagcaccagtggtctagtggtagaatagtaccctgccacggtacagacccgggttcgattcccggctggtgcagctcagccggtgttgagggggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggtaacggagatccaataaggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaggagtgtacatgcctcctccgttttagagctagaaatagcaagttaaaataaggctagtccgttatcaacttgaaaaagtggcaccgagtcggtgccaacaaagcaccagtggtctagtggtagaatagtaccctgccacggtacagacccgggttcgattcccggctggtgcagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggtcaagactaatcttttccttttcttcatcttctcgcttctcctctcactttccagcgcggacatggtgtccaagggcgaagaggtgattaaagagttcatgaggttcaaagtgagaatggagggcagtatgaacggccatgagttcgagatcgaaggtgaaggcgaaggtagaccatacgagggcacccagacggcgaaactgaaagtgacgaagggcggcccactgcccttcgcctgggacattctgagtccacaatttatgtacggttccaaggcatacgttaagcaccccgccgacatccctgattacaaaaagctgtcgttcccggaggggtttaaatgggaaagagtgatgaattttgaggacggagggctcgtcactgtgactcaagactcctcacttcaagacggcaccctaatttacaaggtgaagatgaggggaacaaatttcccccctgatggccctgttatgcagaaaaaaacaatgggatgggaggcatccaccgagagactttacccgcgcgacggagtgctcaaaggcgaaatccatcaggcactgaaactaaaggacggcggccactacctggttgagttcaaaactatatatatggccaagaaacccgtccagttgccgggatattactatgtggacacaaaactggatataacgtcccacaacgaagattacaccatagtggaacagtatgagcgcagtgagggtcgtcaccatctattcctcggacatggcacgggttccacgggctcgggttccagcggcacggcaagctcggaggacaacaacatggcggtgatcaaagagttcatgcggtttaaagtccgtatggagggcagcatgaatgggcacgaatttgaaattgagggagaaggcgagggccgaccatatgaggggacacaaaccgcgaaactaaaagtcaccaaaggtgggccgcttccttttgcctgggacattctctctcctcagttcatgtatggctccaaggcctacgttaaacatccggctgatatccccgattataagaaattgtctttcccggaggggtttaaatgggagcgggttatgaatttcgaggatggtggactggttactgttacacaagattcgagtctgcaggatggcacattgatatacaaagtcaaaatgaggggtacgaattttcccccggacggcccggtgatgcagaaaaaaactatgggctgggaggccagcacggagaggctgtacccgagggacggggttttgaaaggggagatacaccaggcgcttaaactgaaggacggggggcactatctggtggaatttaagacgatatatatggcgaaaaagcccgtgcagctacctggctactactacgtcgataccaaactcgatattacctctcataacgaggactacactatcgtggagcaatatgaaagatcagaaggacgtcaccacctttttctatacggaatggacgagctttacaaaaaggacgagttgtaagcttgtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggaccgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggccaattcccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggcagatcctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtcttgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagcccctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 8 - DNA, 36 bp, synthetic construct
  source 1..36: mol_type = other DNA
atgttgcaggatgttatccacccacaccggctgagc

SEQ ID NO: 9 - DNA, 32 bp, synthetic construct
  source 1..32: mol_type = other DNA
atcatgtgattgcaccccttatctccgttacc

SEQ ID NO: 10 - DNA, 50 bp, synthetic construct
  source 1..50: mol_type = other DNA
ctcagtcctggcataggagtgtacatgccttcctggttcccttaacactg

SEQ ID NO: 11 - DNA, 35 bp, synthetic construct
  source 1..35: mol_type = other DNA
atgttgcaggatgttatccaccccaccggctgagc

SEQ ID NO: 12 - DNA, 32 bp, synthetic construct; identical to SEQ ID NO: 9
  source 1..32: mol_type = other DNA

SEQ ID NO: 13 - DNA, 33 bp, synthetic construct
  source 1..33: mol_type = other DNA
ctcagatgttacttcctggttcccttaacactg

SEQ ID NO: 14 - DNA, 39 bp, synthetic construct
  source 1..39: mol_type = other DNA
atgttgcaggatgttatccaccctcaacaccggctgagc

SEQ ID NO: 15 - DNA, 32 bp, synthetic construct; identical to SEQ ID NO: 9
  source 1..32: mol_type = other DNA

SEQ ID NO: 16 - DNA, 47 bp, synthetic construct
  source 1..47: mol_type = other DNA
ctcagtcctggcataggagtgtacatgtcctggttcccttaacactg

SEQ ID NO: 17 - DNA, 23 bp, synthetic construct
  source 1..23: mol_type = other DNA
ccacccctcaacaccggctgagc

SEQ ID NO: 18 - DNA, 23 bp, synthetic construct
  source 1..23: mol_type = other DNA
ccccttattggatctccgttacc

SEQ ID NO: 19 - DNA, 23 bp, synthetic construct
  source 1..23: mol_type = other DNA
ggagtgtacatgcctcctcctgg

SEQ ID NO: 20 - DNA, 27 bp, synthetic construct
  source 1..27: mol_type = other DNA
tatccatatgacgttccagattacgcc

SEQ ID NO: 21 - DNA, 25 bp, synthetic construct
  source 1..25: mol_type = other DNA
acctcagaattttgcagtgtgtgtg

SEQ ID NO: 22 - DNA, 20 bp, synthetic construct
  source 1..20: mol_type = other DNA
ctgctgttgatggaggactt

SEQ ID NO: 23 - DNA, 20 bp, synthetic construct
  source 1..20: mol_type = other DNA
ctcctggagaagcagaagtt

SEQ ID NO: 24 - DNA, 17 bp, synthetic construct
  source 1..17: mol_type = other DNA
gtatgcttgccgcaaaa

SEQ ID NO: 25 - protein, 419 aa, Glycine max
MLQDVFDQPLPPKAEPQQQHPIWLQDEISSPLSARIFELCNTDFFPETLPNSEVTSSSNCCYEENSSYATTNISVTVDVDNKLNSNSNTVTTPTSTTTTNTNNNTTNSSNLSIIFDSQEEIDNDISASIDFSLSPSFNVPPFLPATSQQEQFDFSSVQHQVQLAACSVVEGFSQYPNDSVAPPFMGAPLPSVFEEDCISSVPSYMALNPSSPCTYLSPGMPPYMPHGPLTTALSTGSSGIFGGNILLGSELQAQELEYQGENGRMYCTDSIQRVFNPPDLQALGTESQQLVPGAGSSASLPEISNLEDSSFKVGKLSVEQRKEKINRYMKKRNERNFSKKIKYACRKTLADSRPRVRGRFAKNDDFGETNRTTSSNHEEDDEEEVVVKDEDDMVDSSDIFAHISGVNSFKCNYSFQSWI

SEQ ID NO: 26 - DNA, 4401 bp, Glycine max
  source 1..4401: mol_type = genomic DNA
gtcaacccaaaatccaactcaagacaaggagagtggcattggcagatggcacccgagagccaagagggtttgcagtgaatgaatggcccaccttatgtctcgctttgacgccagcttgacgcctttagggagaacccctttgccctatatatgaaacataactaattctttgagtctttgacatcctcatactctccatcatcctagctaactactcatcatcatattcataactttcccccctctttctctctctttttccctatggagtgtccttgagcacatgttttaagtcaccaacacacgtgtgaatttcccacattttgcactcctatatagtactctctttctttctagatatagctttaattttgtgtacattttttttgtattaaagtttctggggtgccttgattgcctcgtgcaaagggaaacatacatacatacaccatgttgcaggatgtttttgatcagccactgccaccaaaggccgaaccacaacagcaacatcccattgtaatcacctcatcttgccagcagtctcaatattcatctattatgttaatgcatacatgcatgtgttcttcctaccttaatgacatagtgtaatcctaactcaatattaatttcatgaaaactttacaagtttgttaattgtttccattttggaacgaatctctttctcatatgtttgaatatattcaaatctaaggctaagttgtttgtttacagttgtctcgtgatattggcccacttgtgattctcttctttttttcatgcatgcatgcgtgaatgatatatatatatatatatatatatatatatattattggtttgtttttatgtttgttgcaagtttgatgtattatttgccagtgctaatagaattaaagccattctcaaattcttttccaggcttccctgtcaatttgcatggaagaacgggttcttttctctcaatttatatagttggggagcataatgcttattgactagcaataagaaagaacgtattagagattccgcaataactattagactttgtccttttttatatattatcctatcctaaccaagacaatggcattcaccacaatgtcattttctgagggcaacattgtaaactgcaaagtgatatcaatttctacatgatttccagtgtgatagctagccatttaaacgtagctttcgattattgtggaatatatatctttctgtgtttgtctatttgccaattttgatattcttattcatttagagaggggagaggagacttgaggaacacttcacatgtacagcattatgtgttcttttaagtgccttttaattcctattaaatcagcttccatacactgaacaaacacaacgtagcatggcatgagattccttctttgttacaaacatacaagtaacattgtcacattaaggcataaatgcccatctttttcaatttcctaaaaaagccaaccctcttgttgaatttgttatctgtacatgacaaaatataaacacaactgactagaggacacagccaaccaaatatgtatttgaagttcctcatgatggggtattctacattgatttatttcgttgatttctagacaaagggtttgtgtagtgatgaacataactgatgcttataggcgtgttacttactcactttagaatgctcactatttgatgaagacatgcaataaaccaaagtttagactgataataacaagagaatattatcttttgaaagtttagactctgatcattataggcatggtcccaatttctattttgctgatagaaacgagtctgataagatcacatggccatcccataagggaaaataaatggtgtagaatcagtcttctacctgtttataaagggtctatcaaaattcaaaatgttattatcaatttgtttagtagttatttttggtagtcatgatgcttacattgtttttatatcagtggctgcaggatgaaatttcaagccctcttagtgctcgaattttcgaactttgtaacactgattttttcccagagactttgccaaattctgaggttacttccagctcaaactgttgttatgaggagaactcctcatatgccacaacaaatatatctgtaactgtagatgtagacaataagttgaatagcaatagcaacacagtcaccaccccaactagcactaccaccaccaacaccaacaacaacacaaccaacagtagtaacctgtctattatctttgactcccaagaagaaattgacaatgacatctctgcctccatagacttctcactatctccatctttcaatgttcccccatttctcccagccacatcccagcaagaacagtttgatttctcttctgtgcagcatcaggtccaactagcagcatgttcagttgtggagggcttctctcagtatcccaatgactctgttgcacctccttttatgggggctccattaccttctgtttttgaagaggactgcatatcttctgtcccttcttatatggctcttaacccctcatctccttgcacttatctcagtcctggcatgccaccatacatgcctcatggcccccttactactgccttatcaactggtagttctggaatatttggtgggaacattcttcttggttctgaacttcaggcacaagaattggaataccaaggagaaaatggaagaatgtattgtacagattcaattcagcgggtgtttaaccctccagaccttcaggtatggatgattttcaaactattttgttaagtcatcattgccttttgtagttttgttaacaataccatcaaaatgaagtgttcagttcaaatgttcaatatgctcagtaatgagagaaaaagaagtccattgtccaaggatttttttatttctaatgatgctaagctgttgttgtaagcttgctctttcaggtttctagtctgaaaggttgcagcagtaaggttcttgacaataaagagtgactttacaagaggtgaaaagtttgcaaaagataacggcccacaaatttctaagaagtttatatgccccatagaatatatgcccatatccatcaaaccagtagctagcatatcaaccccttttataatctcattctcacctgtaaaaagtagcataaaatctgagctagctagtagcaggaaggaatcacaactttccacaaaaattgataacaatcatcatcagaaaagacccacttcatgagtggttctttttgcatatcgctttccggaacctctaactaaagctaatgacagtttgttatgaccaattttcctttgcgtagcaagaaagtaactgatattgtttgtgatgtctttcctaattcctaggctcttggcactgagagtcagcaactagtaccaggggctggaagttctgccagtctaccagaaatctcaaacttagaggactcctccttcaaggttggaaaactttctgttgagcaaaggaaagaaaagatcaatagatacatgaagaaaagaaatgaaagaaacttcagcaagaaaattaaggtacactcaagcttcttcccatcttcaaaacagccatatcatgttacatatgcatatgcttataaacaaccactttttattgcatactttcaagttcaactaatgatgaaaccaaatatgcagtatgcctgccgcaaaactctggcagatagccgacctcgagtcagaggaaggtttgcaaagaatgatgactttggggaaaccaatagaactacaagtagcaatcatgaagaagatgatgaagaagaagtaagatttccagggctcattcatttattaaacacttaacaggacattttttattcaacttgcccaagttaaaaaaaaaaatccctatcacttgtaccttgtcaattatttgctgatatagttttttaatcctgcaggtagttgtgaaagatgaagatgatatggttgattcctcagatatctttgctcatatcagtggggtgaactccttcaaatgcaactattccttccagtcctggatttgacttaatagtttgatcagtatacctacatttagcttgtgtagattttttgcatggccatagaccaccccagttttctcatatctggttttgatggatgaggaaactgaataattagggtcctaatactatacaataagtgaatgaagaacatttagaaagaaactgtatacattttttgcagacactatttcttactacttaaatgtattaagaatcacatgttgtattaactttgtctccatgtactagtggagctcattcatgtactctcttttataatatagtgatgaggaactagttttggttacattcaagtgcttaaagcttaaagcccaaggtttaaaatatttttactggctttgaaacttttttttt

SEQ ID NO: 27 - DNA, 1260 bp, Glycine max
  source 1..1260: mol_type = genomic DNA
atgttgcaggatgtttttgatcagccactgccaccaaaggccgaaccacaacagcaacatcccatttggctgcaggatgaaatttcaagccctcttagtgctcgaattttcgaactttgtaacactgattttttcccagagactttgccaaattctgaggttacttccagctcaaactgttgttatgaggagaactcctcatatgccacaacaaatatatctgtaactgtagatgtagacaataagttgaatagcaatagcaacacagtcaccaccccaactagcactaccaccaccaacaccaacaacaacacaaccaacagtagtaacctgtctattatctttgactcccaagaagaaattgacaatgacatctctgcctccatagacttctcactatctccatctttcaatgttcccccatttctcccagccacatcccagcaagaacagtttgatttctcttctgtgcagcatcaggtccaactagcagcatgttcagttgtggagggcttctctcagtatcccaatgactctgttgcacctccttttatgggggctccattaccttctgtttttgaagaggactgcatatcttctgtcccttcttatatggctcttaacccctcatctccttgcacttatctcagtcctggcatgccaccatacatgcctcatggcccccttactactgccttatcaactggtagttctggaatatttggtgggaacattcttcttggttctgaacttcaggcacaagaattggaataccaaggagaaaatggaagaatgtattgtacagattcaattcagcgggtgtttaaccctccagaccttcaggctcttggcactgagagtcagcaactagtaccaggggctggaagttctgccagtctaccagaaatctcaaacttagaggactcctccttcaaggttggaaaactttctgttgagcaaaggaaagaaaagatcaatagatacatgaagaaaagaaatgaaagaaacttcagcaagaaaattaagtatgcctgccgcaaaactctggcagatagccgacctcgagtcagaggaaggtttgcaaagaatgatgactttggggaaaccaatagaactacaagtagcaatcatgaagaagatgatgaagaagaagtagttgtgaaagatgaagatgatatggttgattcctcagatatctttgctcatatcagtggggtgaactccttcaaatgcaactattccttccagtcctggatttga

SEQ ID NO: 28 - protein, 421 aa, Glycine max
MIPISLLLIETSLIRSHSHPRKGKQMDEISSPLSARIFELCNTDFFPEALQNSEVTSSSNCCYEENSSYATTNISVTVDVDNKLNSNSNTVTTPTSTTTTNTNNNTTNSSNLSIFFDSQEEIDNDISASIDFSLSPSFNVPPFLPVSSQQEQFDFSSVQHQVQLAACSVVEGFTQYPTDAVAPPFMGAPLPSVFEEDCISSVPSYMPLNPSSPCTYLSPGMPPYMPHGPLTTALSTDSSGIFGGNILLGSELQTQELEYQGENGRMYCTDSIQRVFNPPDLQALGTESQQLVPGAGGSGTLTPEISNLEDSSFKVGKLSVEQRKEKINRYMKKRNERNFSKKIKYACRKTLADSRPRVRGRFAKNDDFGETNRTTSSNHEEDDEEEVVMKDEDDMVDSSDIFAHISGLNSFKCNYSIQSWI

SEQ ID NO: 29 - DNA, 4011 bp, Glycine max
  source 1..4011: mol_type = genomic DNA
acaccctcatactctccatcatcctagctaactcatcatcatattcataactcccccccctctctctctctctccctccctccttccctatggagtgcccttgagcacatgtttttagtcaccaacacacgtgtgaattttcccacattttgcacttccatatagctcttttctggggtgccttgattgcctcgtgcaaggggatacatacacacacacacacaccatgttgcaggatgtttttgatcagccactgccaccaaaggccgaaccacaacagcaacttccccttgtaataatctccagccagcggtctcattcatatcttccatgttaatgcatgcatgtgttctgcctaccttaattaattagttaatttgtgtcatgactcatgacatggtgtgatcttaacccaaaattcatgaaactttacaagtttgtttgtttccattttggaatctctttctcaggaatatattcaagtctaaggctaagttgtttacaattgtctcgtgatattggaccacttgtgattccattttttcatgcatgcatgaatgatatatatggttgttttatttttatgtttttttgcaagtttgatatattatttcccagtgctaatagaattaaagccattctcaaattcttttccaggcttctctggcaatttgcagggaggaacgggttattttctctcaatttatagagttgggggggggagggggggggggcataatgcttattgactagcaataagaaagaacgtattagagattccgcaataattattagtctttgtccttttttttcttctatattatcctatccaatccaagacaatgccatttaccaaagatgtcattttctgagggcaacattgtaaactgcaagtgatatcaatttctacatgattttcagtgtgatagctagccatttaaacatagctttcgattgttgtggaatatatatctttctgtggttgcctatttgccaattttgatattcttattcatttagagaggggagaggagacttgaggaacacttcacatatatagcattatgtgttcttttaagtgccttttaatttaattcctattaaatcagtttccatacactaaacaaacacaacgtagcatggcatgaggttccttctttgttacaaacatacaagtaacattgtcacgctaaggcataaatgcccatctttttcaatttcctaaaaaaaccaaccctcttgtagaatttatctgtacatgaccaaatataagcacaactgatagaggacacaaccaacctaatctgtatttggagttcctcatgatggggtattgtacattgatttatttcgttgatttctagacaaagggcttgtagtgatgaacataactgatgcttataggcgtgttactcactatttgatgaagacacgcaataaaccaaagtttagactgataataacaagagaatattattatcttttgaaagtttagactctgatcattataggcatgatcccaatttctcttttgctgatagaaacgagtctgataagatcacacagccatccccgtaagggaaaacaaatggtgtagaatcagtcttctacctgtttataaagggtctatcaaaatgttatcaatatgtgtctaatagtttttttggtagtcatgatgcttacattgtttttcatatcactggctgcaggatgaaatttcaagccctcttagtgctcgaatcttcgaactttgtaacactgatttttttccagaggctttgcagaattctgaggttacttccagctcaaactgttgttacgaggagaactcctcatatgccacaacaaatatatctgtaactgtagatgtagacaataagttgaatagcaatagcaacacagtcaccaccccaactagcactaccaccaccaacaccaacaacaacacaaccaacagtagtaacctgtctattttctttgactcccaagaagagattgacaatgacatttctgcctccatagacttctcactctctccatctttcaatgttcccccatttctcccagtctcatcccagcaagaacagtttgatttctcttctgtgcagcatcaggtccaactagcagcatgttcggttgtggagggcttcactcagtatcccactgacgctgttgcacctccttttatgggggctccattaccttctgtttttgaagaggactgcatatcttcagtcccttcttatatgcctcttaacccctcatctccttgcacttatctcagtcctggcatgccaccatacatgcctcatggcccccttactactgccttatcaactgatagttctggaatatttggtgggaacattcttcttggttctgaacttcagacacaagaattggaataccaaggagaaaatggaagaatgtattgtacagattcaattcagcgggtgtttaaccctccagaccttcaggtatgaatgattttcaaactatttagttaagttctccatcattgcctttgttaacaataccatcctaatgaaaagtgttcggttcgttcaatatgctcagtaacgagagaaaaagaagtccttgtccaaggattttttttttttatttcttatgatgctaagctgttgttgttagcttgctctttcaggtttctagtctgaaaggttgcagcagtaaggttcttgactataaagagtggctttacaagaggtgaaaaatctgcaaaagataacggtccacaaatttctaagaagctgatatgccccatagaatatatgcccatatccatccatacagtagctagcatatcaaccccttttataatctcattctcacctgtaaaaagtagcataaaatctgagctagctagtagcaggaaggaatcacaacattccacaaaaattgataacgaacaacatcatcatcagaaaagacccacttcatgacagtttgtgatgatcaattttcctttgcgtagcaagaaagtaactgattttgtttgcaatttctttcctaggcacttggtactgagagccagcaactagtacccggggctggtggttctggcactctaacaccagaaatctcaaacttagaggactcctccttcaaggttggaaaactttctgttgagcaaaggaaagaaaagatcaatagatacatgaagaaaagaaatgaaagaaacttcagcaagaaaattaaggtacagtcaagctccttcccatcttcaaaacagccacatcatcaaacaaaattgaaatcttatcatgttacatatgcatatgcaaggtattccttgcttataaacaaccacttgttattgcatactttcaagttcaactaatgatgcaaccaaatatgcagtatgcctgccgcaaaactctggcagatagccgacctcgagttagaggaaggtttgcaaagaatgatgactttggagaaaccaatagaactactagtagcaatcatgaagaagatgatgaagaagaagtaagatttccagggttcattcatttattaaacacttaacaggacgctttttattcaactatttgttgatatagttttttaaccctgcaggtagttatgaaagatgaagatgatatggttgattcctcagatatctttgctcatatcagtgggctgaactccttcaaatgcaactattccatccagtcctggatttgacttaatagtttgatcagtatacctacatttagcctctgtagattttttgcatggccatataccaccccagatttctcctatctggttttgatggatgaggaaactgaataattggggtcctaatactatacaataagtgaatgaaaaatatatagaaaaaaaactgtatacattttttgcagacactacctttcttactacttgtatgtactaagaatcacatgttgtattaactcaaacgatttcatttgtctccatgtactagtggagctcattcatgtactctcttttataatatagtgataa

SEQ ID NO: 30 - DNA, 1266 bp, Glycine max
  source 1..1266: mol_type = genomic DNA
atgatcccaatttctcttttgctgatagaaacgagtctgataagatcacacagccatccccgtaagggaaaacaaatggatgaaatttcaagccctcttagtgctcgaatcttcgaactttgtaacactgatttttttccagaggctttgcagaattctgaggttacttccagctcaaactgttgttacgaggagaactcctcatatgccacaacaaatatatctgtaactgtagatgtagacaataagttgaatagcaatagcaacacagtcaccaccccaactagcactaccaccaccaacaccaacaacaacacaaccaacagtagtaacctgtctattttctttgactcccaagaagagattgacaatgacatttctgcctccatagacttctcactctctccatctttcaatgttcccccatttctcccagtctcatcccagcaagaacagtttgatttctcttctgtgcagcatcaggtccaactagcagcatgttcggttgtggagggcttcactcagtatcccactgacgctgttgcacctccttttatgggggctccattaccttctgtttttgaagaggactgcatatcttcagtcccttcttatatgcctcttaacccctcatctccttgcacttatctcagtcctggcatgccaccatacatgcctcatggcccccttactactgccttatcaactgatagttctggaatatttggtgggaacattcttcttggttctgaacttcagacacaagaattggaataccaaggagaaaatggaagaatgtattgtacagattcaattcagcgggtgtttaaccctccagaccttcaggcacttggtactgagagccagcaactagtacccggggctggtggttctggcactctaacaccagaaatctcaaacttagaggactcctccttcaaggttggaaaactttctgttgagcaaaggaaagaaaagatcaatagatacatgaagaaaagaaatgaaagaaacttcagcaagaaaattaagtatgcctgccgcaaaactctggcagatagccgacctcgagttagaggaaggtttgcaaagaatgatgactttggagaaaccaatagaactactagtagcaatcatgaagaagatgatgaagaagaagtagttatgaaagatgaagatgatatggttgattcctcagatatctttgctcatatcagtgggctgaactccttcaaatgcaactattccatccagtcctggatttga

SEQ ID NO: 31 - DNA, 3129 bp, Arabidopsis thaliana
  source 1..3129: mol_type = genomic DNA
tataaataaattggttacgataacttccataaccttcattagtttctccagaagaaacctgtaagaaatttctttctcttctctgtctttaaacagatttttattctgttctgtttgattaattattatctcttttgttccagtattttaattctttagattttcttgttcagacatcactagctccactaccacattcaagaagcctccttcaataatctttcccttttcctggggaattttctctctggattcattcccggaaaatcattcacaatttggagaccataaagaacacataaacatgtcgcaggacgtaatttcgtctcatgagcagctctccatggtacgtaataatccgtgcaatgtttattgtttgtcctcgaatctgtttttatgcccttgaatggtttctcttctttgggtttcctcttttctatcatacattgcttaaatgattaaaagcatgaacctttttttcctgaaaattttgcatgtatatatctatgatttattcgcaaggaataaaattattttctgaaatctttgtattttgtaggtaacatcgttttcttaattttcttagttattcgattttatcttttgtcaaattgcttcattccttttactttcttttctgtttctggttattctagatacaattcagttcatattccaaaagtctgaaacgataattagtaaagcaaactttttagtgctaactcttttgtacggtgttcatacgtagttacgtaccttctcgctagccaaccacaaatcgtcacaatacttaatcagatagaatagaacagtttatcagaattcataagatgctacatctcttcttttagtaattccagtaattaaaatttgaacttttatttatactttcgaatagaatcacacgaatttttattagaaatatataatgagtgtatcaaaagcaatgattcttgctcttacctcggcctttatttgttgttaaaaaatcagatgttagtttaaattagggaacgttttggttctaaagaaacgcaagaatccaaaaactttcctgttatggaaactctcccgccttatcgttaatttcttctctagattccctcttgatgaaaaccacatagaaggaacaagatctaatcgtataaatacatagttctctctaaacgactgtgttaagttaaaactaaccttaggtttattcaaagttattgtttcttgtttcacaagaaaatcccaccaattagctttttatgcaagccttcaacgtatgtcaataaacaaacaattagatgcatatatttacttgttctattttctgctaatttgatctttctcctgctgttgttattttgtaggatgagatcacaagtcctctcaccgcacagatcttcgatttctgcgattcacagttatttcaagaaaccttcaatcaaacttctgaagtaacctctgcttcaaacggttgtggctatgtcgaaaacaacaacaccaacaactttccagacaaatccaacagtggttcgaatcaagatcatgaagacaacaacgacaacgctgatttatcaatcatattcgattcacaagatgatttcgataacgatatcacagcttctatcgatttctcttcatccatccagttcccagcctccgatcaactccaagagcagtttgatttcaccggaattcaacttcaccagcctccgaacactctctactcttcctcctctggtgaccttctgcctccgcctttatcggttttcgaagaagattgtctttcttctgtgccaagttacaatcttggttccataaacccttcttctccttcttgctcttttttgggtaataccggtttaccgacttacatgaccgtaaccgggaatatgatgaacaccggtttaggatctggtttttactccgggaatatccatctcggttctgatttcaaaccatcacatgatcaattgatggaaattcaagctgataatggtggattgttctgtccagatccgattaaacccatctttaatccaggagatcatcatctccaggtatatattccaaaccttatatataaataatgaaaggtattgctggcaaacaaattgtgtaatgaaaccagattagtaaaccggtctgatactaaccggttatggggtttgttttcttaatagggacttgacggtgtcgagaaccagaaccacatggtggcacagccggttctcccgcagttagggacggagataaccggtttagacgacccgagtttcaacaaagtcggaaaactctcggcagagcagagaaaagagaagattcataggtatatgaagaagaggaacgagaggaatttcagcaagaaaatcaaggtctttgtgcttaaccatcataaccggattaaaccagtggttatccatataccgatttgttcacatgttttttttgttatattttgttgtagtatgcatgtaggaagacgttggcagatagtcgtccaagagttagaggaagatttgcaaagaatgatgaatttggtgaaccaaatagacaagcctgttcaagccatcatgaagatgatgatgatgatgtaagattaatcttgtttgattacaattagaaaacagattagtttttgtttgatataataaagcgtttctaaacgggggtgtggtttgcaggtgggggtaaaggaagaagaacagttagttgattcttcggacatattttcacacataagtggtgtgaactctttcaaatgcaattatccaatccagtcttggatttgagttggattacttacttggtcttatgatttacaaaaataaaacaaaaatagtattgttatagaggtctgtaatttgtatatttattagtacattttttggccatgactcatagtttgtcgaataagtagaacataagaaaagagagagatgaataatgggtgtaggcaggtaatcatgaataaaattgtagtgagtgagaggggttgtttatgttacatagaacattacaaaattttgtagatatatattatatggtatgataattatgatgtctattaatgtatttgatgggtgttgtataaataataatcatttgaacattataaataagcagttgtgttcaagt

SEQ ID NO: 32 - DNA, 1161 bp, Arabidopsis thaliana
  source 1..1161: mol_type = genomic DNA
atgtcgcaggacgtaatttcgtctcatgagcagctctccatggatgagatcacaagtcctctcaccgcacagatcttcgatttctgcgattcacagttatttcaagaaaccttcaatcaaacttctgaagtaacctctgcttcaaacggttgtggctatgtcgaaaacaacaacaccaacaactttccagacaaatccaacagtggttcgaatcaagatcatgaagacaacaacgacaacgctgatttatcaatcatattcgattcacaagatgatttcgataacgatatcacagcttctatcgatttctcttcatccatccagttcccagcctccgatcaactccaagagcagtttgatttcaccggaattcaacttcaccagcctccgaacactctctactcttcctcctctggtgaccttctgcctccgcctttatcggttttcgaagaagattgtctttcttctgtgccaagttacaatcttggttccataaacccttcttctccttcttgctcttttttgggtaataccggtttaccgacttacatgaccgtaaccgggaatatgatgaacaccggtttaggatctggtttttactccgggaatatccatctcggttctgatttcaaaccatcacatgatcaattgatggaaattcaagctgataatggtggattgttctgtccagatccgattaaacccatctttaatccaggagatcatcatctccagggacttgacggtgtcgagaaccagaaccacatggtggcacagccggttctcccgcagttagggacggagataaccggtttagacgacccgagtttcaacaaagtcggaaaactctcggcagagcagagaaaagagaagattcataggtatatgaagaagaggaacgagaggaatttcagcaagaaaatcaagtatgcatgtaggaagacgttggcagatagtcgtccaagagttagaggaagatttgcaaagaatgatgaatttggtgaaccaaatagacaagcctgttcaagccatcatgaagatgatgatgatgatgtgggggtaaaggaagaagaacagttagttgattcttcggacatattttcacacataagtggtgtgaactctttcaaatgcaattatccaatccagtcttggatttga

SEQ ID NO: 33 - protein, 386 aa, Arabidopsis thaliana
MSQDVISSHEQLSMDEITSPLTAQIFDFCDSQLFQETFNQTSEVTSASNGCGYVENNNTNNFPDKSNSGSNQDHEDNNDNADLSIIFDSQDDFDNDITASIDFSSSIQFPASDQLQEQFDFTGIQLHQPPNTLYSSSSGDLLPPPLSVFEEDCLSSVPSYNLGSINPSSPSCSFLGNTGLPTYMTVTGNMMNTGLGSGFYSGNIHLGSDFKPSHDQLMEIQADNGGLFCPDPIKPIFNPGDHHLQGLDGVENQNHMVAQPVLPQLGTEITGLDDPSFNKVGKLSAEQRKEKIHRYMKKRNERNFSKKIKYACRKTLADSRPRVRGRFAKNDEFGEPNRQACSSHHEDDDDDVGVKEEEQLVDSSDIFSHISGVNSFKCNYPIQSWI

SEQ ID NO: 34 - DNA, 20 bp, synthetic construct
  source 1..20: mol_type = other DNA
ctggcagaacttccagcccc

SEQ ID NO: 35 - DNA, 20 bp, synthetic construct
  source 1..20: mol_type = other DNA
ccaggactgagataagtgca

SEQ ID NO: 36 - DNA, 20 bp, synthetic construct
  source 1..20: mol_type = other DNA
ccaggactgagataagtgca

SEQ ID NO: 37 - DNA, 21 bp, synthetic construct
  source 1..21: mol_type = other DNA
atttatcccgaatggtaacgc

SEQ ID NO: 38 - DNA, 21 bp, synthetic construct
  source 1..21: mol_type = other DNA
taccggagatgtgtttgaagg

SEQ ID NO: 39 - DNA, 21 bp, synthetic construct
  source 1..21: mol_type = other DNA
aacgtgaaacggatttgtcag

SEQ ID NO: 40 - DNA, 21 bp, synthetic construct
  source 1..21: mol_type = other DNA
aaatttggtgaaaccaagacc

SEQ ID NO: 41 - DNA, 19 bp, synthetic construct
  source 1..19: mol_type = other DNA
attttgccgatttcggaac

SEQ ID NO: 42 - DNA, 21 bp, synthetic construct
  source 1..21: mol_type = other DNA
atttatcccgaatggtaacgc

SEQ ID NO: 43 - DNA, 21 bp, synthetic construct
  source 1..21: mol_type = other DNA
taccggagatgtgtttgaagg

SEQ ID NO: 44 - DNA, 21 bp, synthetic construct
  source 1..21: mol_type = other DNA
aacgtgaaacggatttgtcag

SEQ ID NO: 45 - DNA, 21 bp, synthetic construct
  source 1..21: mol_type = other DNA
aaatttggtgaaaccaagacc

SEQ ID NO: 46 - DNA, 19 bp, synthetic construct
  source 1..19: mol_type = other DNA
attttgccgatttcggaac

(19) United States
(12) Patent Application Publication
(10) Pub. No.: US 2025/0283105 A1
(43) Pub. Date: Sep. 11, 2025

(54) USE OF CCT-DOMAIN PROTEINS TO IMPROVE AGRONOMIC TRAITS OF PLANTS

(71) Applicants: Donald Danforth Plant Science Center, St. Louis, MO (US); The United States of America as Represented by the Secretary of Agriculture, Washington, DC (US)

(72) Inventors: Yong-Qiang An, Frontenac, MO (US); Wolfgang Goettel, St Louis, MO (US); Hengyou Zhang, Harbin (CH)

(21) Appl. No.: 18/849,813

(22) PCT Filed: Mar. 23, 2023
(86) PCT No.: PCT/US2023/064890

§ 371 Date: Sep. 23, 2024

Related U.S. Application Data
(60) Provisional application No. 63/323,026, filed on Mar. 23, 2022.

(51) Int. Cl. C12N 15/82; C07K 14/415

(52) U.S. Cl. CPC C12N 15/8261; C07K 14/415

(57) ABSTRACT

Genetically modified plants having improved agronomic traits are disclosed. The plants comprise nucleic acid modifications that modify CCT proteins thereby improving the agronomic trait of the plants, including seed quality, seed oil content and seed protein content. Nucleic acid modification systems and nucleic acid constructs encoding the engineered nucleic acid modification system are also disclosed. Further, methods of improving agronomic traits of plants using the nucleic acid modification systems and nucleic acid constructs.

### CROSS REFERENCE TO RELATED APPLICATIONS

This application claims priority from Provisional Application No. 63/323,026, filed Mar. 23, 2022, the entire contents of which are hereby incorporated by reference.

### GOVERNMENTAL RIGHTS

This invention was made with government support under USDA-ARS 5070-21000-043-000-D and 5070-21000-043-021-A awarded by the United States Department of Agriculture. The government has certain rights in the invention.

### SEQUENCE LISTING

The present application contains a Sequence Listing which has been submitted in .XML format via Patent Center and is hereby incorporated herein by reference in its entirety. Said WIPO Sequence Listing was created on Mar. 23, 2023 is named 077875-751278 Seq. List and is 95.4 kilobytes in size.

### FIELD OF THE INVENTION

The present disclosure provides genetically modified plants having improved agronomic traits.

### BACKGROUND OF THE INVENTION

According the United Nations Food and Agricultural Organization (UN FAO), the world's population will exceed 9.6 billion people by the year 2050, which will require significant improvements in agricultural production to meet growing food demands. At the same time, conservation of resources (such as water, land), reduction of inputs (such as fertilizer, pesticides, herbicides), environmental sustainability, and climate change are increasingly important factors in how food is grown. Improvement of agronomic traits of cultivated plants such as seed quality and yield has proven challenging for conventional paradigms for crop improvement. This challenge is in part due to the complex genetic and environmental factors that can affect agronomic traits in plants. The complex correlation of important agronomic traits poses a great challenge in simultaneously improving more than one desirable agronomic trait to increase the overall economic value of a cultivated plant. For instance, in soybean, one of the most important seed crops grown worldwide, seed protein content and oil content appear to be negatively correlated, posing a great challenge in simultaneously improving both seed quality traits and yield to increase the overall economic value of soybean. Thus, there is a need for cultivated plants with improved agronomic traits.

### SUMMARY OF THE INVENTION

One aspect of the instant disclosure encompasses a genetically modified plant having an improved agronomic trait. The plant comprises a nucleic acid sequence encoding a CCT motif-containing protein (CCT protein) wherein the CCT protein is a single-CCT domain polypeptide, wherein the nucleic acid sequence encoding the CCT protein comprises a nucleic acid modification and wherein the nucleic acid modification modifies the expression of the CCT protein in the plant thereby improving the agronomic trait of the plant.

The agronomic trait can be seed quality, seed protein content, seed protein composition, seed oil content, seed oil composition, yield, seed set, response to photoperiod, abiotic stress tolerance, biotic stress tolerance, flowering time and maturity, regulation of circadian clock light response-related flowering, high latitude adaptation, or any combination thereof. In some aspects, the improved agronomic trait is an agronomic trait of Table 14. In other aspects, the improved agronomic trait is an agronomic trait associated with a QTL of Table 15. In other aspects, the agronomic trait is: (a) seed quality and the CCT protein is encoded by a nucleic acid sequence comprising a gene of Table 5; (b) yield-related traits and the CCT protein is encoded by a nucleic acid sequence comprising a gene of Table 6; (c) response to abiotic/biotic stress tolerance and the CCT protein is encoded by a nucleic acid sequence comprising a gene of Table 7; (d) flowering time and maturity and the CCT protein is encoded by a nucleic acid sequence comprising a gene of Table 8; and (e) development-related traits and the CCT protein is encoded by a nucleic acid sequence comprising a gene of Table 9.

In some aspects, the plant is a legume (Fabaceae). The legume can be common bean, cowpea, soybean, chickpea, pea, or Medicago. In some aspects, the legume is a soybean species (Glycine max, hispida). When the legume is soybean, the agronomic trait can be seed protein, oil content, 100-seed weight, or any combination thereof, and the CCT protein is GmCCT34 (POWR2), GmCCT67 (POWR1), GmCCT35 (POWR3), GmCCT69 (POWR4), any variant thereof, or any combination thereof.

In some aspects, the CCT protein is GmCCT67 (POWR1). In one aspect, the nucleic acid modification reduces the expression of the GmCCT67 protein in the plant. In some aspects, oil content of seeds is increased by about 0.5% to about 5% wt/wt and wherein protein content of seeds is reduced by about 1% wt/wt to about 20% wt/wt. When the CCT protein is POWR1, the nucleic acid modification can increase the expression of the GmCCT67 protein in the plant. In some aspects, oil content of seeds is decreased by about 0.5% to about 5% wt/wt and wherein protein content of seeds is increased by about 1% wt/wt to about 20% wt/wt. In some aspects, the GmCCT67 protein comprises an amino acid sequence comprising at least about 75% or more, at least about 85% or more, at least about 95% or more, or 100% sequence identity with the amino acid sequence of SEQ ID NO: 1.

In some aspects, the GmCCT67 protein is encoded by a nucleic acid sequence comprising at least about 75% or more, at least about 85% or more, at least about 95% or more, or 100% sequence identity with the nucleic acid sequence of SEQ ID NO: 2. In some aspects, the nucleic acid modification in the nucleic acid sequence encoding the GmCCT67 protein comprises a transposable element (TE) insertion. In some aspects, the nucleic acid sequence comprising the TE insertion comprises at least about 75% or more, at least about 85% or more, at least about 95% or more, or 100% sequence identity with the nucleic acid sequence of SEQ ID NO: 3. In some aspects, the nucleic acid modification in the nucleic acid sequence encoding the GmCCT67 protein comprises an expression construct for expression of the GmCCT67 protein, wherein the expression construct comprises a nucleic acid sequence encoding the GmCCT67 protein operably linked to a ubiquitin promoter or a native promoter.

In some aspects, the CCT protein is GmCCT34 (POWR2). When the CCT protein is GmCCT34, the nucleic acid modification reduces the expression of GmCCT34 (POWR2) in the plant such that the oil content of seeds can be increased by about 0.5% to about 5% wt/wt and protein content of seeds can be reduced by about 1% wt/wt to about 20% wt/wt.

In some aspects, the nucleic acid modification increases the expression of GmCCT34 (POWR2) in the plant. The oil content of seeds can be decreased by about 0.5% to about 5% wt/wt and wherein protein content of seeds is increased by about 1% wt/wt to about 20% wt/wt. In some aspects, the GmCCT34 protein comprises an amino acid sequence comprising at least about 75% or more, at least about 85% or more, at least about 95% or more, or 100% sequence identity with the amino acid sequence of SEQ ID NO: 5. In some aspects, the GmCCT34 protein is encoded by a nucleic acid sequence comprising at least about 75% or more, at least about 85% or more, at least about 95% or more, or 100% sequence identity with the nucleic acid sequence of SEQ ID NO: 6.

The nucleic acid modification in the nucleic acid sequence encoding the GmCCT34 protein can also comprise an expression construct for expression of the GmCCT34 protein, wherein the expression construct can comprise a nucleic acid sequence encoding the GmCCT34 protein operably linked to a promoter.

In some aspects, the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein. In one aspect, the nucleic acid modification in the nucleic acid sequence encoding the GmCCT34 protein comprises a nucleic acid modification generated using a CRISPR/Cas programmable nucleic acid modification system targeted to a nucleic acid sequence in a nucleotide sequence encoding the GmCCT34 protein. In some aspects, the nucleic acid modification in the nucleic acid sequence encoding the GmCCT34 protein comprises a nucleic acid sequence of SEQ ID NO: 8 to 10 or any combination thereof. In other aspects, the nucleic acid modification in the nucleic acid sequence encoding the GmCCT34 protein comprises a nucleic acid sequence of SEQ ID NO: 11 to 13 or any combination thereof. In additional aspects, the nucleic acid modification in the nucleic acid sequence encoding the GmCCT34 protein comprises a nucleic acid sequence of SEQ ID NO: 14 to 16 or any combination thereof. In some aspects, the plant is a soybean species (Glycine max, hispida), the CCT protein is GmCCT34 (POWR2), the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein or a nucleic acid sequence of SEQ ID NO: 8 to 16 or any combination thereof, and wherein the nucleic acid modification reduces the expression of the GmCCT34 protein in the plant.

The CCT protein can be GmCCT35 (POWR3). In some aspects, the GmCCT35 protein comprises an amino acid sequence comprising at least about 75% or more, at least about 85% or more, at least about 95% or more, or 100% sequence identity with the amino acid sequence of SEQ ID NO: 25. In some aspects, the GmCCT35 protein is encoded by a nucleic acid sequence comprising at least about 75% or more, at least about 85% or more, at least about 95% or more, or 100% sequence identity with the nucleic acid sequence of SEQ ID NO: 26. In some aspects, the plant is a soybean species (Glycine max, hispida), the CCT protein is GmCCT35 (POWR3), the nucleic acid modification in the nucleic acid sequence encoding the GmCCT35 protein comprises a nucleic acid sequence comprising at least about 75% or more, at least about 85% or more, at least about 95% or more, or 100% sequence identity with the nucleic acid sequence of SEQ ID NO: 27.

In some aspects, the CCT protein is GmCCT69 (POWR4). The GmCCT69 protein can comprise an amino acid sequence comprising at least about 75% or more, at least about 85% or more, at least about 95% or more, or 100% sequence identity with the amino acid sequence of SEQ ID NO: 28. The GmCCT69 protein can be encoded by a nucleic acid sequence comprising at least about 75% or more, at least about 85% or more, at least about 95% or more, or 100% sequence identity with the nucleic acid sequence of SEQ ID NO: 29. In some aspects, the plant is a soybean species (Glycine max, hispida), the CCT protein is GmCCT69 (POWR4), the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 30.

In some aspects, the plant is a soybean species (Glycine max, hispida), wherein; the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; and the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof.

In some aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; and the CCT protein is GmCCT69 (POWR4) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 30.

In other aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; and the CCT protein is GmCCT35 (POWR3) and the nucleic acid modification in the nucleic acid sequence encoding the GmCCT35 protein comprises a nucleic acid sequence comprising at least about 75% or more, at least about 85% or more, at least about 95% or more, or 100% sequence identity with the nucleic acid sequence of SEQ ID NO: 27.

In yet other aspects, the plant is a soybean species (Glycine max, hispida), wherein the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; and the CCT protein is GmCCT35 (POWR3) and the nucleic acid modification in the nucleic acid sequence encoding the GmCCT35 protein comprises a nucleic acid sequence comprising at least about 75% or more, at least about 85% or more, at least about 95% or more, or 100% sequence identity with the nucleic acid sequence of SEQ ID NO: 27.

In some aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; and the CCT protein is GmCCT35 (POWR3) and the nucleic acid modification in the nucleic acid sequence encoding the GmCCT35 protein comprises a nucleic acid sequence comprising at least about 75% or more, at least about 85% or more, at least about 95% or more, or 100% sequence identity with the nucleic acid sequence of SEQ ID NO: 27.

In some aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; the CCT protein is GmCCT35 (POWR3) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 27; and the CCT protein is GmCCT69 (POWR4) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 30.

In some aspects, the plant is Arabidopsis thaliana. When the plant is Arabidopsis, the CCT protein can be AtPOWR1, any variant thereof, or any combination thereof, and a nucleic acid modification can reduce the expression of the AtPOWR1protein in the plant. In some aspects, the oil content of the seeds is increased and wherein the protein content of the seeds is reduced. In some aspects, the AtPOWR1 protein comprises an amino acid sequence comprising at least about 75% or more, at least about 85% or more, at least about 95% or more, or 100% sequence identity with the amino acid sequence of SEQ ID NO: 33. In other aspects, the AtPOWR1 protein is encoded by a nucleic acid sequence comprising at least about 75% or more, at least about 85% or more, at least about 95% or more, or 100% sequence identity with the nucleic acid sequence of SEQ ID NO: 31. In some aspects, the Arabidopsis plant comprises a first T-DNA-insertion mutant of AtPOWR1 (WiscDsLox297300_13A.1, Atcct1), a second T-DNA-insertion mutant of AtPOWR1 (SALK_036731.1; Atcct-2).

Another aspect of the instant disclosure encompasses an engineered nucleic acid modification system for modifying the expression of a CCT protein in a plant. The system comprises a nucleic acid expression construct comprising: a promoter operably linked to a nucleic acid sequence encoding a programmable nucleic acid modification system targeted to a nucleic acid sequence in a nucleotide sequence encoding the CCT protein; or a nucleotide sequence encoding the CCT protein operably linked to a promoter. Expressing the programmable nucleic acid modification system or expressing the CCT protein introduces a nucleic acid modification of the nucleic acid sequence encoding the CCT protein, thereby modifying the expression of a CCT protein in a plant.

In some aspects, the CCT protein is GmCCT34 (POWR2), GmCCT67 (POWR1), GmCCT35 (POWR3), GmCCT69 (POWR4), or any combination thereof. The GmCCT67 (POWR1) protein can be encoded by a nucleic acid sequence comprising at least about 75% or more, at least about 85% or more, at least about 95% or more, or 100% sequence identity with the nucleic acid sequence of SEQ ID NO: 2. In some aspects, a nucleic acid modification can be an expression construct comprising a nucleic acid sequence comprising at least about 75% or more, at least about 85% or more, at least about 95% or more, or 100% sequence identity with the nucleic acid sequence of SEQ ID NO: 4.

In some aspects, the CCT protein is GmCCT34 encoded by a nucleic acid sequence comprising at least about 75% or more, at least about 85% or more, at least about 95% or more, or 100% sequence identity with the nucleic acid sequence of SEQ ID NO: 6. In some aspects, the expression construct for expression of GmCCT34 POWR2 comprises a nucleic acid sequence comprising at least about 75% or more, at least about 85% or more, at least about 95% or more, or 100% sequence identity with the nucleic acid sequence of SEQ ID NO: 7. In another aspect, the nucleic acid expression construct comprises a promoter operably linked to a nucleic acid sequence encoding a programmable nucleic acid modification system targeted to a nucleic acid sequence in a nucleotide sequence encoding the GmCCT34 protein. In yet other aspects the programmable nucleic acid modification system is CRISPR/Cas system comprising a guide RNA (gRNA) having a sequence complementary to a target sequence within the nucleotide sequence encoding the GmCCT34 protein. The gRNA can comprise a nucleic acid sequence of SEQ ID NO: 17, SEQ ID NO: 18, SEQ ID NO: 19, or any combination thereof.

In some aspects, the nucleic acid modification in the nucleic acid sequence encoding the GmCCT34 protein comprises an expression construct for expression of the GmCCT34 protein, wherein the expression construct comprises a nucleic acid sequence encoding the GmCCT34 protein operably linked to a promoter. In some aspects, the nucleic acid expression construct comprises a nucleotide sequence comprising at least about 75% or more, at least about 85% or more, at least about 95% or more, or 100% sequence identity with the nucleotide sequence of SEQ ID NO: 4. The nucleic acid expression construct can comprise a nucleotide sequence encoding the GmCCT34 protein operably linked to a promoter. In some aspects, the nucleic acid expression construct comprises a nucleotide sequence comprising at least about 75% or more, at least about 85% or more, at least about 95% or more, or 100% sequence identity with the nucleotide sequence of SEQ ID NO: 7. In some aspects, the construct can further comprise a nucleic acid delivery vector comprising the nucleic acid expression construct for delivering the nucleic acid expression construct to the target cell.

Yet another aspect of the instant disclosure encompasses one or more nucleic acid constructs encoding an engineered nucleic acid modification system for modifying the expression of a CCT protein in a plant. The engineered nucleic acid modification system can be as described herein above.

An additional aspect of the instant disclosure encompasses a plant comprising one or more nucleic acid constructs encoding an engineered nucleic acid modification system for modifying the expression of a CCT protein in a plant. The nucleic acid constructs can be as described herein above.

One aspect of the instant disclosure encompasses a method of identifying a plant having an improved agronomic trait using marker-assisted selection (MAS). The method comprises identifying in a population of plants one or more plants comprising a molecular marker, wherein the molecular marker demonstrates linkage with a nucleic acid modification that modifies the expression of a CCT protein in the plant. The molecular marker can be a quantitative trait locus (QTL) selected from QTLs of Table 15. In some aspects, the population of plants comprises progeny of a cross between parent plants. In other aspects, a parent plant can be a plant described herein above.

Another aspect of the instant disclosure encompasses a method of generating a genetically modified plant having an improved agronomic trait. The method comprises: introducing one or more nucleic acid constructs encoding an engineered nucleic acid modification system of any one of claims 64-81 into a plant or plant cell; and growing the plant or plant cell for a time and under conditions sufficient for the nucleic acid expression construct to express the programmable nucleic acid modification system or the CCT protein in the plant or plant cell. Expressing the programmable nucleic acid modification system or expressing the CCT protein introduces a nucleic acid modification in the nucleic acid sequence encoding the CCT protein, thereby modifying the expression of a CCT protein in a plant and improving the agronomic trait of the plant.

One aspect of the instant disclosure encompasses a method of improving an agronomic trait of a plant. The method comprises introducing one or more nucleic acid constructs encoding an engineered nucleic acid modification system of any one of claims 64-81 into a plant or plant cell; and growing the plant or plant cell for a time and under conditions sufficient for the nucleic acid expression construct to express the programmable nucleic acid modification system or the CCT protein in the plant or plant cell. Expressing the programmable nucleic acid modification system or expressing the CCT protein introduces a nucleic acid modification in the nucleic acid sequence encoding the CCT protein, thereby modifying the expression of a CCT protein in a plant and improving the agronomic trait of the plant.

Another aspect of the instant disclosure encompasses a kit for improving an agronomic trait of a plant. The kit comprises: one or more genetically modified plant having an improved agronomic trait; one or more nucleic acid constructs encoding an engineered nucleic acid modification system for modifying the expression of a CCT protein in a plant; a plant comprising one or more nucleic acid constructs encoding a programmable nucleic acid modification system for modifying the expression of a CCT protein in a plant; or any combination of (a)-(c). The plants constructs, and systems can be as described herein above

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 depicts sequencing comparison between FN0172932 and the wild type M92-220.

FIG. 2A depicts species tree and the number of identified CCT domain-containing proteins in each species.

FIG. 2B depicts the number of identified CCT domain-containing proteins in each species and constituent domains and organization.

FIG. 3A depicts the chromosomal details of GmCCT genes in soybean genome and microsynteny relationship in representative legumes by showing the microsynteny comparison of 573 GmCCT12/21 and GmCCT13/20 paralogs among soybean, common bean, cowpea, chickpea, pea, and Medicago.

FIG. 3B depicts the chromosomal details of GmCCT genes in soybean genome and microsynteny relationship in representative legumes by showing microsynteny comparison of 573 GmCCT12/21 and GmCCT34/67 paralogs among soybean, common bean, cowpea, chickpea, pea, and Medicago.

FIG. 4A depicts the phylogeny analysis of CCT protein and domains, showing the global phylogenetic tree of all CCT domain-containing proteins.

FIG. 4B depicts the phylogeny analysis of CCT protein and domains, showing the phylogenetic tree constructed by 43-bp CCT domain.

FIG. 4C depicts the phylogeny analysis of CCT protein and domains, showing HMM logos representing amino acids of CCT domains as illustrated in different clusters in FIG. 4A and FIG. 4B. Conserved and cluster-specific amino acids are indicated in green rectangle and red triangles, respectively.

FIG. 5 depicts the phylogenetic tree of GmCCTs in soybean and the expression patterns in circadian clock response. C and T indicate control and treatment. Blue, green, and red dotted rectangles highlight the circadian clock-responsive GmCCTs, condition-specifically expressed GmCCTs, and condition-responsive GmCCTs, respectively.

FIG. 6 depicts the phylogenetic tree of GmCCTs in soybean and the expression patterns in the compartments of developing seeds at globular, heart, cotyledon, early maturation stages, and major vegetative tissues. Blue and green rectangles indicate the conserved expression and divergent expression of GmCCT paralogs. AB—Abaxial; AD—Adaxial; AL—Aleurone; AX—Axis; COT—Cotyledon; EP—Embryo Proper; EPD—Epidermis; ENT—Endothelium; ES—Endosperm; FBUD—Floral Bud; HG—Hourglass; HI—Hilum; II—Inner Integument; OI—Outer Integument; PA—Palisade; PL—Plumule; PY—Parenchyma; RM—Root Meristem; S—Suspensor; SC—Seed Coat; SM—Shoot Meristem; VS—Vascular Bundle; WM—Whole Mount Seed; SDLG—seedling; FLUB—floral bud; STEM—stem; ROOT—root; LEAF—leaf.

FIG. 7 depicts macrosyntenic visualization of syntenic relationships among CCT proteins between legume genomes.

FIG. 8 depicts the CCT proteins with truncated domains.

FIG. 9A shows the generation of GmCCT34 knockout mutant cct34 using CRISPR/Cas9 editing technology and seed composition measurements by an illustration depicting the preferential expression of GmCCT34 in the seed coats of cotyledon and early maturation seeds of Williams 82. Abbreviation: AB—Abaxial; AD—Adaxial; AL—Aleurone; AX—Axis; EP—Embryo Proper; EPD—Epidermis; ENT—Endothelium; ES—Endosperm; HG—Hourglass; HI—Hilum; II—Inner Integument; OI—Outer Integument; PA—Palisade; PL—Plumule; PY—Parenchyma; S—Suspensor; VS—Vascular Bundle.

FIG. 9B depicts a schematic representation of GmCCT34 and the guide RNAs (gRNAs) sequences for gene knockout. PAM sites (NGG for gRNAs on the forward DNA strand and CCN for gRNAs on the reverse DNA strand) are indicated in blue. A 521-bp fragment containing both gRNA2 and gRNA3 targeting sites was used for BsII digestion to confirm the mutation.

FIG. 9C: Screening results for mutations on gRNA2 and gRNA3 targeting sites by BsII digestion. PCR amplicons carrying any mutations on either or both targeting sites showed different patterns of digested products from those (four bands: 248 bp, 144 bp, 108 bp, and 21 bp) of wild type Williams 82 (Wm82). White and red arrows indicate the resulting two-band pattern from the cct34-2 lines carrying two mutations in both gRNA2 and gRNA3; green arrows indicate band pattern of many cct34-4 lines.

FIG. 9D depicts the targeting sequence comparison of cct34-2-2, cct34-4-5, cct34-4-7 with the wild type Wm82 as indicated in FIG. 9C.

FIG. 9E indicates the comparisons of seed oil, protein, and 100-seed weight between FN0172932 (FN) and the wild type (WT), cct34 and the wild type Wm82, respectively.

FIG. 10A depicts phenotype distribution of the seed traits used for the association studies, illustrating the phenotypic distribution of seed oil content.

FIG. 10B depicts phenotype distribution of the seed traits used for the association studies, illustrating the phenotypic distribution of protein content.

FIG. 10C depicts phenotype distribution of the seed traits used for the association studies, illustrating the phenotypic distribution of 100-seed weight.

FIG. 11A depicts GWAS of oil content in the 278 diverse accessions using a GLM model.

FIG. 11B depicts GWAS of oil content in the 278 diverse accessions using a MLMM model.

FIG. 12A depicts the identification of QTL on chr20 for oil, protein and seed weight and functional validation. Manhattan plots illustrating the regional association results for oil, protein, and seed weight. Red solid dots highlight the 321-bp InDel significantly associated with the three traits. The Bonferroni-corrected genome-wide significance threshold is depicted in the horizontal dotted lines. The three most significantly associated SNPs (ss715637271, ss715637273, ss715637274, left to right) from SoySNP50K data set that were identified in the RILs using GWAS approach are indicated with red arrows below the bottom panel.

FIG. 12B depicts the identification of QTL on chr20 for oil, protein and seed weight and functional validation. Gene structure of Glyma.20G085100 harboring the most significant 321-bp InDel and indication of the InDel between two parental lines (Williams82 and PI479752) of RILs.

FIG. 12C depicts the identification of QTL on chr20 for oil, protein and seed weight and functional validation. Sequencing read alignments of Glyma.20G085100 gene model from two high oil/low protein and two low oil/high protein accessions to that of the soybean reference genome from Williams 82 shown. The 321-bp insertion is present in high-oil/low-protein genotypes but absent in two low-oil/high-protein genotypes. Seed oil (Oil), protein (Pro) and 100-seed weight (SW) of each genotype were provided beside the panel.

FIG. 12D depicts the identification of QTL on chr20 for oil, protein and seed weight and functional validation. Box plots showing the allelic effects of the InDel on oil, protein, and 100-seed weight in the association panel.

FIG. 12E depicts the identification of QTL on chr20 for oil, protein and seed weight and functional validation. Box plots showing the comparison of the three seed traits (oil, protein, seed weight) and field yield in NILs polymorphic for the TE, n=12.

FIG. 12F depicts the identification of QTL on chr20 for oil, protein and seed weight and functional validation. Genotypes of TE in four pairs of parental lines where POWR1 locus was successfully mapped in previous studies. PCR amplification using primers flanking the TE give rise to an amplicon of 1228 bp or an amplicon of 907 bp based on the presence or absence of the TE insertion in tested genotypes. Oil and protein levels from corresponding genotypes are given below the image and are highlighted with a gray background for the genotypes carrying the TE insertion. *, **, and *** are used to indicate significance at p<0.05, p<0.01, and p<0.001, respectively, in all figures in the study.

FIG. 13A depicts GWAS and linkage mapping of oil content and protein content using 300 RILs

FIG. 13B depicts GWAS and linkage mapping of oil content and protein content using 300 RILs by showing association and linkage mapping results of protein and oil content. The most significant associations for both traits are provided in the corresponding Manhattan plot.

FIG. 13C depicts GWAS and linkage mapping of oil content and protein content using 300 RILs by showing association and linkage mapping results of protein and oil content. The most significant associations for both traits are provided in the corresponding Manhattan plot.

FIG. 14 depicts PCR-based genotyping of the 321-bp TE in NILs for POWR1. NILs show a 1228-bp PCR amplicon with the 321-bp TE insertion while NILs show a 907-bp fragment without the 321-bp TE insertion.

FIG. 15A depicts gene structure and expression of POWR1, by showing sequence alignment of CCT domain from different plant species.

FIG. 15B depicts sequence comparison between C terminus of POWR1_{+TE} and POWR1_{-TE}. The conserved CCT domain is colored in green. POWR1_{+TE} is 19 amino acids longer than POWR1_{-TE}. Amino acids in red indicate the distinct peptide sequence at the C terminus of POWR1_{+TE} as the result of the 321-bp TE insertion.

FIG. 15C depicts gene structure of POWR1 with and without the 321-bp TE insertion and the position of TE insertion (red arrow) in POWR1. The insertion caused a codon reading frameshift, which truncated the CCT domain (in orange) and generated a longer C terminus with distinct amino acid sequence (in blue).

FIG. 15D depicts a phylogenetic tree showing the evolutionary relationship among the POWR1_{-TE} homologous proteins from monocot and dicot plant species.

FIG. 15E depicts predicted structures of POWR1_{-TE} and POWR1_{+TE} had almost identical N-termini but distinct C-termini.

FIG. 15F depicts the comparable expression levels of POWR1_{-TE} in 40 soybean accessions and POWR1_{+TE} of 132 accessions in seeds at mid-maturation stages.

FIG. 15G depicts the comparison of subcellular localization of POWR1. TE and POWR1_{+TE} in tobacco cells. Scale bar=20 μm.

FIG. 15H depicts the comparison of expression patterns of POWR1_{-TE} and POWR1_{+TE} in different soybean tissues. Y axis indicates the expression levels relative to GmCYP2.

FIG. 15I depicts enriched GO and KEGG terms for the differentially expressed genes between G. max accessions containing POWR1_{-TE} and POWR1_{+TE}.

FIG. 15J depicts relative expression levels of selected genes in seed coat and cotyledon of NILs containing POWR1_{-TE} or POWR1_{+TE}.

FIG. 16A depicts the comparison of promoter sequences between two POWR1 alleles, by showing IGV visualization of read alignment in the 2-kb region upstream of the start codon of POWR1 in the parental lines of the RIL population, PI479752 and Williams 82.

FIG. 16B depicts sequence comparison of promoter sequences between two POWR1 alleles, by revealing nearly identical promoter sequences between two groups carrying POWR1−TE (20 G. soja accessions) and POWR1+TE (51 G. max accessions). No correlation of seed traits with any DNA variants in their promoters.

FIG. 17 depicts the phenotypic changes associated with the transfer of a POWR1−TE from G. soja into G. max. Seed oil content, seed protein content and 100-seed weight of G. max-POWR1−TE accessions are compared to their closest G. soja accessions and G. max-POWR1+TE accessions based on local and global phylogenetic analyses. The average phenotype values for the Korean clusters 1.1 (C1.1) and 1.2 (C1.2) are given. Both S1.3 and S2 only contain one Japanese accession. A representative accession for S3 is shown. NA: data not available.

FIG. 18A depicts the identification of positive transgenic plants by Basta leaf painting assay by showing schematic illustration of the construct (Ubi917::POWR1) that was used for overexpression of POWR1−TE in soybean.

FIG. 18B depicts the basta leaf painting assay showed basta resistance in two transgenic lines and yellowish wilting leaves in control plants.

FIG. 18C depicts PCR verification of three positive transgenic plants using bar-specific and POWR1-cDNA-specific primers.

FIG. 18D depicts another PCR verification of three positive transgenic plants using bar-specific and POWR1-cDNA-specific primers.

FIG. 18E depicts relative seed expression of POWR1 in control and two transgenic plants.

FIG. 19A depicts the seed oil and protein content and weight in transgenic soybean overexpressing (OE) POWR1−TE, by showing seed protein, oil and weight of T2 plants in each of two transgenic events containing Ubi-promoter driven POWR1_{-TE} CDNA.

FIG. 19B depicts seed protein, oil and weight of T1 plants from 18 independent transgenic events. *, **, and *** indicate significance at p<0.05, p<0.01, and p<0.001, respectively.

FIG. 20A depicts the distribution of both POWR1 alleles in soybean population and diversity analyses, by showing PCA of the soybean accessions with assigned germplasm and allele type.

FIG. 20B depicts comparison of seed oil and protein content and 100-seed weight of G. max and G. soja accessions carrying POWR1_{+TE} or POWR1_{-TE}.

FIG. 20C depicts Tajima's D and Ln(π-G. soja)-Ln(π-G. max) between G. max and G. soja population within the 4.1 Mb region. The vertical solid red line indicates the physical position of POWR1.

FIG. 20D depicts another Tajima's D and Ln(π-G. soja)-Ln(π-G. max) between G. max and G. soja population within the 4.1 Mb region. The vertical solid red line indicates the physical position of POWR1.

FIG. 21A depicts the dynamic interspecific introgressions of POWR1, showing global phylogenetic tree consisting of 548 G. soja and G. max accessions using genome-wide SoySNP50K SNPs and 1,000 SNPs in the 154-kb region containing POWR1 respectively. Labels (1, 2, 3, 4) in the local tree indicate four clusters of accessions containing unusual genotypes (G. max-POWR1_{-TE} (1,2,3), G. soja-POWR1_{+TE} (4)) in the tree. The labels in the global tree are corresponding to the labels in the local tree. Notably, cluster 1 in the local tree is split into subclusters 1.1, 1.2, and 1.3 in the global tree.

FIG. 21B depicts the dynamic interspecific introgressions of POWR1, showing a local phylogenetic tree consisting of 548 G. soja and G. max accessions using genome-wide SoySNP50K SNPs and 1,000 SNPs in the 154-kb region containing POWR1 respectively. Labels (1, 2, 3, 4) in the local tree indicate four clusters of accessions containing unusual genotypes (G. max-POWR1_{-TE} (1,2,3), G. soja-POWR1_{+TE} (4)) in the tree. The labels in the global tree are corresponding to the labels in the local tree. Notably, cluster 1 in the local tree is split into subclusters 1.1, 1.2, and 1.3 in the global tree.

FIG. 21C depicts the pairwise nucleotide distance analyses across a 4.1-Mb region of each G. max-POWR1_{-TE} accession with their closest G. soja-POWR1_{-TE} accessions. Their clusters and origins are labeled. The pairwise distance is indicated by a color scale from red (close) and green (distant).

FIG. 21D depicts G. max accessions with POWR1_{-TE} alleles transferred from G. soja. Top of the panel shows representative accessions from clusters 1.1 (C1.1) and 1.2 (C1.2), 1.3 (S1.3) and 3 (S3) that carry POWR1_{-TE} originated from G. soja. The bottom row shows their closest related G. max-POWR1_{+TE} accessions. Each accession PI number with corresponding 100-seed weight (W), seed protein content (P) and seed oil content (O) are provided.

FIG. 21E depicts geographic origins of G. max-POWR1_{-TE} accessions and closest G. soja-POWR1_{-TE} accessions from the local phylogenetic tree and the closest G. max-POWR1_{+TE} accessions from the global tree. Dotted circles include the geographic regions where interspecific transfer might occur.

FIG. 22 depicts a proposed model of POWR1 in soybean domestication. The insertion of the LINE transposon represents an important event in transition from G. soja to G. max during soybean domestication. Following TE insertion event, the offspring or diversified populations from the plant containing POWR1+TE were expanded likely from the selection for bigger seeds by ancient farmers. Selection for the larger seed together with other human-favorite domestication traits such as seed shattering resistance and loss of seed dormancy resulted in complete fixation of POWR1+TE in all modern G. max accessions with increased oil but reduced protein content in seeds because of its pleiotropy on these traits. The interspecific transfers of POWR1−TE from G. soja to G. max during the post-domestication was likely driven by local needs for high-protein soybean in Asia. Fixation of POWR1+TE in G. max contributes to much larger seeds for modern G. max with higher oil and lower protein content than those of contemporary G. soja.

FIG. 23A depicts the vector and transgenic plant by showing diagram for the vector used for transformation.

FIG. 23B depicts the vector and transgenic plant by showing PCR examination for selected lines containing native promoter-driven POWR1−TE. PCR produced 266 bp in transgenic plants, but not in non-transformed soybean. Wm82 plants is used as a negative control.

FIG. 24 depicts the frequency of POWR1 alleles in a diverse population consisting of 3,956 accessions and the allele effects on protein, oil and seed weight from analyzing their whole genome resequencing data.

FIG. 25A depicts the subcellular localization of GmCCT34.

FIG. 25B depicts another subcellular localization of GmCCT34.

FIG. 26 depicts the seed oil-protein content phenotype of Arabidopsis thaliana T-DNA insertion mutants of the GmPOWR ortholog gene AT1G04500. The top panel shows the AtPOWR1 gene structure with exon regions highlights as a gray box, the arrowheads representing the T-DNA insertion locations for two T-DNA lines, WiscDsLox297300_13A.1 and SALK_036731.1, respectively. The red rectangle shows the CCT domain location spanning exons three and four. The bar graphs show the oil phenotypes. * denotes the statistical significance (p-value <0.05).

FIG. 27 depicts AtPOWR1 expression in the seed coat tissues with red color indicating the AtPOWR1 expression in the seed coat.

### DETAILED DESCRIPTION

The present disclosure is based in part on the identification and characterization of genes encoding CCT motif-containing proteins (CCT proteins) and their comprehensive roles in the regulation of a variety of development and physiological processes critical for multiple agronomically important traits in agricultural plants such as legumes. For instance, the inventors surprisingly discovered a role for a subfamily of CCT proteins in regulating seed protein, seed oil accumulation, and seed weight and field seed yield in economically important legumes such as soybean. The inventors further demonstrated the ability to genetically manipulate these agronomic traits by manipulating expression of the identified CCT proteins. Accordingly, the present disclosure encompasses plants with improved agronomic traits, and compositions and methods for modifying the expression of CCT proteins in a plant to improve an agronomic trait. The present disclosure also encompasses methods of marker-assisted selection (MAS) plant breeding to improve agronomic traits of a plant using molecular markers identified by the inventors through extensive experimentation.

### I. Genetically Modified Plants

One aspect of the present disclosure encompasses a genetically modified plant having an improved agronomic trait. The plant comprises a nucleic acid sequence encoding a CCT motif-containing protein (CCT protein). The nucleic acid sequence encoding the CCT protein comprises a nucleic acid modification that modifies the expression of the CCT protein, thereby improving one or more agronomic traits of the plant. The present disclosure also encompasses agricultural products produced by any of the described genetically modified plants.

### (a) Plants

The present disclosure provides a genetically modified plant having an improved agronomic trait. The plant comprises a nucleic acid sequence encoding a CCT protein. The nucleic acid sequence comprises a nucleic acid modification that modifies the expression of the CCT protein in the plant. As explained in Section I(b) below, CCT proteins are associated with many developmental functions which affect agronomic traits. Accordingly, modifying the expression of the CCT protein in the plant can be used to improve an agronomic trait of the plant. CCT proteins, nucleic acid sequences encoding CCT proteins, and nucleic acid modifications that modify the expression of the CCT protein in the plant can be as described in Section I(b) herein below.

As used herein, a “plant” refers to any of various photosynthetic, eukaryotic multi-cellular organisms of the kingdom Plantae, characteristically producing embryos, containing chloroplasts, having cellulose cell walls and lacking locomotion. As used herein, a “plant” includes any plant or part of a plant at any stage of development, including seeds, suspension cultures, plant cells, embryos, meristematic regions, callus tissue, leaves, roots, shoots, gametophytes, sporophytes, pollen, microspores, and progeny thereof. Also included are cuttings, and cell or tissue cultures. As used in conjunction with the present disclosure, plant tissue includes, without limitation, whole plants, plant cells, plant organs, e.g., leaves, stems, roots, meristems, plant seeds, protoplasts, callus, cell cultures, and any groups of plant cells organized into structural and/or functional units.

Non-limiting examples of suitable plants may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

In some embodiments, plants may include, for example, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia spp.), almond (Prunus amygdalus), sugar beets (Beta vulgaris), sugarcane (Saccharum spp.), oats, barley, vegetables, ornamentals, and conifers.

Non-limiting examples of suitable vegetable plants may include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Non-limiting examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Non-limiting examples of suitable conifer plants may include, for example, loblolly pine (Pinus taeda), slash pine (Pinus elliotii), ponderosa pine (Pinus ponderosa), lodgepole pine (Pinus contorta), Monterey pine (Pinus radiata), Douglas-fir (Pseudotsuga menziesii), Western hemlock (Isuga canadensis), Sitka spruce (Picea glauca), redwood (Sequoia sempervirens), silver fir (Abies amabilis), balsam fir (Abies balsamea), Western red cedar (Thuja plicata), and Alaska yellow-cedar (Chamaecyparis nootkatensis).

Non-limiting examples of suitable forage and turf grass may include, for example, alfalfa (Medicago s sp.), orchard grass, tall fescue, perennial ryegrass, creeping bent grass, and redtop.

Non-limiting examples of suitable crop plants and model plants may include, for example, Arabidopsis, corn, rice, alfalfa, sunflower, canola, soybean, cotton, peanut, sorghum, wheat, tobacco, and lemna.

In some aspects, the plant is a legume (fabacea). Non-limiting examples of suitable leguminous plants may include, for example, guar, locust bean, fenugreek, soybean (Glycine), garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.), Lotus, trefoil, lens, and false indigo.

In some aspects, the plant is a soybean (Glycine sp.). Soybean is one of the most important seed crops grown worldwide. It was domesticated from wild soybean (G. soja) in East Asia about 6,000-9,000 years ago. Domestication and improvement have shaped soybean as the most important dual-function crop to provide both highly valuable seed protein and oil, which together account for almost all of soybean economic value. Non-limiting examples of Glycine sp. include Glycine hispida, Glycine max, and Glycine soja. In another aspect, the plant is Glycine hispida. In some aspects, the soybean plant is a domesticated soybean plant. In one aspect, the plant is Glycine max).

### (b) Agronomic Traits

Any agronomic trait of a plant can be improved by regulating the expression of one or more CCT protein provided the trait depends on the expression of a CCT protein. Non-limiting examples of agronomic traits that can be improved using compositions and methods of the instant disclosure can be an agronomic trait of Table 14. In some aspects, the agronomic trait is seed quality, seed protein content, seed protein composition, seed oil content, seed oil composition, yield, seed set, response to photoperiod, abiotic stress tolerance, biotic stress tolerance, flowering time and maturity, regulation of circadian clock light response-related flowering, high latitude adaptation, or any combination thereof.

In some aspects, the plant is soybean. In some aspects, the agronomic trait is seed quality, seed protein content, seed protein composition, seed oil content, seed oil composition, yield, seed set, response to photoperiod, abiotic stress tolerance, biotic stress tolerance, flowering time and maturity, regulation of circadian clock light response-related flowering, high latitude adaptation, or any combination thereof.

Seed protein content, oil content, and yield are considered as three of the most important traits in soybean improvement. On average, commodity-type soybean varieties contain about 40% seed protein and 20% seed oil. However, the three traits vary greatly in wild soybean populations and often correlate with each other. Seed protein frequently shows a negative correlation with seed oil content and yield. However, its underlying genetic mechanism remains largely unknown. The complex correlation of the three important traits poses a great challenge in simultaneously improving both the soybean seed quality traits and yield to increase the overall economic value of soybean. In addition, cultivated soybean also contains higher seed yield and oil content, but lower protein content than their ancestry, wild soybean. The identification of the CCT proteins in soybean that underlie these important traits by the inventors after extensive experimentation provides the genetic and molecular basis underlying the three traits and their trait correlation. CCT proteins that underlie seed oil content, seed protein content, seed weight, or any combination thereof can be as described in Section I(c).

### (c) CCT Family of Proteins

A plant of the instant disclosure comprises a nucleic acid sequence encoding a CCT motif-containing protein (CCT protein), any variant thereof, or any combination thereof. A CCT protein variant can comprise a naturally occurring variant of a CCT protein, an ortholog of a CCT protein, a paralog of a CCT protein, a CCT protein comprising a loss-of-function mutation, a CCT protein comprising altered expression in the plant, a CCT protein comprising an introduced mutation, or any combination thereof. Non-limiting examples of CCT protein variants include a naturally occurring variant of the CCT protein, an ortholog of the CCT protein, a paralog of the CCT protein, a CCT protein comprising a loss-of-function mutation, a CCT protein comprising altered expression in the plant, a CCT protein comprising an introduced mutation, or any combination thereof.

Proteins comprising a CCT motif (CCT proteins) were initially identified in three proteins in Arabidopsis thaliana, namely CO (CONSTANS), COL (CO-LIKE) and TOC1 (TIMING OF CAB1). CCT proteins play comprehensive roles in the regulation of a variety of development and physiological processes. The CCT motif comprises about a 43-amino acid conserved sequence in the carboxy-terminus of the proteins. CCT proteins form a large family of proteins in plants with demonstrated roles in adaptation or agronomic traits. Proteins comprising CCT domains are generally classified into three subfamilies: (1) CMF (CCT motif family) containing a single CCT domain, (2) COL proteins carrying an additional one or two B-box (BBOX) domains, and (3) PRR (Pseudo Response Regulator) proteins also containing a response regulator (REC) domain. Accordingly, a CCT protein of the instant disclosure can be a CCT protein classified into the CMF sub-family of CCT proteins, a CCT protein classified into the COL sub-family of CCT proteins, a CCT protein classified into the PRR sub-family of CCT proteins, any variants thereof, or any combination thereof. In some aspects, the CCT protein is a protein classified in the CMF sub-family of CCT proteins. In some aspects, the CCT protein is a protein classified in the COL sub-family of CCT proteins. In some aspects, the CCT protein is a protein classified in the PRR sub-family of CCT proteins.

A CCT protein can be a single-CCT domain polypeptide, a 1 or 2×BBOX-CCT domain polypeptide, a REC-CCT domain polypeptide, a TIFY CCT-ZnF_GATA domain polypeptide, a CCT protein comprising non-canonical domains, any variants thereof, or any combination thereof. Non-limiting examples of CCT proteins comprising non-canonical domains include DUF740-DUF740-CCT in Vang06g17920 from adzuki bean, Adaptin_N-CCT in Psat0s3732g0120 from pea, S_TKc-CCT in Ca. 14621 from chickpea, any variant thereof, or any combination thereof.

CCT proteins of the instant disclosure can be selected from a CCT protein of Table 2 any variants thereof, or any combination thereof. Genes interacting with and genes in the biological pathways underlying the CCT genes can also be genetically modified to improve the traits.

As explained in Section I(a) herein above, CCT proteins are used to improve agronomic traits. In some aspects, the improved agronomic trait is an agronomic trait associated with a QTL of Table 15. In some aspects, the agronomic trait is seed quality, and the CCT protein is encoded by a nucleic acid sequence comprising a gene of Table 5. In some aspects, the agronomic trait is seed set and the CCT protein is encoded by a nucleic acid sequence comprising a gene of Table 6. In some aspects, the agronomic trait is abiotic/biotic stress tolerance and the CCT protein is encoded by a nucleic acid sequence comprising a gene of Table 7. In some aspects, the agronomic trait is flowering time and the CCT protein is encoded by a nucleic acid sequence comprising a gene of Table 8. In some aspects, the agronomic trait is development-related traits and the CCT protein is encoded by a nucleic acid sequence comprising a gene of Table 9.

In some aspects, the plant is a soybean plant. In some aspects, a CCT protein of the instant disclosure is a CCT protein of Table 1. In some aspects, the agronomic trait is seed oil content, seed protein content, seed weight, or any combination thereof. In some aspects, the CCT protein is a protein of Table 10. In some aspects, a CCT protein of the instant disclosure is GmCCT05 or any variant thereof. In some aspects, a CCT protein of the instant disclosure is GmCCT34 (POWR2), GmCCT67 (POWR1), GmCCT35 (POWR3), GmCCT69 (POWR4), any variant thereof, or any combination thereof.

In some aspects, the CCT protein is GmCCT67 (POWR1). When the CCT protein is GmCCT67 (POWR1), reducing the expression of the GmCCT67 protein can increase the level of oil in soybean seeds. In some aspects, reducing the expression of the GmCCT67 protein in a soybean plant increases the level of oil in soybean seeds by about 3%, 4%, 5%, 6%, 7%, 8%, 9%, 10%, 11%, 12%, 13%, 14%, 15%, 16%, 1% 7, 18%, 19%, or about 20% w/w or more when compared to the level of oil in seeds of the plant before the level of expression of the GmCCT67 protein is reduced in the plant. When the CCT protein is GmCCT67 (POWR1), reducing the expression of the GmCCT67 protein can also reduce the level of protein in soybean seeds. In some aspects, reducing the expression of the GmCCT67 protein in a soybean plant reduces the level of protein in soybean seeds by about 3%, 4%, 5%, 6%, 7%, 8%, 9%, 10%, 11%, 12%, 13%, 14%, 15%, 16%, 17%, 18%, 19%, or about 20% w/w or more when compared to the level of protein in seeds of the plant before the level of expression of the GmCCT67 protein is reduced in the plant.

In some aspects, the GmCCT67 protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 1. In some aspects, the GmCCT67 (POWR1) protein comprises an amino acid sequence comprising at least about 75% or more, at least about 85% or more, at least about 95% or more, or 100% sequence identity with the amino acid sequence of SEQ ID NO: 1. In some aspects, GmCCT67 is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 2. In some aspects, the GmCCT67 (POWR1) protein is encoded by a nucleic acid sequence comprising at least about 75% or more, at least about 85% or more, at least about 95% or more, or 100% sequence identity with the nucleic acid sequence of SEQ ID NO: 2.

In some aspects, the nucleic acid modification in the nucleic acid sequence encoding the GmCCT67 protein comprises a transposable element (TE) insertion. In some aspects, the nucleic acid sequence comprising the TE insertion comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 3. In some aspects, the nucleic acid sequence encoding the GmCCT67 CCT protein comprises at least about 75% or more, at least about 85% or more, at least about 95% or more, or 100% sequence identity with the nucleic acid sequence of SEQ ID NO: 3.

In some aspects, the nucleic acid modification in the nucleic acid sequence encoding the GmCCT67 protein comprises an expression construct for expression of the GmCCT67 protein, wherein the expression construct comprises a nucleic acid sequence encoding the GmCCT67 protein operably linked to a promoter. In some aspects, the promoter is a ubiquitin promoter or a native promoter.

In some aspects, the expression construct for expression of GmCCT67 comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO:4. In some aspects, the expression construct for expression of GmCCT67 POWR1 comprises a nucleic acid sequence comprising at least about 75% or more, at least about 85% or more, at least about 95% or more, or 100% sequence identity with the nucleic acid sequence of SEQ ID NO: 4.

In some aspects, the CCT protein is GmCCT34 (POWR2). When the CCT protein is GmCCT34 (POWR2), reducing the expression of the GmCCT34 protein can reduce the level of oil in soybean seeds. In some aspects, reducing the expression of the GmCCT34 protein in a soybean plant increases the level of oil in soybean seeds by about 3%, 4%, 5%, 6%, 7%, 8%, 9%, 10%, 11%, 12%, 13%, 14%, 15%, 16%, 17%, 18%, 19%, or about 20% w/w or more when compared to the level of oil in seeds of the plant before the level of expression of the GmCCT34 protein is reduced in the plant. When the CCT protein is GmCCT34 (POWR2), reducing the expression of the GmCCT34 protein can also reduce the level of protein in soybean seeds. In some aspects, reducing the expression of the GmCCT34 protein in a soybean plant reduces the level of protein in soybean seeds by about 3%, 4%, 5%, 6%, 7%, 8%, 9%, 10%, 11%, 12%, 13%, 14%, 15%, 16%, 17%, 18%, 19%, or about 20% w/w or more when compared to the level of protein in seeds of the plant before the level of expression of the GmCCT34 protein is reduced in the plant.

In some aspects, the GmCCT34 (POWR2) protein comprises an amino acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 5. In some aspects, the GmCCT34 (POWR2) protein comprises an amino acid sequence comprising at least about 75% or more, at least about 85% or more, at least about 95% or more, or 100% sequence identity with the amino acid sequence of SEQ ID NO: 5. In some aspects, GmCCT34 (POWR2) is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 6. In some aspects, the GmCCT34 (POWR2) protein is encoded by a nucleic acid sequence comprising at least about 75% or more, at least about 85% or more, at least about 95% or more, or 100% sequence identity with the nucleic acid sequence of SEQ ID NO: 6.

In some aspects, the nucleic acid modification in the nucleic acid sequence encoding the GmCCT34 protein comprises an expression construct for expression of the GmCCT34 protein, wherein the expression construct comprises a nucleic acid sequence encoding the GmCCT34 protein operably linked to a promoter. In some aspects, the promoter is a ubiquitin promoter or a native promoter. In some aspects, the expression construct comprises a nucleic acid sequence encoding the GmCCT34 protein a GmCCT34 variant selected from a wild soybean (G. soja, PI479752 accession).

In some aspects, the expression construct for expression of GmCCT34 POWR2 comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7. In some aspects, the expression construct for expression of GmCCT34 POWR2 comprises a nucleic acid sequence comprising at least about 75% or more, at least about 85% or more, at least about 95% or more, or 100% sequence identity with the nucleic acid sequence of SEQ ID NO: 7.

In some aspects, the nucleic acid modification in the nucleic acid sequence encoding the GmCCT34 protein comprises a nucleic acid modification generated using a CRISPR/Cas programmable nucleic acid modification system targeted to a nucleic acid sequence in a nucleotide sequence encoding the GmCCT34 protein. In some aspects, the nucleic acid modification in the nucleic acid sequence encoding the GmCCT34 protein comprises a nucleic acid sequence of SEQ ID NO: 8 to 16 or any combination thereof. In some aspects, the nucleic acid modification in the nucleic acid sequence encoding the GmCCT34 protein comprises a nucleic acid sequence of SEQ ID NO: 8 to 10 or any combination thereof. In one aspect, the nucleic acid modification in the nucleic acid sequence encoding the GmCCT34 protein comprises a nucleic acid sequence of SEQ ID NO: 11 to 13. In another aspect, the nucleic acid modification in the nucleic acid sequence encoding the GmCCT34 protein comprises a nucleic acid sequence of SEQ ID NO: 14 to 16.

In some aspects, the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein.

In some aspects, The genetically modified plant of claim 8, wherein the CCT protein is GmCCT35 (POWR3). In some aspects, the GmCCT35 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 25. In some aspects, the GmCCT35 protein comprises an amino acid sequence comprising at least about 75% or more, at least about 85% or more, at least about 95% or more, or 100% sequence identity with the amino acid sequence of SEQ ID NO: 25. In additional aspects, the GmCCT35 protein is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 26. In additional aspects, the GmCCT35 protein is encoded by a nucleic acid sequence comprising at least about 75% or more, at least about 85% or more, at least about 95% or more, or 100% sequence identity with the nucleic acid sequence of SEQ ID NO: 26. In yet other aspects, the plant is a soybean species (Glycine max, hispida), the CCT protein is GmCCT35 (POWR3), the nucleic acid modification in the nucleic acid sequence encoding the GmCCT34 protein comprises a nucleic acid sequence comprising at least about 75% or more, at least about 85% or more, at least about 95% or more, or 100% sequence identity with the nucleic acid sequence of SEQ ID NO: 27.

In some aspects, the CCT protein is GmCCT69 (POWR4). In some aspects, the GmCCT69 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 28. In some aspects, the GmCCT69 protein comprises an amino acid sequence comprising at least about 75% or more, at least about 85% or more, at least about 95% or more, or 100% sequence identity with the amino acid sequence of SEQ ID NO: 28. In additional aspects, the GmCCT69 protein is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 29. In additional aspects, the GmCCT69 protein is encoded by a nucleic acid sequence comprising at least about 75% or more, at least about 85% or more, at least about 95% or more, or 100% sequence identity with the nucleic acid sequence of SEQ ID NO: 29. In yet other aspects, the plant is a soybean species (Glycine max, hispida), the CCT protein is GmCCT69 (POWR4), the nucleic acid modification in the nucleic acid sequence encoding the GmCCT69 protein comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 30. In yet other aspects, the plant is a soybean species (Glycine max, hispida), the CCT protein is GmCCT69 (POWR4), the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 30.

The mutations in POWR3 (GmCCT35) and POWR 4 (GmCCT69) genes were generated by using CRISPR-Cas9 mediated gene editing approach. For POWR 3, the gRNAs were designed to target exon 2 and 3 regions. The CRISPR-Cas9 mediated 4 be deletion (in exon 3 by using gRNA-ctggcagaacttccagcccc SEQ ID NO: 34), and 39 bp deletion (in exon 2 by using gRNA-ccaggactgagataagtgca SEQ ID NO: 35) were generated. Similarly, for POWR4, exon 2 region was targeted by gRNA-ccaggactgagataagtgca SEQ ID NO: 36, which generated a 39 bp deletion.

In some aspects, the CCT protein is AtPOWR1, any variant thereof, or any combination thereof. In some aspects, the nucleic acid modification reduces the expression of the AtPOWR1 protein in the plant. When the nucleic acid modification reduces the expression of the AtPOWR1 protein in the plant, the oil content of the seeds can be increased and the protein content of the seeds can be reduced. In some aspects, the AtPOWR1 protein comprises an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 33. In some aspects, the AtPOWR1 protein comprises an amino acid sequence comprising at least about 75% or more, at least about 85% or more, at least about 95% or more, or 100% sequence identity with the amino acid sequence of SEQ ID NO: 33. In other aspects, the AtPOWR1 protein is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 31. In other aspects, the AtPOWR1 protein is encoded by a nucleic acid sequence comprising at least about 75% or more, at least about 85% or more, at least about 95% or more, or 100% sequence identity with the nucleic acid sequence of SEQ ID NO: 31.

### (d) Aspects of Plants

One aspect of the present disclosure encompasses a genetically modified plant having an improved agronomic trait. The plant comprises a nucleic acid sequence encoding a CCT motif-containing protein (CCT protein) selected from CCT proteins of Table 2, a variant of any thereof, or any combination thereof. The nucleic acid sequence encoding the CCT protein comprises a nucleic acid modification, wherein the nucleic acid modification modifies the expression of the CCT protein in the plant thereby improving the agronomic trait of the plant. The nucleic acid modification can be a nucleic acid sequence comprising a single nucleotide polymorphism of Table 4, Table 10, or any combination thereof.

A CCT protein variant can comprise a naturally occurring variant of the CCT protein, an ortholog of the CCT protein, a paralog of the CCT protein, a CCT protein comprising a loss-of-function mutation, a CCT protein having altered expression in the plant, a CCT protein comprising an introduced mutation, a functional fragment, or any combination thereof. In some aspects, the CCT protein is a single-CCT domain polypeptide, a 1 or 2×BBOX-CCT domain polypeptide, a REC-CCT domain polypeptide, a TIFY CCT-ZnF_GATA domain polypeptide, a CCT protein comprising one or more non-canonical domains, any variants thereof, or any combination thereof. The CCT protein comprising non-canonical domains can be DUF740-DUF740-CCT in Vang06g17920 from adzuki bean, Adaptin_N-CCT in Psat0s3732g0120 from pea, S_TKc-CCT in Ca. 14621 from chickpea, any variants thereof, or any combination thereof. In some aspects, the CCT protein is a single-CCT domain polypeptide.

In some aspects, the CCT protein is a CCT protein of Table 1. In some aspects, the CCT protein is GmCCT05 and wherein the agronomic trait is drought tolerance. In some aspects, the agronomic trait is seed protein, oil content, 100-seed weight, or any combination thereof, and the CCT protein is GmCCT34 (POWR2), GmCCT67 (POWR1), GmCCT35 (POWR3), GmCCT69 (POWR4), any variant thereof, or any combination thereof. In one aspect, the CCT protein is GmCCT35 (POWR3). In another aspect, the CCT protein is GmCCT69 (POWR4).

The agronomic trait can be seed quality, seed protein content, seed protein composition, seed oil content, seed oil composition, yield, seed set, response to photoperiod, abiotic stress tolerance, biotic stress tolerance, flowering time and maturity, regulation of circadian clock light response-related flowering, high latitude adaptation, or any combination thereof. In some aspects, the improved agronomic trait is an agronomic trait of Table 14. In some aspects, the improved agronomic trait is an agronomic trait associated with a QTL of Table 15. In some aspects, the agronomic trait is (a) seed quality and the CCT protein is encoded by a nucleic acid sequence comprising a gene of Table 5; (b) yield-related traits and the CCT protein is encoded by a nucleic acid sequence comprising a gene of Table 6; (c) response to abiotic/biotic stress tolerance and the CCT protein is encoded by a nucleic acid sequence comprising a gene of Table 7; (d) flowering time and maturity and the CCT protein is encoded by a nucleic acid sequence comprising a gene of Table 8; and (e) development-related traits and the CCT protein is encoded by a nucleic acid sequence comprising a gene of Table 9.

In some aspects, the CCT protein is GmCCT67 (POWR1). A nucleic acid modification can reduce the expression of the GmCCT67 protein in the plant. When the nucleic acid modification reduces the expression of the GmCCT67 protein in the plant, the oil content of the seeds is increased by about 0.5% to about 5% wt/wt and wherein the protein content of the seeds is reduced by about 1% wt/wt to about 20% wt/wt. Alternatively, a nucleic acid modification can increase the expression of the GmCCT67 protein in the plant. When the nucleic acid modification increases the expression of the GmCCT67 protein in the plant, the oil content of the seeds can be decreased by about 0.5% to about 5% wt/wt and wherein the protein content of the seeds is increased by about 1% wt/wt to about 20% wt/wt.

The GmCCT67 protein can comprise an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 1, and can be encoded by a nucleic acid sequence comprising at least about 75% or more, at least about 85% or more, at least about 95% or more, or 100% sequence identity with the nucleic acid sequence of SEQ ID NO: 2. The GmCCT67 protein can also comprise an amino acid sequence comprising at least about 75% or more, at least about 85% or more, at least about 95% or more, or 100% sequence identity with the amino acid sequence of SEQ ID NO: 1, and can be encoded by a nucleic acid sequence comprising at least about 75% or more, at least about 85% or more, at least about 95% or more, or 100% sequence identity with the nucleic acid sequence of SEQ ID NO: 2.

In some aspects, the nucleic acid modification in the nucleic acid sequence encoding the GmCCT67 protein comprises a transposable element (TE) insertion. In one aspect, the nucleic acid sequence comprising the TE insertion comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 3. In one aspect, the nucleic acid sequence comprising the TE insertion comprises at least about 75% or more, at least about 85% or more, at least about 95% or more, or 100% sequence identity with the nucleic acid sequence of SEQ ID NO: 3.

In some aspects, the nucleic acid modification in the nucleic acid sequence encoding the GmCCT67 protein comprises an expression construct for expression of the GmCCT67 protein, wherein the expression construct comprises a nucleic acid sequence encoding the GmCCT67 protein operably linked to a ubiquitin promoter or a native promoter. In one aspect, the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 4. In one aspect, the expression construct comprises a nucleic acid sequence comprising at least about 75% or more, at least about 85% or more, at least about 95% or more, or 100% sequence identity with the nucleic acid sequence of SEQ ID NO: 4.

In some aspects, the CCT protein is GmCCT34 (POWR2). When the CCT protein is GmCCT34, the nucleic acid modification reduces the expression of GmCCT34 (POWR2) in the plant, and the oil content of the seeds is increased by about 0.5% to about 5% wt/wt and wherein the protein content of the seeds is reduced by about 1% wt/wt to about 20% wt/wt.

The GmCCT34 protein can comprise an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 5, and can be encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 6. The GmCCT34 protein can comprise an amino acid sequence comprising at least about 75% or more, at least about 85% or more, at least about 95% or more, or 100% sequence identity with the amino acid sequence of SEQ ID NO: 5, and can be encoded by a nucleic acid sequence comprising at least about 75% or more, at least about 85% or more, at least about 95% or more, or 100% sequence identity with the nucleic acid sequence of SEQ ID NO: 6.

The nucleic acid modification in the nucleic acid sequence encoding the GmCCT34 protein can comprise an expression construct for expression of the GmCCT34 protein, wherein the expression construct comprises a nucleic acid sequence encoding the GmCCT34 protein operably linked to a promoter. In some aspects, the expression construct for expression of GmCCT34 POWR2 comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7. In some aspects, the expression construct for expression of GmCCT34 POWR2 comprises a nucleic acid sequence comprising at least about 75% or more, at least about 85% or more, at least about 95% or more, or 100% sequence identity with the nucleic acid sequence of SEQ ID NO: 7.

In some aspects, the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein. In one aspect, the nucleic acid modification in the nucleic acid sequence encoding the GmCCT34 protein comprises a nucleic acid modification generated using a CRISPR/Cas programmable nucleic acid modification system targeted to a nucleic acid sequence in a nucleotide sequence encoding the GmCCT34 protein. The nucleic acid modification in the nucleic acid sequence encoding the GmCCT34 protein can comprise a nucleic acid sequence of SEQ ID NO: 8 to 10 or any combination thereof, a nucleic acid sequence of SEQ ID NO: 11 to 13 or any combination thereof, or a nucleic acid sequence of SEQ ID NO: 14 to 16 or any combination thereof.

The plant can be a legume (Fabaceae) such as common bean, cowpea, soybean, chickpea, pea, or Medicago. In some aspects, the legume is a soybean species (Glycine max, hispida). In some aspects, the CCT protein is GmCCT67 (POWR1) and wherein the nucleic acid modification reduces the expression of the GmCCT67 protein in the plant. In other aspects, the CCT protein is GmCCT34 (POWR1) and wherein the nucleic acid modification reduces the expression of the GmCCT34 protein in the plant.

In some aspects, the plant is a soybean species (Glycine max, hispida), the CCT protein is GmCCT67 (POWR1), the nucleic acid modification in the nucleic acid sequence encoding the GmCCT67 protein comprises an expression construct for expression of the GmCCT67 protein, wherein the expression construct comprises a nucleic acid sequence encoding the GmCCT67 protein operably linked to a promoter, and wherein the nucleic acid modification increases the expression of the GmCCT67 protein in the plant. In some aspects, the oil content of the seeds is decreased by about 0.5% to about 5% wt/wt and wherein the protein content of the seeds is increased by about 1% wt/wt to about 20% wt/wt.

The plant can be a soybean species (Glycine max, hispida), the CCT protein is GmCCT67 (POWR1), the nucleic acid modification in the nucleic acid sequence encoding the GmCCT67 protein comprises a transposable element (TE) insertion, and wherein the nucleic acid modification reduces the expression of the GmCCT67 protein in the plant. In some aspects, the oil content of the seeds is increased by about 0.5% to about 5% wt/wt and wherein the protein content of the seeds is reduced by about 1% wt/wt to about 20% wt/wt.

In some aspects, the plant is a soybean species (Glycine max, hispida), the CCT protein is GmCCT34 (POWR2), the nucleic acid modification in the nucleic acid sequence encoding the GmCCT34 protein comprises an expression construct for expression of the GmCCT34 protein, wherein the expression construct comprises a nucleic acid sequence encoding the GmCCT34 protein operably linked to a promoter, and wherein the nucleic acid modification increases the expression of the GmCCT34 protein in the plant. In one aspect, the oil content of the seeds is decreased by about 0.5% to about 5% wt/wt and wherein the protein content of the seeds is increased by about 1% wt/wt to about 20% wt/wt.

In some aspects, GmCCT34 (POWR2), the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein or a nucleic acid sequence of SEQ ID NO: 8 to 16 or any combination thereof, and wherein the nucleic acid modification reduces the expression of the GmCCT34 protein in the plant. In one aspects, the oil content of the seeds is increased by about 0.5% to about 5% wt/wt and wherein the protein content of the seeds is reduced by about 1% wt/wt to about 20% wt/wt.

In some aspects, the plant is a soybean species (Glycine max, hispida), the CCT protein is GmCCT35 (POWR3), the nucleic acid modification in the nucleic acid sequence encoding the GmCCT34 protein comprises a nucleic acid sequence comprising at least about 75% or more, at least about 85% or more, at least about 95% or more, or 100% sequence identity with the nucleic acid sequence of SEQ ID NO: 27.

In other aspects, the plant is a soybean species (Glycine max, hispida), the CCT protein is GmCCT69 (POWR4), the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 30.

In yet other aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof.

In additional aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; and the CCT protein is GmCCT69 (POWR4) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 30.

In some aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; and the CCT protein is GmCCT35 (POWR3) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 27.

In additional aspects, the plant is a soybean species (Glycine max, hispida), wherein the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; and the CCT protein is GmCCT35 (POWR3) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 27.

In some aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; and the CCT protein is GmCCT69 (POWR4) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 30.

In additional aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; and the CCT protein is GmCCT35 (POWR3) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 27.

In yet other aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; and the CCT protein is GmCCT35 (POWR3) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 27.

In some aspects, the plant is a soybean species (Glycine max, hispida), wherein the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof; the CCT protein is GmCCT35 (POWR3) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 27; and the CCT protein is GmCCT69 (POWR4) and the nucleic acid modification in the nucleic acid sequence encoding the GmCCT69 protein comprises a nucleic acid sequence comprising at least about 75% or more, at least about 85% or more, at least about 95% or more, or 100% sequence identity with the nucleic acid sequence of SEQ ID NO: 30.

In some aspects, the plant is Arabidopsis thaliana. When the plant is Arabidopsis thaliana, the CCT protein can be AtPOWR1, any variant thereof, or any combination thereof. In some aspects, the nucleic acid modification reduces the oil content of the seeds is increased and wherein the protein content of the seeds is reduced. In some aspects, the AtPOWR1 protein comprises an amino acid sequence comprising at least about 75% or more, at least about 85% or more, at least about 95% or more, or 100% sequence identity with the amino acid sequence of SEQ ID NO: 33. In additional aspects, the AtPOWR1 protein is encoded by a nucleic acid sequence comprising at least about 75% or more, at least about 85% or more, at least about 95% or more, or 100% sequence identity with the nucleic acid sequence of SEQ ID NO: 31. In yet other aspects, the Arabidopsis plant comprises a first T-DNA-insertion mutant of AtPOWR1 (WiscDsLox297300_13A.1, Atcct1), a second T-DNA-insertion mutant of AtPOWR1 (SALK_036731.1; Atcct-2).

### II. Engineered Nucleic Acid Modification System

One aspect of the present disclosure encompasses an engineered nucleic acid modification system for modifying the expression of a CCT protein in a plant. Non-limiting examples of suitable protein expression modification systems include programmable nucleic acid modification systems, an expression construct encoding a protein or variants thereof, and any combination thereof.

In some aspects, the nucleic acid modification system is an expression construct comprising a nucleotide sequence encoding the CCT protein operably linked to a promoter. Expression constructs comprising a nucleotide sequence encoding the CCT protein operably linked to a promoter can be as described in Section I(c).

In some aspects, the nucleic acid modification system is a programmable nucleic acid modification system targeted to a sequence within a gene encoding the CCT protein. As used herein, a “programmable nucleic acid modification system” is a system capable of targeting and modifying the nucleic acid or modifying the expression or stability of a nucleic acid to alter a protein or the expression of a protein encoded by the nucleic acid. The programmable nucleic acid modification system can comprise an interfering nucleic acid molecule or a nucleic acid editing system. The programmable protein expression modification system is specifically targeted to a sequence within a gene encoding the CCT protein.

In some aspects, the programmable expression modification system comprises an interfering nucleic acid (RNAi) molecule having a nucleotide sequence complementary to a target sequence within a gene encoding the CCT protein used to inhibit expression of the CCT protein. RNAi molecules generally act by forming a heteroduplex with a target RNA molecule, which is selectively degraded or “knocked down,” hence inactivating the target RNA. Under some conditions, an interfering RNA molecule can also inactivate a target transcript by repressing transcript translation and/or inhibiting transcription. An interfering RNA is more generally said to be “targeted against” a biologically relevant target, such as a protein, when it is targeted against the nucleic acid encoding the target. For example, an interfering RNA molecule has a nucleotide (nt) sequence which is complementary to an endogenous mRNA of a target gene sequence. Thus, given a target gene sequence, an interfering RNA molecule can be prepared which has a nucleotide sequence at least a portion of which is complementary to a target gene sequence. When introduced into cells, the interfering RNA binds to the target mRNA, thereby functionally inactivating the target mRNA and/or leading to degradation of the target mRNA.

Interfering RNA molecules include, inter alia, small interfering RNA (siRNA), microRNA (miRNA), piwi-interacting RNA (piRNA), long non-coding RNAs (long ncRNAs or lncRNAs), and small hairpin RNAs (shRNA). lncRNAs are widely expressed and have key roles in gene regulation. Depending on their localization and their specific interactions with DNA, RNA and proteins, lncRNAs can modulate chromatin function, regulate the assembly and function of membraneless nuclear bodies, alter the stability and translation of cytoplasmic mRNAs, and interfere with signaling pathways. Piwi-interacting RNA (piRNA) is the largest class of small non-coding RNA molecules expressed in animal cells. piRNAs regulate gene expression through interactions with piwi-subfamily Argonaute proteins. SiRNA are double-stranded RNA molecules, preferably about 19-25 nucleotides in length. When transfected into cells, siRNA inhibit the target mRNA transiently until they are also degraded within the cell. MiRNA and siRNA are biochemically and functionally indistinguishable. Both are about the same in nucleotide length with 5′-phosphate and 3′-hydroxyl ends, and assemble into an RNA-induced silencing complex (RISC) to silence specific gene expression. siRNA and miRNA are distinguished based on origin. siRNA is obtained from long double-stranded RNA (dsRNA), while miRNA is derived from the double-stranded region of a 60-70nt RNA hairpin precursor. Small hairpin RNAs (shRNA) are sequences of RNA, typically about 50-80 base pairs, or about 50, 55, 60, 65, 70, 75, or about 80 base pairs in length, that include a region of internal hybridization forming a stem loop structure consisting of a base-pair region of about 19-29 base pairs of double-strand RNA (the stem) bridged by a region of single-strand RNA (the loop) and a short 3′ overhang. shRNA molecules are processed within the cell to form siRNA which in turn knock down target gene expression. shRNA can be incorporated into plasmid vectors and integrated into genomic DNA for longer-term or stable expression, and thus longer knockdown of the target mRNA.

Interfering nucleic acid molecules can contain RNA bases, non-RNA bases, or a mixture of RNA bases and non-RNA bases. For example, interfering nucleic acid molecules provided herein can be primarily composed of RNA bases but also contain DNA bases or non-naturally occurring nucleotides. The interfering nucleic acids can employ a variety of oligonucleotide chemistries. Non-limiting examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone.

In some aspects, the programmable nucleic acid modification system is a nucleic acid editing system. Such modification system can be used to edit DNA or RNA sequences to repress transcription or translation of an mRNA encoded by the gene, and/or produce mutant proteins with reduced activity or stability. Non-limiting examples of programmable nucleic acid editing systems include, without limit, an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, a ribozyme, or a programmable DNA binding domain linked to a nuclease domain. Other suitable programmable nucleic acid modification systems will be recognized by individuals skilled in the art.

Such systems rely for specificity on the delivery of exogenous protein(s), and/or a guide RNA (gRNA) or single guide RNA (sgRNA) having a sequence which binds specifically to a gene sequence of interest. When the programmable nucleic acid modification system comprises more than one component, such as a protein and a guide nucleic acid, the multi-component modification system can be modular, in that the different components may optionally be distributed among two or more nucleic acid constructs as described herein. The system components can be delivered by a plasmid or viral vector or as a synthetic oligonucleotide. More detailed descriptions of programmable nucleic acid editing systems can be as described further below.

In some aspects, the programmable nucleic acid modification system is a CRISPR/Cas tool modified for transcriptional regulation of a locus. In some aspects, the programmable nucleic acid modification system is a CRISPR/Cas transcriptional regulator driven by cell-specific promoters using a catalytically dead effector (dCAS9) to modulate transcription of a nucleic acid sequence encoding a CCT protein.

In some aspects, the programmable nucleic acid modification system is a CRISPR/Cas system comprising a guide RNA (gRNA) having a sequence complementary to a target sequence within the nucleotide sequence encoding the CCT protein. In some aspects, the CCT protein is a GmCCT34 protein. In some aspects, the GmCCT34 protein is encoded by a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 5. In some aspects, the GmCCT34 (POWR2) protein is encoded by a nucleic acid sequence comprising at least about 75% or more, at least about 85% or more, at least about 95% or more, or 100% sequence identity with the nucleic acid sequence of SEQ ID NO: 5. When the programmable nucleic acid modification system is a CRISPR/Cas system and the CCT protein is a GmCCT34 protein, the gRNA can comprise a nucleic acid sequence of SEQ ID NO: 17, SEQ ID NO: 18, SEQ ID NO: 19, or any combination thereof.

In some aspects, the CCT protein is a GmCCT35 protein. When the programmable nucleic acid modification system is a CRISPR/Cas system and the CCT protein is a GmCCT35 protein, the gRNA can comprise a nucleic acid sequence of SEQ ID NO: 34, SEQ ID NO: 35, or a combination thereof.

In some aspects, the CCT protein is a GmCCT69 protein. When the programmable nucleic acid modification system is a CRISPR/Cas system and the CCT protein is a GmCCT69 protein, the gRNA can comprise a nucleic acid sequence of SEQ ID NO: 36.

Another aspect of the present disclosure encompasses an engineered nucleic acid modification system for modifying the expression of a CCT protein in a plant. The system comprises a nucleic acid expression construct comprising: a promoter operably linked to a nucleic acid sequence encoding a programmable nucleic acid modification system targeted to a nucleic acid sequence in a nucleotide sequence encoding the CCT protein; or a nucleotide sequence encoding the CCT protein operably linked to a promoter; and wherein expressing the programmable nucleic acid modification system or expressing the CCT protein introduces a nucleic acid modification of the nucleic acid sequence encoding the CCT protein, thereby modifying the expression of a CCT protein in a plant. In some aspects, the engineered nucleic acid modification system further comprises a nucleic acid delivery vector comprising the nucleic acid expression construct for delivering the nucleic acid expression construct to the target cell.

The CCT protein can be GmCCT34 (POWR2), GmCCT67 (POWR1), GmCCT35 (POWR3), GmCCT69 (POWR4), or any combination thereof. In some aspects, the CCT protein is GmCCT67 (POWR1) encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 2. In some aspects, the CCT protein is GmCCT67 (POWR1) encoded by a nucleic acid sequence comprising at least about 75% or more, at least about 85% or more, at least about 95% or more, or 100% sequence identity with the nucleic acid sequence of SEQ ID NO: 2. The GmCCT67 (POWR1) protein can comprise an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the amino acid sequence of SEQ ID NO: 1. The GmCCT67 (POWR1) protein can comprise an amino acid sequence comprising at least about 75% or more, at least about 85% or more, at least about 95% or more, or 100% sequence identity with the amino acid sequence of SEQ ID NO: 1. The nucleic acid expression construct can comprise a nucleotide sequence encoding a GmCCT67 protein operably linked to a promoter. In some aspects, the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 4. In some aspects, the expression construct comprises a nucleic acid sequence comprising at least about 75% or more, at least about 85% or more, at least about 95% or more, or 100% sequence identity with the nucleic acid sequence of SEQ ID NO: 4.

In some aspects, the CCT protein is GmCCT34 encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 6. In some aspects, the CCT protein is GmCCT34 encoded by a nucleic acid sequence comprising at least about 75% or more, at least about 85% or more, at least about 95% or more, or 100% sequence identity with the nucleic acid sequence of SEQ ID NO: 6. The GmCCT34 can comprise an amino acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% or more, or 100% sequence identity with the amino acid sequence of SEQ ID NO: 5. The GmCCT34 can comprise an amino acid sequence comprising at least about 75% or more, at least about 85% or more, at least about 95% or more, or 100% sequence identity with the amino acid sequence of SEQ ID NO: 5.

In some aspects, the nucleic acid modification in the nucleic acid sequence encoding the GmCCT34 protein comprises an expression construct for expression of the GmCCT34 protein, wherein the expression construct comprises a nucleic acid sequence encoding the GmCCT34 protein operably linked to a promoter. The expression construct for expression of GmCCT34 POWR2 can comprise a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7. The expression construct for expression of GmCCT34 POWR2 can comprise a nucleic acid sequence comprising at least about 75% or more, at least about 85% or more, at least about 95% or more, or 100% sequence identity with the nucleic acid sequence of SEQ ID NO: 7.

The nucleic acid expression construct can also comprise a promoter operably linked to a nucleic acid sequence encoding a programmable nucleic acid modification system targeted to a nucleic acid sequence in a nucleotide sequence encoding the GmCCT34 protein. The programmable nucleic acid modification system can be CRISPR/Cas system comprising a guide RNA (gRNA) having a sequence complementary to a target sequence within the nucleotide sequence encoding the GmCCT34 protein. In some aspects, the gRNA comprises a nucleic acid sequence of SEQ ID NO: 17, SEQ ID NO: 18, SEQ ID NO: 19, or any combination thereof.

In some aspects, the nucleic acid modification in the nucleic acid sequence encoding the GmCCT34 protein comprises an expression construct for expression of the GmCCT34 protein, wherein the expression construct comprises a nucleic acid sequence encoding the GmCCT34 protein operably linked to a promoter. The nucleic acid expression construct can comprise a nucleotide sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence of SEQ ID NO: 4. The nucleic acid expression construct can comprise a nucleotide sequence comprising at least about 75% or more, at least about 85% or more, at least about 95% or more, or 100% sequence identity with the nucleotide sequence of SEQ ID NO: 4. In some aspects, the nucleic acid expression construct comprises a nucleotide sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleotide sequence of SEQ ID NO: 7. In some aspects, the nucleic acid expression construct comprises a nucleotide sequence comprising at least about 75% or more, at least about 85% or more, at least about 95% or more, or 100% sequence identity with the nucleotide sequence of SEQ ID NO: 7.

i. CRISPR Nuclease Systems.

The programmable targeting nuclease can be an RNA-guided CRISPR endonuclease system. The CRISPR system comprises a guide RNA or sgRNA to a target sequence at which a protein of the system introduces a double-stranded break in a target nucleic acid sequence, and a CRISPR-associated endonuclease. The gRNA is a short synthetic RNA comprising a sequence necessary for endonuclease binding, and a preselected ˜20 nucleotide spacer sequence targeting the sequence of interest in a genomic target. Non-limiting examples of endonucleases include Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, a codon-optimized version thereof, or a modified version thereof, or any combination thereof.

The CRISPR nuclease system may be derived from any type of CRISPR system, including a type I (i.e., IA, IB, IC, ID, IE, or IF), type II (i.e., IIA, IIB, or IIC), type III (i.e., IIIA or IIIB), or type V CRISPR system. The CRISPR/Cas system may be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system may be a type II Cas9 protein, a type V Cpf1 protein, or a derivative thereof. In some aspects, the CRISPR/Cas nuclease is Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises an RNA recognition and/or RNA binding domain, which interacts with the guide RNA. A protein of the CRISPR system also comprises at least one nuclease domain having endonuclease activity. For example, a Cas9 protein may comprise a RuvC-like nuclease domain and an HNH-like nuclease domain, and a Cpf1 protein may comprise a RuvC-like domain. A protein of the CRISPR system may also comprise DNA binding domains, helicase domains, RNase domains, protein-protein interaction domains, dimerization domains, as well as other domains.

A protein of the CRISPR system may be associated with guide RNAs (gRNA). The guide RNA may be a single guide RNA (i.e., sgRNA), or may comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA may comprise GN17-20GG). The gRNA may also comprise a scaffold sequence that forms a stem loop structure and a single-stranded region. The scaffold region may be the same in every gRNA. In some aspects, the gRNA may be a single molecule (i.e., sgRNA). In other aspects, the gRNA may be two separate molecules. Those skilled in the art are familiar with gRNA design and construction, e.g., gRNA design tools are available on the internet or from commercial sources.

A CRISPR system may comprise one or more nucleic acid binding domains associated with one or more, or two or more selected guide RNAs used to direct the CRISPR system to one or more, or two or more selected target nucleic acid loci. For instance, a nucleic acid binding domain may be associated with one or more, or two or more selected guide RNAs, each selected guide RNA, when complexed with a nucleic acid binding domain, causing the CRISPR system to localize to the target of the guide RNA.

ii. CRISPR Nickase Systems.

The programmable targeting nuclease can also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase, in combination with a guide RNA of the system, may create a single-stranded break or nick in the target nucleic acid sequence. Alternatively, a CRISPR nickase in combination with a pair of offset gRNAs may create a double-stranded break in the nucleic acid sequence.

A CRISPR nuclease of the system may be converted to a nickase by one or more mutations and/or deletions. For example, a Cas9 nickase may comprise one or more mutations in one of the nuclease domains, wherein the one or more mutations may be D10A, E762A, and/or D986A in the RuvC-like domain, or the one or more mutations may be H840A (or H839A), N854A and/or N863A in the HNH-like domain.

iii. ssDNA-Guided Argonaute Systems.

Alternatively, the programmable targeting nuclease may comprise a single-stranded DNA-guided Argonaute endonuclease. Argonautes (Agos) are a family of endonucleases that use 5′-phosphorylated short single-stranded nucleic acids as guides to cleave nucleic acid targets. Some prokaryotic Agos use single-stranded guide DNAs and create double-stranded breaks in nucleic acid sequences. The ssDNA-guided Ago endonuclease may be associated with a single-stranded guide DNA.

The Ago endonuclease may be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago endonuclease may be Natronobacterium gregoryi Ago (NgAgo). Alternatively, the Ago endonuclease may be Thermus thermophilus Ago (TtAgo). The Ago endonuclease may also be Pyrococcus furiosus (PfAgo).

The single-stranded guide DNA (gDNA) of an ssDNA-guided Argonaute system is complementary to the target site in the nucleic acid sequence. The target site has no sequence limitations and does not require a PAM. The gDNA generally ranges in length from about 15-30 nucleotides. The gDNA may comprise a 5′ phosphate group. Those skilled in the art are familiar with ssDNA oligonucleotide design and construction.

iv. Zinc Finger Nucleases.

The programmable targeting nuclease may be a zinc finger nuclease (ZFN). A ZFN comprises a DNA-binding zinc finger region and a nuclease domain. The zinc finger region may comprise from about two to seven zinc fingers, for example, about four to six zinc fingers, wherein each zinc finger binds three nucleotides. The zinc finger region may be engineered to recognize and bind to any DNA sequence. Zinc finger design tools or algorithms are available on the internet or from commercial sources. The zinc fingers may be linked together using suitable linker sequences.

A ZFN also comprises a nuclease domain, which may be obtained from any endonuclease or exonuclease. Non-limiting examples of endonucleases from which a nuclease domain may be derived include, but are not limited to, restriction endonucleases and homing endonucleases. The nuclease domain may be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. The type II-S nuclease domain may be modified to facilitate dimerization of two different nuclease domains. For example, the cleavage domain of FokI may be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain may comprise Q486E, 1499L, and/or N496D mutations, and the other modified FokI domain may comprise E490K, 1538K, and/or H537R mutations.

v. Transcription Activator-Like Effector Nuclease Systems.

The programmable targeting nuclease may also be a transcription activator-like effector nuclease (TALEN) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays may be engineered via modular protein design to target any DNA sequence of interest. Other transcription activator-like effector nuclease systems may comprise, but are not limited to, the repetitive sequence, transcription activator like effector (RipTAL) system from the bacterial plant pathogenic Ralstonia solanacearum species complex (Rssc). The nuclease domain of TALEs may be any nuclease domain as described above in Section II (i).

vi. Meganucleases or Rare-Cutting Endonuclease Systems.

The programmable targeting nuclease may also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. Non-limiting examples of meganucleases that may be suitable for the instant disclosure include I-Scel, I-Crel, I-Dmol, or variants and combinations thereof. A meganuclease may be targeted to a specific nucleic acid sequence by modifying its recognition sequence using techniques well known to those skilled in the art.

The programmable targeting nuclease can be a rare-cutting endonuclease or derivative thereof. Rare-cutting endonucleases are site-specific endonucleases whose recognition sequence occurs rarely in a genome, such as only once in a genome. The rare-cutting endonuclease may recognize a 7-nucleotide sequence, an 8-nucleotide sequence, or longer recognition sequence. Non-limiting examples of rare-cutting endonucleases include NotI, AscI, PacI, AsiSI, SbfI, and FseI.

vii. Optional Additional Domains.

The programmable targeting nuclease may further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). The NLS may be located at the N-terminus, the C-terminal, or in an internal location of the fusion protein.

A cell-penetrating domain may be a cell-penetrating peptide sequence derived from the HIV-1 TAT protein. The cell-penetrating domain may be located at the N-terminus, the C-terminal, or in an internal location of the fusion protein.

A programmable targeting nuclease may further comprise at least one linker. For example, the programmable targeting nuclease, the nuclease domain of the targeting nuclease, and other optional domains may be linked via one or more linkers. The linker may be flexible (e.g., comprising small, non-polar (e.g., Gly) or polar (e.g., Ser, Thr) amino acids). Non-limiting examples of suitable linkers are well known in the art, and programs to design linkers are readily available (Crasto et al., Protein Eng., 2000, 13 (5): 3096-312). In alternate aspects, the programmable targeting nuclease, the cell cycle regulated protein, and other optional domains may be linked directly.

A programmable targeting nuclease may further comprise an organelle localization or targeting signal that directs a molecule to a specific organelle. A signal may be a polynucleotide or polypeptide signal, or may be an organic or inorganic compound sufficient to direct an attached molecule to a desired organelle. Organelle localization signals can be as described in U.S. Patent Publication No. 20070196334, the disclosure of which is incorporated herein in its entirety.

### III. Nucleic Acid Constructs

A further aspect of the present disclosure provides a system of one or more nucleic acid constructs encoding the components of the engineered nucleic acid modification system described above in Section II.

Any of the multi-component systems described herein are to be considered modular, in that the different components may optionally be distributed among two or more nucleic acid constructs as described herein. The nucleic acid constructs may be DNA or RNA, linear or circular, single-stranded or double-stranded, or any combination thereof. The nucleic acid constructs may be codon-optimized for efficient translation into protein, and possibly for transcription into an RNA donor polynucleotide transcript in the cell of interest. Codon optimization programs are available as freeware or from commercial sources.

The nucleic acid constructs can be used to express one or more components of the system for later introduction into a cell to be genetically modified. Alternatively, the nucleic acid constructs can be introduced into the cell to be genetically modified for expression of the components of the system in the cell. In some aspects, the nucleic acid constructs transiently express the various components of the system. Transiently expressing the system in a plant overcomes the cumbersome regulatory hurdles required for traditionally genetically modified crops.

Expression constructs generally comprise DNA coding sequences operably linked to at least one promoter control sequence for expression in a cell of interest. Promoter control sequences may control expression of the transposase, the programmable targeting nuclease, the donor polynucleotide, or combinations thereof in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. As explained above, methylation of the MeSWEET10a gene can be targeted in leaves by specifically expressing the system in leaves using a leaf-specific promoter, allowing for fine-tuning pathogen resistance and normal plant growth and development.

Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Non-limiting examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter.

Promoters may also be plant-specific promoters, or promoters that may be used in plants. A wide variety of plant promoters are known to those of ordinary skill in the art, as are other regulatory elements that may be used alone or in combination with promoters. Preferably, promoter control sequences control expression in cassava, such as promoters disclosed in Wilson et al., 2017, The New Phytologist, 213 (4): 1632-1641, the disclosure of which is incorporated herein in its entirety.

Promoters may be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604,121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter may be a promoter which is induced by one or more, but not limited to one of the following: abiotic stresses such as wounding, cold, desiccation, ultraviolet-B, heat shock or other heat stress, drought stress or water stress. The promoter may further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize lvr2 gene promoter; heat-inducible promoters such as heat tomato hsp80-promoter from tomato.

Tissue-specific promoters may include, but are not limited to, fiber-specific, green tissue-specific, root-specific, stem-specific, flower-specific, callus-specific, pollen-specific, egg-specific, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262:12202, 1987; Baszczynski et al., Plant Mol. Biol. 14:633, 1990), Brazil Nut albumin (Pearson et al., Plant Mol. Biol. 18:235-245, 1992), legumin (Ellis et al., Plant Mol. Biol. 10:203-214, 1988), Glutelin (rice) (Takaiwa et al., Mol. Gen. Genet. 208:15-22, 1986; Takaiwa et al., FEBS Letts. 221:43-47, 1987), Zein (Matzke et al., Plant Mol Biol, 143:323-32, 1990), napA (Stalberg et al., Planta 199:515-519, 1996), Wheat SPA (Albanietal, Plant Cell, 9:171-184, 1997), sunflower oleosin (Cummins et al., Plant Mol. Biol. 19:873-876, 1992)], endosperm specific promoters [e.g., wheat LMW and HMW, glutenin-1 (Mol Gen Genet 216:81-90, 1989; NAR 17:461-2), wheat a, b, and g gliadins (EMBO3: 1409-15, 1984), Barley Itrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116 (1): 53-62, 1998), Biz2 (EP99106056.7), Synthetic promoter (Vicente-Carbajosa et al., Plant J. 13:629-640, 1998), rice prolamin NRP33, rice-globulin Glb-1 (Wu et al., Plant Cell Physiology 39 (8) 885-889, 1998), rice alpha-globulin REB/OHP-1 (Nakase et al., Plant Mol. Biol. 33:513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93:8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3].

Any of the promoter sequences may be wild type or may be modified for more efficient or efficacious expression. The DNA coding sequence also may be linked to a polyadenylation signal (e.g., SV40 polyA signal, bovine growth hormone (BGH) polyA signal, etc.) and/or at least one transcriptional termination sequence. In some situations, the complex or fusion protein may be purified from the bacterial or eukaryotic cells.

Nucleic acids encoding one or more components of an engineered DNA methylation system and/or transcription activation system may be present in a construct. Suitable constructs include plasmid constructs, viral constructs, and self-replicating RNA (Yoshioka et al., Cell Stem Cell, 2013, 13:246-254). For instance, the nucleic acid encoding one or more components of an engineered DNA methylation system and/or transcription activation system may be present in a plasmid construct.

Non-limiting examples of suitable plasmid constructs include pUC, pBR322, pET, pBluescript, and variants thereof. Alternatively, the nucleic acid encoding one or more components of an engineered DNA methylation system and/or transcription activation system may be part of a viral vector (e.g., lentiviral vectors, adeno-associated viral vectors, adenoviral vectors, and so forth).

The plasmid or viral vector may comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasmid or viral vector may further comprise RNA processing elements such as glycine tRNAs, or Csy4 recognition sites. Such RNA processing elements can, for instance, intersperse polynucleotide sequences encoding multiple gRNAs under the control of a single promoter to produce the multiple gRNAs from a transcript encoding the multiple gRNAs. When a cys4 recognition cite is used, a vector may further comprise sequences for expression of Csy4 RNAse to process the gRNA transcript. Additional information about vectors and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

In some aspects, the nucleic acid modification comprises an expression construct for expression of POWR1, wherein the construct comprises a nucleotide sequence encoding the CCT protein operably linked to a promoter. In some aspects, the CCT protein is GmCCT67. In some aspects, the promoter is a ubiquitin promoter.

In some aspects, the expression construct for expression of GmCCT67 POWR1 comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO:4. In some aspects, the expression construct for expression of GmCCT67 POWR1 comprises a nucleic acid sequence comprising at least about 75% or more, at least about 85% or more, at least about 95% or more, or 100% sequence identity with the nucleic acid sequence of SEQ ID NO: 4.

In some aspects, the expression construct for expression of GmCCT34 POWR2 comprises a nucleic acid sequence comprising about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the nucleic acid sequence of SEQ ID NO: 7. In some aspects, the expression construct for expression of GmCCT34 POWR2 comprises a nucleic acid sequence comprising at least about 75% or more, at least about 85% or more, at least about 95% or more, or 100% sequence identity with the nucleic acid sequence of SEQ ID NO: 7.

### IV. Methods

A further aspect of the present disclosure encompasses a method of generating a genetically modified plant having an improved agronomic trait. The method comprises introducing one or more nucleic acid constructs encoding an engineered nucleic acid modification system for modifying the expression of a CCT protein in a plant into a plant or plant cell. The plant or plant cell is then grown under conditions whereby the nucleic acid expression construct expresses the programmable nucleic acid modification system or the CCT protein in the plant or plant cell. Expressing the programmable nucleic acid modification system or expressing the CCT protein introduces a nucleic acid modification in the nucleic acid sequence encoding the CCT protein, thereby modifying the expression of a CCT protein in a plant and improving the agronomic trait of the plant. The CCT protein and the plant can be as described in Section I. The engineered nucleic acid modification system for modifying the expression of a CCT protein in a plant can be as described in Section II, and nucleic acid constructs expressing the engineered nucleic acid modification system can be as described in Section III.

Another aspect of the present disclosure encompasses a method of improving an agronomic trait of a plant. The method comprises introducing one or more nucleic acid constructs encoding an engineered nucleic acid modification system for modifying the expression of a CCT protein in a plant into a plant or plant cell, growing the plant or plant cell under conditions whereby the nucleic acid expression construct expresses the programmable nucleic acid modification system or the CCT protein in the plant or plant cell. Expressing the programmable nucleic acid modification system or expressing the CCT protein introduces a nucleic acid modification in the nucleic acid sequence encoding the CCT protein, thereby modifying the expression of a CCT protein in a plant and improving the agronomic trait of the plant. The CCT protein and the plant can be as described in Section I. The engineered nucleic acid modification system for modifying the expression of a CCT protein in a plant can be as described in Section II, and nucleic acid constructs expressing the engineered nucleic acid modification system can be as described in Section III.

### (a) Marker-Assisted Selection

Yet another aspect of the present disclosure encompasses a method of identifying a plant having an improved agronomic trait of a plant using marker-assisted selection (MAS). The method comprises identifying in a population of plants one or more plants comprising a molecular marker that demonstrates linkage with a nucleic acid modification that modifies the expression of a CCT protein in the plant. Through extensive experimentation, the inventors identified genetic markers that are linked to nucleic acid sequences encoding CCT proteins wherein the nucleic acid modification modifies the expression of the CCT protein in the plant thereby improving the agronomic trait of the plant.

Molecular markers suitable for a method of the instant disclosure are known in the art and include, without limitation, restriction fragment length polymorphisms (RFLPs), isozyme markers, allele specific hybridization (ASH), amplified variable sequences of plant genome, self-sustained sequence replication, simple sequence repeat (SSR), single base-pair change (single nucleotide polymorphism, SNP), random amplification of polymorphic DNA (RAPDs), SSCPs (single stranded conformation polymorphisms); amplified fragment length polymorphisms (AFLPs), a quantitative trait locus (QTL), and microsatellites DNA. In some aspects, the molecular marker is a QTL selected from SNPs of Table 15. In some aspects, the population of plants is a progeny of a cross between parent plants. In some aspects, a parent plant is a plant described in Section I.

Molecular markers can be used in a variety of plant breeding applications. Molecular markers can be used to increase the efficiency of identifying progeny plants of a cross between parent plants using marker-assisted selection (MAS), wherein one or more of the progeny plants comprise a favorable nucleic acid modification. As used herein, the term “favorable nucleic acid modification” is a nucleic acid modification that modifies the expression of the CCT protein in the plant thereby improving the agronomic trait of the plant.

A molecular marker that demonstrates linkage with a locus affecting a desired phenotypic trait provides a useful tool for the selection of the trait in a plant population. This is particularly true with traits that are difficult to phenotype due to their dependence on environmental conditions. This category includes traits related to an improved agronomic trait. This category also includes traits that are very expensive to phenotype because of laborious artificial inoculation or maintenance of managed stress environments. Another category of traits includes those which are associated with destruction of plant per se. Destructive phenotyping has been a bottleneck to implement MAS for the seed quality traits. Because DNA marker assays are not environmentally dependent, are robust, reliable, less laborious, less costly and take up less physical space than field phenotyping, much larger populations can be assayed, increasing the chances of finding a recombinant with the target segment from the donor line moved to the recipient line. The closer the linkage, the more useful the marker, as recombination is less likely to occur between the marker and the gene causing the trait, which can result in false positives. Having flanking markers decreases the chances that false positive selection will occur as a double recombination event would be needed. The ideal situation is to have a marker in the gene itself, so that recombination cannot occur between the marker and the gene. Such a marker is called a ‘perfect marker’.

When a gene is introgressed by MAS, it is not only the gene that is introduced but also the flanking regions. This is referred to as “linkage drag.” In the case where the donor plant is highly unrelated to the recipient plant, these flanking regions carry additional genes that may code for agronomically undesirable traits. This “linkage drag” may also result in negative agronomic characteristics even after multiple cycles of backcrossing into the elite plant line. The size of the flanking region can be decreased by additional backcrossing, although this is not always successful, as breeders do not have control over the size of the region or the recombination breakpoints. In classical breeding it is usually only by chance that recombinations are selected that contribute to a reduction in the size of the donor segment. Even after 20 backcrosses in backcrosses of this type, one may expect to find a sizeable piece of the donor chromosome still linked to the gene being selected. With markers however, it is possible to select those rare individuals that have experienced recombination near the gene of interest. In 150 backcross plants, there is a 95% chance that at least one plant will have experienced a crossover within 1 cM of the gene, based on a single meiosis map distance. Markers will avow unequivocal identification of those individuals. With one additional backcross of 300 plants, there would be a 95% chance of a crossover within 1 cM single meiosis map distance of the other side of the gene, generating a segment around the target gene of less than 2 cM based on a single meiosis map distance. This can be accomplished in two generations with markers, while it would have required on average 100 generations without markers. When the exact location of a gene is known, flanking markers surrounding the gene can be utilized to select for recombinations in different population sizes. For example, in smaller population sizes, recombinations may be expected further away from the gene, so more distal flanking markers would be required to detect the recombination.

(b) Introduction into the Cell

The method comprises introducing a nucleic acid construct expressing an engineered protein into a cell of interest. As explained above, an engineered protein can be encoded on more than one nucleic acid sequence. Accordingly, a method of the instant disclosure comprises introducing more than one nucleic acid construct into the cell.

The one or more nucleic acid constructs described above may be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, viral vectors, magnetofection, lipofection, impalefection, optical transfection, Agrobacterium tumefaciens mediated foreign gene transformation, proprietary agent-enhanced uptake of nucleic acids, and delivery via liposomes, immunoliposomes, virosomes, or artificial virions. The choice of means of introducing the system into a cell can and will vary depending on the cell, or the system or nucleic acid nucleic acid constructs encoding the system, among other variables.

### (c) Culturing a Cell

The method further comprises culturing a cell under conditions suitable for expressing the engineered protein. Methods of culturing cells are known in the art. In some aspects, the cell is from an animal, fungi, oomycete or prokaryote. In some aspects, the cell is a plant cell, plant, or plant part. When the cell is in tissue ex vivo, or in vivo within a plant or within a plant part, the plant part and/or plant may also be maintained under appropriate conditions for insertion of the donor polynucleotide. In general, the plant, plant part, or plant cell is maintained under conditions appropriate for cell growth and/or maintenance. Those of skill in the art appreciate that methods for culturing plant cells are known in the art and may and will vary depending on the cell type. Routine optimization may be used, in all cases, to determine the best techniques for a particular cell type.

### V. Kits

A further aspect of the present disclosure provides kits comprising one or more genetically modified plant having an improved agronomic trait, an engineered nucleic acid modification system for modifying the expression of a CCT protein in a plant, one or more nucleic acid constructs encoding an engineered nucleic acid modification system for modifying the expression of a CCT protein in a plant, a plant comprising the one or more nucleic acid constructs encoding a programmable nucleic acid modification system, or any combination thereof.

The genetically modified plant having an improved agronomic trait can be as described in Section I. The engineered nucleic acid modification system for modifying the expression of a CCT protein in a plant can be as described in Section II. The one or more nucleic acid constructs encoding an engineered nucleic acid modification system for modifying the expression of a CCT protein in a plant can be as described in Section III. A plant comprising the one or more nucleic acid constructs encoding a programmable nucleic acid modification system can be as described in Section I herein above.

The kits may further comprise transfection reagents, cell growth media, selection media, in vitro transcription reagents, nucleic acid purification reagents, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

A “genetically modified” plant refers to a plant in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, the term “engineered” when applied to a targeting protein refers to targeting proteins modified to specifically recognize and bind to a nucleic acid sequence at or near a target nucleic acid locus. A “genetically modified” plant refers to a cell in which the nuclear, organellar or extrachromosomal nucleic acid sequences of a cell have been modified, i.e., the cell contains at least one nucleic acid sequence that has been engineered to contain an insertion of at least one nucleotide, a deletion of at least one nucleotide, and/or a substitution of at least one nucleotide.

The term “nucleic acid modification” refers to processes by which a specific nucleic acid sequence in a polynucleotide is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

As used herein, “protein expression” includes but is not limited to one or more of the following: transcription of a gene into precursor mRNA; splicing and other processing of the precursor mRNA to produce mature mRNA; mRNA stability; translation of the mature mRNA into protein (including codon usage and tRNA availability); production of a mutant protein comprising a mutation that modifies the activity of the protein, including the calcium channel activity; and glycosylation and/or other modifications of the translation product, if required for proper expression and function. The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

The term “Molecular marker” shall refer to any type of nucleic acid based marker, including but not limited to, Restriction Fragment Length Polymorphism (RFLP), Simple Sequence Repeat (SSR), Random Amplified Polymorphic DNA (RAPD), Cleaved Amplified Polymorphic Sequences (CAPS), Amplified Fragment Length Polymorphism (AFLP), Single Nucleotide Polymorphism (SNP), Sequence Characterized Amplified Region (SCAR), Sequence Tagged Site (STS), Single Stranded Conformation Polymorphism (SSCP), Inter-Simple Sequence Repeat (ISR), Inter-Retrotransposon Amplified Polymorphism (IRAP), Retrotransposon-Microsatellite Amplified Polymorphism (REMAP), an RNA cleavage product (such as a Lynx tag), and the like.

The term “allele” as used herein refers to one of two or more different nucleotide sequences that occur at a specific locus.

An allele, a nucleic acid modification, or a CCT protein is “associated with” an agronomic trait when it is linked to it and when the presence of the allele, nucleic acid modification, or CCT protein is an indicator that the desired trait will occur in a plant comprising the allele, nucleic acid modification, or CCT protein.

“Backcrossing” refers to the process whereby hybrid progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. The initial cross gives rise to the F1 generation: the term “BC1” then refers to the second use of the recurrent parent; “BC2” refers to the third use of the recurrent parent, and so on.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, an “elite line” is any line that has resulted from breeding and selection for superior agronomic performance.

A “favorable allele” is the allele at a particular locus that confers, or contributes to, a desirable phenotype, e.g., increased GS tolerance, or alternatively, is an allele that allows the identification of plants with decreased GS tolerance that can be removed from a breeding program or planting (“counterselection”). A favorable allele of a marker is a marker allele that segregates with the favorable phenotype, or alternatively, segregates with the unfavorable plant phenotype, therefore providing the benefit of identifying plants.

“Genome” refers to the total DNA, or the entire set of genes, carried by a chromosome or chromosome set.

The terms “phenotype”, or “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait”. In other cases, a phenotype is the result of several genes.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple led, or, more generally, the term genotype can be used to refer to an individual's genetic make-up for all the genes in its genome.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leaves, stems, pollen, or cells, that can be cultured into a whole plant.

A “haplotype” is the genotype of an individual at a plurality of genetic loci, i.e. a combination of alleles. Typically, the genetic loci described by a haplotype are physically and genetically linked, i.e., on the same chromosome segment. The term “haplotype” can refer to sequence, polymorphisms at a particular locus, such as a single marker locus, or sequence polymorphisms at multiple loci along a chromosomal segment in a given genome. The former can also be referred to as “marker haplotypes” or “marker alleles”, while the latter can be referred to as “long-range haplotypes”.

A “heterotic group” comprises a set of genotypes that perform well when crossed with genotypes from a different heterotic group. Inbred lines are classified into heterotic groups, and are further subdivided into families within a heterotic group, based on several criteria such as pedigree, molecular marker-based associations, and performance in hybrid combinations. The two most widely used heterotic groups in the United States are referred to as “Iowa Stiff Stalk Synthetic” (BSSS) and “Lancaster” or “Lancaster Sure Crop” (sometimes referred to as NSS, or Iron-Stiff Stalk).

The term “heterozygous” means a genetic condition wherein different alleles reside at corresponding loci on homologous chromosomes.

The term “homozygous” means a genetic condition wherein identical alleles reside at corresponding loci on homologous chromosomes.

The term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines.

“Hybridization” or “nucleic acid hybridization” refers to the pairing of complementary RNA and DNA strands as well as the pairing of complementary DNA single strands.

The term “hybridize” means the formation of base pairs between complementary regions of nucleic acid strands.

The term “inbred” means a line that has been bred for genetic homogeneity.

The term “indel” refers to an insertion or deletion, wherein one line may be referred to as having an insertion relative to a second line, or the second line may be referred to as having a deletion relative to the first line.

The term “introgression” or “introgressing” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background. For example, the GS locus described herein may be introgressed into a recurrent parent that has increased GS tolerance. The recurrent parent line with the introgressed gene or locus then has increased GS tolerance.

As used herein, the term “linkage” is used to describe the degree with which one marker locus is associated with another marker locus or some other locus (for example, a GS locus). The linkage relationship between a molecular marker and a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units for cM). In some aspects, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10 (e.g., about 9%, 8%, 7%, 6%, 5%, 4%, 3%, 2%, 1%, 0.75%, 0.5%, 0.25%, or less) are also said to be “proximal to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 CM or less from each other.

The term “linkage disequilibrium” refers to a non-random segregation of genetic loci or traits for both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same chromosome.) As used herein, linkage can be between two markers, or alternatively between a marker and a phenotype. A marker locus can be “associated with” (linked to) a trait, e.g., decreased green snap. The degree of linkage of a molecular marker to a phenotypic trait is measured, e.g., as a statistical probability of co-segregation of that molecular marker with the phenotype.

Linkage disequilibrium is most commonly assessed using the measure r2. When r2=1, complete LD exists between the two marker loci, meaning that the markers have not been separated by recombination and have the same allele frequency. Values for r2 above ⅓ indicate sufficiently strong LD to be useful for mapping. Hence, alleles are in linkage disequilibrium when r2 values between pairwise marker loci are greater than or equal to 0.33, 0.4, 0.5, 0.6, 0.7, 0.8, 0.9, or 1.0.

As used herein, “linkage equilibrium” describes a situation where two markers independently segregate, i.e., sort among progeny randomly. Markers that show linkage equilibrium are considered unlinked (whether or not they lie on the same chromosome).

A “marker” is a nucleotide sequence or encoded product thereof (e.g., a protein) used as a point of reference. For markers to be useful at detecting recombinations, they need to detect differences, or polymorphisms, within the population being monitored. For molecular markers, this means differences at the DNA level due to polynucleotide sequence differences (e.g. SSRs, RFLPs, AFLPs, SNPs). The genomic variability can be of any origin, for example, insertions, deletions, duplications, repetitive elements, point mutations, recombination events, or the presence and sequence of transposable elements. Molecular markers can be derived from genomic or expressed nucleic acids (e.g., ESTs) and can also refer to nucleic acids used as probes or primer pairs capable of amplifying sequence fragments via the use of PCR-based methods.

Markers corresponding to genetic polymorphisms between members of a population can be detected by methods well established in the art. These include, e.g., DNA sequencing, PCR-based sequence specific amplification methods, detection of FLPs, detection of isozyme markers, detection of polynucleotide polymorphisms by allele specific hybridization (ASH), detection of amplified variable sequences of the plant genome, detection of self-sustained sequence replication, detection of SSRs, detection of SNPs, or detection of FLPs. Well established methods are also known for the detection of expressed sequence tags (ESTs) and SSR markers derived from EST sequences and RAPDs.

A “marker allele”, alternatively an “allele of a marker locus”, can refer to one of a plurality of polymorphic nucleotide sequences found at a marker locus in a population that is polymorphic for the marker locus.

“Marker assisted selection” (or MAS) is a process by which phenotypes are selected based on marker genotypes.

“Marker assisted counter-selection” is a process by which marker genotypes are used to identify plants that will not be selected, allowing them to be removed from a breeding program or planting.

A “marker locus” is a specific chromosome location in the genome of a species when a specific marker can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

A “marker probe” is a nucleic add sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e. genotype) the particular allele that is present at a marker locus.

The term “molecular marker” may be used to refer to a molecular marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g., SNP technology is used in the examples provided herein.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

A “polymorphism” is a variation in the DNA that is too common to be due merely to new mutation. A polymorphism must have a frequency of at least 1% in a population. A polymorphism can be a single nucleotide polymorphism, or SNP, or an insertion/deletion polymorphism, also referred to herein as an “indel”.

The term “progeny” refers to the offspring generated from a cross.

A “progeny plant” is generated from a cross between two plants.

A “reference sequence” is a defined sequence used as a basis for sequence comparison. The reference sequence is obtained by genotyping a number of lines at the locus, aligning the nucleotide sequences in a sequence alignment program (e.g. Sequencher), and then obtaining the consensus sequence of the alignment.

A “single nucleotide polymorphism (SNP)” is an allelic single nucleotide-A, T, C or G-variation within a DNA sequence representing one locus of at least two individuals of the same species. For example, two sequenced DNA fragments representing the same locus from at least two individuals of the same species, contain a difference in a single nucleotide.

The term “quantitative trait locus (QTL)” means a locus that controls to some degree numerically representable traits that are usually continuously distributed.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences may also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs may be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Comprehensive Genome-Wide Analysis of CCT Domain Family Genes GmCCT34 Identified as Involving in Seed Protein and Oil Accumulation in Soybean

CCT domain is included in a large family of proteins in plants with demonstrated roles in adaptation or agronomic traits, however, such an important family in economically important legumes has yet to be systematically investigated. In the current study, a combination of comparative genomics, transcriptomics, and population genomics was used to comparatively investigate CCTs in legumes with a prioritized analysis on GmCCTs in soybean and conducted gene functional validation with fast-neutron mutation and gene editing analyses.

Four subfamilies of CCT domain-containing proteins were identified with conserved domain constitution and arrangement across plant species. The soybean genome contained 69 CCT-domain proteins, approximately two times of those in other legumes. Whole-genome duplication was a major driven force of GmCCT family expansion. Further analysis has revealed domain sequence divergence, domain shuffling, and syntenic CCTs in legumes. GmCCTs were rich in natural variation and twelve have the signature of artificial selection. GmCCTs exhibited diversified expression patterns with some showing specificities to circadian clock or environment stressors, or in certain seed tissues.

The current studies demonstrated a newly discovered role of CCT regulating seed protein and oil accumulation and seed weight. The current results provided an overview of molecular evolution, phylogeny, conserved and novel functions of GmCCTs, shedding insight into the role of CCT domain proteins for legume improvement.

### INTRODUCTION

Advance in DNA sequencing has been greatly promoting the accumulation of reference genome sequences in individual species. Gene function characterization in model plant species advanced the annotation and prediction of functions for orthologous genes in crop species of the lineage. Comparative genomics studies provided genomic evidence of indicating conservation in the evolutionary process of genomes and functionality of orthologous genes across species falling within the same genus or family, especially for those with insufficient experimental evidence in underexplored species. Information gathered from systematic studies of gene families, ortholog groups, and gene functions across species provided important insights into the evolutionary processes and functions of gene families of importance.

The CCT motif genes were initially identified in three proteins in Arabidopsis thaliana, namely CO (CONSTANS), COL (CO-LIKE) and TOC1 (TIMING OF CAB1) and they generally contained 43-amino acid conserved sequence in the carboxy-terminus of the proteins. CCT genes generally were classified CCT family into three subfamilies, CMF (CCT motif family) containing a single CCT domain, COL proteins carrying an additional one or two B-box (BBOX) domains, and PRR (Pseudo Response Regulator) proteins also containing a response regulator (REC) domain. Extensive studies revealed that CCT proteins played important roles in the regulation of flowering by controlling photoperiod response or circadian clock and abiotic stress responses or plant development. CCT domains played a role in DNA binding and it was also required for the interaction of CO with COP1 or NF-YB2 to affect flowering time. The results suggested comprehensive roles of CCT family genes involved in the regulation of a variety of development and physiological processes in the model plant Arabidopsis. The knowledge gained from the studies would be helpful to infer the roles of CCT orthologs in other species with the potential to facilitate crop improvement.

However, knowledge about the function of the CCT family genes and the agricultural significance in crop species was so far limited to cereal crops. For example, Ghd7 and Ghd7.1 (BBOX-CCT) from rice and ZmCCT and ZmCCT9 (a single CCT) from maize underly respective major QTLs for rice or maize adaptation from tropical cultivation to longer-day higher latitudes, some of which were subjected to artificial selection. These genes were also critical for multiple agriculturally important traits that can favor human needs such as higher grain production. Despite the importance, the evolution and biological/agricultural significance of the CCT family have yet been systematically explored.

Legumes (Fabaceae) comprised the most economically important bean species that can be used for both grain and forage and has a contributing role to the ecosystem by nitrogen fixation, whereas non-legume crops rarely do. Legumes' grains account for 33% of the protein needs of humans and have been a major plant-based protein provider to meet a great demand for a legume-rich diet. However, legumes were less researched, lagging greatly behind the cereals in both yielding and planting acreages. In legumes, soybean was the most cultivated legume crop with dual uses for both vegetable oil and high-quality proteins, and was also deemed to be a model legume providing tremendous insights into legume research. Protein and oil content accumulation was investigated primarily in soybean in the last decade, mainly via genetic approaches, while rare gene underlying the mechanism has been identified. Therefore, the mechanism of protein and oil accumulation remains largely unclear, hindering the practical improvement of protein and oil. Thus far, the genetic or molecular link between CCT genes and seed proteins has yet to be reported. Currently, the rapid advances in genomic technologies have led to the development of the reference genome assemblies for several legumes (legumeinfo.org). The availability of the reference genomes provided an unprecedented opportunity to explore and compare CCT motif-containing genes to seek opportunities of using them for legume improvement.

In the present study, a detailed overview is provided of the quantities, evolution, phylogeny of CCT proteins in eight representative legume genomes with a focus on soybean genome, along with the comparisons with those in Arabidopsis, and two cereals, rice and maize. Further provided is a detailed characterization of expression patterns of soybean CCT family genes in different tissues or under different conditions. The natural variation of GmCCTs was explored and the candidacy for previously identified QTLs associated with a variety of agronomically important traits in soybean was predicted. Finally, the role of GmCCT34 involved in protein and oil accumulation was experimentally validated using both fast neutron mutation and gene editing approaches. The results presented here provide a fundamental frame for and new insight into the evolution and biological mechanism of CCT genes in soybean and legumes.

### Materials and Methods

### A. Identification of CCT Genes in Soybean, Legumes, and Other Species

The complete protein profiles for soybean (Glycine max, Wm82.a2.v1), eight legumes (adzuki bean (Vigna angularis, 3.0), chickpea (Cicer arietinum), common bean (Phaseolus vulgaris, 2.0), cowpea (Vigna unguiculata, v1.0), Medicago (Medicago truncatula, Mt4.0v1), pea (Pisum sativum, v1.0), peanut (Arachis hypogaea), pigeon pea (Cajanus cajan), and Arabidopsis (Arabidopsis thaliana, TAIR10), and two monocot species (maize (Zea maize, Ensembl-18), rice (Oryza sativa, v7_JGI)) were downloaded from the Phytozome v12 and Legume Information System. Protein profiles from chlorophyte species including Chlamydomonas reinhardtii v5.5, Dunaliella salina v1.0, Coccomyxa subellipsoidea C-169 v2.0, Micromonas pusilla CCMP1545 V3.0, Micromonas sp. RCC299 v3.0, Ostreococcus lucimarinus v2.0 were downloaded from the Phytozome for comparison. The HMM file of the CCT domain (PF06203) was downloaded from Pfam database. Search for the CCT domain proteins (p<1^{e-10}) in each protein dataset was carried out with the HMM file using HMMER software (v3.3.1). The search hits obtained from the HMMER were manually checked in the online database SMART (Simple Modular Architecture Research Tool) to ensure the presence of CCT domains (p<1e-10). Sequence alignment of the CCT complete protein sequences or extracted CCT domain sequences were performed using ClustalX2.1 followed with unrooted phylogenetic tree construction using MEGA 7.0 with the neighbor-joining method and 1000-bootstrapping analysis.

### B. Expression Analysis

The raw sequencing data for the transcriptomes of seed tissues and compartments in developing seeds of cv. Williams82 at different developmental stages, generated by Goldberg-Harada laboratories, and the sequencing data for circadian clock, abiotic/biotic stress analyses (PRJNA285677, PRJNA288296, PRJNA259941, PRJNA432861, PRJNA207354, PRJNA285880, PRJNA348534) were retrieved from NCBI SRA database and re-analyzed. The raw sequencing reads were aligned to the Williams 82 soybean reference genome (Wm82.a2.v1) with TopHat (v2.1.1). Transcript abundance for each gene was estimated using Cufflinks followed by normalization across samples using the quartile method in Cuffdiff. The heatmap was drawn in R with the function heatmap.2 from the gplots package.

### C. Genotyping and Genetic Diversity Analyses

Genotyping and genetic diversity analysis for GmCCTs were carried out using the 32mSNPs identified in a panel consisting of 1,556 diverse soybean genomes. SNPs and indels with a minimum allele frequency of greater than 0.01 were reported. Genetic diversity (Pi) was calculated in the wild and landrace soybean subpopulations with 10-kb window 5-kb step window as previously described. Pi value for a CCT was calculated by the 5-kb window that harboring the gene, and the ratio of Pi-wild versus Pi-landrace greater than 4 was deemed as a putative selective sweep. The Phyton version of MCScan was used to identify gene blocks and syntenic genes in genomes across the species. OrthoFinder was used to identify single orthologs across the species and the single orthologs were used to construct species phylogenetic tree. Whole-genome duplication data were downloaded from the Plant Genome Duplication Database and duplicated segment pairs ≥2 Mb were illustrated as background events in Circos. Information for the previously-identified QTLs in the last decades (1992-2018) were retrieved from the SoyBase, including those associated with flowering time and maturity, seed composition traits (such as oil, protein content, fatty acids, and amino acids), development (such as plant height, lodging, pubescence density, root length, branching, canopy height, leaflet length, yield-related traits (seed set, seed weight, seed yield), as well as responses to abiotic and biotic stressors (such as Phytophthora sojae, Spodoptera litura, Helicoverpa zea, Fusarium solani f. sp. glycines infection, Sclerotinia sclerotiorum, Heterodera glycines; drought, flooding). The genomic intervals of the QTLs, traits, flanking markers, authors, and publication titles were included in Table 15. QTL intervals greater than 10 Mb were not included in the analysis.

### D. Fast-Neutron Mutant Identification

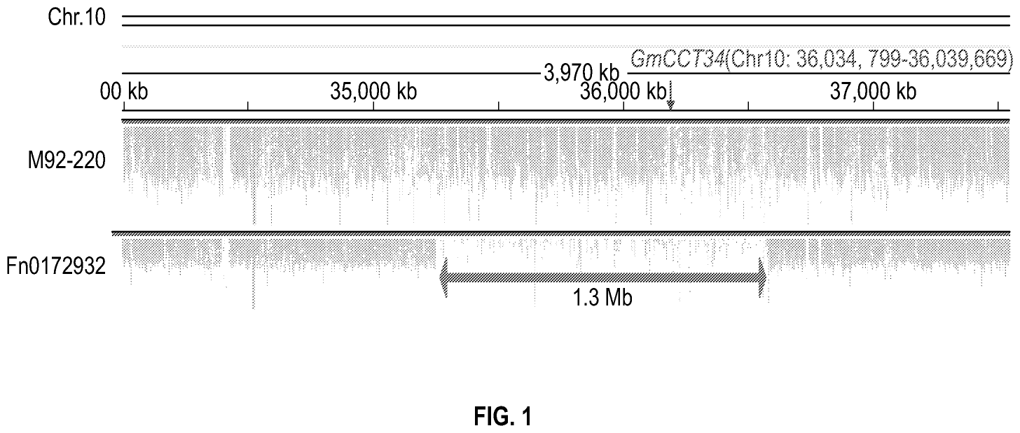

Fast neutron (FN) mutant line FN0172932 was selected from the M2 generation of irradiated elite line M92-220 in 2007 and further planted for homozygous mutants (FIG. 1). FN-induced genomic deletion region in M4-generation FN0172932 was previously determined using Comparative Genomic Hybridization (CGH) and further validated by whole genome sequencing (Illumine NovaSeq PE150, depth of coverage=16). The 1.3-Mb deletion region contains 52 gene models (Glyma. Wm82.a2) Plants were grown in the environment-controlled greenhouse in the Donald Danforth Plant Science Center with regular management (day 25° C./night 22° C., 40% humidity, 16 h/8 h day length for light/dark). Seed protein and oil content were measured on a pre-calibrated Perten DA 7250 analyzer (Perten Instruments, Inc., Springfield, IL, USA). Table 16 below provides details of POWR CCT-subfamily genes and their knockout and overexpression mutants. Table 17 provided field performance details about the POWR1 (CCT-subfamily gene) overexpression mutants.

TABLE 17
Details of POWR CCT-Subfamily Genes and
Their Knockout and Overexpression Mutants

| | | | | Oil |
|---|---|---|---|---|
| | | | CRISPR-MEDIATED | Phenotype |
| Sr. | | Gene | MUTATION | (% |
| No. | Gene ID | Name | COMBINATIONS | change) |
| 1 | Glyma.20G085100 | POWR1 | POWR1, 2, 3, 4 | ~3.78% ↑ |
| | | | POWR1 OVER- | ~1.34% ↓ |
| | | | EXPRESSION | |
| 2 | Glyma.10G134400 | POWR2 | POWR1, 2, 3 | ~3.91% ↑ |
| | | | POWR2, 3 | ~3.44% ↑ |
| | | | POWR2 | ~0.69% ↑ |
| 3 | Glyma.10G190300 | POWR3 | POWR1, 3 | ~3.84% ↑ |
| 4 | Glyma.20G200400 | POWR4 | POWR 1, 2, 4 | ~4.29% ↑ |

Note:
Arrows (↑ and ↓) indicate the increase or decrease in oil content in the seeds of the mutants grown in the greenhouse condition.

TABLE 17
Field performance details about the POWR1
(CCT-subfamily gene) overexpression mutants

| | | | Oil | field performance | |
|---|---|---|---|---|---|
| Sr. | | | Phenotype | (preliminary data) | |
| No. | Gene ID | Line Description | (% change) | Yield | Maturity |
| 1 | Glyma.20G085100 | Ubi promoter driving | ~2.0% ↓ | high | Early |
| | (POWR1) | POWR1 expression | | | (~25 days) |
| | | transgenic line | | | |
| | | Native promoter | ~2.00% ↓ | high | Early |
| | | driving POWR1 | | | (~21 days) |
| | | expression transgenic | | | |
| | | line | | | |

### E. Generation of Gene Edited Soybean Lines

Three guide-RNA (gRNA) sequences specific to the exons of GmCCT34, one for exon 1, two for exon 2, were designed using the web tool CRISPOR. The gRNA sequences were synthesized and annealed to the CRISPR/Cas9 expression vector and transformed into soybean cv. Williams 82 by the Wisconsin Crop Innovation Center using an Agrobacterium-mediated transformation protocol. A pair of primers specific to the vector was used to confirm positive transformants via PCR amplification (Forward: CTGCTGTTGATGGAGGACTT SEQ ID NO: 22; Reverse: CTCCTGGAGAAGCAGAAGTT SEQ ID NO: 23). T1 seeds from 10 independent TO plants were obtained and further grown in the environment-controlled greenhouse in the Donald Danforth Plant Science Center with the same condition as earlier mentioned. Unifoliate leaves were sampled from T1 plants to confirm the gene editing via PCR amplification followed by restriction enzyme digestion (Bs/I). Editing-generated deletion was further confirmed using Sanger sequencing. T2 seeds from the two homologous cct34 mutants were used to measure the seed composition traits as mentioned above.

### F. Subcellular Localization Analyses

The assay was performed through transient expression in Nicotiana benthamiana following a known method. The full-length CCT34 coding sequence (CDS), CCT34 lacking the CCT domain, and the CCT domain only were subcloned into the expression vector to generate UBQ10: YFP-CCT34, UBQ10: YFP-CCT34ACCT, and UBQ10: YFP-CCT, respectively. UBQ10: YFP was used as the empty vector. The vectors were individually transformed into Agrobacterium tumefaciens, and cultures of each construct were infiltrated into young leaves of N. benthamiana plants (4˜6 weeks) using a 3-ml syringe without the needle. Leaves were imaged 48 h after infiltration. Imaging was carried out a Leica TCS SP8 confocal microscope using the 63× water immersion lens. Samples were excited with a 514-nm laser line and 649-nm laser line to detect YFP and chlorophyll signals, respectively. Fluorescence emission was collected for best signals of indicated fluorescent probes. This experiment was repeated twice.

### G. Arabidopsis Mutant Analysis

Two independent T-DNA insertions mutant lines (WiscDsLox297300_13A.1 (cct1) and SALK_036731.1 (cct2)) were obtained from ARBC (Arabidopsis Biological Resource Center). These two T-DNA insertion regions lie with different sites of the 3′end of the CDS of AT1G04500 (FIG. 29), the closest homolog of soybean GmCCT67 (POWR1) and GmCCT34 (POWR2). The homozygous mutants were identified by PCR with specific primer sets listed in Table 18.

TABLE 18
Primers used

| T-DNA mutant | Left Primer | Right Primer |
|---|---|---|
| line ID | (5′-3′) | (5′-3′) |
| SALK_036731.1 | ATTTATCCCGAAT | TACCGGAGATGTG |
| | GGTAACGC | TTTGAAGG |
| | SEQ ID NO: 37 | SEQ ID NO: 38 |
| WiscDsLox297300_ | AACGTGAAACGGA | AAATTTGGTGAAA |
| 13A.1 | TTTGTCAG | CCAAGACC |
| | SEQ ID NO: 39 | SEQ ID NO: 40 |

| | |
|---|---|
| T-DNA specific | 5′-ATTTTGCCGATTTCGGAAC-3 |
| Border | SEQ ID NO: 41 |
| Primer-LBb1.3 | |

### Results

### A. Identification of CCT Proteins in Soybean and Other Species

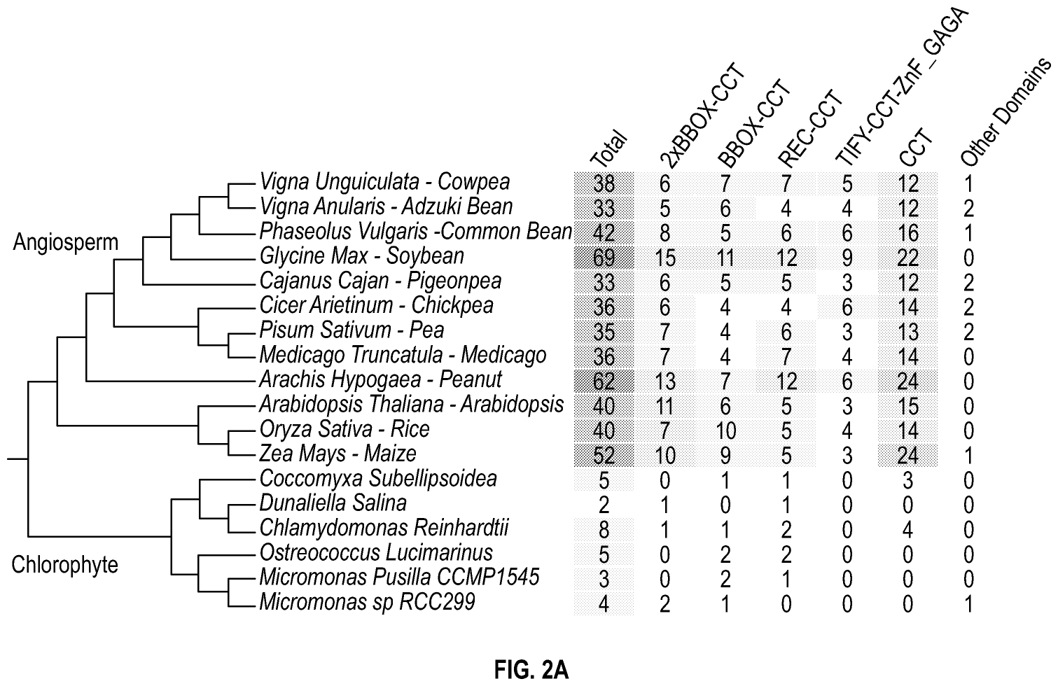

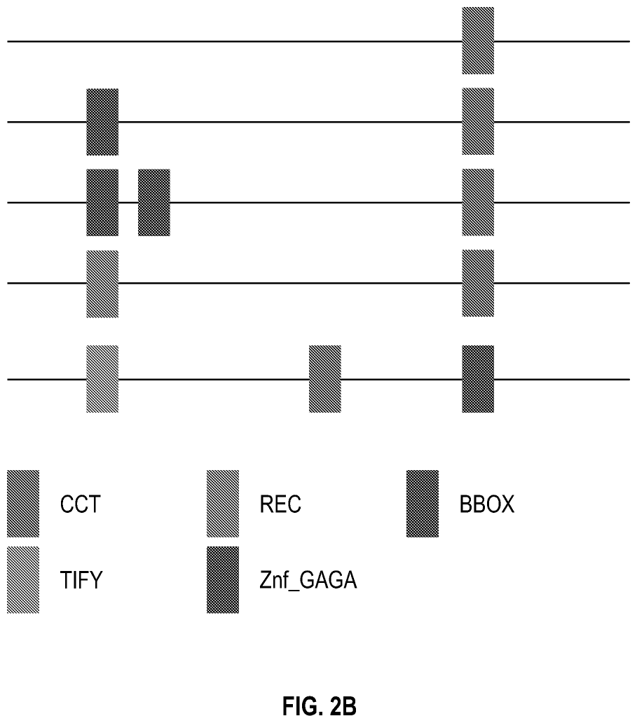

The CCT domain is a highly conserved basic module with ˜43 amino acids at the protein's C-terminus. The Hidden Markov Model (HMM) and the CCT domain (Pfam ID-PF06203) were used to search for the CCT proteins in selected plant species covering all members of the plant kingdom, including algae, mosses, ferns, conifers, and flowering plants. A set of 543 CCTs across the 24 plant species were identified (Table 2), including 69 soybean CCT domain-containing proteins (FIG. 2A, Table 1) and a range from 33 to 62 in other legumes, 40 and 52 CCT proteins, respectively, in the cereal crops rice and maize, and 13 to 29 in non-angiosperm land plants. (FIG. 2A). Traditionally, CCT proteins are classified into three subfamilies according to their constituent domains: single CCT (CCT Motif Family (CMF)), 1-2×BBOX-CCT (CONSTANTlike (COL) Family), and REC-CCT (Pseudo-Response Regulator Family). The present disclosure identified an additional protein group that carries the CCT domain, TIFY-CCT-ZnF_GATA. In these proteins, the CCT domain was located between two different domains, TIFY and ZnF_GATA. It is irrational to exclude the possibility that the CCT domain is involved in the function. Therefore, TIFY-CCT-ZnF_GATA was included in the analysis (FIG. 2B). The numbers of CCT protein genes in the tetraploids soybean and peanut were nearly doubled those in other diploid legumes. The CCT genes identified in Arabidopsis and the two cereal crops were generally more than those in legumes except for common bean and peanut. A small number of CCT genes (2-8) were present in chlorophyte species.

### B. Conservation in Domain Composition and Organization

To better understand the characteristics of CCT domain-containing proteins in plant species, the inventors analyzed domain features in sequences. According to constituent domains, CCT proteins could be classified into four subfamilies including single CCT, 1-2×BBOX-CCT, REC-CCT, TIFY-CCT-ZnF_GATA (FIG. 2B). All four subfamilies can be identified in higher plant species, suggesting a highly conserved domain architecture of CCT proteins among higher species (FIG. 2B). In contrast, only one or two of the four subfamilies were identified in the chlorophyte species that contain significantly fewer CCTs. Other than these canonical domains, CCT proteins carrying non-canonical domains were also identified, such as DUF740-DUF740-CCT in Vang06g17920 from adzuki bean, Adaptin_N-CCT in Psat0s3732g0120 from pea, S_TKc-CCT in Ca. 14621 from chickpea. Non-typical CCT proteins were not identified in soybean and model plants Arabidopsis. All identified CCT genes in this study were summarized in Table 2.

The inventors observed that the total numbers of CCT genes in soybean and peanut are approximately 2-3 times of those in other legumes or higher species, and so do for the subfamily. For example, the soybean genome contains 22 single-CCT proteins, which is more than those in legumes (12-16), Arabidopsis (15), and rice (14) (FIG. 2A). Interestingly, four subfamily members per species are generally in proportion across the higher plant species, approximately 2:1:1:2 for 1-2×BBOX-CCT:REC-CCT:TIFY-CCT-Zn_GAGA:single CCT (FIG. 2B). Differing from the three families containing the CCT domain in the C terminus, TIFY-CCT-ZnF_GATA subfamily contains the CCT in the middle of the sequences.

### C. Evolution and Expansion of CCT Family in Legumes

To gain insight into the evolution of CCT proteins in soybean and legumes, individual phylogenetic trees using the CCT proteins from each species were constructed. It was observed that the majority of the CCT proteins in soybean (68 of 69, 98.6%) and peanut (60 of 62, 96.8%) tree were clustered in pairs, leaving 1-2 unpaired CCT proteins (1.45-3.23%). In contrast, legumes and two cereal species have unpaired CCTs ranging from 6-12, representing 15.0-33.3% of total CCT members. A phylogenetic tree encompassing all investigated CCT proteins in plants was next re-reconstructed. Phylogeny analysis indicated that nearly every CCT family member from legumes corresponds to or evolutionarily closes to a pair of CCT paralogs from soybean or peanut, with each set of orthologous proteins forming individual ortholog-based subclades. These results and approximate ratio of 2 in the numbers of CCTs from soybean and peanut relative to those in other legumes suggested that whole-genome duplication (WGD) was likely the cause of the striking increase in the number of CCTs in soybean or peanut. The possible synteny in genomic regions harboring GmCCT genes were further examined. The 69 GmCCTs were mapped to all 20 chromosomes and the majority were distributed in the distal telomeric regions. Chromosome 13 contains the maximum number of GmCCTs (7) followed by chromosomes 4, 6, and 8 each having 6 members. It was striking that 33 pairs of GmCCTs (66 of 69, 95.7%) were located within syntenic genomic regions. This result and high bootstrap values for the GmCCT pairs in the soybean phylogenetic tree collectively suggested that the paired GmCCTs were paralogs that have been retained from large-scale duplication events such as whole-genome duplication or segmental duplication. This notion should also be applicable to peanut because of the segmental allotetraploid in the peanut genome. In addition, two identified pairs of tandemly duplicated GmCCTs in the soybean genome (GmCCT9/10; GmCCT18/19) also fell within segmental duplicated regions between chromosomes 4 and 6, suggesting that the tandem duplication occurred prior to the soybean-specific WGD. These results well demonstrated that polyploidization, especially the lineage-specific tetraploid in soybean and peanut, was a major evolutionary driven force of CCT expansion.

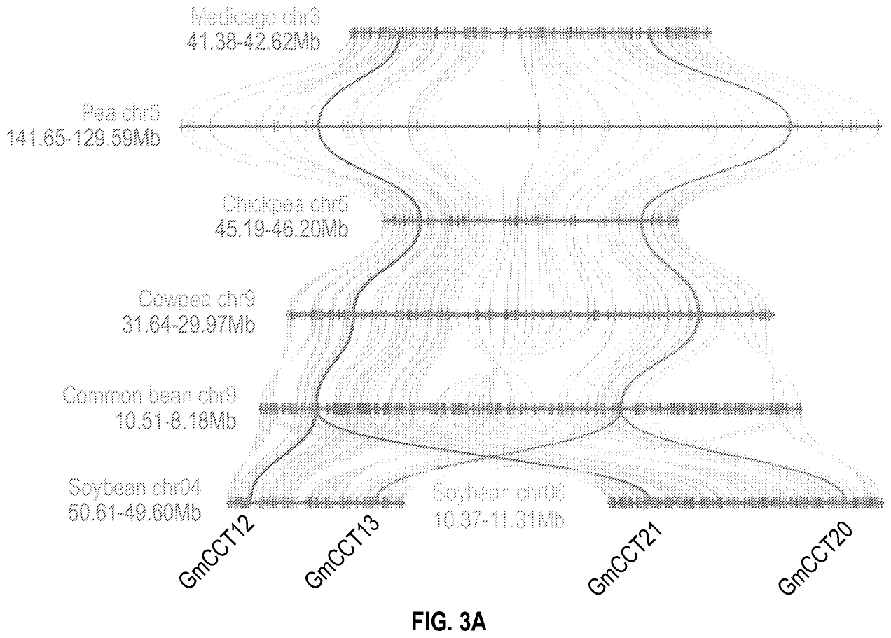

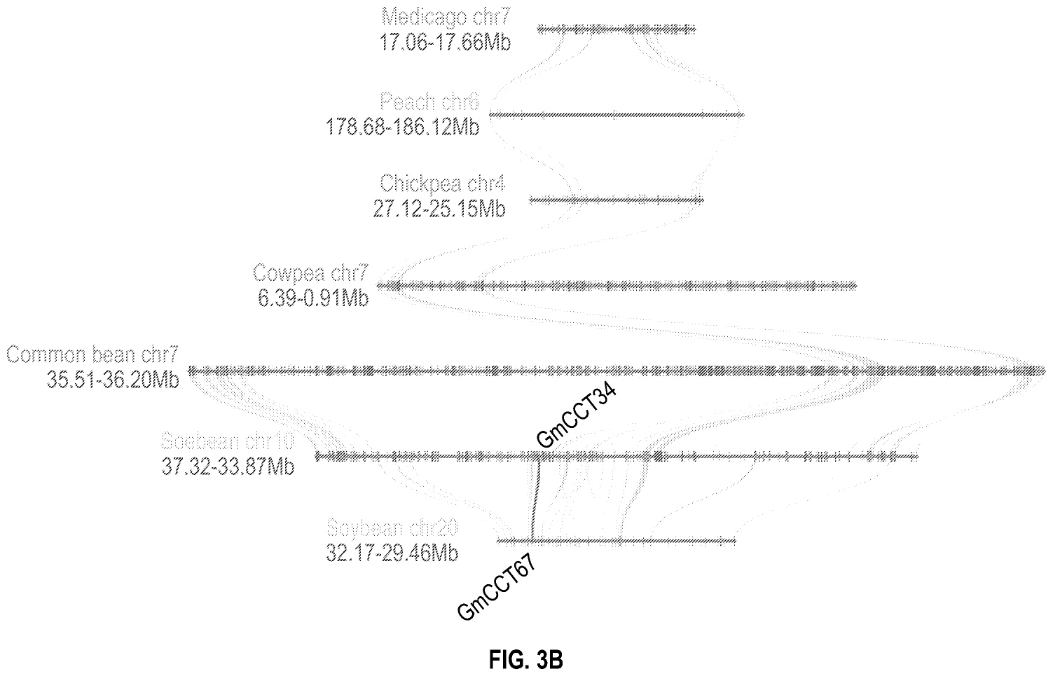

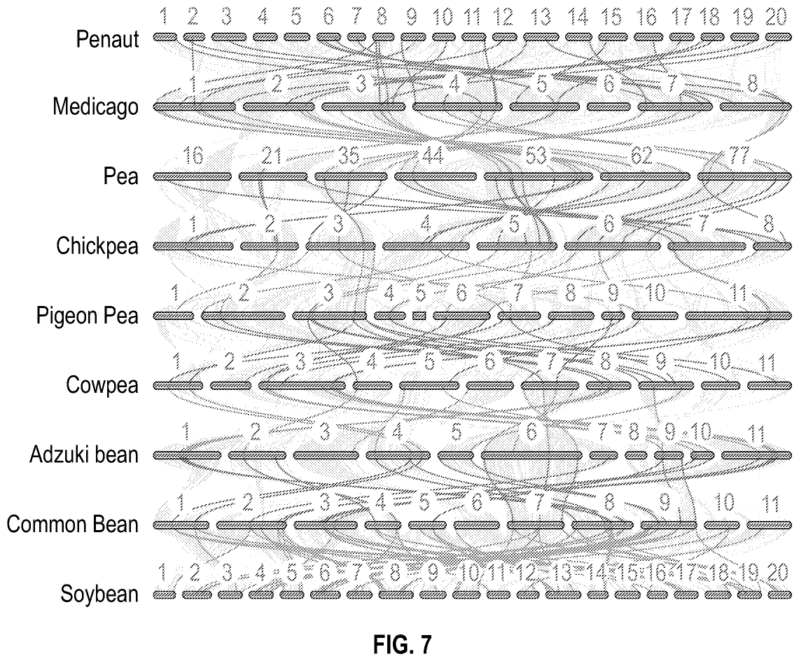

To understand the evolution of CCT proteins in related legume species, the syntenic CCT-associated genes and genomic regions were analyzed among selected closely related legume species, including Medicago, pea, chickpea, cowpea, common bean, and soybean. The syntenic analysis among leguminous CCTs revealed that 58 (84%) of the GmCCTs have at least one syntenic CCTs in legume genomes (Table 19; FIG. 7). For most legume CCT proteins, each corresponds to a pair of GmCCT paralogs, such as paralogs GmCCT12/21 in the syntenic regions of single CCT orthologous genes in five legumes (common bean, cowpea, chickpea, pea, Medicago) (FIG. 3A; Table 19; FIG. 7). This analysis also led to the identification of soybean-specific GmCCT without syntenic CCT homologs in other legumes, such as the pair of GmCCT34/67 (FIG. 3B; Table 19).

TABLE 19
List of legume CCTs syntenic with GmCCTs

| Glycine | | | | | | |
|---|---|---|---|---|---|---|
| Max CCT | | | chickpe Ca | | | |
| gene IDs | G. max | Adzukibean | CDC | Commonbean | Cowpea | Medicago |
| GmCCT01 | Glyma.01G221100.1 | Vang07g06400.1 | Ca_02437.1 | Phvul.002G158500.1 | Vigun02g177100.1 | Medtr5g010120.1 |
| GmCCT02 | Glyma.02G152900.1 | Vang06g17920.2 | . | Phvul.007G166100.1 | Vigun07g116500.2 | . |
| GmCCT03 | Glyma.02G215600.1 | Vang01g09310.1 | . | Phvul.008G182900.1 | Vigun08g109700.1 | Medtr5g066510.1 |
| GmCCT04 | Glyma.02G223700.1 | . | Ca_14236.1 | Phvul.008G190200.1 | . | Medtr5g069480.5 |
| GmCCT05 | Glyma.02G230800.1 | Vang01g08550.1 | Ca_17243.1 | Phvul.008G257300.1 | Vigun08g124000.1 | Medtr5g072780.1 |
| GmCCT06 | Glyma.03G209800.1 | Vang04g15980.1 | Ca_00927.1 | Phvul.001G203000.1 | Vigun01g187100.1 | Medtr7g108150.1 |
| GmCCT07 | Glyma.03G261300.1 | Vang09g08740.1 | Ca_01359.1 | Phvul.009G258300.1 | Vigun09g004700.1 | Medtr7g118260.2 |
| GmCCT08 | Glyma.04G058900.1 | Vang05g09750.1 | Ca_01694.1 | Phvul.009G085300.1 | Vigun09g217500.1 | Medtr3g105710.1 |
| GmCCT09 | Glyma.04G096200.1 | Vang0017s00340.2 | Ca_01471.1 | Phvul.009G035400.1 | Vigun09g180900.1 | Medtr3g100050.1 |
| GmCCT10 | Glyma.04G096300.1 | Vang0017s00360.1 | Ca_01470.1 | Phvul.009G035300.1 | Vigun09g180800.1 | Medtr3g100040.1 |
| GmCCT11 | Glyma.04G166300.1 | Vang09g02620.1 | Ca_18243.1 | Phvul.009G185500.1 | Vigun09g076300.1 | Medtr3g037390.1 |
| GmCCT12 | Glyma.04G228300.1 | Vang0084s00090.1 | Ca_04056.1 | Phvul.009G045000.1 | Vigun09g140300.1 | Medtr3g092780.2 |
| GmCCT13 | Glyma.04G236400.1 | Vang0090s00480.2 | Ca_03974.1 | Phvul.009G053300.2 | Vigun09g150200.1 | Medtr3g091340.1 |
| GmCCT14 | Glyma.05G025000.1 | Vang11g11310.1 | Ca_13165.1 | Phvul.003G188400.1 | Vigun03g337800.1 | Medtr4g108880.1 |
| GmCCT16 | Glyma.05G209800.1 | Vang10g06540.1 | Ca_05769.1 | Phvul.002G291600.2 | Vigun03g042600.1 | Medtr8g098725.1 |
| GmCCT15 | Glyma.05G233700.1 | Vang10g01650.1 | . | Phvul.002G316100.1 | Vigun03g017700.1 | Medtr8g104190.1 |
| GmCCT17 | Glyma.06G059600.1 | Vang05g09750.1 | Ca_01694.1 | Phvul.009G085300.1 | Vigun09g217500.1 | Medtr3g105710.1 |
| GmCCT18 | Glyma.06G097900.1 | Vang0017s00340.2 | Ca_01471.1 | Phvul.009G035400.1 | Vigun09g180900.1 | Medtr3g100050.1 |
| GmCCT19 | Glyma.06G098000.1 | Vang0017s00360.1 | Ca_01470.1 | Phvul.009G035300.1 | Vigun09g180800.1 | Medtr3g100040.1 |
| GmCCT20 | Glyma.06G127900.1 | Vang0090s00480.2 | Ca_03974.1 | Phvul.009G053300.2 | Vigun09g150200.1 | Medtr3g091340.1 |
| GmCCT21 | Glyma.06G136600.1 | Vang0084s00090.1 | Ca_04056.1 | Phvul.009G045000.1 | Vigun09g140300.1 | Medtr3g092780.2 |
| GmCCT22 | Glyma.06G196200.1 | Vang09g02620.1 | Ca_18243.1 | Phvul.009G185500.1 | Vigun09g076300.1 | Medtr3g037390.1 |
| GmCCT23 | Glyma.07G049400.1 | Vang0238s00030.1 | . | Phvul.010G119700.2 | Vigun10g152100.2 | Medtr8g024260.1 |
| GmCCT24 | Glyma.07G091400.1 | Vang02g04910.1 | . | Phvul.004G157300.1 | Vigun04g021700.1 | . |
| GmCCT25 | Glyma.07G182700.1 | Vang0064ss00450.1 | Ca_05292.1 | Phvul.002G213800.2 | Vigun03g124600.1 | Medtr4g093730.1 |
| GmCCT26 | Glyma.08G016500.1 | Vang10g06540.1 | Ca_05769.1 | Phvul.002G291600.2 | Vigun03g042600.1 | Medtr8g098725.1 |
| GmCCT27 | Glyma.08G041100.1 | Vang10g01650.1 | Ca_22732.1 | Phvul.002G316100.1 | Vigun03g017700.1 | Medtr8g104190.1 |
| GmCCT28 | Glyma.08G067200.1 | Vang0064ss00450.1 | Ca_05292.1 | Phvul.002G213800.2 | Vigun03g124600.1 | Medtr4g093730.1 |
| GmCCT29 | Glyma.08G168900.1 | . | Ca_13576.1 | . | . | Medtr2g068730.1 |
| GmCCT30 | Glyma.08G221800.1 | Vang01g08240.1 | Ca_17522.1 | Phvul.008G254600.1 | Vigun08g127200.1 | Medtr4g008090.1 |
| GmCCT31 | Glyma.08G255200.1 | Vang04g02640.1 | . | Phvul.008G022800.1 | Vigun05g024400.1 | Medtr7g018170.1 |
| GmCCT46 | Glyma.09G184600.1 | Vang02g04910.1 | . | Phvul.004G157300.1 | Vigun04g021700.1 | . |
| GmCCT32 | Glyma.10G021400.1 | Vang06g17920.2 | . | Phvul.007G166100.1 | Vigun07g116500.2 | . |
| GmCCT33 | Glyma.10G048100.1 | Vang0206s00130.1 | Ca_14041.1 | Phvul.007G162500.1 | Vigun07g078600.2 | Medtr1g067110.1 |
| GmCCT34 | Glyma.10G134400.1 | . | . | . | Vigun07g011700.1 | . |
| GmCCT35 | Glyma.10G190300.1 | Vang06g09620.1 | . | Phvul.L001944.1 | Vigun07g181300.1 | Medtr1g073350.1 |
| GmCCT36 | Glyma.10G274300.1 | Vang06g00840.1 | Ca_05487.1 | Phvul.007G027200.1 | Vigun07g270500.1 | Medtr1g110870.1 |
| GmCCT37 | Glyma.11G022600.1 | Vang07g06400.1 | Ca_02437.1 | Phvul.002G158500.1 | Vigun02g177100.1 | . |
| GmCCT39 | Glyma.12G074400.1 | Vang0010ss00750.1 | Ca_14630.1 | Phvul.011G069800.1 | Vigun11g150000.1 | Medtr4g061823.1 |
| GmCCT40 | Glyma.12G196100.1 | Vang09g03140.1 | Ca_14148.1 | Phvul.005G113300.1 | Vigun05g225000.4 | Medtr2g088900.1 |
| GmCCT44 | Glyma.13G009300.1 | Vang03g16900.1 | Ca_07641.1 | Phvul.006G005200.3 | . | Medtr3g082630.1 |
| GmCCT43 | Glyma.13G050300.1 | Vang02g13360.1 | . | Phvul.004G046601.1 | Vigun04g068400.1 | . |
| GmCCT42 | Glyma.13G093800.1 | Vang11g14640.1 | Ca_03275.1 | Phvul.003G149000.1 | Vigun03g379600.1 | Medtr4g128930.1 |
| GmCCT41 | Glyma.13G100700.1 | Vang11g15310.1 | Ca_03219.1 | Phvul.003G141200.1 | Vigun03g387700.1 | Medtr4g127420.1 |
| GmCCT45 | Glyma.13G135900.1 | Vang0206s00130.1 | Ca_14041.1 | Phvul.007G162500.1 | Vigun07g078600.1 | Medtr1g067110.1 |
| GmCCT47 | Glyma.13G306400.1 | Vang09g03140.1 | Ca_14148.1 | Phvul.005G113300.1 | Vigun05g225000.1 | Medtr2g088900.1 |
| GmCCT48 | Glyma.13G331000.1 | Vang0021ss01680.1 | Ca_02579.1 | Phvul.L002465.1 | Vigun05g253600.1 | Medtr2g096080.1 |
| GmCCT49 | Glyma.14G182800.1 | Vang01g09310.1 | Ca_14968.1 | Phvul.008G182900.1 | Vigun08g109700.1 | Medtr5g066510.1 |
| GmCCT50 | Glyma.14G190400.1 | Vang01g09960.2 | Ca_14236.1 | Phvul.008G190200.1 | Vigun08g116900.1 | Medtr5g069480.1 |
| GmCCT51 | Glyma.14G197900.1 | Vang01g08550.1 | Ca_17243.1 | . | . | Medtr5g072780.1 |
| GmCCT52 | Glyma.15G219800.1 | . | . | . | . | . |
| GmCCT53 | Glyma.15G258200.1 | Vang0022ss02670.1 | Ca_13576.1 | Phvul.005G049801.1 | Vigun01g054600.1 | Medtr2g068730.1 |
| GmCCT54 | Glyma.16G018000.1 | . | . | Phvul.010G119700.1 | Vigun10g152100.2 | Medtr8g024260.1 |
| GmCCT55 | Glyma.16G050900.1 | Vang04g13110.1 | Ca_05985.1 | Phvul.001G087300.1 | Vigun01g075800.1 | Medtr7g083540.1 |
| GmCCT56 | Glyma.16G067000.1 | . | . | . | Vigun01g065500.1 | Medtr7g032240.1 |
| GmCCT57 | Glyma.17G059100.1 | Vang11g15310.1 | Ca_03219.1 | Phvul.003G141200.2 | Vigun03g387700.1 | Medtr4g127420.1 |
| GmCCT58 | Glyma.17G066600.1 | Vang11g14640.1 | Ca_03275.1 | Phvul.003G149000.1 | Vigun03g379600.1 | Medtr4g128930.1 |
| GmCCT59 | Glyma.17G102200.1 | Vang11g11310.1 | Ca_13165.1 | Phvul.003G188400.1 | Vigun03g337800.1 | Medtr4g108880.1 |
| GmCCT60 | Glyma.18G278100.1 | Vang04g02640.1 | Ca_22148.1 | Phvul.008G022800.1 | Vigun05g024400.1 | Medtr7g018170.1 |
| GmCCT61 | Glyma.19G039000.1 | Vang02g13360.1 | . | Phvul.004G046601.1 | Vigun04g068400.1 | . |
| GmCCT62 | Glyma.19G079600.1 | Vang04g12460.1 | . | Phvul.008G147400.1 | Vigun01g065500.1 | . |
| GmCCT63 | Glyma.19G099700.1 | Vang04g13110.1 | Ca_05985.1 | Phvul.001G087300.1 | Vigun01g075800.1 | Medtr7g083540.1 |
| GmCCT64 | Glyma.19G207100.1 | Vang04g15980.1 | Ca_00927.1 | Phvul.001G203000.1 | Vigun01g187100.1 | Medtr7g108150.1 |
| GmCCT65 | Glyma.19G260400.1 | Vang09g08740.1 | Ca_01359.1 | Phvul.009G258300.1 | Vigun09g004700.1 | Medtr7g118260.1 |
| GmCCT66 | Glyma.20G060400.1 | . | Ca_07641.1 | . | . | Medtr3g082630.1 |
| GmCCT67 | Glyma.20G085100.1 | Vang06g25290.1 | Ca_20458.1 | Phvul.007G270600.1 | Vigun07g011700.1 | . |
| GmCCT68 | Glyma.20G115600.1 | Vang06g00840.1 | Ca_05487.1 | Phvul.007G027200.1 | Vigun07g270500.1 | Medtr1g110870.1 |
| GmCCT69 | Glyma.20G200400.1 | Vang06g09620.1 | . | Phvul.L001944.1 | Vigun07g181300.1 | Medtr1g073350.1 |
| GmCCT38 | Glyma.U033700.1 | Vang0010ss00750.1 | Ca_14630.1 | Phvul.011G069800.1 | Vigun11g150000.1 | Medtr4g061823.1 |

| Glycine | | | | | |
|---|---|---|---|---|---|
| Max CCT | | | | | |
| gene IDs | Pea | Peanut | Pigeon | G. max | Arabidopsis |
| GmCCT01 | Psat2g184400.1 | arahy.B2EAID.1 | C.cajan_13216.1 | Glyma.11G022600.1 | . |
| GmCCT02 | . | . | C.cajan_10458.1 | Glyma.10G021400.1 | AT2G47890.1 |
| GmCCT03 | . | . | . | Glyma.14G182800.1 | . |
| GmCCT04 | . | arahy.TFJ3CW.1 | C.cajan_29262.1 | Glyma.14G190400.1 | . |
| GmCCT05 | Psat2g054040.2 | arahy.XNQT3T.1 | C.cajan_05381.1 | Glyma.14G197900.1 | . |
| GmCCT06 | Psat3g024360.1 | arahy.7A5VUD.1 | C.cajan_10578.1 | Glyma.19G207100.1 | . |
| GmCCT07 | . | arahy.Y6ILE8.1 | C.cajan_10962.1 | Glyma.19G260400.1 | . |
| GmCCT08 | . | arahy.ZS9VWH.1 | C.cajan_32168.1 | Glyma.06G059600.1 | . |
| GmCCT09 | . | . | C.cajan_17917.1 | Glyma.06G097900.1 | . |
| GmCCT10 | . | . | C.cajan_17916.1 | Glyma.06G098000.1 | . |
| GmCCT11 | Psat5g199360.1 | . | . | Glyma.06G196200.1 | . |
| GmCCT12 | Psat5g071920.1 | arahy.A9G6ZW.1 | . | Glyma.06G136600.1 | AT5G24470.1 |
| GmCCT13 | Psat5g077440.1 | arahy.BVW291.1 | C.cajan_08842.1 | Glyma.06G127900.1 | AT5G53420.1 |
| GmCCT14 | Psat4g049800.1 | arahy.2F1UPX.2 | . | Glyma.17G102200.1 | AT5G61380.1 |
| GmCCT16 | Psat7g031600.1 | arahy.BU1BMX.1 | C.cajan_11568.1 | Glyma.08G016500.1 | AT5G41380.1 |
| GmCCT15 | Psat7g011960.1 | . | C.cajan_15626.1 | Glyma.08G041100.1 | . |
| GmCCT17 | . | . | C.cajan_32168.1 | Glyma.04G058900.1 | . |
| GmCCT18 | . | arahy.0U49HS.1 | C.cajan_17917.1 | Glyma.04G096200.1 | . |
| GmCCT19 | . | . | C.cajan_17916.1 | Glyma.04G096300.1 | . |
| GmCCT20 | Psat5g077440.1 | arahy.5WR99K.1 | C.cajan_08842.1 | Glyma.04G236400.1 | AT5G53420.1 |
| GmCCT21 | Psat5g071920.1 | arahy.A9G6ZW.1 | . | Glyma.04G228300.1 | . |
| GmCCT22 | Psat5g199360.1 | . | C.cajan_08715.1 | Glyma.04G166300.1 | . |
| GmCCT23 | Psat4g164080.1 | . | C.cajan_39868.1 | Glyma.16G018000.1 | . |
| GmCCT24 | . | arahy.QP34EV.1 | C.cajan_08639.1 | Glyma.09G184600.3 | . |
| GmCCT25 | Psat7g101680.1 | . | C.cajan_22947.1 | Glyma.08G067200.1 | . |
| GmCCT26 | Psat7g031600.1 | arahy.AGT1RS.1 | C.cajan_11568.1 | Glyma.05G209800.1 | . |
| GmCCT27 | Psat7g011960.1 | arahy.8MV22J.1 | C.cajan_15626.1 | Glyma.05G233700.1 | . |
| GmCCT28 | Psat7g101680.1 | . | C.cajan_22947.1 | Glyma.07G182700.1 | . |
| GmCCT29 | . | . | . | Glyma.15G258200.1 | . |
| GmCCT30 | Psat7g260880.1 | arahy.QG9XFC.1 | C.cajan_32117.1 | . | AT1G51600.1 |
| GmCCT31 | Psat3g193520.1 | arahy.S3IANQ.1 | C.cajan_20131.1 | Glyma.18G278100.1 | . |
| GmCCT46 | . | arahy.WEZ503.1 | C.cajan_08639.1 | Glyma.07G091400.1 | . |
| GmCCT32 | . | . | C.cajan_10458.1 | Glyma.02G152900.1 | . |
| GmCCT33 | Psat6g124840.1 | . | C.cajan_41432.1 | Glyma.13G135900.1 | . |
| GmCCT34 | . | arahy.22FLX8.1 | C.cajan_02757.1 | Glyma.10G134400.1 | . |
| GmCCT35 | . | arahy.EF48U6.1 | C.cajan_22520.1 | Glyma.20G200400.2 | . |
| GmCCT36 | Psat6g228360.1 | arahy.WVH2H8.1 | C.cajan_31872.1 | Glyma.20G115600.1 | AT1G68520.1 |
| GmCCT37 | . | . | C.cajan_13216.1 | Glyma.01G221100.1 | . |
| GmCCT39 | Psat7g221360.1 | arahy.IR754M.1 | C.cajan_21434.1 | Glyma.U033700.1 | . |
| GmCCT40 | Psat5g265480.1 | . | C.cajan_36557.1 | Glyma.13G306400.4 | . |
| GmCCT44 | Psat5g105240.1 | . | . | Glyma.13G009300.1 | . |
| GmCCT43 | . | arahy.LV381L.1 | C.cajan_26721.1 | Glyma.19G039000.1 | . |
| GmCCT42 | . | . | C.cajan_03622.1 | Glyma.17G066600.1 | AT5G57660.1 |
| GmCCT41 | Psat4g013600.2 | arahy.0QJD35.1 | C.cajan_03709.1 | Glyma.17G059100.1 | . |
| GmCCT45 | Psat6g124840.1 | . | . | Glyma.10G048100.1 | AT5G02810.1 |
| GmCCT47 | Psat5g265480.1 | . | C.cajan_36557.1 | Glyma.12G196100.1 | . |
| GmCCT48 | Psat5g282000.1 | arahy.KU7VJQ.1 | C.cajan_13596.1 | . | . |
| GmCCT49 | . | . | . | Glyma.02G215600.1 | AT4G24470.1 |
| GmCCT50 | Psat2g060680.2 | arahy.NK4UTA.1 | C.cajan_29262.1 | Glyma.02G223700.1 | . |
| GmCCT51 | Psat2g054040.1 | arahy.XNQT3T.1 | C.cajan_05381.1 | Glyma.02G230800.2 | AT1G07050.1 |
| GmCCT52 | . | . | . | Glyma.15G219800.1 | . |
| GmCCT53 | . | arahy.9Y5DYJ.1 | . | Glyma.08G168900.1 | . |
| GmCCT54 | . | arahy.PEG0RN.1 | . | Glyma.07G049400.1 | . |
| GmCCT55 | Psat3g094880.1 | arahy.P8XRWE.1 | C.cajan_17635.1 | Glyma.19G099700.1 | . |
| GmCCT56 | . | arahy.FCSG49.1 | C.cajan_00921.1 | . | AT5G14370.1 |
| GmCCT57 | . | arahy.0QJD35.1 | C.cajan_03709.1 | Glyma.13G100700.1 | AT5G57180.2 |
| GmCCT58 | . | arahy.6J3MKV.1 | C.cajan_03622.1 | Glyma.13G093800.1 | AT5G57660.1 |
| GmCCT59 | Psat4g049800.1 | arahy.D90B1M.1 | . | Glyma.05G025000.1 | AT5G61380.1 |
| GmCCT60 | Psat3g193520.1 | arahy.669CET.1 | C.cajan_20131.1 | Glyma.08G255200.1 | . |
| GmCCT61 | . | . | C.cajan_26721.1 | Glyma.13G050300.1 | . |
| GmCCT62 | . | arahy.857KFG.1 | . | . | . |
| GmCCT63 | . | arahy.P8XRWE.1 | C.cajan_17635.1 | Glyma.16G050900.1 | . |
| GmCCT64 | Psat3g024360.1 | arahy.7A5VUD.1 | C.cajan_10578.1 | Glyma.03G209800.1 | AT2G47890.1 |
| GmCCT65 | . | . | C.cajan_10962.1 | Glyma.03G261300.1 | AT2G46790.1 |
| GmCCT66 | Psat5g105240.1 | . | . | . | . |
| GmCCT67 | . | . | C.cajan_02757.1 | Glyma.10G134400.1 | . |
| GmCCT68 | Psat6g228360.1 | arahy.WVH2H8.1 | C.cajan_31872.1 | Glyma.10G274300.1 | . |
| GmCCT69 | Psat6g131680.1 | arahy.EF48U6.1 | C.cajan_22520.1 | Glyma.10G190300.1 | AT2G33350.1 |
| GmCCT38 | Psat7g221360.1 | arahy.IR754M.1 | C.cajan_21434.1 | Glyma.12G074400.1 | . |

D. Functional Insights into GmCCT Proteins

Given the conservation in CCT domain architecture and protein sequences within the clusters, a phylogenetic tree was reconstructed using the complete CCT proteins from four species (soybean, Arabidopsis, rice, maize) to infer functions since many CCT proteins from the latter three species have been functionally characterized. The phylogeny of the four-species CCT tree agrees with the global tree and soybean trees mentioned earlier. it was defined into six clusters (I to VI) by tropology. CCT proteins with demonstrated roles in the regulation of flowering-related traits were clustered in different clusters, implying extensive roles of CCT proteins associated with flowering in soybean. These genes included ZmCCT and ZmCCT9, all five PRR proteins (PRR1, PRR 3, PRR 5, PRR 7, PRR 9) from Arabidopsis, two rice REC-CCT genes (Ghd7 and Ghd7.1), six COL family members (CO, COL1-5) associated with flowering time and shoot branching, Arabidopsis COL12 (BBX10) that associated with branching and flowering time and two COL9 (BBX7) with a role in flowering. Clustering of the CCT genes with different roles in some clusters inferred the implicit functions for the phylogenetically-clustered proteins, such as ASML2 involved in the induction of sugar-inducible genes and FITNESS and CIA associated with drought tolerance. This analysis indicated the function conservation and diversity of CCT families; whether the family possesses functions beyond those mentioned above merits more attention.

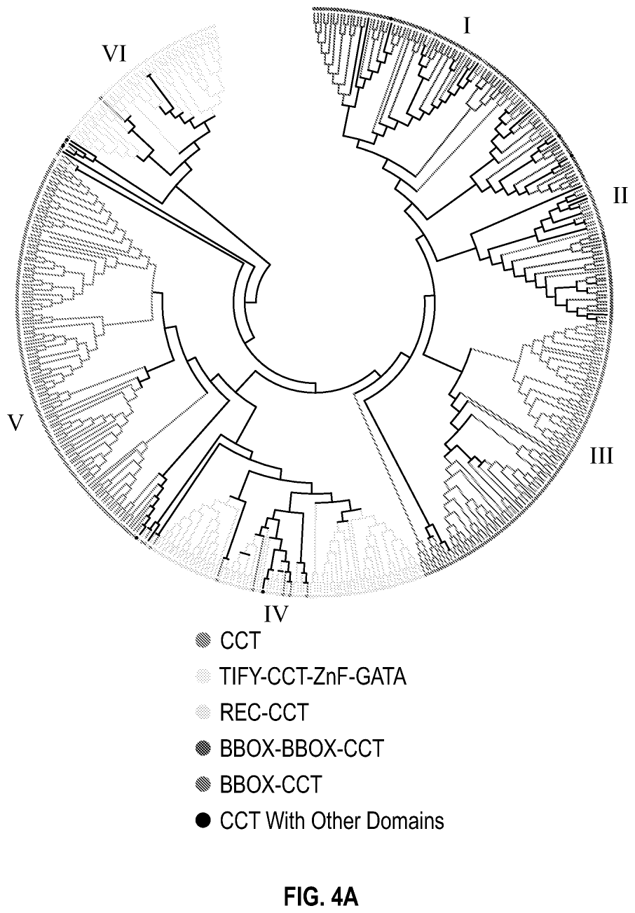

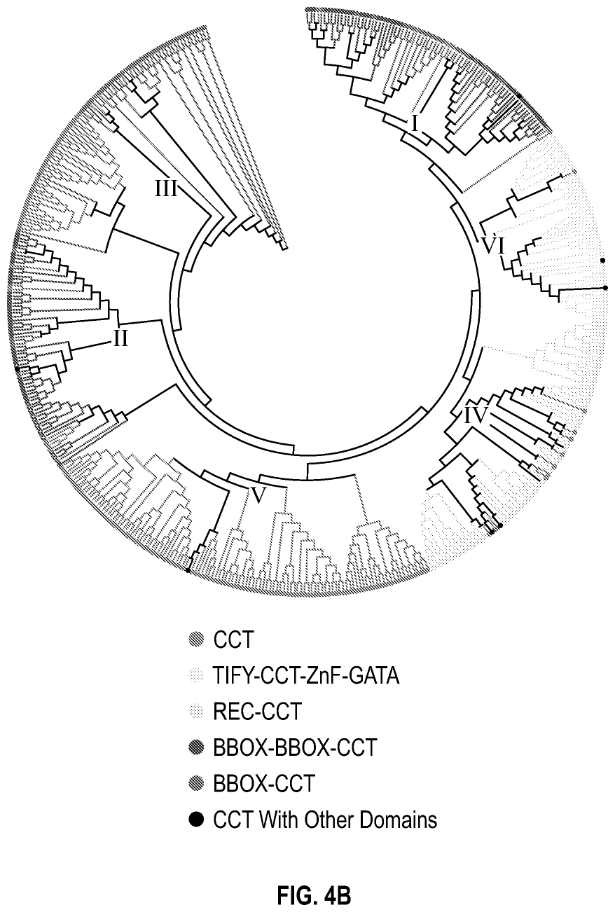

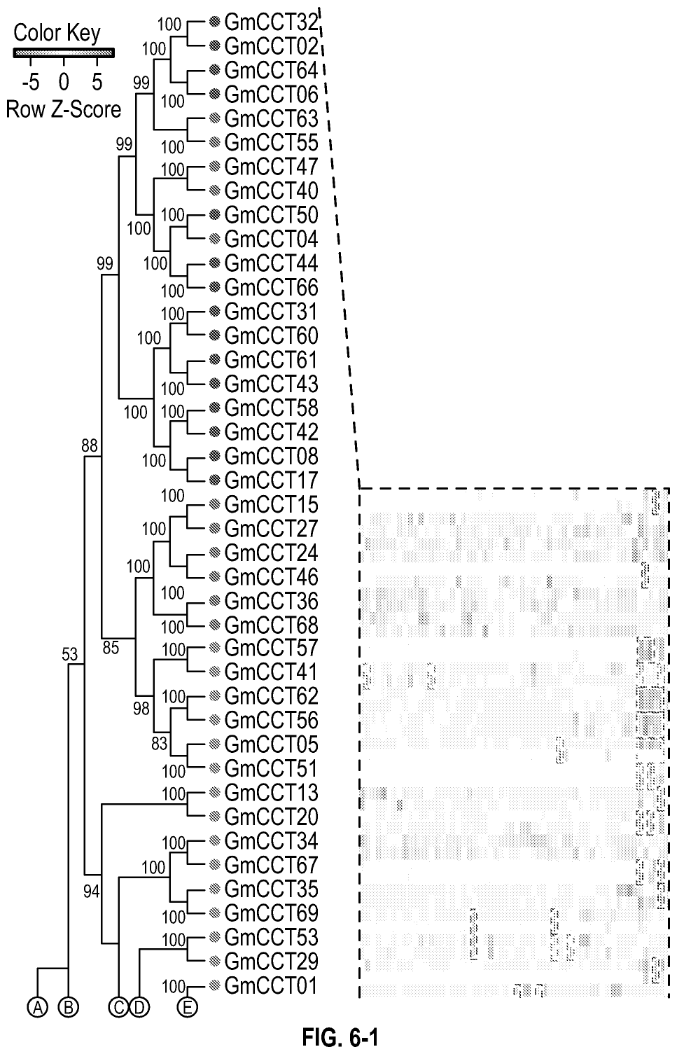

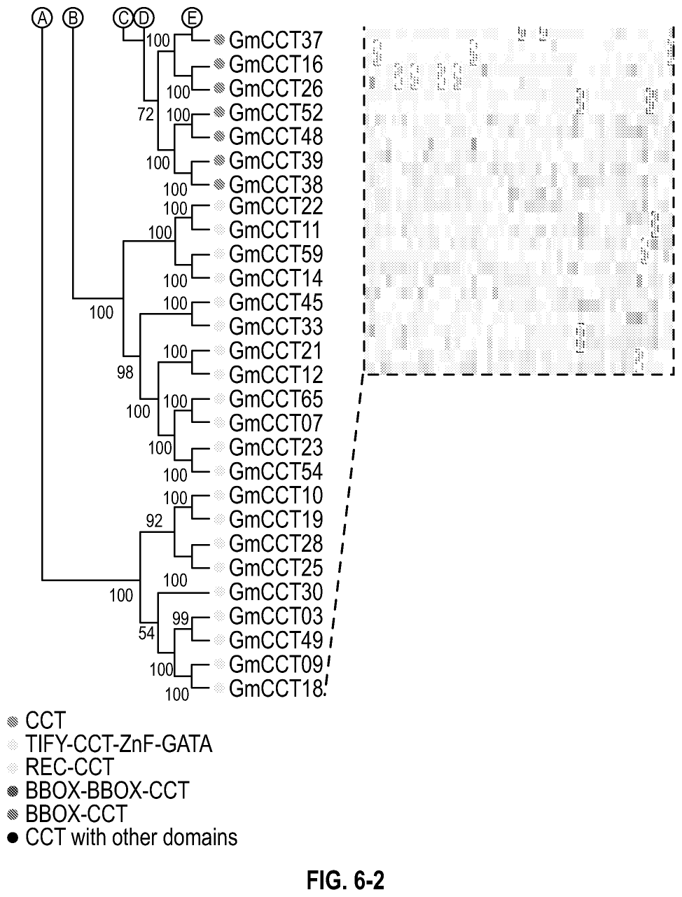

The above results suggested orthologous relationships for those closely clustered CCT genes. The syntenic relationship of the leguminous CCTs was investigated next. The syntenic analysis revealed that 58 (84%) of the GmCCTs had at least one syntenic CCTs in legume genomes (Table 3; FIG. 4A-4C). For most legume CCT proteins, each corresponded to a pair of GmCCT paralogs, such as paralogs GmCCT12/21 in the syntenic regions of single CCT orthologous genes in five legumes (common bean, cowpea, chick-pea, pea, Medicago) (FIG. 3B; Table 3; FIG. 6). The numbers of CCTs in legumes that were in synteny with GmCCTs varied greatly, such as over 90% for adzuki bean and common bean, 72.72-76.19% for cowpea, Medicago, and pigeon pea, and 22.58-45.71% for pea, chickpea, and peanut. This observation was consistent with the phylogeny of these legumes (FIG. 2) where soybean, adzuki bean, and common bean are phylogenetically close and all are relatively far from Medicago and pigeon pea and chickpea, and much further from peanut (Wang et al. 2017), reflecting that evolutions of the CCT proteins in the individual species complied to respective genome evolution histories after legume divergence. The syntenic CCT proteins across legumes were likely originated from common ancestral CCT proteins. This analysis also led to the identification of soybean-specific GmCCT without syntenic CCT homologs in legumes, such as the pair of GmCCT34/67 (FIG. 3B; Table 3).

### E. Conservation and Diversification of CCT Domain Sequence

Phylogeny analyses in individual CCT trees and the global CCT tree indicated that domain architecture-oriented topology where CCT proteins carrying the same constituent domains were closely clustered. It was observed that six BBOX-CCT (GmCCT15, 27, 24, 46, 36, 68) and single CCT (GmCCT57, 41, 62, 56, 05, 51) proteins were respectively grouped closely and formed a strayed cluster that was separated from the main clusters consisting of the majority of other BBOX-CCT and single CCT proteins. It appeared that the existence of strayed clusters comprising single-CCT with BBOX-CCT proteins was common for all other investigated higher plant species, although they varied in tropology. The phylogenetic relationship of the strayed members in plant species was determined. Intriguingly, in the global tree, those strayed single-CCT or BBOX-CCT proteins identified in the individual trees were clustered together and formed a large combined strayed cluster (FIG. 6). Within the large strayed cluster, single-CCT or BBOX-CCT proteins were separately clustered by monocots and dicots. These results suggested that these strayed CCT orthologs originated from common ancestral genes and have been retained following the divergence of rosids. Separation of those single-CCT or BBOX-CCT proteins from major clusters suggested possibly distinct roles from thus far known functions for most identified CCT genes such as photoperiod-associated flowering time control or yield-related traits. For example, BBX15/COL16 and FITNESS protein in this cluster were related to chlorophyll accumulation and H2O2-related defense and CIA2 involved in protein import into the chloroplast.

The global tree suggested the phylogenetic relationship of the entire CCT protein sequences, the phylogeny of CCT domains alone across the species was further investigated. Surprisingly, the tropology was highly congruent between the protein and domain trees, including the strayed clusters (cluster III) and singletons (such as black dots, red dots in clusters I, IV, VI) dispersed in non-self subfamilies (FIG. 4A and FIG. 4B). This observation suggested that the CCT domains from the same cluster of domain tree were likely originated from common ancestral CCT domains and then co-evolved with respective protein sequences while remained diversified among clusters (I-VI) or subfamilies. With this observation, it was hypothesized that those protein singletons (black or red dots) were likely derived from phylogenetically-close members from the same domain cluster via addition or loss of one or two domains. For example, the single-CCT proteins in cluster IV of the global tree were likely derived from the loss of REC domain in one of phylogenetically close REC-CCTs or common ancestral proteins. This analysis suggested phylogenetic diversification of CCT proteins in plant species which in part enriched CCT family diversity and explained the origin of a few CCT proteins.

Notably, single-CCT genes were found in all six clusters (FIG. 4A). In clusters I, II, IV, and VI, consist of only a few individual single-CCTs, likely representing recent deletions of the non-CCT domain in these genes. It is also likely that several 1×BBOX-CCTs in the two 2×BBOX-CCT clusters (I and II) likewise represent the deletion of a single BBOX domain. Cluster III, however, contains a large number of single-CCTs that form two clades in the domain phylogeny (FIG. 4B). These likely represent an ancient deletion of the BBOX domain in this clade prior to the origin of the angiosperms.

Interestingly, CCTs containing non-canonical domains were rare and dispersed across several clusters, likely representing singleton insertion events, for example, DUF740-CCT in Vang06g17920 (adzuki bean), Adaptin_N-CCT in Psat0s3732g0120 (pea), S_TKc-CCT in Ca.14621 (chickpea) (FIG. 4A, 4B). Non-typical CCT proteins were not identified in soybean and Arabidopsis. All identified CCT genes in this study were summarized in Table 1. HMM logos were next prepared, representing each cluster (I-VI) from the domain tree to analyze the amino acids across the clusters (FIG. 6C). Most of the amino acids were conserved in the CCT domain across the six clusters, with high conservation observed for seven amino acids (Arginine (R) 1, R15, Tyrosine (Y) 23, R26, Alanine (A) 30, R35, and Phenylalanine (F) 40). Also, cluster-specific conserved amino acids were identified. For example, F8 in clusters V and VI, while Lysine (K) 22 was highly conserved in IV, with some exceptions (FIG. 6C). These conserved amino acids across the clusters could likely represent the essential roles of CCT family genes in DNA binding or forming functional complexes. In contrast, the amino acids specific to one or certain clusters might associate with the DNA binding specificity representing functional variation in the CCT family. The results indicated that the CCT domain sequences are conserved in plant species with diversified function specificities plausibly facilitated by some uniquely conserved amino acids.

All these six groups were identified as angiosperms. To further investigate the origin of these clusters, their membership in a range of non-angiosperms were identified, including charophyte algae, mosses, ferns, and gymnosperms (Table 20). All six clusters could be identified in each of the land plant lineages; however, two groups (I and VI) were absent from all of the chlorophyte species. This indicates that most of these groups arose early in plant evolution except for one of the 2×BBOX-CCT groups (I) and the TIFY-CCT-Zn_GATA group (VI), which first appeared in the bryophytes. Additionally, within the chlorophytes, Cluster IV (REC-CCT) was missing from all species except Chlamydomonas, and Cluster III (1×BBOX) was missing from Micromonas and Dusinella. These results indicated that individual chlorophyte lineages may have lost these genes or their sequences sufficiently diverged that the current search model could not identify them. Along with the increased number of CCTs from chlorophytes to bryophytes, the CCT domain gene family is ancient and underwent substantial expansion and diversification in the land plant lineage.

TABLE 20
CCT Genes in Other Species

| Species | I | II | III | IV | V | VI | Unclustered |
|---|---|---|---|---|---|---|---|
| spikemoss | 1 | 2 | 3 | 3 | 2 | 2 | 0 |
| liverwort | 0 | 1 | 2 | 2 | 1 | 2 | 0 |
| Angiosperm | 85 | 75 | 89 | 93 | 129 | 62 | 5 |
| fern | 3 | 3 | 4 | 6 | 6 | 1 | 1 |
| gymnosperm | 4 | 2 | 4 | 4 | 4 | 2 | 0 |
| Bryophyte | 9 | 7 | 24 | 10 | 10 | 10 | 0 |
| chlorophyte | 0 | 7 | 2 | 6 | 1 | 0 | 2 |

### F. Function Diversification of CCT Proteins

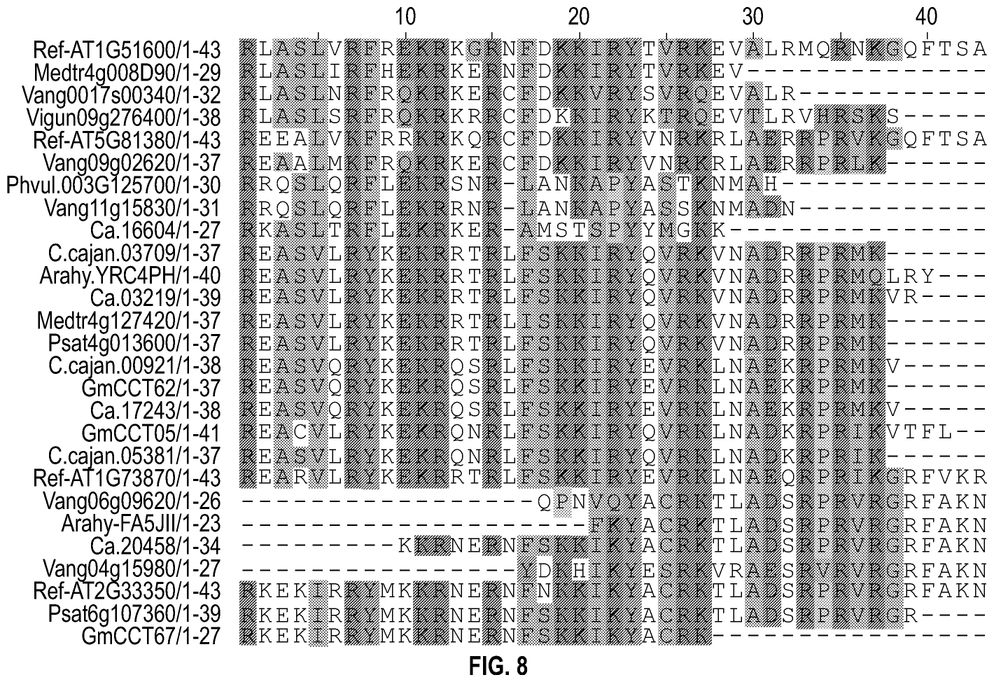

Given the conservation in CCT domain architecture and protein sequences within the clusters, a phylogenetic tree was reconstructed using CCT proteins from four species (soybean, Arabidopsis, rice, maize) to infer functions since many CCT proteins from the latter three species have been functionally characterized. The phylogeny of the four-species CCT tree was in agreement with the global tree and soybean trees mentioned earlier, and was defined into six clusters (I to VI) by tropology. Cluster I consisted of single-CCT proteins while rare of which were characterized. ZmCCT was located within a monocot-specific subcluster and it involved in maize adaptation from short-photoperiod tropical environments (Southern Mexico) to Northern long-day environments. Whether the phylogenetically close GmCCTs such as GmCCT38 possessed relevant roles warranted experimental determination. ASML2 was highly expressed in Arabidopsis stem and perhaps functioned as a transcriptional activator in the regulation of a subset of sugar-inducible genes, two homologs in soybean GmCCT29 and GmCCT53 were likely to have the similar function because both were found to be highly expressed in soybean stems (FIGS. 4A-4C and 8).

Cluster II represented REC-CCT domain proteins and the REC was conserved domains for PRR (PSEUDORESPONSE REGULATOR) proteins that were mainly studied in Arabidopsis but rarely in other species. All five PRR proteins (REC-CCT) from Arabidopsis (PRR1 (TOC1), PRR3, PRR5, PRR7, PRR9) were clustered in this cluster. The cluster also contained two rice REC-CCT proteins (Ghd7 and Ghd7.1) functioning in the regulation of flowering time (heading date)-associated adaptation with potential in enhancing yield potential (grain number) (Xue et al. 2008; Yan et al. 2013). These and other studies (Weller and Ortega 2015) suggested conserved functions of REC-CCTs in the regulation of circadian clock and light response-related flowering. With these results, it was deduced that many of those uncharacterized PRR proteins (REC-CCT) in legumes likely function related to circadian clock responses or photoperiodic flowering control. It was known that legumes originate differently, either temperate regions with long daylength (i.e. pea, chickpea) or lower latitudes with short-day photoperiod (i.e. soybean, cowpea, common bean). In the light of above results, the REC-CCT genes had the potential to widen the latitudinal range of legume cultivation (FIG. 6C).

Cluster III mainly comprised 2×BBOX-CCT proteins mixed with several other subfamily members. Six COL family members (CO, COL1-5) associated with flowering time, shoot branching and ZmCCT9 associated with high latitude adaptation were clustered in this cluster. In soybean, GmCCT proteins in this cluster except for GmCCT61 and GmCCT43 exhibited similar spatial expression patterns as Arabidopsis CO in floral bud, leaf, and stem, suggesting a possible conserved role in flowering time regulation. Proteins carrying single-CCT domain from cluster IV were phylogenetically close to FITNESS and CIA and might have functions relevant to chloroplast development or ROS homeostasis-associated drought tolerance. In cluster V, two BBOX-CCT proteins (GmCCT40 and GmCCT47) were phylogenetically closed to Arabidopsis COL12 (BBX10) that associated with branching and flowering time; and two 2×BBOX-CCT proteins (GmCCT44 and GmCCT66) were close to COL9 (BBX7) with a role in flowering. The four-species CCT tree provided an overall picture of functional diversity in plant species and insight into the putative role of GmCCTs.

### G. GmCCTs Exhibited Expression Specificities to Circadian Clock, Environmental Stress, or Tissues

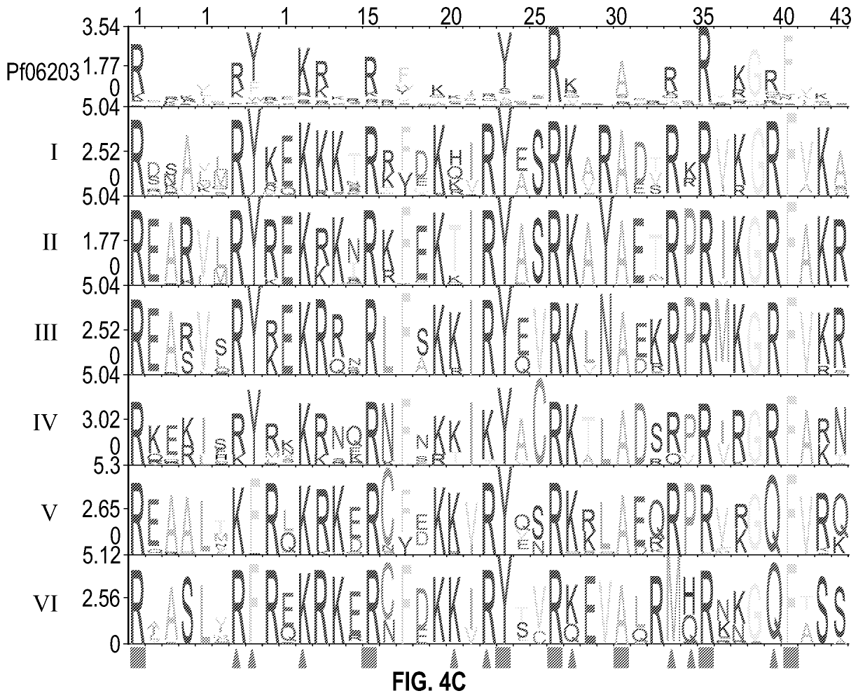

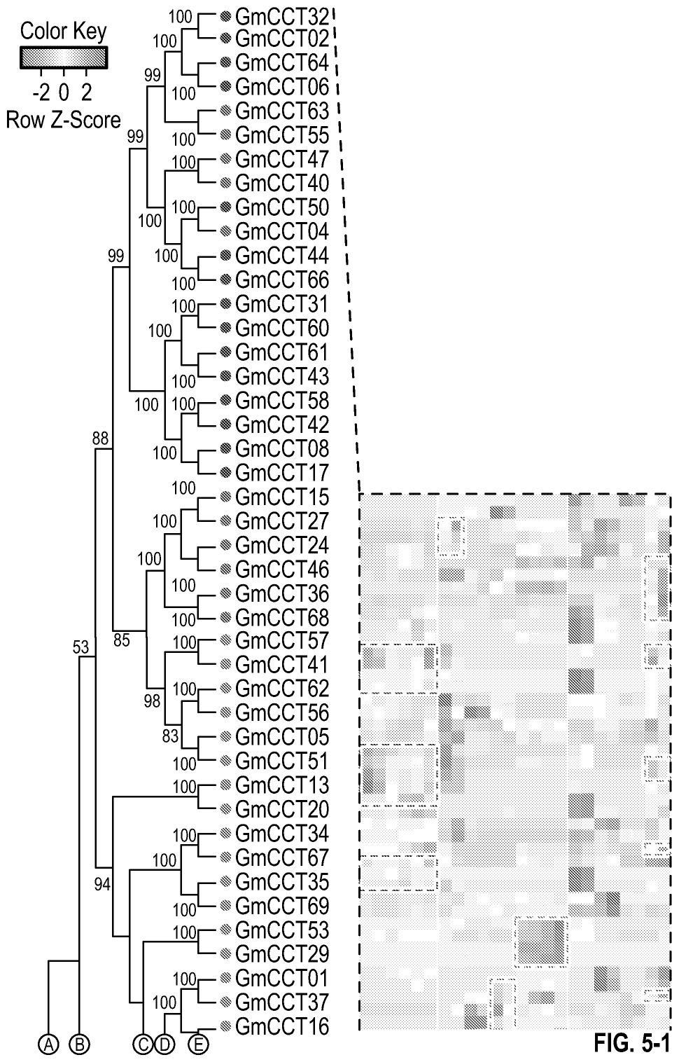

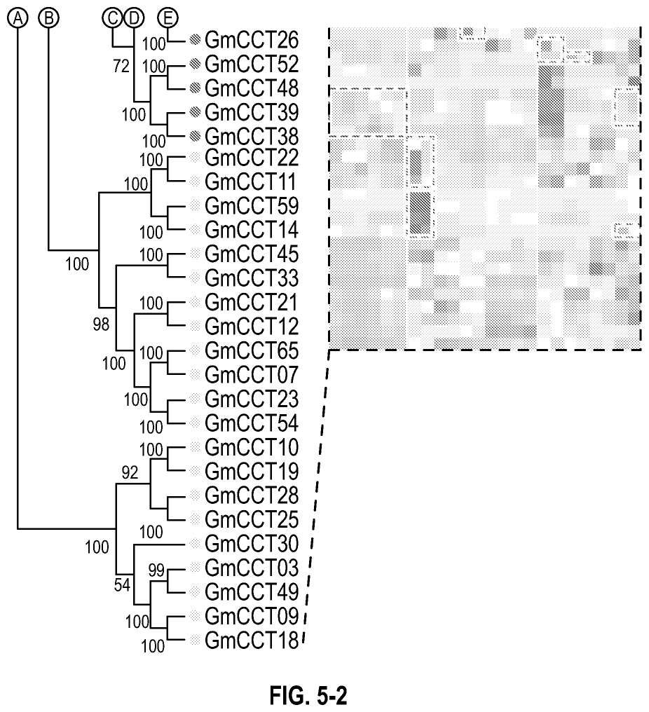

To gain more insight into the roles of GmCCT genes, the expression profiles in different conditions including circadian rhythm, abiotic stress (drought, Zn, low temperature, O3), and biotic stress (cutworm, F. graminearum, reniform nematode, and aphid) were investigated. It was revealed that sixteen GmCCTs showed varying circadian clock responses in a Zeitgeber time (ZT) interval of 20 h, with four REC-CCTs and two single-CCT proteins highly expressed during ZT8-12 h and three pairs of BBOX-CCT paralogs exhibiting high expression during early and late ZT points of the period (FIG. 5), suggesting relevant roles in circadian rhythm and likely photoperiodic flowering time control. In addition, it was identified that GmCCT genes were responsive to the challenges of drought, salt, cutworm or F. graminearum (FIG. 5). Two pairs of GmCCTs (GmCCT34/67, GmCCT35/69) exhibited relative insensitivity to elevated temperature but were inducible to 03 stress. Interestingly, the circadian clock responsive CCT genes were rarely identified to be responsive to abiotic and biotic stress and vice versa for the stress-responsive GmCCTs, implying functional specialization of the GmCCTs.

Given the agronomic importance of seed quality traits for soybean in current breeding programs, the analysis was extended to investigate the expression in different seed compartments (i.e. inner/outer integument of the seed coat, suspensor, endosperm) at different seed development stages (globular, heart, cotyledon, early-maturation) along with major vegetative organs (seedlings, leaves, floral bud, stem, root), aimed to understand whether GmCCTs correlate with seed compartment profiles. Overall, most of the single-CCT proteins were expressed in seed compartment tissues while 1-2×BBOX-CCT showed tissue-specific expressions in non-seed vegetative tissues, such as GmCCT02 preferentially expressed in stems (STEM) and GmCCT47 exclusively expressed in floral bud (FLUB) (FIG. 6). It was noteworthy that the four O3-responsive GmCCT genes (GmCCT34/67, GmCCT35/69) were also preferentially expressed in the seed coat compartments (seed coat outer integument at the cotyledon stage (COT-OI) and seed coat parenchyma at the early maturation stage (EM_SC_PY)) (FIG. 6). The parenchyma cell was the innermost part of seed coat that was in direct contact with the embryo, and it contained components related to nutrient transport and metabolism to support embryo growth during seed filling. The four GmCCT genes may play roles associated with seed development or storage reserves accumulation, which was rarely reported in plants. GmCCT genes encoding TIFY-CCT-ZnF-GATA and REC-CCT did not appear obvious expression specificity in the tested seed compartment tissues.

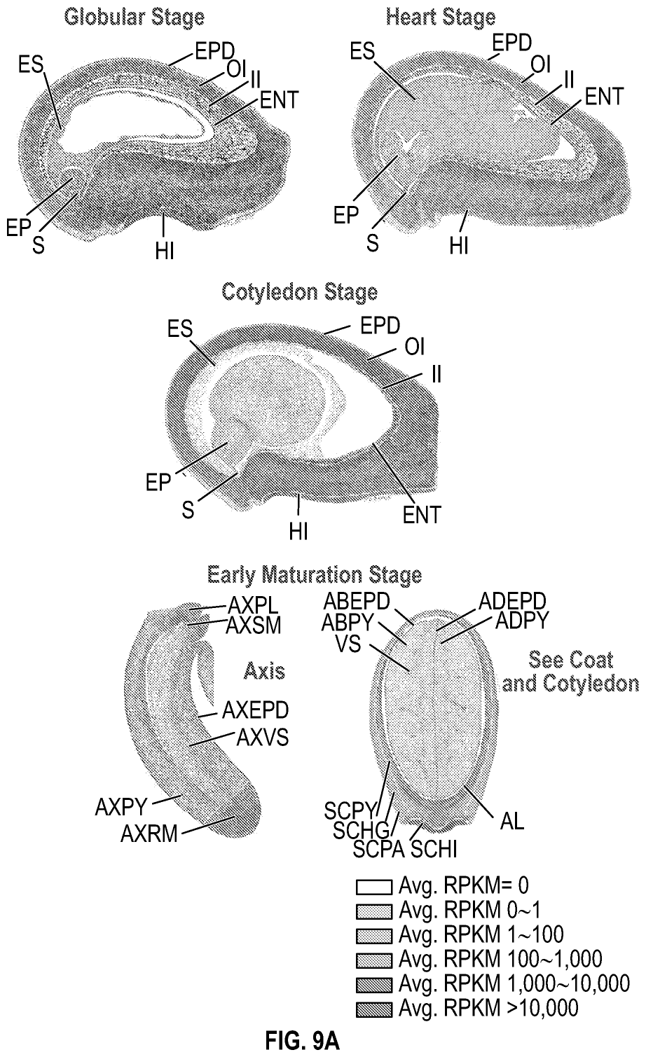

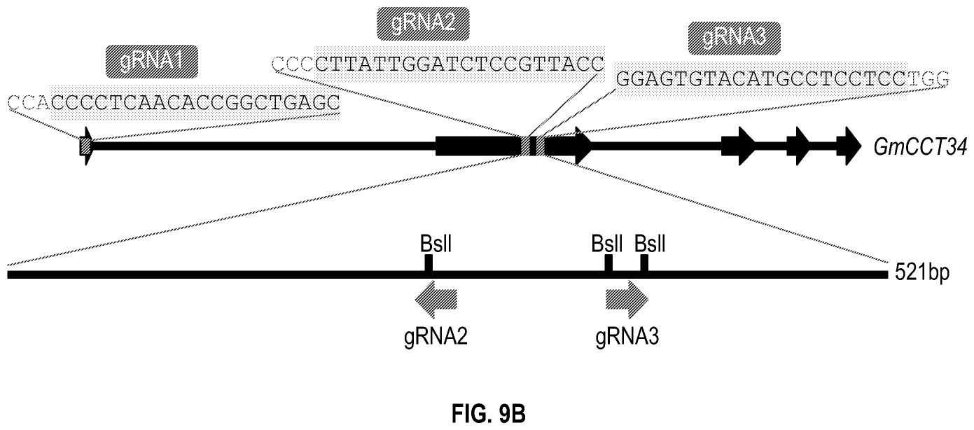

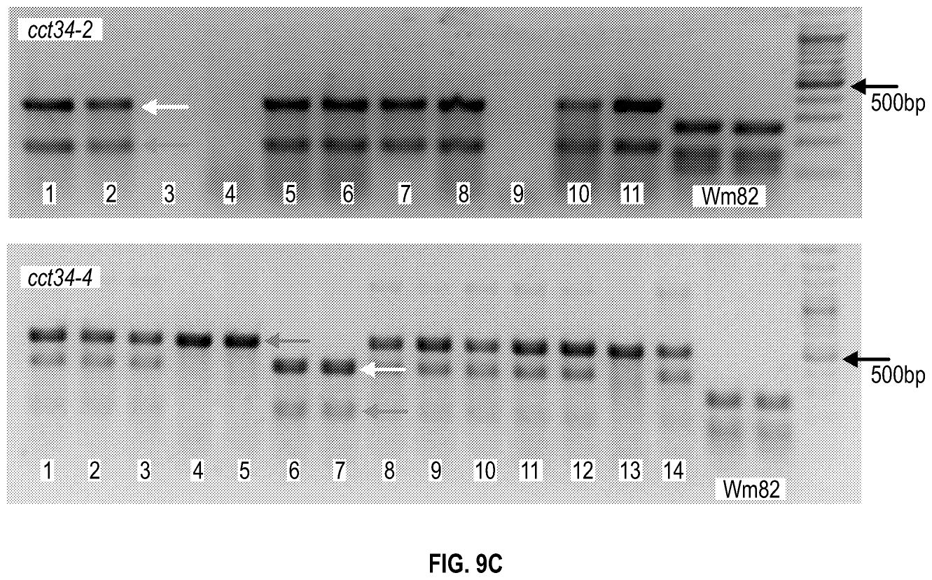

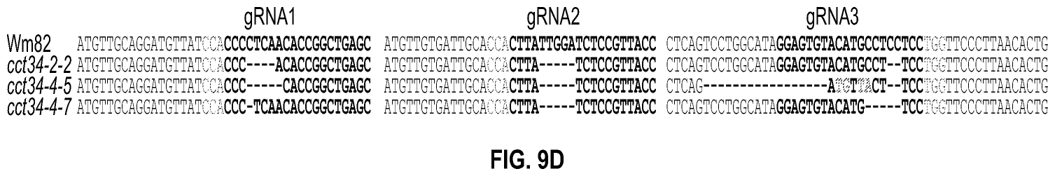

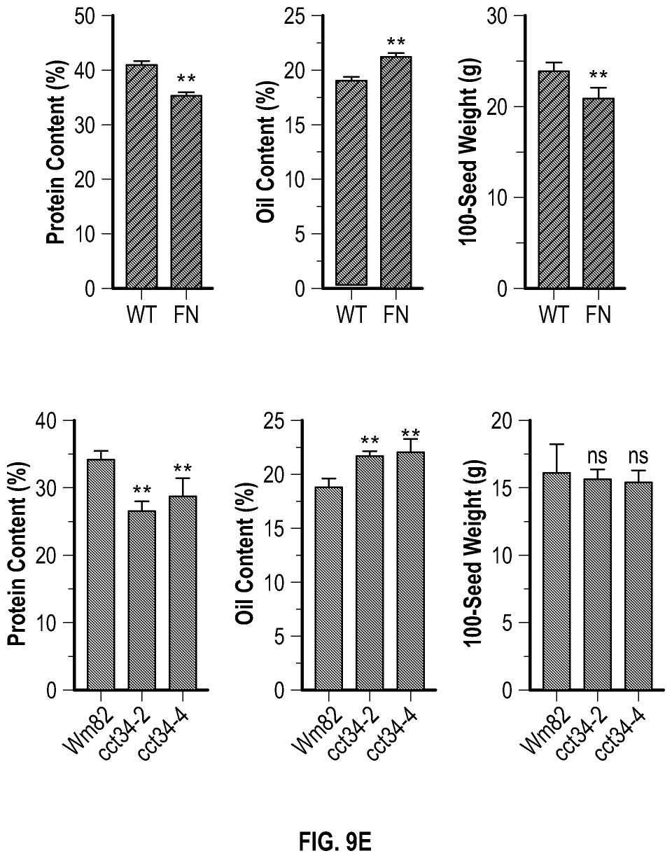

It was also observed there was conserved and divergent expression for GmCCT paralogs across tissues, circadian clock response, and environmental stress. For example, seed coat-specific expressed paralogs GmCCT34/67 and circadian clock responsive gene pair GmCCT24/46 showed similar expression patterns, while GmCCT56/62 exhibited different circadian clock responses (FIGS. 8, 9). Similar expression pattern and conserved protein sequences in the paralogous GmCCT genes suggested that they preserved the ancient biological functions. They likely had retained the similar promoter elements essential for expression specificity. The divergent expression may lead to sub-functionalization or neo-functionalization in different tissues or environment responses, enriching the functional diversity of GmCCT family.

### H. Exploring Natural Variation in GmCCTs and Co-Located QTLs

To explore the natural variation in GmCCT family, it was examined in the coding sequences within a panel of 1,556 soybean genomes from diverse genetic backgrounds. After investigation, four types of variants were identified that may cause amino acid changes in 58 (84.1%) of 69 GmCCTs. In total, 250 variants (minor allele frequency >0.01) were identified, including 214 non-synonymous SNPs, 5 SNPs causing alternative splicing, 30 indels ranging from 3-28 nucleotides, and 2 nonsense SNP mutations that caused premature proteins (Table 4). The variants that cause protein sequence changes might be responsible for morphological or physiological changes. For example, GmCCT67, also known as POWR1, contains a 321-bp indel in the CCT domain that h likely abolishes the function. The indel in POWR1 accounts for the significant variation in seed protein and oil content. Other than this, GmCCT17 (2×BBOX-CCT) is phylogenetically close to COL3 and COL4 and associated with abiotic stress tolerance and flowering. The GmCCT17 carried an SNP in the 1st exon causing the premature stop codon in 28 diverse accessions, 22 of which (78.6%) originated from Northern China (north of the Shandong province (36.6° N)). It is intriguing to determine if the variant contributes to latitudinal adaption. The diverse variants revealed here can be valuable for gene functional characterization or breeding purposes.

Previously published QTLs were incorporated into the analysis and assessed co-locationship between GmCCT genes with the QTLs. Total of 66 (95.7%) of 69 GmCCTs reside in genomic regions were identified that had 680 non-redundant reported QTLs, including 220 for seed quality traits, 131 for seed set, 119 for abiotic/biotic stress tolerance, 51 for flowering time and maturity, and 158 for development-related traits (Tables 5-9). It has been demonstrated that many PRR homologous proteins underly the major QTLs for heading time and grain yield. Seven (58.3%) of 12 REC-CCT proteins (PRRs) were situated in QTLs associated with the first flower, photoperiod sensitivity, reproductive stage length, R8 full maturity and plant height or seed yield (one of Tables 5-9). Intriguingly, six of the 16 (37.5%) circadian clock-responsive GmCCTs (GmCCT11, 22, 05, 68, 43, 60) were located within the flowering-related QTLs (FIG. 5; one of Tables 5-9). All four proteins that were preferentially expressed in seed coat compartments were located within QTLs for seed quality or seed set traits (one of Tables 5-9).

Further, the genetic diversity (TT) for GmCCTs resident loci were analyzed and 12 GmCCTs (GmCCT06, GmCCT14, GmCCT20, GmCCT26, GmCCT32, GmCCT41, GmCCT42, GmCCT59, GmCCT61, GmCCT63, GmCCT64, GmCCT67) were located within selective sweep regions (Table 4). Many of these merited further attention, for example, GmCCT05 (a FITNESS homolog) co-located within a QTL associated with drought tolerance. The most noteworthy is GmCCT67, located within multiple QTLs, including four QTLs for protein, four for oil content, one for seed weight, and one for yield (Table 10). It was recently proven that the major QTL cqPro20 controls protein, oil, and seed weight simultaneously and is subjected to strong artificial selection, which strongly supports the diversity analysis. Whether other QTL-colocalized genes carry advantageous mutations targeted by human selection deserves experimental determination.

### I. GmCCT Genes are Stress-Responsive

CCT genes regulate a plethora of functions in plants. Expression profiles of GmCCT genes (n=69) were investigated in response to various abiotic and biotic signals, including circadian rhythm, abiotic stress (drought, Zn, low temperature, 03), and biotic stress (cutworm, F. graminearum, reniform nematode, and aphid). A set of sixteen GmCCTs showed varying circadian clock responses in a Zeitgeber time (ZT) interval of 20 h, including four REC-CCTs, two single-CCT proteins, and three pairs of BBOX-CCT paralogs (FIG. 5), suggesting relevant roles in circadian rhythm and likely photoperiodic flowering time control. In addition, GmCCT genes that were responsive to the challenges of drought, salt, cutworm, or F. graminearum (FIG. 5) were also identified, such as two pairs of GmCCTs (GmCCT34/67, GmCCT35/69) exhibiting relative insensitivity to elevated temperature but were inducible to 03 stress. Further, phylogenetically close genes were identified, particularly the paired GmCCT paralogs that retained similar expression patterns or exhibited divergent expression. For example, GmCCTs (64, 06, 63) showed similar expression responses to drought, and GmCCT56/62 exhibits different circadian clock responses (FIG. 5), which may enrich the functional diversity of the GmCCT family during evolution to cope with diverse environment responses.

### J. GmCCT34 Involved in Seed Protein and Oil Content Accumulation

Considering the agronomic importance of soybean seed quality traits, the expression profiles of GmCCTs were investigated in different seed compartments (i.e., inner/outer integument, seed coat, suspensor, and cotyledon) and at different seed development stages (globular, heart, cotyledon, early-maturation). Also, the expression of these genes were analyzed in major vegetative organs (seedlings, leaves, floral bud, stem, root), aimed at additional GmCCTs involved in seed compartment profiles. Overall, a correlation was observed between tree topology and expression profile, suggesting sequence co-evolution with spatial expression. Most of the single-CCT proteins were expressed in seed compartment tissues. In contrast, 1-2×BBOX-CCT showed tissue-specific expressions in non-seed vegetative tissues. For example, GmCCT02 was preferentially expressed in stems (STEM), and GmCCT47 exclusively expressed in the floral bud (FLUB) (FIG. 6). GmCCT genes encoding TIFY-CCT-ZnF-GATA and REC-CCT did not appear to have apparent expression specificity in the tested seed compartment tissues.

Remarkably, the cluster of four O3-responsive GmCCT genes (GmCCT34/67, GmCCT35/69) were preferentially expressed in the seed coat [seed coat outer integument at the cotyledon stage (COT-OI) and seed coat parenchyma at the early maturation stage] (FIG. 6). The parenchyma cells are the innermost part of seed coat that is in direct contact with the embryo. It contains nutrient transport and metabolism components to support embryo growth during seed filling. It was recently demonstrated that GmCCT67 (POWR1) regulates protein and oil accumulation, seed weight, and field yield. Given the conserved expression pattern in the seed coat, it was reasoned that the other three GmCCTs might function similarly in seed quality traits.

### K. GmCCT34 Involved in Seed Protein and Oil Content Accumulation

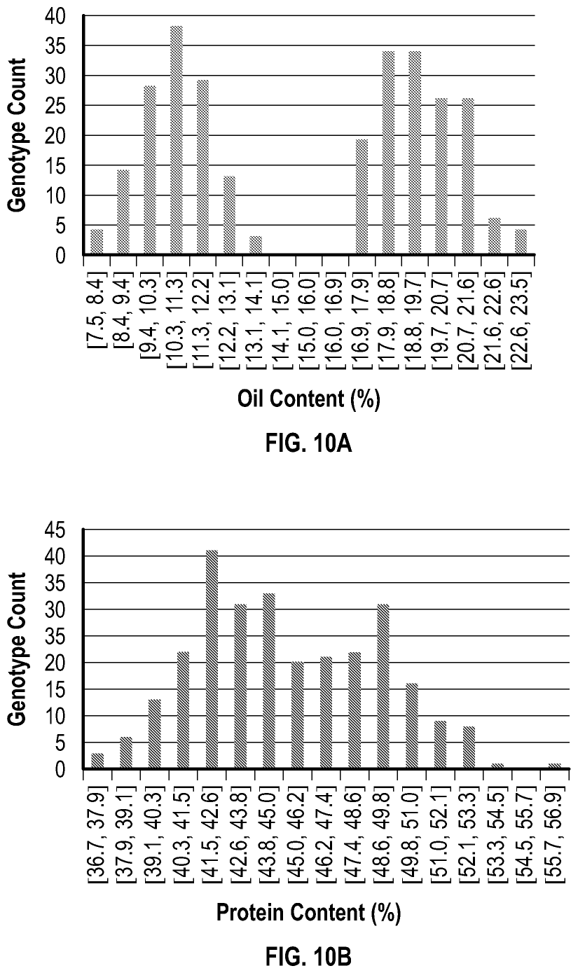

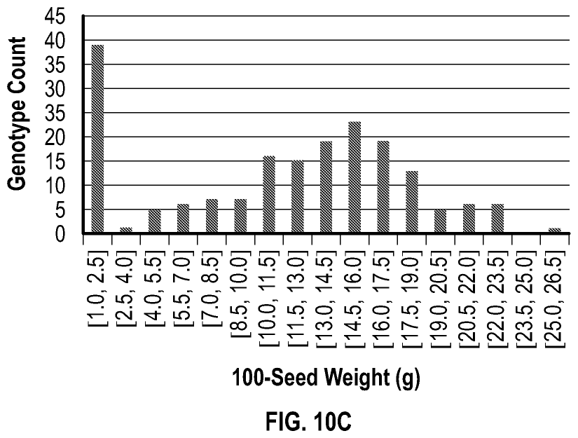

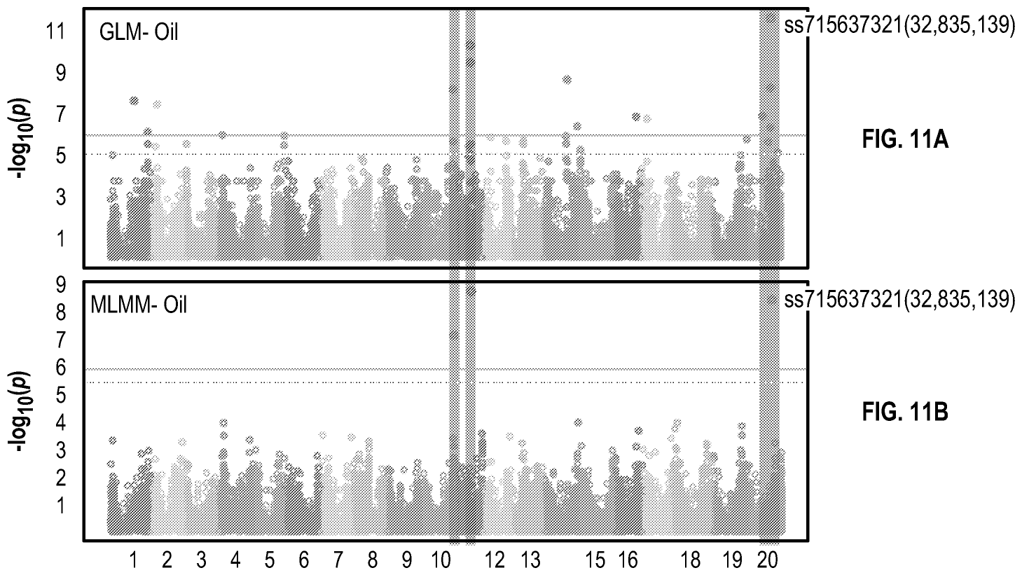

A high expression of GmCCT34 was analyzed specific to seed coat tissues (FIG. 1A, FIGS. 10, 11A). To test if GmCCT, other than POWR1, is involved in the regulation of the seed composition in soybean, a fast neutron mutant FN0172932 was identified lacking a 1.3-Mb genomic region (Chr10: 35253890-36584337). Interestingly, the Gmcct34 mutant (FN0172932) M4 seeds contain an average of ˜5.5% less protein (p<0.001) and ˜2.241% more oil content (p<0.001) than the wild-type (WT) seeds (FIG. 9E), suggesting its role involved in regulating protein and oil accumulation.

Additionally, to confirm if the absence of GmCCT34 causes the low protein-high oil phenotypic change in the fast neutron (FN) mutant, GmCCT34 knockout lines were generated in soybean cv. Williams82 (Wm82) background using CRISPR/Cas9-mediated gene editing. Two mutant lines (cct34-2, cct34-4) homozygous for GmCCT34 with nucleotide deletions of varying lengths simultaneously at designed targeted sites in the 1st and 2nd exons were identified (FIG. 9B-9D). Consistent with the protein-oil content in FN mutant result, T2 seeds of homologous cct34 edited lines showed significantly low protein and high oil accumulation. Gmcct34 seeds contained ˜7.86% (p<0.001) low protein content and ˜2.85% (p<0.001) higher oil content than the WT Wm82 (FIG. 9E). Differing from GmCCT67 (POWR1) that CCT domain-truncated POWR1 is responsible for greater 100-seed weight than the wild type, the 100-seed weight was reduced in both FN0172932 and cct34 mutants, although the latter was not statistically significant. These results demonstrated and validated that the seed coat-preferentially expressed GmCCT34 regulates seed protein and oil accumulation in soybean.

Given the observed changes, it was reasoned that the CCT domain plays an important role for the GmCCT34's function. subcellular localization assays were next carried out of intact and fragmented GmCCT34 containing or lacking the CCT domain. The analysis clearly illustrated that GmCCT34 carrying intact CCT domain (UBQ10:YFP-CCT34) and the CCT domain only (UBQ10:YFP-CCT) were located exclusively in the nucleus (FIGS. 2A and 2B), whereas, GmCCT34 lacking the CCT domain (UBQ10:YFP-CCT34ACCT), like the empty vector (UBQ10:YFP-Emptyvector), expressed in the nucleus and cytoplasm. This result indicated the essential role of CCT domain to direct GmCCT34 in the nucleus and that removal of CCT domain in GmCCT34 likely abolished the function in enhancing protein accumulation.

### L. Arabidopsis CCT-Clade Protein Regulates Protein-Oil Content in Seeds

Beyond the four seed-coat GmCCTs from soybean, the phylogenic analysis clustered a set of homologs from selected species with POWR1 and GmCCT34 into a distinct clade, it was asked whether those from non-legume plants remain similar function, the function of the Arabidopsis CCT gene, AT1G04500, was investigated for its involvement in regulating seed protein-oil composition. Two homozygous Arabidopsis T-DNA-insertion mutants were isolated as ATcct-1 and ATcct-2. The T-DNA insertion in these mutants occurred before and after the CCT domain, respectively, indicating that the CCT domain is dysfunctional (FIG. 10A-10C). Similar to Gmcct34, the seed composition analysis of the ATcct mutants revealed a higher oil and lower protein content compared with the wild type seeds (FIG. 10A-10B). These results suggest a conserved function of the CCTs between soybean and Arabidopsis in regulating protein and oil accumulation.

### M. Arabidopsis CCT-Clade Protein Regulates Protein-Oil Content in Seeds

Function of the Arabidopsis CCT gene, AT1G04500, was investigated for its involvement in regulating seed oil composition. Like GmPOWR 1234 genes, there is only a single CCT domain found in Arabidopsis AT1G04500 gene (hence after AtPOWR1). The gene expression analysis showed that the AtPOWR1 is highly expressed in the seed coat tissues (FIG. 11A-11B, red color indicating the AtPOWR1 expression).

There is no information on the function(s) of AtPOWR1 concerning the regulation of seed protein-oil content. To know if this Arabidopsis gene also functions similarly to the GmPOWR genes, two homozygous T-DNA-insertion mutants were isolated (WiscDsLox297300_13A.1 and SALK_036731.1, (labeled as cct-1 and cct-2). The T-DNA insertion in these mutants occurred before and after the CCT domain, respectively, indicating that the CCT domain is dysfunctional. Similar to GmPOWR mutants, the seed composition analysis of the AtPOWR1 or ATcct mutants revealed a higher oil content compared with the wild type seeds These results suggested a conserved function of the CCTs between soybean and Arabidopsis in regulating oil accumulation in seeds.

### DISCUSSION

### A. Evolution of CCT Proteins

The results indicated that CCT domain-containing proteins with four subfamilies were conserved in land plants, in both domain architecture and constitution. Three of the four domain structures of CCT proteins can date back to an ancestral origin in chlorophyte species and were significantly expanded in land species after the long evolutionary divergence. The long evolutionary history after speciation has caused a great deal of sequence divergence because of mutation. Highly divergent domain sequences across the subfamilies suggested possibly distinct functions. “Membership” switch was also identified by observing possible addition or loss of domains between the subfamilies, which could be a result of domain shuffling due to unequal chromosomal crossover or transposon participation. The domain rearrangement might be a cause for the presence of all four subfamilies in land species compared with the absence of 1-3 subfamilies in chlorophyte species. Domain shuffling also increased the emergence of novel CCT genes with functional innovation, such as those uncanonical CCT proteins identified in this study.

It was also demonstrated that WGD during the plant evolution was the major driven force of CCT family expansion, which led to striking high numbers in higher plants compared to dramatic lower number chlorophyte species. CCT domain possessed a role in DNA binding by which the protein can affect downstream gene expression levels and associated functions. It appeared that CCT genes in soybean and peanut were retained after WGD based on the tree topology, which can be explained by previous studies demonstrating that functions of signaling or regulatory genes were more likely retained following WGD events relative to the genome-wide average. Most of the CCT paralogs remain similar expression patterns in tissues or environmental conditions after WGD, suggesting functions in common. On the other hand, approximately 50% of paralogs genome-wide in soybean are differentially expressed with possibly divergent functions. For example, ABSCISIC ACID INSENSITIVE 3a (ABI3a) retains functions associated with seed migration and dormancy while GmABI3b was neofunctionalized like GmLEC2 in modulating seed fatty acid biosynthesis in soybean. Similarly, many GmCCT paralogous pairs likely experienced expression divergence, suggesting they have undergone differentiation. Given the reported functions such photoperiod adaptation and drought tolerance, expansion of CCT genes with divergent functions may enable plants more resilient to the change of environmental factors such as latitudinal photoperiod or drought conditions.

### B. More GmCCTs Might be Involved in Soybean Flowering Control

In general, legumes have their respective origins at lower (soybean, cowpea, pigeon pea, and common bean) or higher (such as chickpea, pea) latitudes, and their cultivation have been expanded to regions beyond the origins after domestication and modern improvement. The underlying mechanism was partially revealed in soybean by investigation of E series genes and Dof11/GmPRR37 that contribute to latitudinal adaptation. For example, Gmprr37 lacking the CCT domain (mutant allele in the Williams 82 genome) confers early flowering, which enables soybean to be adaptive at a higher latitude with the long-day condition (ref). However, the underlying mechanism remains largely unknown in legumes. The present study provides a list of GmCCTs with varying photoperiodic responses that may be involved in soybean latitudinal adaptation. The orthologs in Brassicaceae and Fabidae deserve further attention to exploring the functions and potential for crop improvement extensively. Nevertheless, the analysis enables a comprehensive inventory of GmCCT genes with a variety of predicted functions, which is useful in improving the discovery of function for the syntenic orthologs in legumes, particularly in the circumstances that rare CCT underlying flowering-related QTLs and protein accumulation has been identified in legumes.

C. The Family Contains a Distinct CCT Clade with the Potential for Seed Traits Improvement

The demand for plant-based protein has been increasing worldwide because of rapid global population growth, and legumes are certainly the major providers of adequate plant-based protein worldwide. Finding genes that regulate seed protein levels is critical to leverage them for improvement. However, few genes controlling protein content have been identified in soybean, and much less in other legumes, hindering their use for substantial protein improvement.

Here, the instant systematic study identified four GmCCTs explicitly expressed in seed coat tissues wherein nutrient-transporting elements are active, suggesting a relevant role in nutrient accumulation. Indeed, both fast neutron and CRISPR-Cas9 mutation analyses showed that GmCCT34 reduced protein and increased oil content and seed weight. As additional strong evidence, the present contemporary assay demonstrated that GmCCT67 underlies the major QTL cqPro-20 controlling protein and oil levels in seeds. These results clearly demonstrate the role of both genes from the clade in regulating protein and oil content. Likewise, the other two seed-coat-specific GmCCTs (GmCCT35/GmCCT69) that are phylogenetically closest to GmCCT34/GmCCT67 likely function similarly. It is unexpected that mutation in the Arabidopsis ortholog AT1G04500 also affected protein and oil content, suggesting that the function is conserved between soybean and Arabidopsis, which diverged approximately 90 MYA (ref). In this context, legumes are much closer (˜59 MYA) to soybean than Arabidopsis. Therefore, legume CCTs from this clade likely have similar functions associated with protein accumulation, although the spatial expression pattern and function need to be elucidated. Nevertheless, the present analysis identified this set of CCT genes, with two of which being functionally validated, may contribute to protein or oil content improvement by genetic engineering in legumes and other grain crops.

### D. The Possible Mechanism for Regulating Seed Nutrient Accumulation

The four GmCCTs were highly expressed in developing seed coat tissues, such as parenchyma, during early and cotyledon stages. The parenchyma is the innermost part of the seed coat with direct contact with cotyledon. It contains transporters facilitating the nutrient transfer, such as a sugar transporter GmSWEET39, involved in sucrose transporting for oil and protein accumulation. The two stages represent the key period of seed filling when photosynthetic accumulates and is delivered from maternal tissues to filial cotyledon to support a developmental embryo. Therefore, relatively high expression of the four GmCCTs in the tissue at the stages suggests their stage and tissue-prominent function, which regulate biological processes associated with nutrient transport in the seed coat.

Studies in Arabidopsis demonstrated that CCT domains have DNA binding activity and are required for its interaction with COP1 or NF-YB2 in binding the promoter of FT to regulate flowering time. The present studies demonstrated that GmCCT34/GmCCT67 might function like transcription factors as knockout of the CCT domains abolished their exclusive expressions in the nucleus. Therefore, it is likely that the GmCCTs regulate an array of genes associated with nutrient transport as inferred by its primary expression in the seed coat. In addition, CCT genes are identified to activate the expression of a subset of sugar-inducible genes such as SUS2, and sugar can serve as the precursor for lipid biosynthesis. Overall, the present data suggested a plausible role of the CCTs affecting sugar transport capacity in the seed coat, which influences its supply for the biosynthesis of protein or oil in the cotyledon. Further studies must determine the detailed regulation mechanism and their roles in protein-oil negative correlation.

### E. Functional Conservation and Mutation

Studies have shown that many CCT genes have conserved functions after specification in cereals and Arabidopsis, such as a role of photoperiod-associated flowering time control. In general, legumes had their respective origins at lower (soybean, cowpea, pigeon pea, and common bean) or higher (such as chickpea, pea) latitudes and their cultivation have been expanded to regions beyond the origins after domestication and modern improvement. In soybean, identification of flowering time controlling genes in legumes and soybeans such as E series genes and FT gene family provided one perspective of the mechanism of flowering time control, whereas the mechanism underlying latitudinal adaptation remained largely unclear. GmCCT genes played conserved roles in photoperiodic response as revealed in recent studies. For example, Gmprr37 lacking the CCT domain (mutant allele in the Williams 82 genome) conferred early flowering which enabled soybean to be adaptive at a higher latitude with the long-day condition. The instant study provided a list of GmCCTs with varying photoperiodic responses that may possibly be involved in soybean latitudinal adaptation, which needs experimental determination. Given the conserved function in affecting flowering time, the syntenic orthologs in legumes deserve further attention. Nevertheless, the analysis enabled a comprehensive inventory of GmCCT genes with a variety of predicted functions, which was useful in improving the discovery of function for the syntenic orthologs in legume, particularly in the circumstances that rare CCT underlying flowering-related QTLs and protein accumulation has been identified in legumes.

Mutations occurred in the CCT proteins caused substantial phenotypic changes. Thus far, the discovery of the agriculturally important CCTs in grain crops owes to the identification of the sequence variation in a natural population, such as Gmprr37 lacking the CCT domain, and transposable element interfered ZmCCT9. The current study identified truncated CCT proteins in the reference genomes of cultivated legumes and cereals and numerous variants in the soybean natural population with many of which were likely subjected to artificial selection. It was possible that many of these play roles in domestication syndrome traits. For example, seed coat-exclusively expressed GmCCT67 lacking the CCT motif was located within a sweep region and likely involved in seed quality traits in soybean, which was supported by the alternative assay where the knockout of its syntenic gene GmCCT34 significantly increased oil accumulation while reduced protein content in soybean seeds. The mutated CCT identified in legumes and soybean natural population might under selective pressure for the responsible phenotypic variation and can be prioritized for further examination.

### Gmcct34 Possesses a New Role in Seed Composition Accumulation

Previous studies demonstrated that CCT domains had DNA binding activity and were required for its interaction with COP1 or NF-YB2 in binding the promoter of FT to regulate flowering time, and a CCT gene can also activate the expression of a subset of sugar-inducible genes such as SUS2. Sugar can serve as the precursor for lipid biosynthesis. On the other hand, the parenchyma was the innermost part of the seed coat with direct contact with cotyledon, and it contained transporters facilitating nutrients transfer, such as a sugar transporter GmSWEET39 involved in sucrose transporting for oil and protein accumulation. Considering these and the results presented herein, GmCCT34 perhaps associated with many genes involved in nutrients transport such as sucrose or amino acids into the cotyledon for storage reserves accumulation. The CCT domain might play a key role as disrupted CCT domain in cct34 might abolish its DNA binding function and associated biological pathways in oil and protein accumulation and seed weight. Seed oil often positively correlates with seed weight, an important yield component, while both negatively correlate with protein content in soybean, and the negative correlation poses a challenge for improving protein while maintaining satisfied yield. The synergistic changes in protein and seed weight in cct34 seeds may offer an opportunity to improve both traits simultaneously, although the mechanism remains to be uncovered. Further, GmCCT34 likely had no syntenic orthologs in legumes, therefore, the function involved in protein and oil accumulation might be lineage-specific to soybean.

### CONCLUSIONS

Plant-specific CONSTANS, CONSTANS-LIKE, and TIMING OF CAB EXPRESSION1 (CCT) domain-containing proteins regulate diverse functions associated with plant growth, development, responses, and agronomic traits. The soybean genome contained 69 CCT-domain proteins preferentially retained after whole-genome duplication. Recently, the CCT-domain gene has been shown to regulate the protein content in soybean seeds. The present studies analyzed the role of four closely related CCT-family subcluster genes; GmCCT34, GmCCT35, GmCCT67, and GmCCT69. These genes were identified as highly conserved in seed coat and flower or reproductive tissues across plant species. Interestingly, the orthologues of these genes are present in early land plants and exhibit reproductive tissues-specific expression. The present disclosure evaluated the role of these four CCT-family subcluster genes in soybean seed protein-oil content. Notably, the GmCCT transgenic, gene-edited, and fast neutron mutant seed analysis showed that these genes contained significantly lower protein and higher oil content than the wild-type seeds. The present results provided deeper insight into the CCT gene family evolution, phylogeny, and functions. Overall, the present disclosure showed that protein-oil-regulating CCT genes could be a potential source for seed quality improvement in soybean and other crops.

### Example 2. POWR1, a Key Domestication Gene Pleiotropically Regulating Seed Quality and Yield in Soybean

Seed protein and oil content, weight and field yield were the major traits impacting the economic value of soybean. the present multidisciplinary study revealed that a CCT (CONSTANS, CO-like, and TOC1) gene, POWR1 (Seed Protein-Oil-Weight-Regulator 1), underlied a major QTL on chromosome 20, and pleiotropically regulated these important seed traits. A transposable element (TE) insertion truncated its CCT domain and altered its exclusive localization in the nucleus. The POWR1 was specifically expressed in the seed coat of developing seeds and preferentially regulated expression of nutrient transporting and lipid metabolism genes. Study revealed that a dynamic POWR1 allele transfer occurred post domestication. However, TE insertion was completely associated with the transition from G. soja to G. max. It was hypothesized that POWR1 was a key domestication gene and played an important role in pleiotropically regulating the seed quality and yielding traits likely through a seed-coat specific transcriptional regulatory program. Selection for larger seeds fixed POWR1_{+TE} allele in cultivated soybean and contributed to shaping cultivated soybean with higher seed yield/weight/oil content and relatively lower protein.

### INTRODUCTION

Soybean [Glycine max (L.) Merr.] is one of the most important seed crops grown worldwide. It was domesticated from wild soybean (G. soja Sieb. & Zucc.) in East Asia about 6,000-9,000 years ago. Domestication and improvement have shaped soybean as the most important dual-function crop to provide both highly valuable seed protein and oil, which together account for almost all of soybean economic value.

Seed protein content, oil content and yield were considered as three of the most important traits in soybean improvement. On average, commodity-type soybean varieties contained about 40% seed protein and 20% seed oil. However, the three traits vary greatly in soybean nature population and often inter-relate with each other. Seed protein frequently showed a negative correlation with seed oil content and yield; however, its underlying genetic mechanism remain largely unknown. The complex correlation of the three important traits posed a great challenge in simultaneously improving both the soybean seed quality traits and yield to increase the overall economic value of soybean. In addition, cultivated soybean also contained a higher seed yield and oil content, but lower protein content than their ancestry wild soybean. It was important to illustrate the genetic and molecular basis underlying the three traits and their trait correlation, and to understand how those interrelated and important traits have been selected over the course of soybean domestication and improvement for soybean.

Through a combination of genomics, genetics, and molecular biology approaches, it was uncovered that a CCT-domain gene, POWR1 (Seed Protein-Oil-Weight-Regulator 1), underlied a large-effect protein and oil QTL on chr20 that has been pursed for the past three decades. It was demonstrated in the current study that a TE (transposable element) insertion in the conserved CCT domain was the causative variant contributing to the large variation in seed protein and oil in soybean population. Expression of the high-protein POWR1 allele in soybean was supportive of its function and potential in present-day needed high protein breeding worldwide. The study provided an insight into the molecular and genetic basis underlying the important seed traits and their correlation, and its key role in soybean domestication and improvement.

### Results

### A. A 321-Bp TE Insertion is Likely the Causative Variant of a Major QTL on Chr20 Controlling Seed Oil and Protein Content and Seed Weight

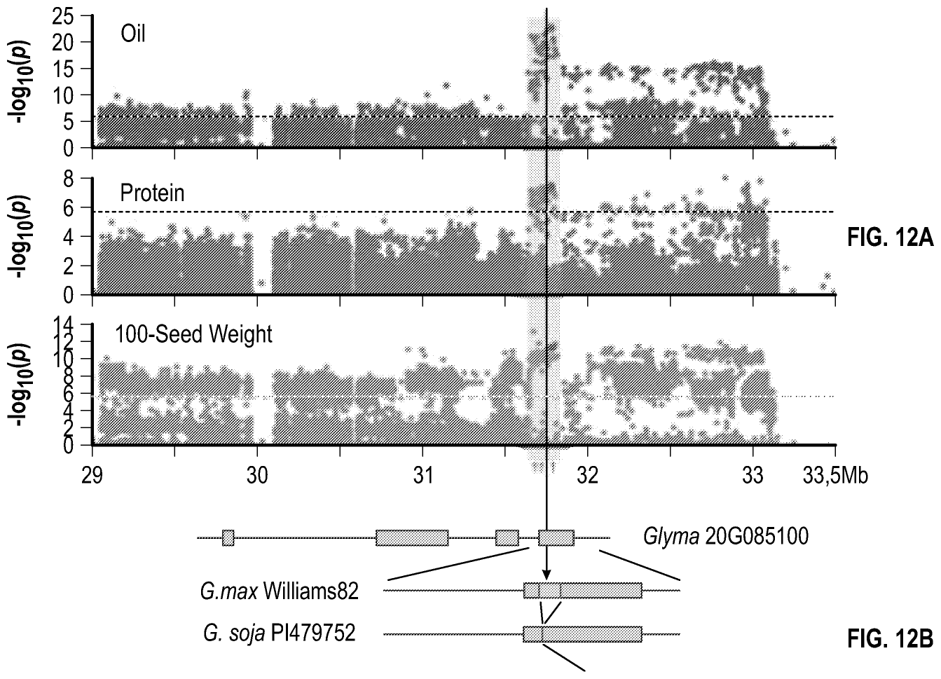

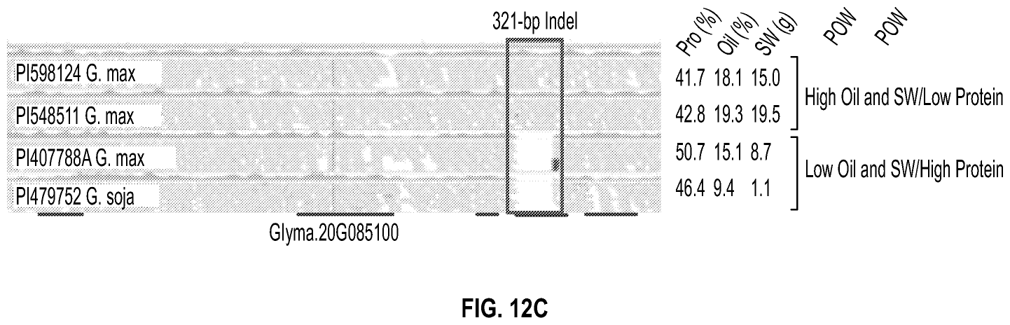

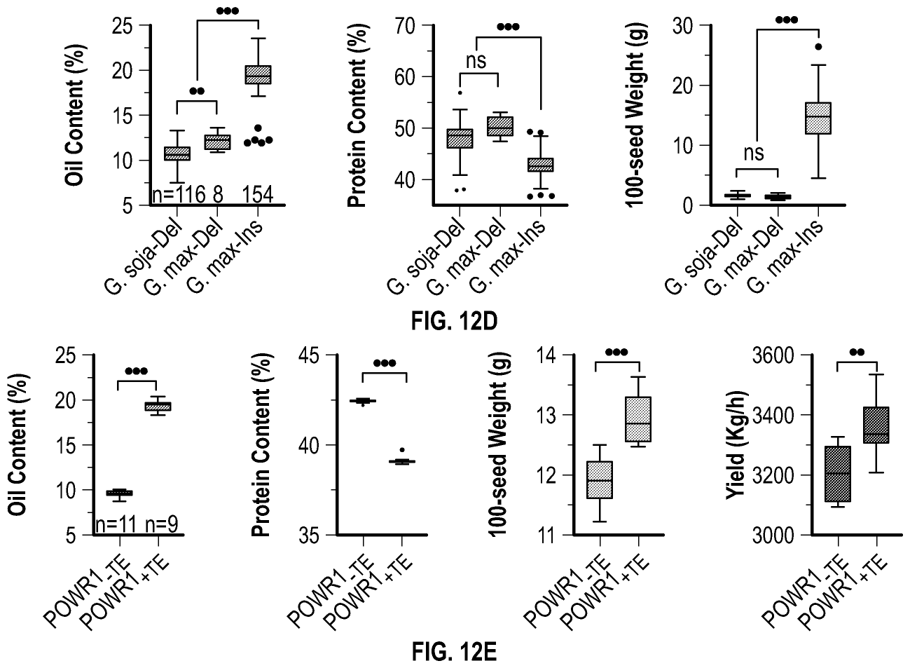

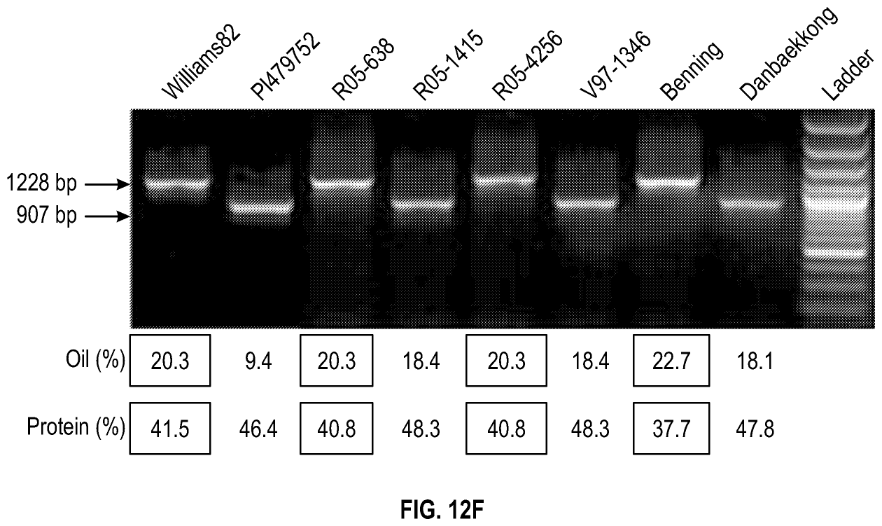

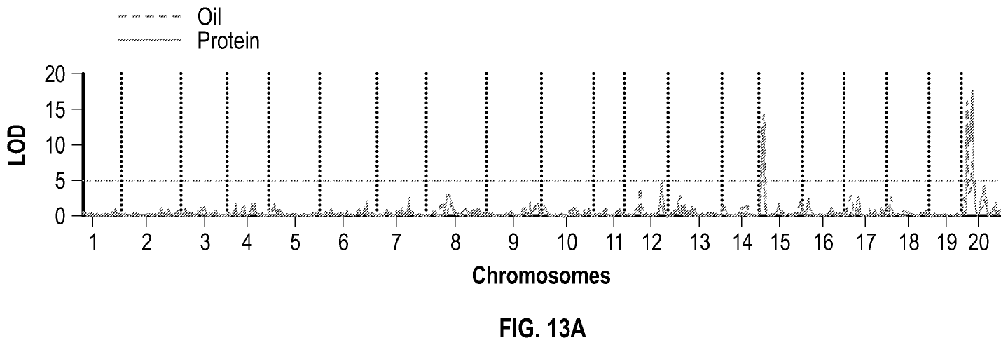

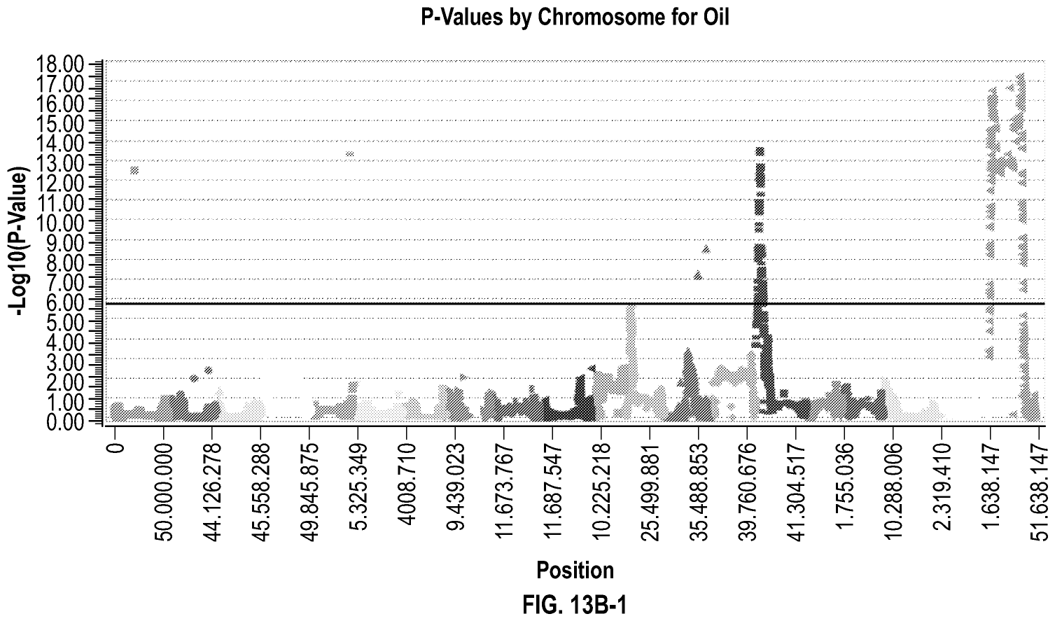

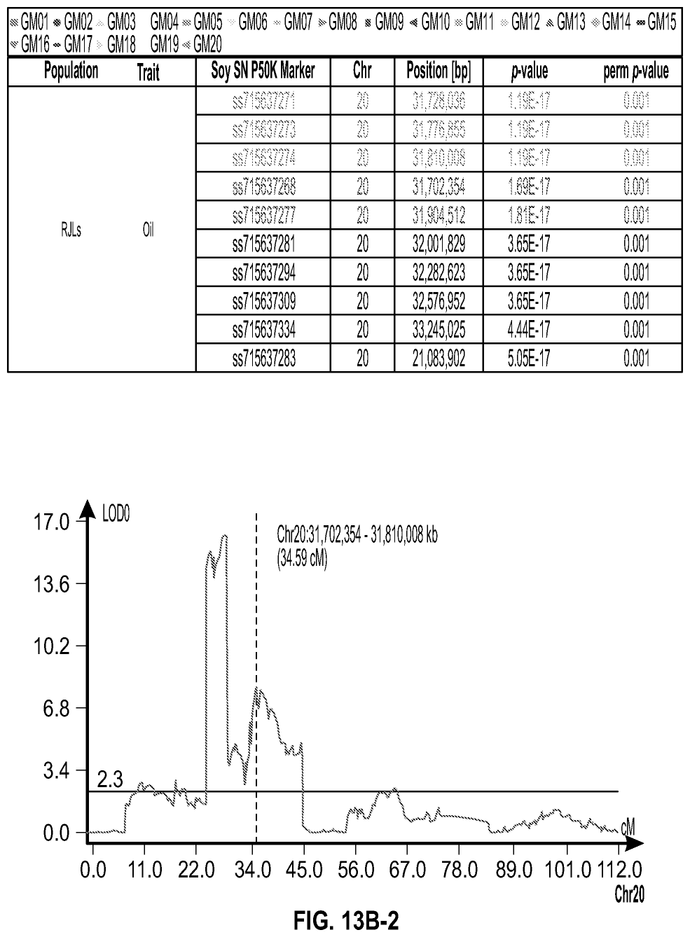

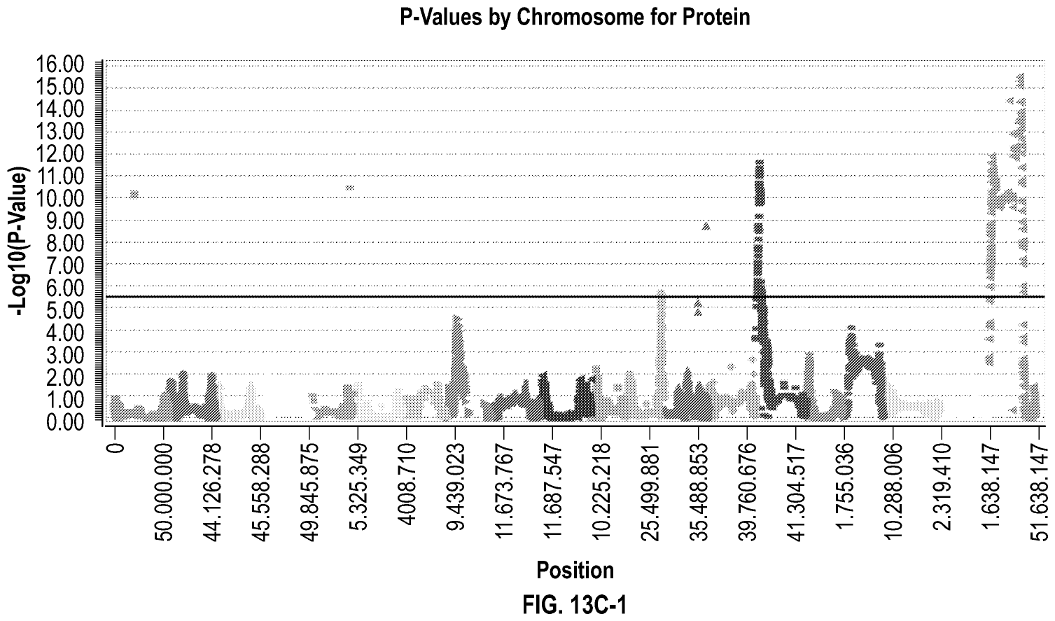

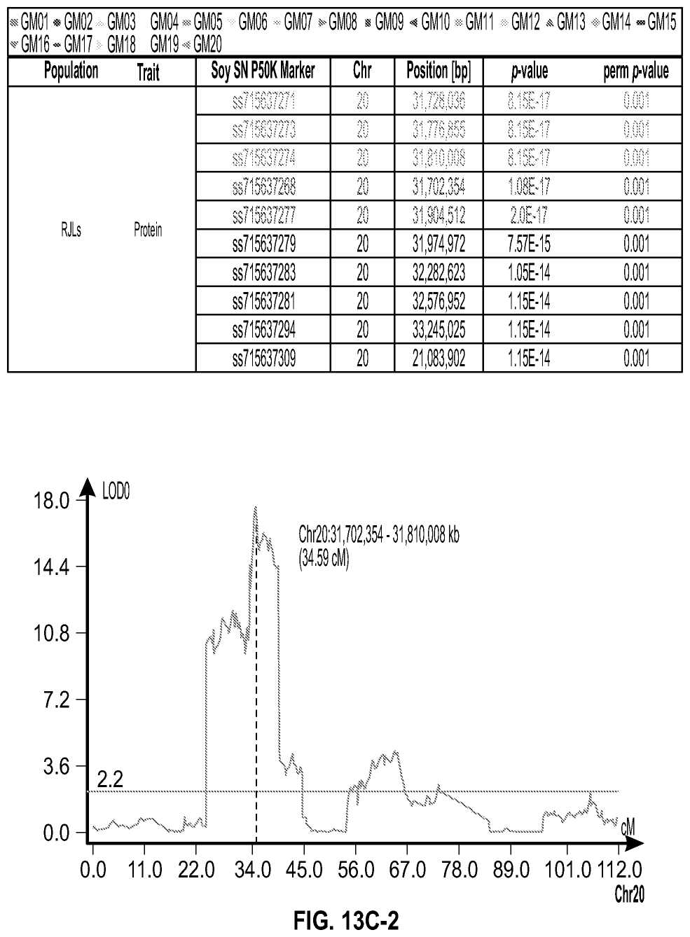

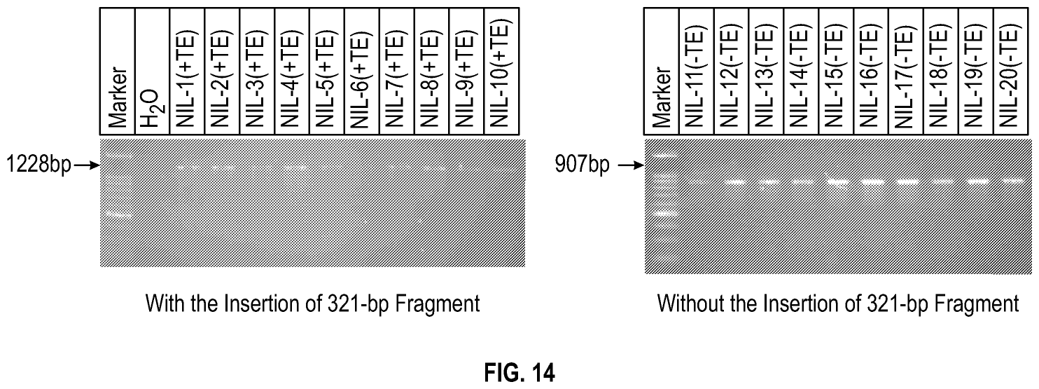

Genome-wide association studies (GWASs) using GLM and MLMM models with 38,066 genome-wide SNPs (Single Nucleotide Polymorphisms) identified three significant loci on chromosomes 10, 11, 20 for oil content with a values less than 0.05 in a panel of 278 diverse soybean accessions (FIGS. 13A and 14B). The most significant SNP (ss715637321 on chr20: 32,835,139) on the chr20 coincided with a genomic region where high-effect protein and oil QTLs have been repeatedly mapped to in the last three decades, but their underlying variant remains unknown. The current analysis was focused on the QTL on chr20 QTL and delimited the locus to an approximately 4-Mb region (chr20: 29,050,000-33,120,000) that expanded from the most significant SNP (FIGS. 11A and 11B). To uncover its underlying causative DNA variant, whole genome resequencing data of the 278 accessions was analyzed. The association study with the SNPs and InDels (Insertions and Deletions) present in the 4-Mb region identified a prominent cluster of 25 significant associations for oil that spanned a 154-kb region (chr20: 31,658,904-31,812,853) (FIG. 12A). Out of the 25 highly significant DNA variants (23 SNPs and 2 InDels with p≤1×10⁻¹⁷), a 321-bp InDel showed the most significant association (p=6.17×10⁻²⁴) (FIGS. 12A, 12B and 12C). The 321-bp InDel was also among the significant associations with protein content and 100-seed weight in the association analyses at a single nucleotide resolution (FIG. 12A; Table 10). None of these DNA variants located in coding regions of the 12 genes in the 154-kb region except for the 321-bp InDel present in Glyma.20G085100 (Table 10).

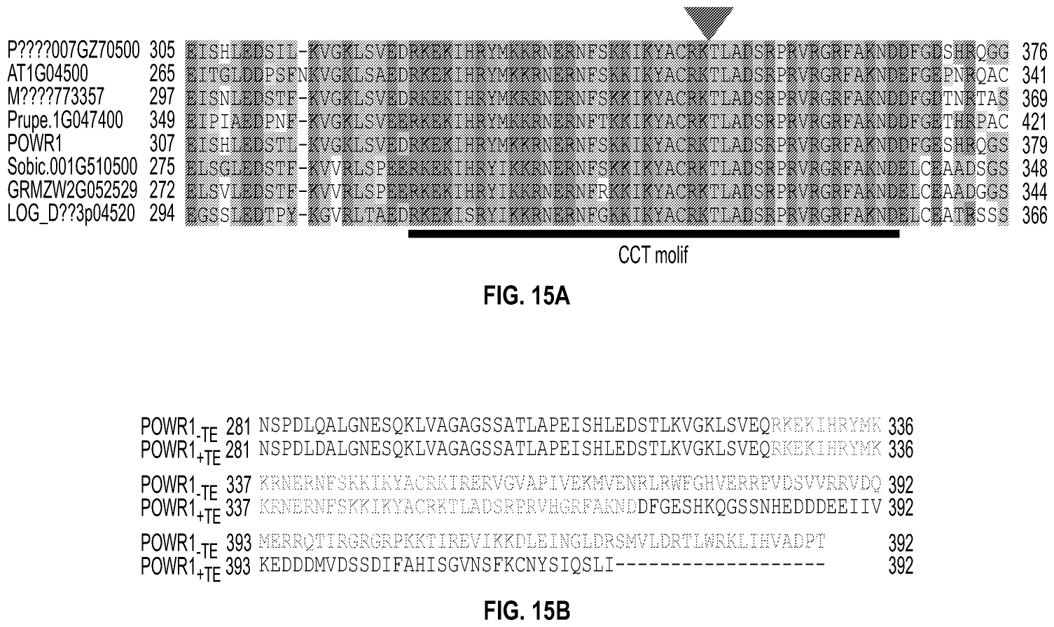

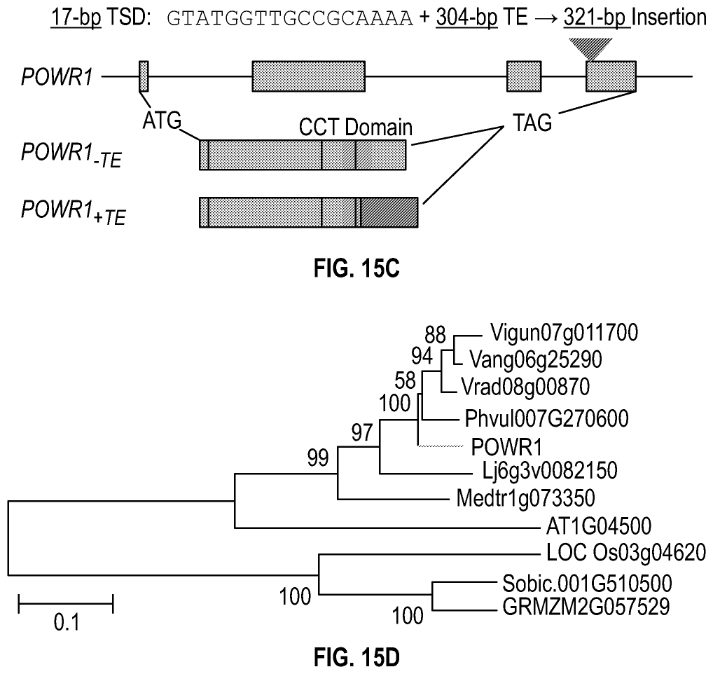

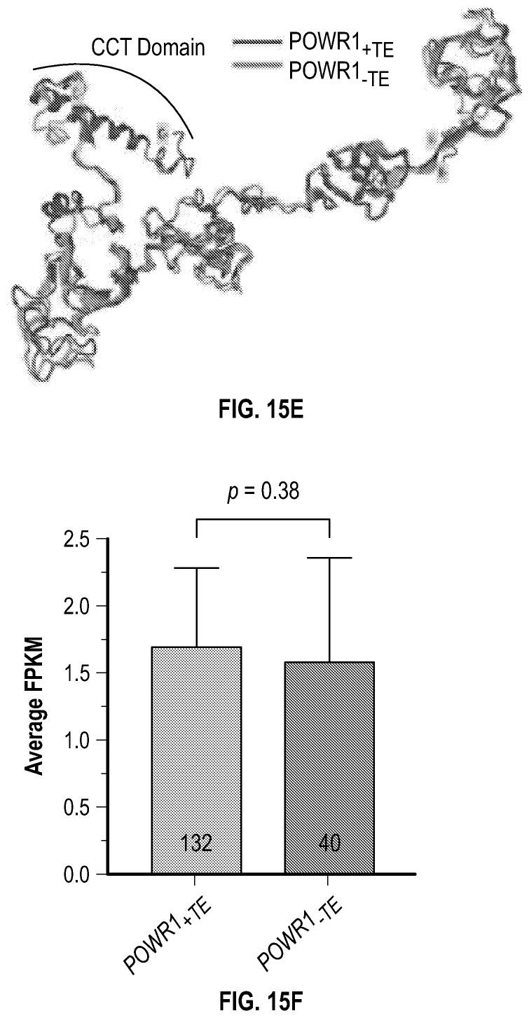

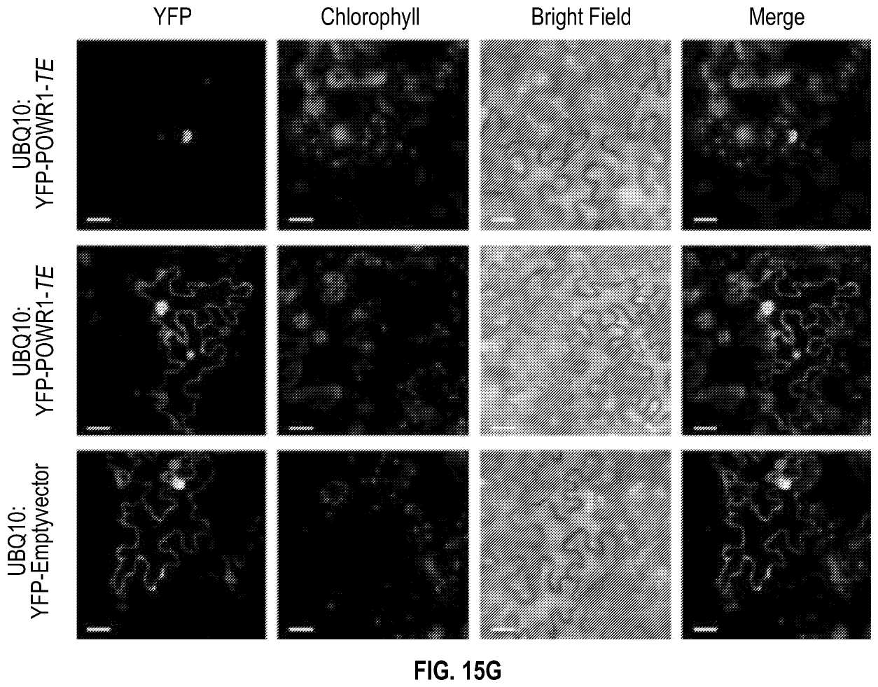

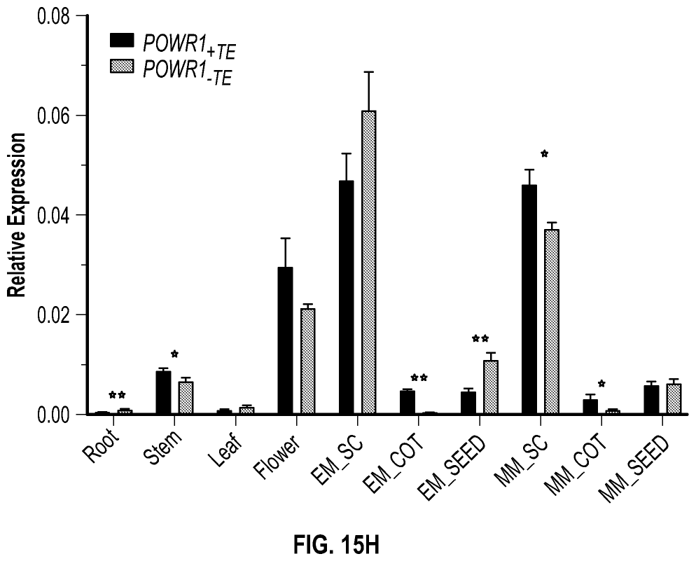

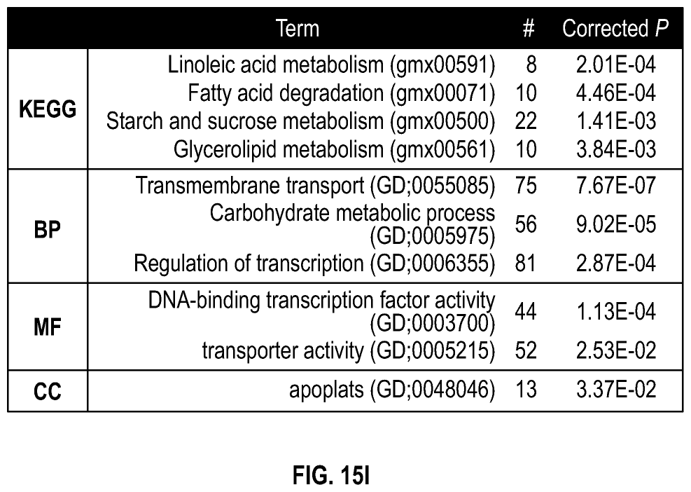

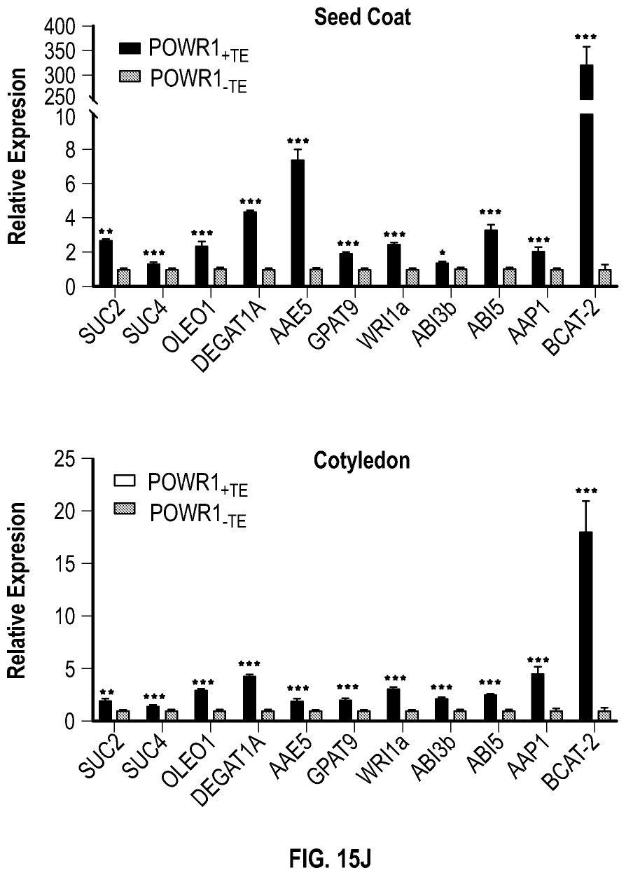

The seed oil and protein content, and seed weight were next examined in the panel of the accessions by splitting them into G. max-Del, G. max-Ins, and G. soja-Del. Interestingly, no G. soja accession containing the insertion allele was observed in the panel. However, both Del-carrying G. soja and G. max accessions were dramatically lower in oil (by 7.1 and 8.2%) and seed weight (by 14.0 and 14.59 g of 100-see weight) and higher in protein (by 5.1 and 7.3%) than G. max-Ins accessions. In contrast, no or relatively small differences (1.5% for oil, 2.2% for protein, 0.2 g for 100-seed weight on average) were present between G. soja-Del and G. max-Del for the three seed traits, suggesting that the observed phenotypic differences were primarily contributed by the InDel allelic variation rather than the overall difference between G. max and G. soja. (FIG. 15D). These results further supported that the chr20 QTL is associated with seed oil, protein, and 100-seed weight, and the 321-bp InDel in Glyma. 20G085100 was likely the causative variant for the chr20 QTL.

BLAST (Basic Local Alignment Search Tool) revealed that the 321-bp InDel sequence was highly homologous to the terminal sequence of a LINE (Long INterspersed Elements) transposon element (TE), which belongs to the GmI1 family. This gene was designated POWR1 for seed Protein, Oil, Weight Regulator 1. The POWR1 alleles with and without the 321-bp insertion were named POWR1_{+TE} and POWR1_{-TE}, respectively.

### B. The TE Insertion Likely Underlies the High-Effect Protein and Oil QTLs on Chr20 in Multiple RIL Populations

A genotype analysis was conducted on a bi-parental population of 300 recombinant inbred lines (RILs) generated from Williams 82 (G. max, HOLP (High Oil, Low Protein)) and PI479752 (G. soja, LOHP (Low Oil, High Protein) with the SoySNP50K array and further conducted both GWAS (GWAS_{RIL}) and linkage mapping. Linkage mapping identified two major QTLs on chr15 and chr20. The QTL on chr20 had a large effect and explained 21.9% of total oil variation and 23.4% of total protein variation. Both linkage mapping and GWAS revealed that the TE located in the most significant protein and oil QTL intervals on Chr20 (FIG. 13A). GWAS_{RIL} identified three adjacent SNPs on chr20 (ss715637271, ss715637273, ss715637274) that had the most significant, equal associations (p=1.19×10⁻¹⁷) with oil and protein content. They were all located within the 154-kb region identified above in association analysis using the panel of 278 diverse soybean accessions (FIG. 12A, FIGS. 13B, 13C). The TE insertion was located between two of the three peak SNPs (ss715637273, ss715637274). Whole genome sequencing and PCR genotyping results verified the presence of the TE insertion in Williams82 and absence in PI479752 (FIGS. 12C, 12F) and showed 100% co-segregation of the TE insertion with HOLP in 30 selected RILs containing either high protein or high oil (Table 11). Consistent with its effects in natural population used for GWAS described above, RILs carrying TE insertion contained 5.2% higher oil (p=4.00×10⁻¹³) and 6.2% lower protein (p=3.53×10⁻¹⁰) than those RILs lacking the insertion with statistical significance (Table 11). GWAS_{RIL} and linkage mapping from the RIL population provided additional evidence supporting that the 321-bp insertion as the causative variant for the oil and protein QTL on Chr20.

Large-effect protein and/or oil QTLs have been identified in the genomic regions containing POWR1 in multiple bi-parental RIL mapping populations, but their causative variants have remained unknown. A genotype analysis was conducted on the TE in parents of 15 mapping populations previously used for protein or oil QTL mapping. The results revealed that parents of seven populations (3 G. max×G. soja, 4 G. max×G. max) were polymorphic for the TE, while parents of eight populations (G. max×G. max) were not (FIG. 12F; Table 9). Notably, the oil and/or protein QTL on the chr20 region was only identified in populations whose parents were polymorphic for the TE insertion but not in populations whose parents were not polymorphic. In all seven pairs of parental lines, the high-oil parent carried the TE insertion while the low-oil parent lacked it (Table 12). These results further supported that the TE variation was likely the DNA variant underlying these previously mapped QTLs on chr20.

C. POWR1 Associated with Seed Field Yield in Addition to Seed Weight, Protein, and Oil Content

Correlation of the TE allele and seed traits were further investigated by analyzing a set of near-isogenic G. max lines (NILs) at the QTL on chr20. The polymorphism of the TE insertion in NILs was experimentally confirmed, and the variation completely correlated with the phenotypic variation of seed protein and oil content and seed weight (FIG. 14). Consistently, NILs lacking the TE (POWR1_{-TE}) exhibited significantly 3.29% higher in seed protein (p<0.001), 1.95% lower in seed oil (p<0.001), and 1.04 g reduced 100-seed weight (p<0.001) than those carrying the 321-bp insertion (POWR1_{+TE}) (FIG. 12E). Importantly, those POWR1_{+TE}-carrying lines had 150.3 kg/ha higher yield than POWR1_{-TE} lines (p<0.01), suggesting that POWR1_{+TE} played an important role in increasing yield potential in addition to the three seed traits (oil, protein, and seed weight).

D. POWR1_{+TE} Encodes a Truncated CCT Domain Protein with Altered Nuclear Localization

POWR1_{-TE} encoded a protein containing a highly conserved CCT (CONSTANS, CO-like, and TOC1)-domain at the C-terminus. It was present in both dicot and monocot species, suggesting its ancient origin in plants (FIGS. 15A and 15D). POWR1_{-TE} in wild soybean PI479752 contained an intact CCT domain of 44 amino acids, whereas POWR1_{+TE} in cultivated soybean Williams 82 contained the TE insertion in Exon 4 encoding part of the CCT motif (FIGS. 15A, 15B and 15C). The LINE transposon in POWR1_{+TE} is 304 bp in size and generated a 17-bp target site duplication (SEQ ID NO: 24; GTATGCTTGCCGCAAAA) upon insertion (FIG. 15C). Consequently, this 304-bp insertion resulted in a reading frameshift and produced POWR1_{+TE} containing a truncated CCT domain (27 amino acids short) and a distinct amino acid sequence at the C-terminus relative to POWR1_{-TE} (FIG. 15B). LINE transposons did not require excision to replicate. The mutation generated by the insertion should be stable. None of its closely related CCT genes in examined legume species contained the TE insertion (FIGS. 15A, 15D), suggesting that the TE insertion occurred in soybean and had a lineage-specific role in soybean.

The TE insertion caused little overall structural change in the predicted 3D protein structure between POWR1_{+TE} and POWR1_{-TE} except for their C-terminal end harboring the CCT domain (FIG. 15E). The second half of the CCT-motif contained a putative nuclear localization signal. The subcellular localization of POWR1_{-TE} was examined and determined if the TE insertion altered subcellular localization of POWR1_{+TE}. Transient expression of the two protein alleles in tobacco (Nicotiana benthaminana) leaves revealed that POWR1_{-TE} was exclusively localized in the nucleus (FIG. 15G), suggesting that POWR1 is a transcription-associated factor, in consistence with the fact that many CCT-domain proteins are transcription co-factors. However, POWR1_{+TE}, like the empty vector, was localized in both nucleus and cytoplasm, implying that the CCT domain is a functional element in its subcellular localization, and the TE insertion might affect function of POWR1 through disrupting its subcellular localization pattern.

E. POWR1_{-TE} and POWR 1_{+TE} Preferentially Expressed in Seed Coat and Flowers in a Similar Expression Pattern.

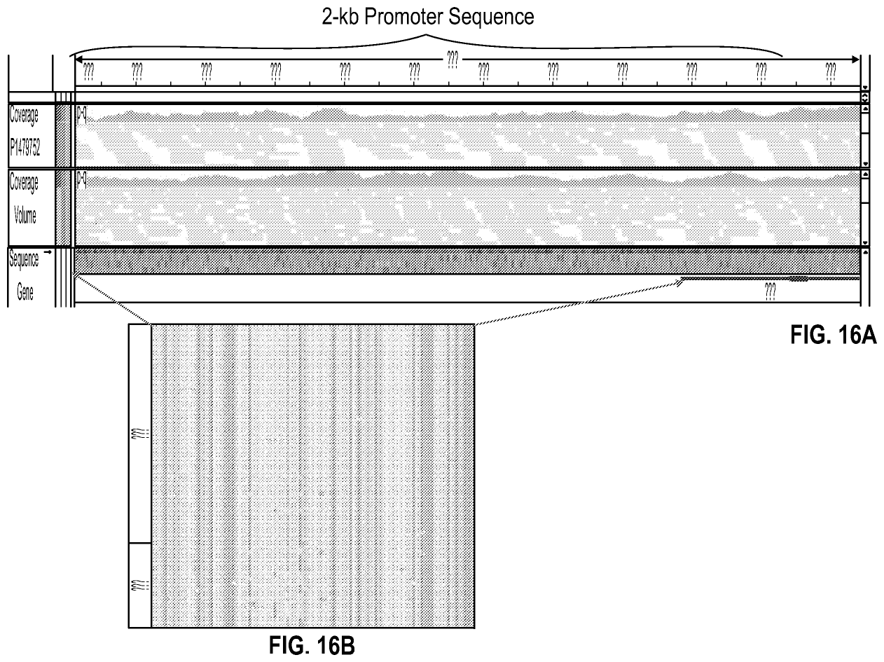

Gene expression analysis revealed that both POWR1 alleles preferentially expressed in flowers and developing seed coat at the early and middle maturation stages. They also had a similar expression pattern in the tissues (FIG. 15H), suggesting that the TE insertion unlikely affected their expression pattern. Comprising transcriptomes of mid-maturation seeds from 132 soybean accessions containing POWR1_{+TE} and 40 containing POWR1_{-TE} revealed no significant expression difference between POWR1_{+TE} and POWR1_{-TE} (FIG. 15F). Sequence variation in the 2-kb promoter sequences was not observed between POWR1_{+TE} in Williams 82 and POWR1_{-TE} in PI479752, the RIL parental lines analyzed above (FIG. 16A-16B). Thus, both gene expression and sequence comparisons suggest that TE insertion cause variation of seed traits likely through altering protein activity, not gene expression. Preferential expression in seed coat implied a possible role of POWR1 in nutrient transport in seed coat, a major function of seed coat.

### F. POWR1 Affects Genes and Pathways Involved in Seed Composition Traits and Seed Weight

To gain insight into molecular mechanism underlying how POWR1 regulates the seed traits, the transcriptomes of mid-maturation seeds were compared between four and six G. max accessions carrying POWR1_{-TE} and POWR1_{+TE}, respectively. As expected, the two genotypic groups had no significant difference in POWR1 expression (Table 13). The transcriptomic comparison identified a total of 1,163 differentially expressed genes (DEGs) associated with TE insertion. KEGG and GO terms related to metabolisms of fatty acid, lipid, and starch and sucrose, transmembrane transport, carbohydrate metabolism, regulation of transcription (biological process) and apoplast (cellular component) were significantly enriched for the DEGs (FIG. 15I). This result is consistent with the preferential expression of POWR1 in seed coat tissues that are mainly responsible for transporting multiple nutrients to support metabolic activities in cotyledon for seed development (FIG. 15H), as well as its pleiotropic effects on multiple seed traits including oil and protein content and seed weight.

Expression analysis revealed that a set of regulatory and metabolic genes involved in protein and oil production were differentially regulated in the seed coat and/or cotyledon of NILs for POWR1 (+TE/−TE) at the mid-maturation stage (FIG. 15J). For example, lipid biosynthesis genes (DGAT1, AAE, GAPT9) and sugar transporter genes (SUC2, SUS4) were significantly increased in POWR1_{+TE} relative to POWR1_{-TE} background in both seed coat and cotyledon tissues. The most striking increase was observed for BCAT2, which is involved in branched-chain amino acid metabolism, suggesting its contributing role for relatively lower protein content in POWR1_{+TE} than in POWR1_{-TE}. The regulators (WRI1, ABI3b, and ABI5) involved in seed development and size, as well as oil accumulation, were also upregulated in POWR1_{+TE} relative to in POWR1_{-TE}, suggesting that these regulators might act downstream of POWR1. Differential expression of these genes in the NILs suggests that they are likely part of the transcriptional regulatory cascade underlying POWR1 regulation of the seed traits.

### G. Expression of POWR 1-TE in Transgenic Soybean Increased Protein and Reduced Oil Content and Seed Weight

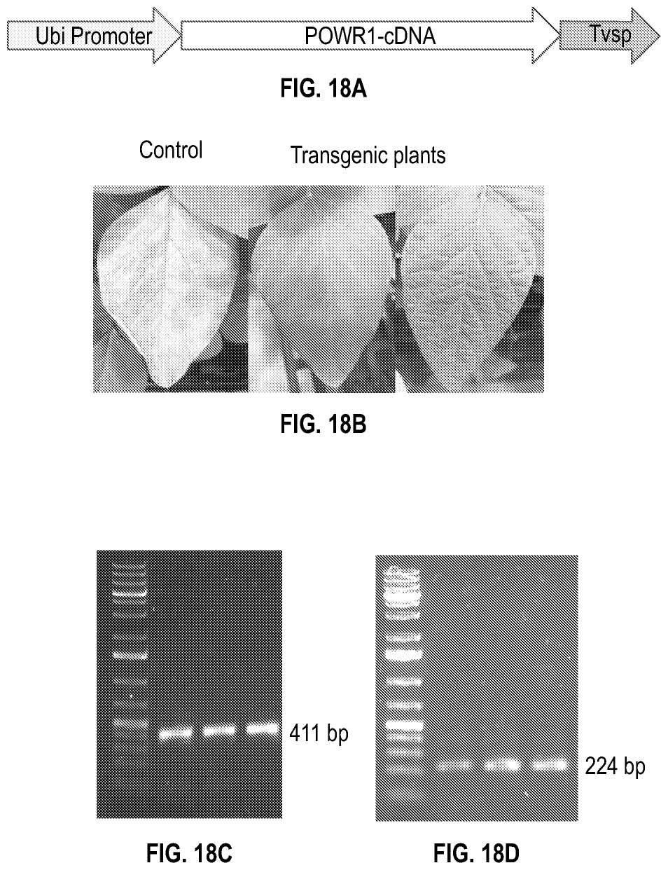

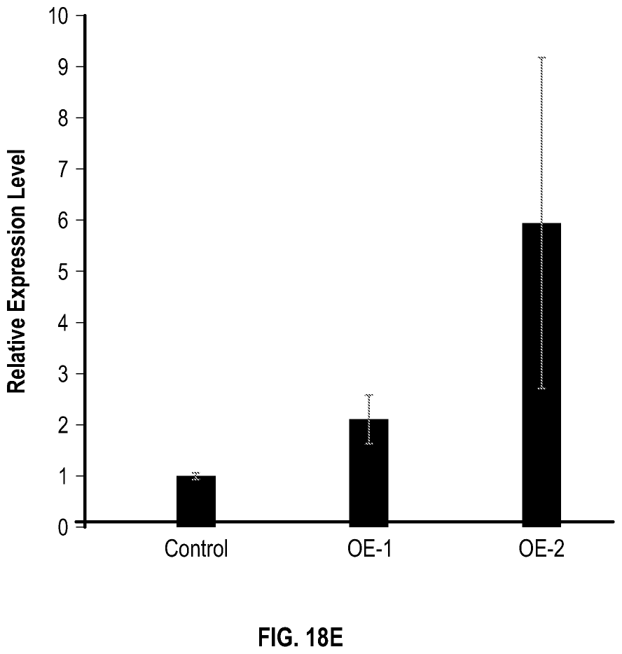

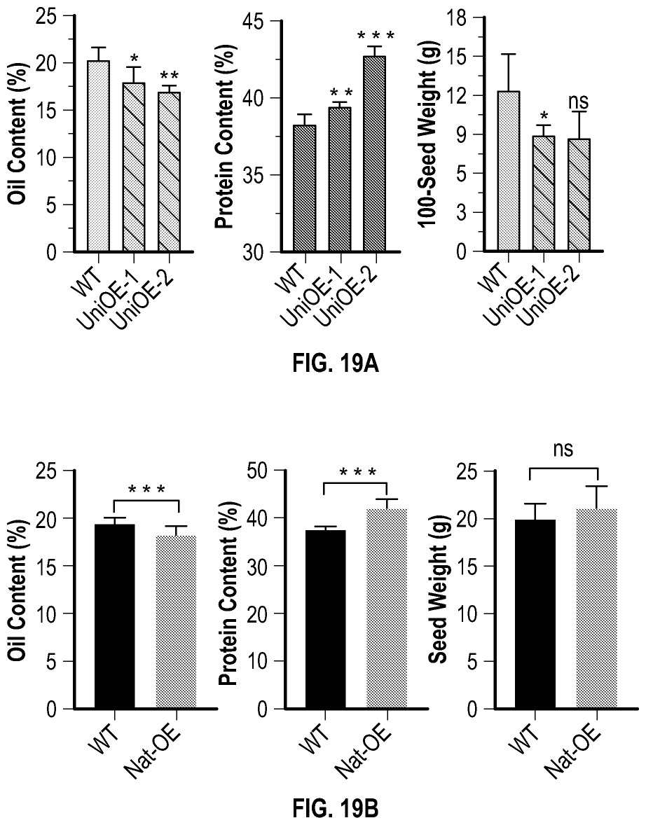

To examine the function of POWR1_{-TE}, the intact POWR1_{-TE} cDNA was introduced driven by a strong and constitutive expressing Ubiquitin promoter and the 1.9-kb POWR1 native promoter into POWR1_{+TE} G. max background (cultivars. Maverick and Williams 82, respectively). Two events overexpressing (OE) Ubiquitin promoter-driven POWR1 transgenic seeds (UbiOE1 and 2) were obtained, and qRT-PCR confirmed its high expression in OE plants (FIG. 18E). The UbiOE1 and UbiOE2 seeds contained significantly higher seed protein content (p<0.01) by 2.50% and lower seed oil by 2.36% (p<0.05) and 100-seed weight (p<0.05) by 3.57 g compared with those in non-transgenic control seeds (FIG. 19A). Eighteen (18) independent T1 transgenic plants were analyzed, it was observed that soybean containing native promoter-driven POWR1_{-TE} (Nat-OE) contained significantly higher seed protein by 4.39% and significantly lower seed oil by 1.31%, but had no statistically significant change in seed weight (FIG. 19B). The results clearly supported that POWR1 controls seed oil and protein content and seed weight in soybean, and it can be manipulated to alter soybean protein, oil and seed weight for seed quality improvement.

### H. POWR1 is a Domestication Gene

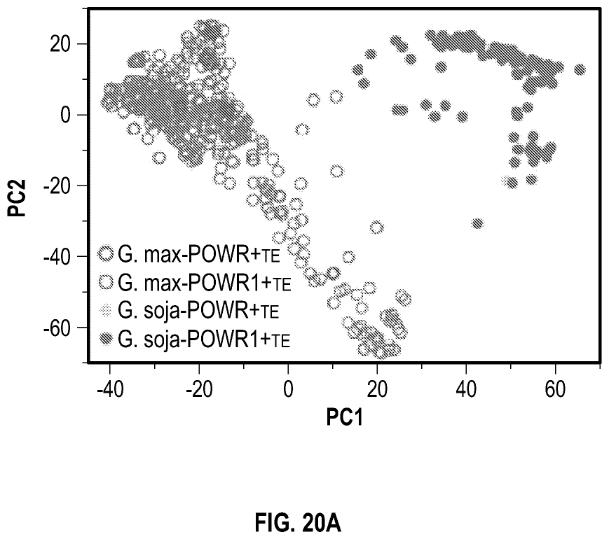

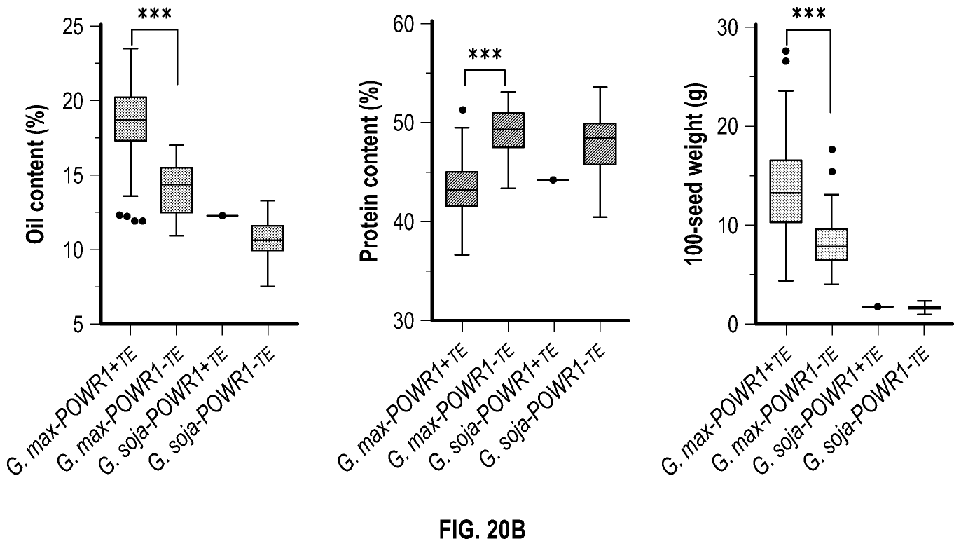

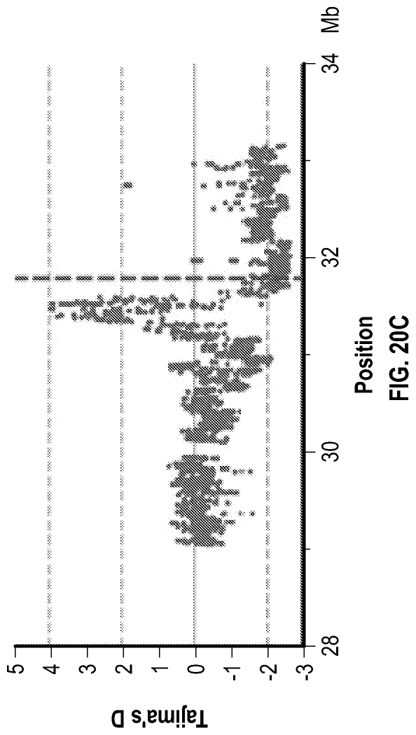

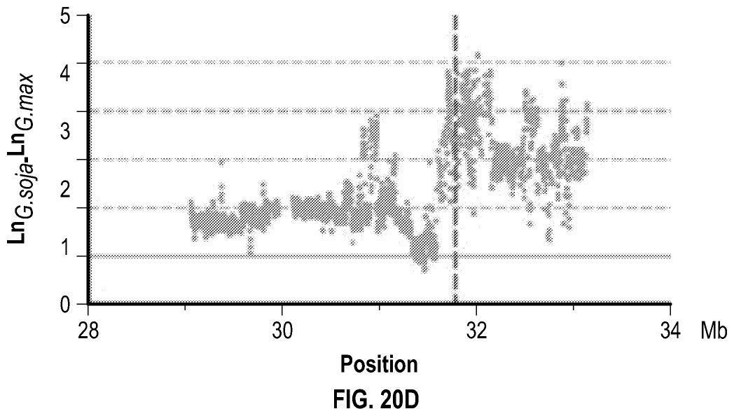

Next, the distribution of POWR1 alleles in an expanded soybean population consisting of 548 diverse accessions was evaluated. Principal component analysis (PCA) revealed that the majority of 150 G. soja accessions were clustered together as one group exterior to the group consisting of 398 G. max accessions (FIG. 20A). After allele assignment, it was found a nearly complete association of POWR1_{-TE} and POWR1_{+TE} alleles with G. soja and G. max populations, respectively, with a few exceptions. Specifically, 94.7% (377 of 398) of G. max possessed the POWR1_{+TE} allele, while all G. soja but one (149 of 150) carried the POWR1_{-TE} allele (FIG. 20A). In agreement with earlier results, the POWR1_{-TE} allele was associated with 4.47% lower oil and 5.73% higher protein contents, and 5.08 g lower seed weight than POWR1_{+TE} allele in G. max accessions significantly (p<0.001). This pattern of allelic effects on the seed traits remained in G. soja groups (1.56% for oil, 3.65% for protein, 0.12 g for seed weight) (FIG. 20B). A genomic scan revealed that POWR1 was located within an approximately 520-kb selective sweep region (chr20: 31,641,057-32,160,913) as inferred by Tajima's D of <−2 (FIG. 20C) and high G. soja/G. max IT In-ratios (larger than 2.4) (chr20: 31,654,290-32,157,761) (FIG. 20D). These results indicated that POWR1 was a domestication gene contributing to the phenotypic variation of the seed traits, and that POWR1_{+TE} was subjected to artificial selection likely for higher seed weight and oil during soybean domestication.

### I. Dynamic Interspecific Allele Transfer During Post-Domestication

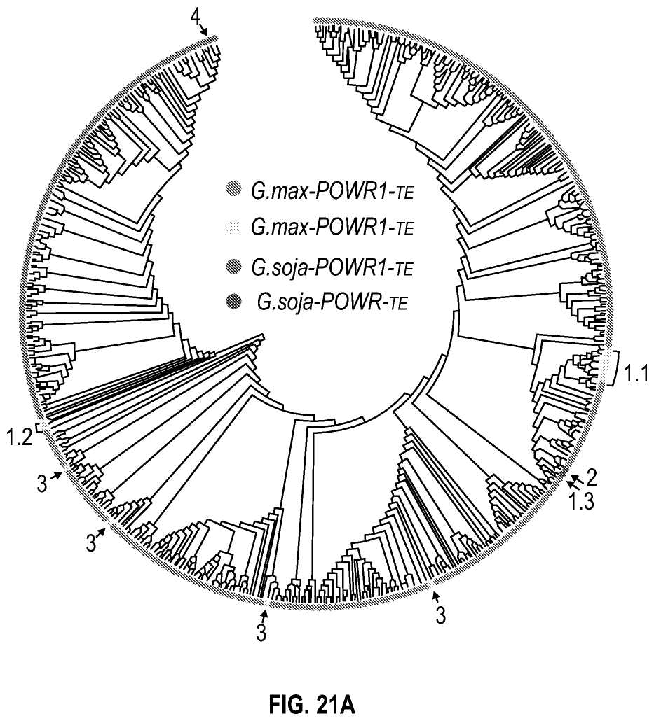

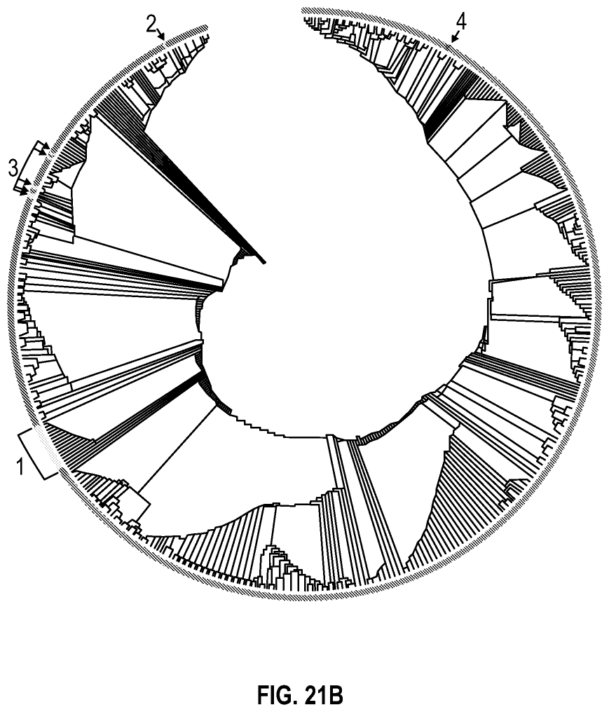

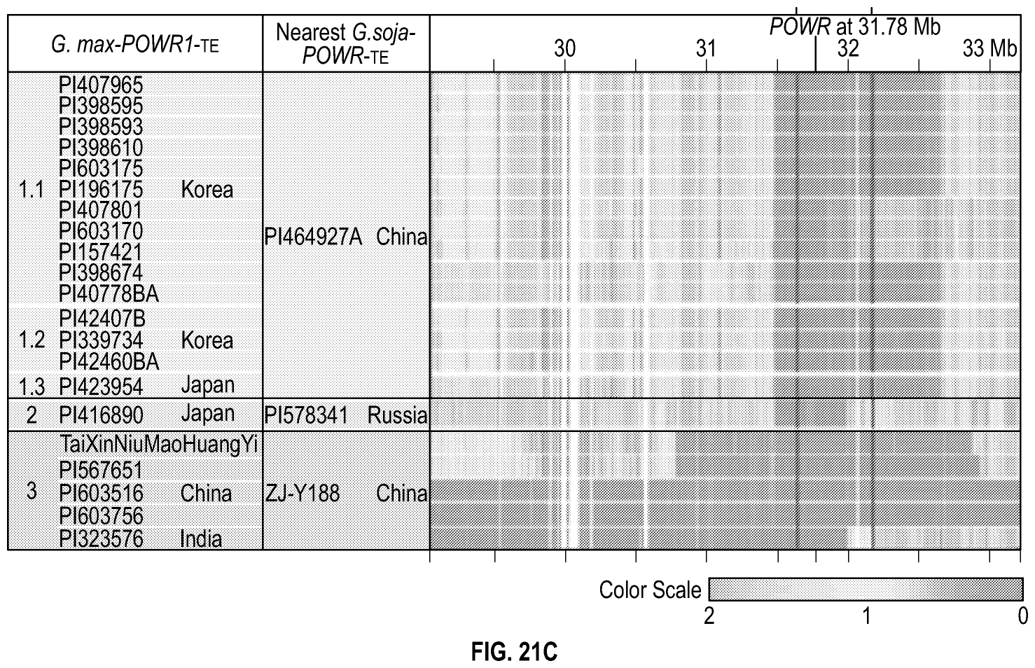

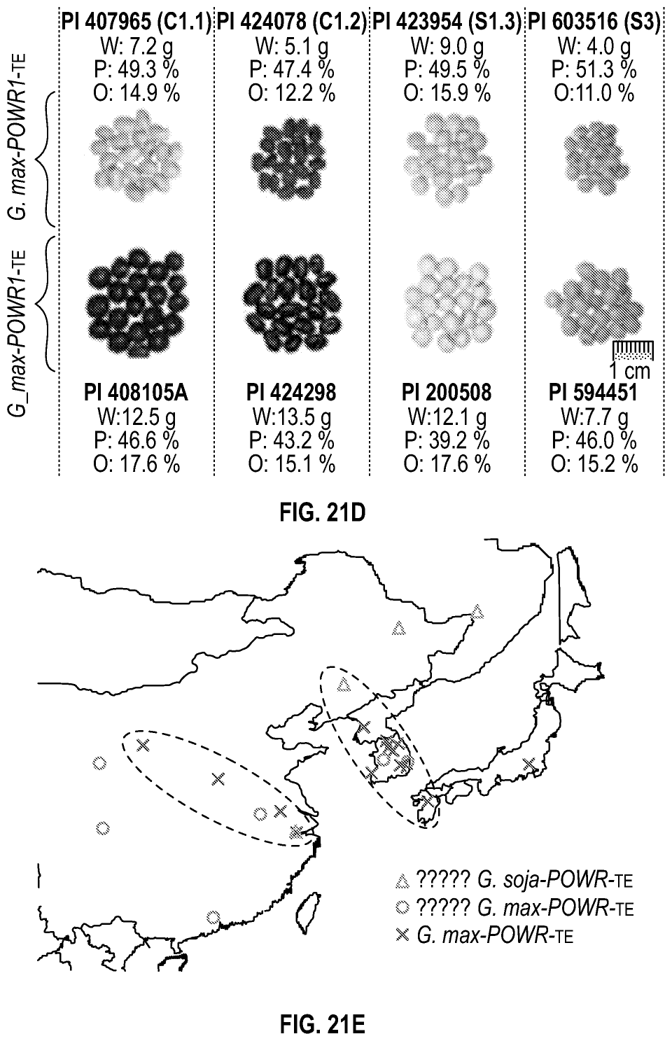

It was observed that twenty-one G. max-POWR1_{-TE} accessions and one G. soja-POWR1_{+TE} accession had POWR1 alleles contrasting the majority of G. max-POWR1_{+TE} and G. soja-POWR1_{-TE} accessions (FIG. 20A). To learn about the origin of the unusual presence of POWR1 alleles in these exceptional accessions, a global phylogenetic tree was constructed using the genome-wide SoySNP50K SNPs and a local phylogenetic tree using the whole genome resequencing-generated SNPs in the 154-kb region for the 548 accessions (FIG. 12A). The global tree exhibited similarity to the PCA result (FIGS. 20A, 21A). All G. max accessions (G. max-POWR1_{+TE} and G. max-POWR1_{-TE} (clusters 1.1, 1.2, 1.3, 2, 3)) clustered together and were separated from all G. soja accessions (G. soja-POWR1_{-TE} and G. soja-POWR1_{+TE} (singleton 4)), regardless of the TE variation (FIG. 21A). However, in the local phylogenetic tree, all G. max-POWR1_{-TE} accessions changed from the G. max cluster as seen in the global tree to the more diverse G. soja clusters (clusters 1, 2, 3) while the G. soja-POWR1_{+TE} accession (singleton 4) switched to the G. max cluster (FIG. 21B), indicating that transfers of POWR1 alleles occurred between G. soja and G. max after domestication and produced the G. soja-POWR1_{+TE} accession and the G. max-POWR1_{-TE} accessions. Without including these accessions with post-domestication allele transfer, all remaining G. soja accessions carried POWR1_{-TE} and all G. max accessions contained the POWR1_{+TE} allele. The complete association of POWR1_{+TE} with G. max and POWR1_{-TE} with G. soja and its function in controlling seed weight and yield, an important domestication syndrome, support that POWR1_{+TE} was subjected to strong and exclusive selection during the domestication and plays a key role in soybean domestication.

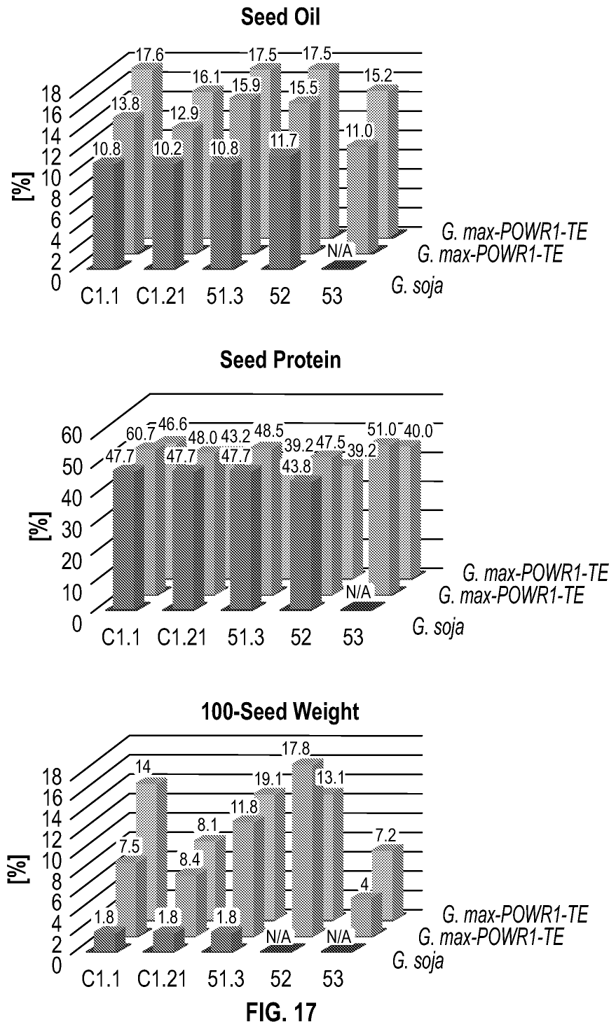

All G. max-POWR 1-TE were clearly clustered into three clusters (clusters 1, 2, 3) in G. soja clade of the local tree, while the accessions from each of these clusters were split into different, far related clusters in the global tree, such as cluster 1 to 1.1, 1.2, 1.3 and scatted distribution of cluster 3 accessions in G. max clade. This suggested that the fragments harboring POWR1_{-TE} were transferred into diverse G. max accessions likely from G. soja accessions, hence producing those G. max-POWR1_{-TE} accessions with diverse genetic backgrounds, as shown by their scatted distribution in the global tree (FIG. 21A). To gain insight into the allele transfer, the pairwise genetic distance across the 4.1-Mb region between each of the 21 G. max-POWR1_{-TE} accessions, and their phylogenetically closest G. soja accessions (PI464927A, PI578341, and Zj-Y188) in the local tree, was calculated and plotted to detect possible transferred regions harboring POWR1_{-TE} (FIG. 21C). Pairwise distance analysis showed diverse patterns of highly identical sequences with variable lengths within the region among the three clusters. Briefly, a region (roughly 1.2 Mb long) with high sequence identity with shared one end or both ends was identified in the cluster 1 while cluster 3 had the transferred fragments carrying the POWR1_{-TE} at variable lengths, and cluster 2 had the shortest transferred fragment containing the POWR1_{-TE} (˜ 500 kb long). The results supported that the POWR1_{-TE} in those G. max accessions likely originated from post-domestication allele transfer events and went through multiple chromosomal crossovers. Next, these accessions were mapped to their geographic origins and revealed close geographic proximity of G. max-POWR1_{-TE} with their phylogenetically closest G. soja-POWR1_{-TE} (in the local tree) and G. max-POWR1_{+TE} (in the global tree) in multiple geographic locations (South Korea, Japan, China) of East Asia (FIG. 21E), implying that the allele transfers likely took place within these regions. Indeed, despite an average decrease of 2.7% oil content and 3.2 g 100-seed weight, those G. max-POWR1_{-TE} from East Asia contained 6.5% higher protein content than their closely related G. max-POWR1_{+TE} accessions (FIG. 21D, FIG. 17), in accordance with needs for high-protein soy-food in East Asia.

### DISCUSSION

Significant efforts have been dedicated to identifying gene(s) and variant(s) causative for the QTL on chr20 in the past three decades, because of its strong association with multiple seed traits including seed weight, oil and protein content and yield, which represented the economically most important traits in soybean. Having taken advantage of whole genome re-sequencing data from 278 highly diverse accessions, the single nucleotide-resolution association analysis together with high-confident biparental genetic mapping, it was uncovered that a single gene, POWR1, underlies the QTL for the seed traits and a TE insertion in POWR1 was the causative allele (FIG. 12F). It is further supported by results from transgenic soybean experiments, subcellular localization studies of the two POWR1 alleles, and gene expression analyses. Association of POWR1_{+TE} with higher field yield in near isogenic lines is likely achieved through regulating seed weight because of their positive correlation. Although the pleiotropic effect of POWR1 on the important seed traits posed a challenge to improve all traits simultaneously, it offered an opportunity to use appropriate alleles for improving oil content, protein content or their balance. For example, soybean containing high protein content were developed by transferring POWR1_{-TE} from one of the few unusual G. max germplasms into elite lines carrying a POWR1_{+TE} allele.

It has been shown that CCT domain containing genes mainly function in photoperiod-related adaptation in Arabidopsis and cereals. However, the present study demonstrated that POWR1 regulates oil, protein and seed weight/yield in soybean. In consistence with its function, it was revealed that POWR1 was preferentially expressed in the coat, a tissue that played a key role in transporting nutrient into cotyledon in storage reserve production and seed filling. The TE insertion in the CCT domain disrupted the exclusive localization of POWR1 in the nucleus but caused little change in its expression in seeds and other seed compartments and tissues. Thus, TE insertion increased oil and seed weight likely through altering its protein function, not its expression. Given the role of CCT domain in DNA binding and protein interaction, the transcriptome and real-time RT-PCR showed that POWR1 is likely involved in regulating the expression of genes involved in oil and protein metabolism, nutrient transporting and regulating seed development. For example, ABI5 with a known role in determining seed size and BCAT2 with a function in protein degradation had significantly higher expression in a POWR_{+TE} background, in accordance with the result that seeds carrying POWR_{+TE} had lower protein content, higher oil content and larger seed weight. Based on the expression analysis, POWR1_{-TE} may act upstream of these metabolic genes, transporter genes and regulators (including WRI1a, ABI5), which collectively affects the three seed traits.

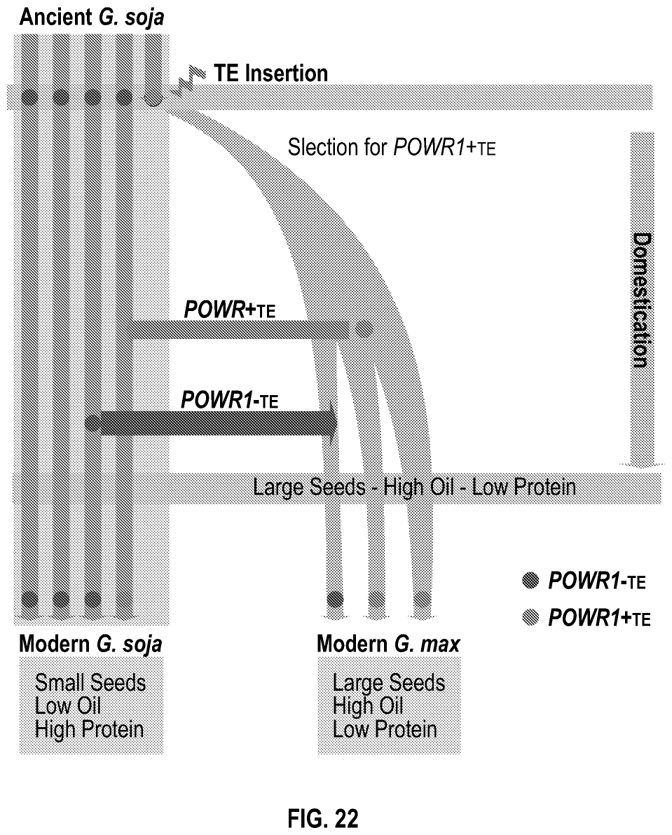

Larger seed size was suggested as an earlier selected domestication trait for several cereal crops, and it was likely true for soybean as well. Recent archaeological studies suggested that arose of increased oil content in soybean seed might be no later than seed enlargement, suggesting that oil content increase might occur earlier than or simultaneously with seed size enlargement. Nearly complete fixation of POWR_{+TE} in G. max and the complete absence in G. soja in this 548-accession population was also identified in a larger population consisting nearly 4000 soybean accessions being sequenced recently (FIG. 22). Thus, TE insertion in POWR1 should be among the key events during transitioning from G. soja to G. max. Selection for soybean with larger seeds and higher seed yield likely led to fix the POWR_{+TE} in modern G. max. However, it is unlikely that oil as a non-visible oil trait was the driving force for selection in early soybean domestication. Thus, oil increase could simply be the by-product since it is pleiotropically controlled by POWR1_{+TE} (FIG. 22). The resulting decrease in protein content in seed due to the preferential selection for POWR1_{+TE} might not have significant impact in ancient agriculture. However, it created present-day challenges for the animal feed industry and compromises seed protein content that was desired and increasingly demanded for human consumption. As the low-protein phenotype was fixed in G. max, transfer of the high-protein allele (POWR1_{-TE}) from G. soja into G. max may increase the seed protein content as needed. This represented a reversal of the domestication process, and introgression and selection for POWR1_{-TE} can be seen in Asian breeding programs which were likely driven by need for high protein soybean in Asia. A soybean accession with TE insertion that was annotated as G. soja was also observed. However, this accession was likely from a hybridization event between G. max and G. soja. Given an outcrossing rate of up to 19% for G. soja and up to 6% for G. max, natural gene flow and introgression from cultivated soybean to their wild relatives might be a common source such as arise of semi-wild soybean (FIG. 22).

The instant study provided strong evidence supporting that POWR1 played a key role in soybean domestication and pleiotropically regulates seed protein, oil, and weight, likely seed yield. However, many QTLs for the seed traits and several domestication genes including a recently identified GmSWEET gene underlying a QTL on Chr15 have been identified. Soybean seed oil, protein, seed weight and field yield phenotypic values were the accumulative effects of those QTLs across the soybean genome. It was still largely unknown about how POWR1 and other domestication genes were selected during soybean domestication in shaping modern cultivated soybean, and its interaction with other associated QTLs in determining the phenotypic value of those traits. This enabled better understanding of soybean domestication process and the molecular mechanism controlling those seed traits. A comprehensive investigation of these loci and their relationship with POWR1 may enable better understanding of soybean domestication process and their underlying molecular mechanism controlling those seed traits.

### Materials and Methods

### A. Plant Materials

A panel of 548 soybean accessions (398 cultivated soybean G. max and 150 wild soybean G. soja (Siebold & Zuccarini)) from the genetic resources information network (GRIN) database of U.S. National Plant Germplasm System (https://npgsweb.ars-grin.gov/) was used in this study. Out of 548 accessions, 278 accessions (116 G. soja and 162 G. max) with variations in seed oil (7.5-23.5%), seed protein (36.7-56.9%) and 100-seed weight (1.0-26.5 g) were used for association analysis (FIG. 13A-13C). An F_{6:7} population of 300 recombinant inbred lines (RILs) from a genetic cross between G. max cv. Williams 82 and G. soja PI479752 was used for genetic linkage mapping. Seed oil content among the RILs varied from 9.82-20.47% and 37.64-47.99% for protein content. Seeds of the parents and RILs were planted at the USDA-ARS farms in Beltsville, Maryland, in 2012 and 2015 with two replications in a randomized block design. The highly homozygous (>99%) near-isogenic lines (NILs) were created from a F7 plant heterozygous for POWR1 from a cross of G03-3101×LD00-2817P. Plant growth and phenotype measurements were performed as previously described. The NILs homozygous at the POWR1 locus were planted in replicated field trials in nine environments (one in Arkansas, Missouri, North Carolina, and six in Tennessee) in 2016 and 2017 with randomized complete block design. The TE variations in NIL lines were validated by a PCR assay with a pair of PCR primers flanking the InDel. All soybean plants including the transgenic lines used for DNA genotyping and quantification of seed traits were grown in the Donald Danforth Plant Science Center greenhouses (St. Louis, MO, USA).

### J. Measurement of Seed Traits

Phenotypic data including seed protein and oil content (%), 100-seed weight (g) for the panel of 548 accessions were acquired from the Germplasm Resources Information Network. Oil and protein content of the RIL population, the transgenic plants and all other soybean plants were measured using the near-infrared reflectance (NIR) spectroscopy using a DA 7250 NIR analyzer (Perten Instruments, Sweden) unless specified. Approximately 50 seeds per line were analyzed and measured twice. For NILs, approximately 20 g seeds were grounded to powder and also measured with Perten DA 7250 analyzer. Seed trait measurements were averaged over all replications and locations for both NIL groups and compared.

### K. Sample Sequencing, Read Alignment, and Variant Calling

A total of 91 diverse G. soja accessions which represent over 90% diversity of wild soybeans in the US soybean collection were re-sequenced using the Illumina NextSeq500 sequencer. For the remaining 457 accessions in the association panel and newly published soybean re-sequencing data, raw sequencing reads were retrieved from the NCBI SRA database. All quality-controlled reads were aligned to the G. max reference genome (Williams 82.a2 v1) with BWA. DNA variants including SNPs and InDels were called using the GATKs pipeline. The resulting variants were filtered using GATKs VariantFiltration with following parameters: read depth ≥5 reads, SNP quality ≥50, and at least 2 SNPs in a 10-bp window were allowed. Read alignments were visualized using the Integrative Genomics Viewer. The resulting 28,708 SNP and 131 InDel markers in a 4.1-Mb region (29-33.15 Mb) were used to carry out regional association analyses. Whole developing seeds at the mid-maturation stage were collected in environmental controlled greenhouses and multiple seeds per accession were pooled for transcriptome sequencing, as previously described. Transcriptome analysis was performed with TopHat and Cufflinks, and the FPKMs across samples were normalized with the quantile method in Cuffdiff.

### L. Association and Linkage Mapping

DNA variants were quality controlled before being used for genome-wide or regional association analysis with TASSEL5 with following criteria: a minimum minor SNP allele frequency of 0.05, a maximum proportion of heterozygous sites of 0.2, and a minimum number of accessions per site of 85%. Five principal components as determined in TASSEL5 were used for population structure (Q). Kinship (K) was calculated using centered IBS method in TASSEL5. GLM (general linear model) and MLMM (mixed linear model) were used for genome-wide association mapping and regional association analysis, as implemented in TASSEL. For the RIL population, GLM without population structure Q, or GLM with Q, or MLM with Q and kinship K, returned almost identical mapping associations for oil and protein using 19,848 SNPs from the SoySNP50K-set. The Bonferroni-corrected genome-wide significance threshold was calculated as 0.05/SNP count. Linkage mapping was carried out using Windows QTL Cartographer v2.5 and QTLs were detected using the composite interval mapping with 1,000 permutations for each test as previously described.

### M. Genetic Diversity Analyses

Principal Component Analysis (PCA) of the association panel was conducted in TASSEL using the SoySNP50K SNPs. The wild soybean and cultivated soybean accessions from the 548 accessions were used to calculate Tajima's D and the pairwise nucleotide diversity IT was calculated in TASSEL5. Regions accounting for the top 15% In-ratios (which corresponds to an In-ratio threshold of about 2.4) or Tajima's D of <−2 were considered as domesticated.

### N. Phylogenetic Tree and Sequence Alignment Analyses

The unrooted Neighbor-Joining phylogenetic tree was constructed with the 548 accessions using MEGA7 with the Maximum Likelihood method based on the Tamura-Nei model. A total of 19,284 genome-wide SNPs were used for the global tree and 1,023 SNPs within the 154-kb domestication region were used for the local tree. Multiple DNA and protein alignments were performed in Clustal Omega. Structures of the proteins were predicted by I-TASSER, were compared with RaptorX (TMscore 0.797), and visualized with iCn3D.

### O. RNA Extraction and Expression Analyses

Soybean NILs for the POWR1 locus were used for expression analyses. Soybean leaves, roots, and stem tissues were collected at 4 weeks after planting. Fully-open flowers were collected after their emergence. Early maturation seeds (25˜50 mg weight) and middle maturation seeds (100˜125 mg weight) were collected, and half of them were dissected to obtain seed coat and cotyledon tissues separately. RNA was extracted as described previously. Expression levels of genes of interest were determined and normalized to that of GmCYP2 (Glyma. 12G024700) with the BioRad CFX384 Real Time PCR System using SsoAdvanced Universal SYBR® Green Supermix. Primers for each gene are listed in Supplemental Table 11. Experiments were performed with both biological and technical triplicates. For POWR1 expression levels in soybean transgenic lines, seeds at early maturation (25˜50 mg weight) were collected and used for RNA extraction. Transcriptome sequencing and analysis were performed as previously described.

### P. DNA Vector Construction and Soybean Transformation

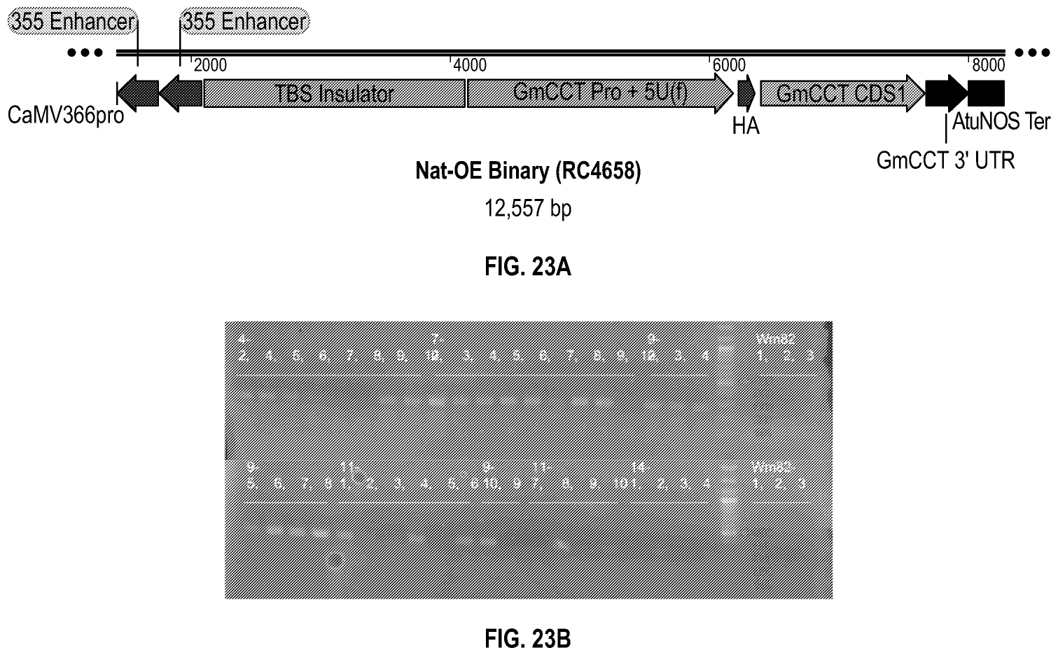

A vector (backbone pMU106) containing synthetic cDNA of POWR1_{-TE} allele from PI479752 driven by the Ubi917 promoter, pUbi: POWR1_{-TE} was constructed (FIG. 18A) and transformed into G. max cv. Maverick carrying POWR1_{+TE} using an improved Agrobacterium mediated transformation protocol as previously described. The presence of the construct in transgenic plants was confirmed by Basta leaf-painting (FIG. 18B) and PCR assay (FIGS. 18C, 18D). Expression level of POWR1 in transgenic plants was confirmed by qRT-PCR in developing seeds at the early maturation stage (FIG. 18E). With the same strategy, the cDNA of POWR1_{-TE} allele driven by its 1.9-kb native promoter sequence was cloned into a customized expression vector (backbone pAGM4673) and transformed into soybean using the Agrobacterium mediated transformation at Wisconsin Crop Innovation Center (Madison, WI) (FIG. 23A). The spectinomycin resistance was used as selection marker, followed by PCR (FIG. 23B) determination using the primers specific to the vector sequences were used to determine positive TO plants and the primers (F: TATCCATATGACGTTCCAGATTACGCC (SEQ ID NO: 20); R: ACCTCAGAATTTTGCAGTGTGTGTG (SEQ ID NO: 21)) spanning the vector and CDS to identify T1 positive transformants. T1 seeds were used to measure protein, oil and weight. For transient expression, synthesized cDNAs of POWR1_{-TE} and POWR1_{+TE} were cloned into the Gateway entry vector pcr8/Topo. These constructs were moved into plant gateway expression vectors UBQ10:YFP-GW with LR clonase.

### Q. Transient Expression and Microscopy Analyses

POWR1_{-TE} and POWR1_{+TE} expression localization were observed through transient expression in N. benthamiana using the method of Li. Briefly, UBQ10:YFP-POWR1_{-TE}, UBQ10:YFP-POWR1_{+TE}, and UBQ10:YFP in A. tumefaciens were infiltrated into young leaves of N. benthamiana plants (4˜6 weeks) using a 3-ml syringe without the needle. Leaves were imaged 48 h after infiltration.

Confocal images were obtained with a Leica TCS SP8 confocal microscope using the 63× water immersion lens. Samples were excited with a 514-nm laser line and 649-nm laser line to detect YFP and chlorophyll signals, respectively. Fluorescence emission was collected for best signals of indicated fluorescent probes.

### Example 3. Study Design

The current study aimed to comprehensively understand CCT domain-containing genes for their role in protein-oil accumulation in soybean seeds to facilitate functional genomics research and soybean improvement. The current study explored the evolution, expansion, and domain composition of CCTs by comparing them with a diverse range of plant species. The current study subsequently highlighted natural variation, overlap with known agriculturally important QTLs, and expression pattern diversity of CCTs in soybean. To gain a comprehensive picture of CCT genes in soybean, the QTLs that were identified in the last three decades (1992-2018) were analyzed. These QTLs were reported for their involvement in controlling soybean agronomic traits, including seed set and quality, flowering time, and stress response regulation were incorporated into the QTL co-localization analysis. The expression profiles of these selected genes were assessed in developing seed tissues and their response to various abiotic and biotic stressors. It was further uncovered a set of GmCCT genes and proved their roles in regulating seed protein and oil accumulation. The results shed light on the evolution and potential functions of CCT genes in soybean. Moreover, the present study provided a set of genes to understand little-known mechanisms of protein regulation and improve protein content in soybean and other grain crops.

### Fast-Neutron Mutant Identification

Fast neutron (FN) mutant line FN0172932 was selected from the M2 generation of irradiated elite line M92-220 in 2007 and further planted for homozygous mutants (FIG. 1). FN-induced genomic deletion region in M4-generation FN0172932 was previously determined using Comparative Genomic Hybridization (CGH) and further validated by whole genome sequencing (Illumine NovaSeq PE150, depth of coverage=16). The 1.3-Mb deletion region contains 52 gene models (Glyma. Wm82.a2) (Table 15). Plants were grown in the environment-controlled greenhouse in the Donald Danforth Plant Science Center with regular management (day 25° C./night 22° C., 40% humidity, 16 h/8 h day length for light/dark). Seed protein and oil content were measured on a pre-calibrated Perten DA 7250 analyzer (Perten Instruments, Inc., Springfield, IL, USA). Table 21 below provides details of POWR CCT-subfamily genes and their knockout and overexpression mutants. Table 22 provided field performance details about the POWR1 (CCT-subfamily gene) overexpression mutants.

TABLE 21
Details of POWR CCT-Subfamily Genes and
Their Knockout and Overexpression Mutants

| | | | | Oil |
|---|---|---|---|---|
| | | | CRISPR-MEDIATED | Phenotype |
| Sr. | | Gene | MUTATION | (% |
| No. | Gene ID | Name | COMBINATIONS | change) |
| 1 | Glyma.20G085100 | POWR1 | POWR1, 2, 3, 4 | ~3.78% ↑ |
| | | | POWR1 OVER- | ~1.34% ↓ |
| | | | EXPRESSION | |
| 2 | Glyma.10G134400 | POWR2 | POWR1, 2, 3 | ~3.91% ↑ |
| | | | POWR2, 3 | ~3.44% ↑ |
| | | | POWR2 | ~0.69% ↑ |
| 3 | Glyma.10G190300 | POWR3 | POWR1, 3 | ~3.84% ↑ |
| 4 | Glyma.20G200400 | POWR4 | POWR 1, 2, 4 | ~4.29% ↑ |

Note:
Arrows (↑ and ↓) indicate the increase or decrease in oil content in the seeds of the mutants grown in the greenhouse condition.

TABLE 22
Field performance details about the POWR1
(CCT-subfamily gene) overexpression mutants

| | | | Oil | field performance | |
|---|---|---|---|---|---|
| Sr. | | | Phenotype | (preliminary data) | |
| No. | Gene ID | Line Description | (% change) | Yield | Maturity |
| 1 | Glyma.20G085100 | Ubi promoter driving | ~2.0% ↓ | high | Early |
| | (POWR1) | POWR1 expression | | | (~25 days) |
| | | transgenic line | | | |
| | | Native promoter | ~2.00% ↓ | high | Early |
| | | driving POWR1 | | | (~21 days) |
| | | expression transgenic | | | |
| | | line | | | |

### Generation of Gene-Edited Soybean Lines

Three guide-RNA (gRNA) sequences specific to the exons of GmCCT34, one for exon 1, and two for exon 2, were designed using the web tool CRISPOR. The gRNA sequences were synthesized and annealed to the CRISPR/Cas9 expression vector and transformed into soybean cv. Williams 82 by the Wisconsin Crop Innovation Center using an Agrobacterium-mediated transformation protocol. A pair of primers specific to the vector was used to confirm positive transformants via PCR amplification (Forward: CTGCTGTTGATGGAGGACTT SEQ ID NO: 22; Reverse: CTCCTGGAGAAGCAGAAGTT SEQ ID NO: 23). T1 seeds from 10 independent TO plants were obtained and further grown in the environment-controlled greenhouse in the Donald Danforth Plant Science Center with the same condition as earlier mentioned. Unifoliate leaves were sampled from T1 plants to confirm the gene editing via PCR amplification followed by restriction enzyme digestion (Bsll). Editing-generated deletion was further confirmed using Sanger sequencing. As mentioned above, T2 seeds from the two homologous cct34 mutants were used to measure the seed composition traits. The PCR and sequencing validation was repeated twice.

### Subcellular Localization Analyses

The assay was performed through transient expression in Nicotiana benthamiana following a known method. The full-length CCT34 coding sequence (CDS), CCT34 lacking the CCT domain, and the CCT domain only were subcloned into the expression vector to generate UBQ10:YFP-CCT34, UBQ10: YFP-CCT34ΔCCT, and UBQ10: YFP-CCT, respectively. UBQ10: YFP was used as the empty vector. The vectors were individually transformed into Agrobacterium tumefaciens, and cultures of each construct were infiltrated into young leaves of N. benthamiana plants (4-6 weeks) using a 3-mL syringe without the needle. Leaves were imaged 48 h after infiltration. Imaging was carried out a Leica TCS SP8 confocal microscope using the 63× water immersion lens. Samples were excited with a 514-nm laser line and 649-nm laser line to detect YFP and chlorophyll signals, respectively. Fluorescence emission was collected for best signals of indicated fluorescent probes. This experiment was repeated twice.

### Arabidopsis Mutant Analysis

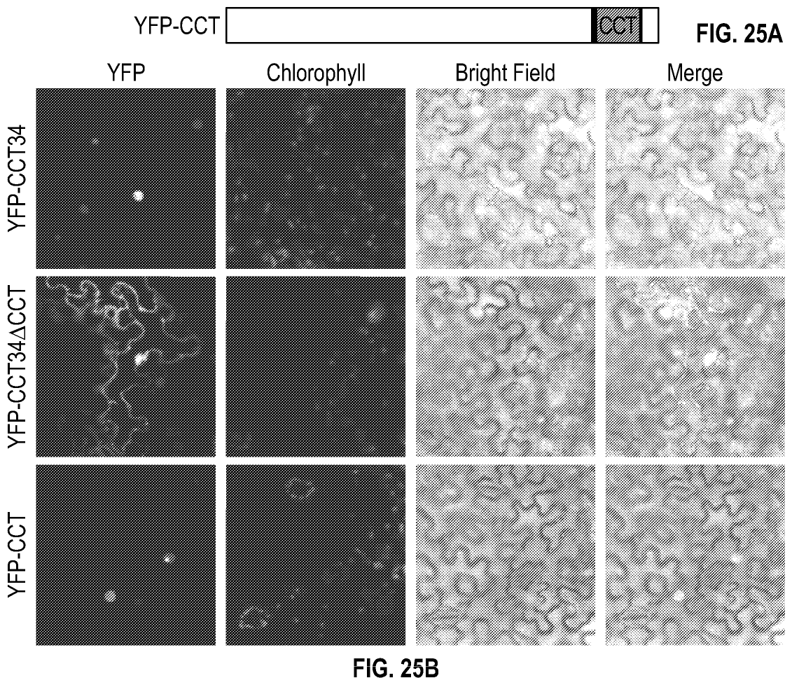

Two independent T-DNA insertions mutant lines (WiscDsLox297300_13A.1 (cct1) and SALK_036731.1 (cct2)) were obtained from ARBC (Arabidopsis Biological Resource Center). These two T-DNA insertion regions lie with different sites of the 3′end of the CDS of AT1G04500 (FIG. 25A), the closest homolog of soybean GmCCT67 (POWR1) and GmCCT34. The homozygous mutants were identified by PCR with specific primer sets listed in Table 18.

### Example 4. Results

### Cct Domains are Ancient and Diverse Across Plant Species

The CCT domain is a highly conserved basic module with ˜43 amino acids at the protein's C-terminus. The Hidden Markov Model (HMM) and the CCT domain (Pfam ID-PF06203) were used to search for the CCT proteins in selected plant species covering all members of the plant kingdom, including algae, mosses, ferns, conifers, and flowering plants. A set of 543 CCTs across the 24 plant species were identified (Table 19), including 69 soybean CCT domain-containing proteins (FIG. 2A, Table 21) and a range from 33 to 62 in other legumes, 40 and 52 CCT proteins, respectively, in the cereal crops rice and maize, and 13 to 29 in non-angiosperm land plants. (FIG. 2A). Traditionally, CCT proteins are classified into three subfamilies according to their constituent domains: single CCT (CCT Motif Family (CMF)), 1-2×BBOX-CCT (CONSTANTlike (COL) Family), and REC-CCT (Pseudo-Response Regulator Family). The present disclosure identified an additional protein group that carries the CCT domain, TIFY-CCT-ZnF_GATA. In these proteins, the CCT domain was located between two different domains, TIFY and ZnF_GATA. It is irrational to exclude the possibility that the CCT domain is involved in the function. Therefore, TIFY-CCT-ZnF_GATA was included in the analysis (FIG. 2B). The numbers of CCT protein genes in the tetraploids soybean and peanut were nearly doubled those in other diploid legumes. The CCT genes identified in Arabidopsis and the two cereal crops were generally more than those in legumes except for common bean and peanut. A small number of CCT genes (2-8) were present in chlorophyte species.

Phylogenetic analysis and phylogenetic trees generated from the CCT domain sequence identified six distinct clusters (FIG. 4A, 4B). These six clusters often, but not always, reflected the traditional domain-based classification system. Clusters I-III contained all of the members of the 1-2×BBOX-CCT subfamily, with Clusters I and II almost exclusively comprised of 2×BBOX-CCT genes and Cluster III containing the majority of 1×BBOX-CCTs. Clusters IV, V, and VI almost exclusively contained REC-CCT, single-CCT, and TIFY-CCT-Zn_GATA genes, respectively.

Notably, single-CCT genes were found in all six clusters (FIG. 4A). In clusters I, II, IV, and VI, consist of only a few individual single-CCTs, likely representing recent deletions of the non-CCT domain in these genes. It is also likely that several 1×BBOX-CCTs in the two 2×BBOX-CCT clusters (I and II) likewise represent the deletion of a single BBOX domain. Cluster III, however, contains a large number of single-CCTs that form two clades in the domain phylogeny (FIG. 4B). These likely represent an ancient deletion of the BBOX domain in this clade prior to the origin of the angiosperms.

Interestingly, CCTs containing non-canonical domains were rare and dispersed across several clusters, likely representing singleton insertion events, for example, DUF740-CCT in Vang06g17920 (adzuki bean), Adaptin_N-CCT in Psat0s3732g0120 (pea), S_TKc-CCT in Ca.14621 (chickpea) (FIG. 4A, 4B). Non-typical CCT proteins were not identified in soybean and Arabidopsis. All identified CCT genes in this study were summarized in Table 20. HMM logos were next prepared, representing each cluster (I-VI) from the domain tree to analyze the amino acids across the clusters (FIG. 4C). Most of the amino acids were conserved in the CCT domain across the six clusters, with high conservation observed for seven amino acids (Arginine (R) 1, R15, Tyrosine (Y) 23, R26, Alanine (A) 30, R35, and Phenylalanine (F) 40). Also, cluster-specific conserved amino acids were identified. For example, F8 in clusters V and VI, while Lysine (K) 22 was highly conserved in IV, with some exceptions (FIG. 4A and FIG. 4B). These conserved amino acids across the clusters could likely represent the essential roles of CCT family genes in DNA binding or forming functional complexes. In contrast, the amino acids specific to one or certain clusters might associate with the DNA binding specificity representing functional variation in the CCT family. The results indicated that the CCT domain sequences are conserved in plant species with diversified function specificities plausibly facilitated by some uniquely conserved amino acids.

All these six groups were identified as angiosperms. To further investigate the origin of these clusters, their membership in a range of non-angiosperms were identified, including charophyte algae, mosses, ferns, and gymnosperms (Table 7). All six clusters could be identified in each of the land plant lineages; however, two groups (I and VI) were absent from all of the chlorophyte species. This indicates that most of these groups arose early in plant evolution except for one of the 2×BBOX-CCT groups (I) and the TIFY-CCT-Zn_GATA group (VI), which first appeared in the bryophytes. Additionally, within the chlorophytes, Cluster IV (REC-CCT) was missing from all species except Chlamydomonas, and Cluster III (1×BBOX) was missing from Micromonas and Dusinella. These results indicate that individual chlorophyte lineages may have lost these genes or their sequences sufficiently diverged that the present search model could not identify them. Along with the increased number of CCTs from chlorophytes to bryophytes, the CCT domain gene family is ancient and underwent substantial expansion and diversification in the land plant lineage.

TABLE 23
CCT Genes in Other Species

| Species | I | II | III | IV | V | VI | Unclustered |
|---|---|---|---|---|---|---|---|
| spikemoss | 1 | 2 | 3 | 3 | 2 | 2 | 0 |
| liverwort | 0 | 1 | 2 | 2 | 1 | 2 | 0 |
| Angiosperm | 85 | 75 | 89 | 93 | 129 | 62 | 5 |
| fern | 3 | 3 | 4 | 6 | 6 | 1 | 1 |
| gymnosperm | 4 | 2 | 4 | 4 | 4 | 2 | 0 |
| Bryophyte | 9 | 7 | 24 | 10 | 10 | 10 | 0 |
| chlorophyte | 0 | 7 | 2 | 6 | 1 | 0 | 2 |

### Soybean CCT Gene Family

The 69 soybean CCT-containing genes identified here were designated as GmCCT01 to GmCCT69 based on the chromosomal coordinates. The 69 GmCCTs were mapped to all 20 chromosomes, and the majority were distributed in the distal telomeric regions (Table 21). Chromosome 13 contains the maximum number of GmCCTs (7) followed by chromosomes 4, 6, and 8, each having six members. Interestingly, 33 pairs of GmCCTs (66 of 69, 95.7%) were located within syntenic genomic regions. Additionally, the high bootstrap values for the GmCCT pairs in the soybean phylogenetic tree (FIG. 3) suggest that the paired GmCCTs are paralogs that have been retained from large-scale duplication events such as whole-genome duplication (WGD) or segmental duplication. This notion should also apply to peanut CCT genes because of the segmental allotetraploid in the peanut genome. In addition, two pairs of tandemly duplicated GmCCTs in the soybean genome (GmCCT9/10; GmCCT18/19) that were also fell within segmentally duplicated regions between chromosomes 4 and 6, suggesting that the tandem duplication occurred prior to the soybean specific WGD. These results showed that polyploidization, especially the lineage-specific tetraploid in soybean, is a major evolution-driven force of CCT expansion.

To understand the evolution of CCT proteins in related legume species, the syntenic CCT-associated genes and genomic regions were analyzed among selected closely related legume species, including Medicago, pea, chickpea, cowpea, common bean, and soybean. The syntenic analysis among leguminous CCTs revealed that 58 (84%) of the GmCCTs have at least one syntenic CCTs in legume genomes (Table 23; FIG. 7). For most legume CCT proteins, each corresponds to a pair of GmCCT paralogs, such as paralogs GmCCT12/21 in the syntenic regions of single CCT orthologous genes in five legumes (common bean, cowpea, chickpea, pea, Medicago) (FIG. 3A; Table 23; FIG. 7). This analysis also led to the identification of soybean-specific GmCCT without syntenic CCT homologs in other legumes, such as the pair of GmCCT34/67 (FIG. 3B; Table 23).

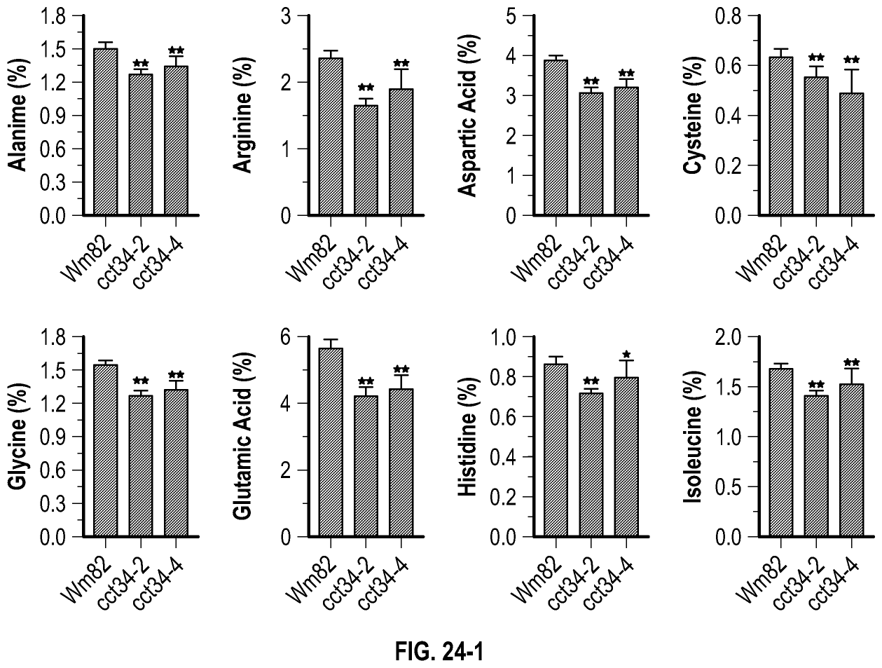

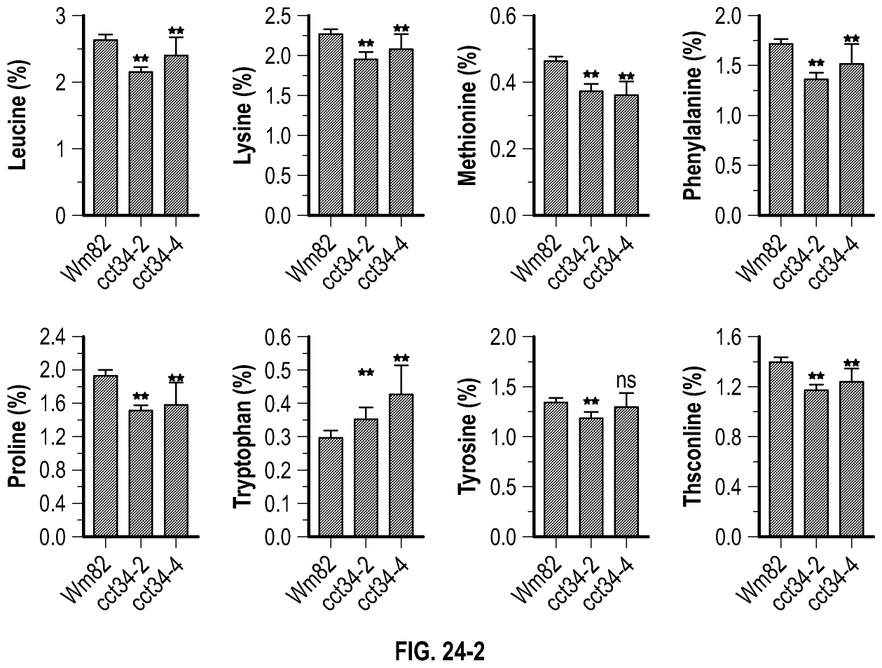

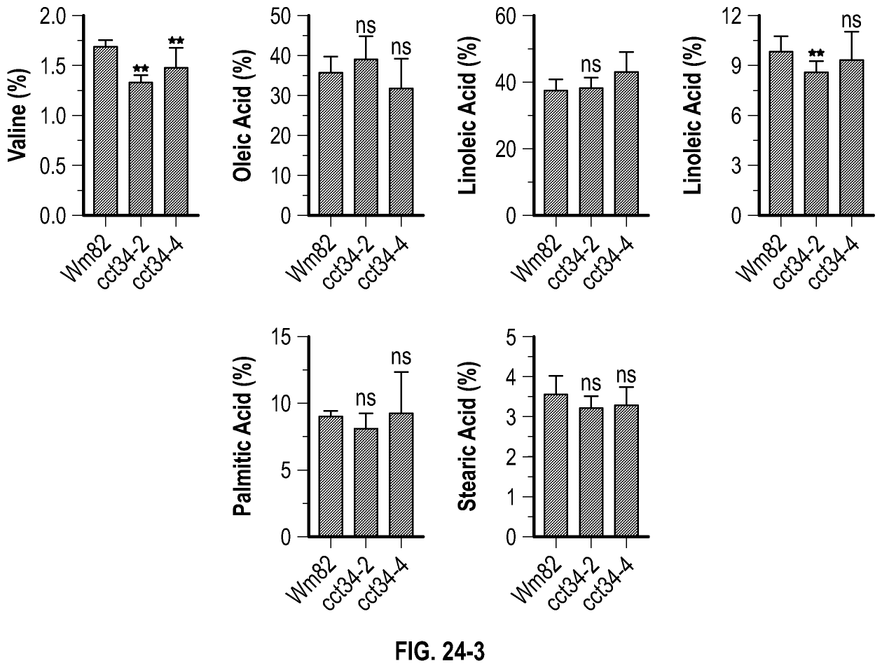

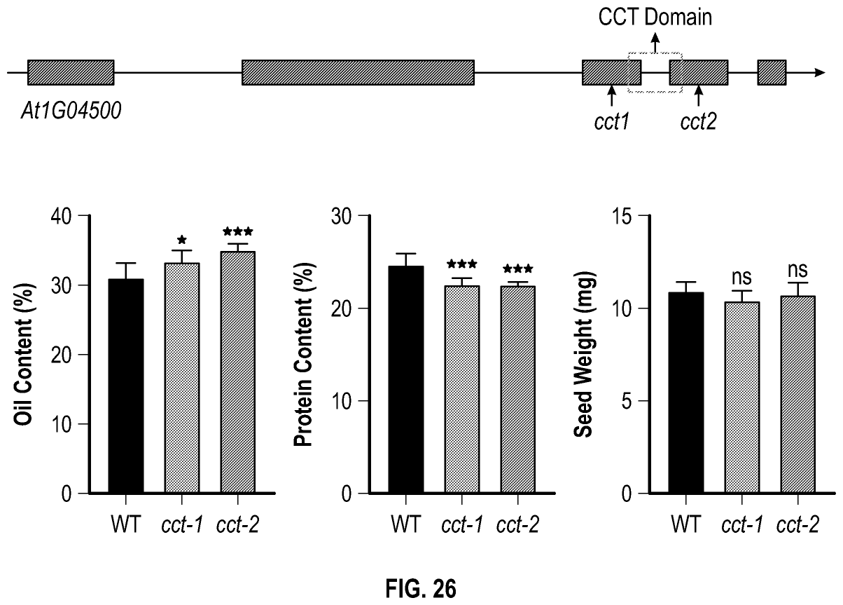

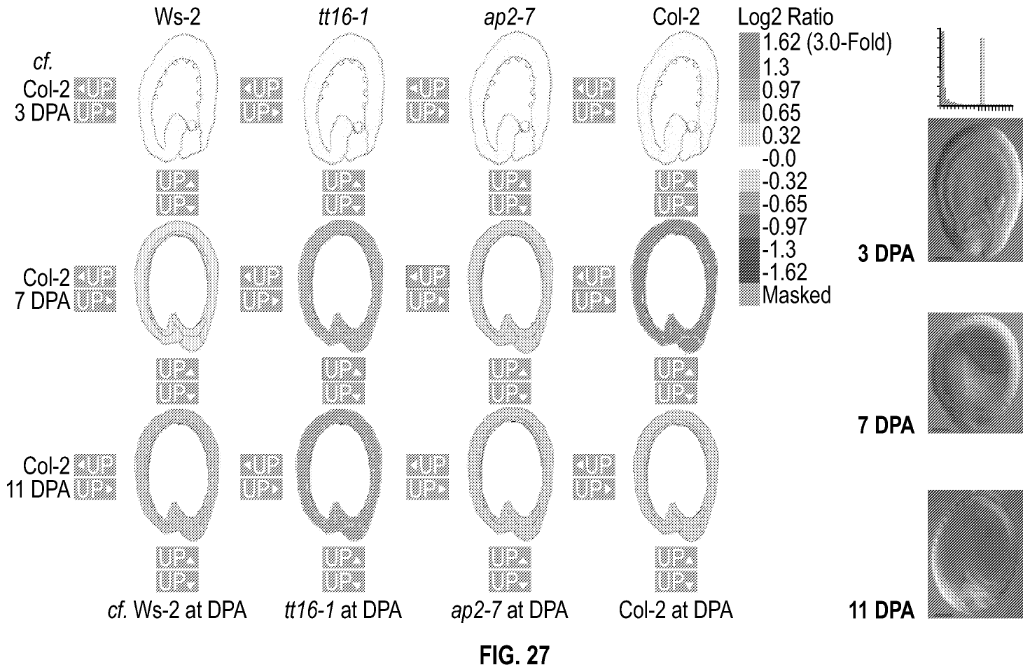

The frequency of POWR1 alleles in a diverse population consisting of 3,956 accessions and the allele effects on protein, oil and seed weight from analyzing their whole genome resequencing data (FIG. 24). The subcellular localization of GmCCT34 was shown in FIG. 25A and FIG. 25B. Function of the Arabidopsis CCT gene, AT1G04500, was investigated for its involvement in regulating seed oil composition. Like GmPOWR 1234 genes, there is only a single CCT domain found in Arabidopsis AT1G04500 gene (hence after AtPOWR1). The gene expression analysis showed that the AtPOWR1 is highly expressed in the seed coat tissues (FIG. 26 and FIG. 27, red color indicating the AtPOWR1 expression).

There is no information on the function(s) of AtPOWR1 concerning the regulation of seed protein-oil content. To know if this Arabidopsis gene also functions similarly to the GmPOWR genes, two homozygous T-DNA-insertion mutants were isolated (WiscDsLox297300_13A.1 and SALK_036731.1, (labeled as cct-1 and cct-2). The T-DNA insertion in these mutants occurred before and after the CCT domain, respectively, indicating that the CCT domain is dysfunctional. Similar to GmPOWR mutants, the seed composition analysis of the AtPOWR1 or ATcct mutants revealed a higher oil content compared with the wild type seeds These results suggested a conserved function of the CCTs between soybean and Arabidopsis in regulating oil accumulation in seeds (FIG. 26 and FIG. 27).

### Use of Cct-Domain Proteins to Improve Agronomic Traits of Plants

TABLE 1
Information of 69 CCT domain-containing proteins in soybean genome

| | | | | | Best match | Ara |
|---|---|---|---|---|---|---|
| Name | Gmax ID (v2.0) | Domain organization | Protein aa | Genomic coordinates | Ara orthologs | symbol |
| GmCCT01 | Glyma.01G221100 | CCT | 280 | Chr01: 55018467-55020621 | AT5G41380 | |
| GmCCT02 | Glyma.02G152900 | BBOX-BBOX-CCT | 341 | Chr02: 15702105-15705227 | AT2G47890 | |
| GmCCT03 | Glyma.02G215600 | TIFY-CCT-ZnF_GATA | 311 | Chr02: 40195550-40200143 | AT4G24470 | ZIM |
| GmCCT04 | Glyma.02G223700 | BBOX-CCT | 406 | Chr02: 41140728-41148151 | AT5G48250 | |
| GmCCT05 | Glyma.02G230800 | CCT | 231 | Chr02: 41829531-41831187 | AT1G07050 | |
| GmCCT06 | Glyma.03G209800 | BBOX-BBOX-CCT | 350 | Chr03: 41649706-41654690 | AT2G47890 | |
| GmCCT07 | Glyma.03G261300 | REC-CCT | 693 | Chr03: 45482095-45487185 | AT5G24470 | PRR5 |
| GmCCT08 | Glyma.04G058900 | BBOX-BBOX-CCT | 310 | Chr04: 4830133-4831900 | AT5G24930 | COL4 |
| GmCCT09 | Glyma.04G096200 | TIFY-CCT-ZnF_GATA | 310 | Chr04: 8627545-8632713 | AT4G24470 | ZIM |
| [symbol] mCCT10 | Glyma.04G096300 | TIFY-CCT-ZnF_GATA | 351 | Chr04: 8635553-8654734 | AT3G21175 | ZML1 |
| GmCCT11 | Glyma.04G166300 | REC-CCT | 561 | Chr04: 41751974-41757731 | AT5G61380 | TOC1 |
| GmCCT12 | Glyma.04G228300 | REC-CCT | 692 | Chr04: 49758484-49763637 | AT5G24470 | PR |
| GmCCT13 | Glyma.04G236400 | CCT | 282 | Chr04: 50475854-50481115 | AT5G53420 | |
| GmCCT14 | Glyma.05G025000 | REC-CCT | 576 | Chr05: 2174491-2182185 | AT5G61380 | TO |
| GmCCT15 | Glyma.05G233700 | BBOX-CCT | 366 | Chr05: 41087670-41089500 | AT1G25440 | |
| GmCCT16 | Glyma.05G209800 | CCT | 312 | Chr05: 39176944-39179334 | AT5G41380 | |
| GmCCT17 | Glyma.06G059600 | BBOX-BBOX-CCT | 311 | Chr06: 4519731-4521199 | AT5G24930 | CO |
| GmCCT18 | Glyma.06G097900 | TIFY-CCT-ZnF_GATA | 305 | Chr06: 7769865-7775571 | AT4G24470 | ZI [symbol] |
| GmCCT19 | Glyma.06G098000 | TIFY-CCT-ZnF_GATA | 352 | Chr06: 7777860-7785228 | AT3G21175 | ZM |
| GmCCT20 | Glyma.06G127900 | CCT | 279 | Chr06: 10510305-10515402 | AT5G53420 | |
| GmCCT21 | Glyma.06G136600 | REC-CCT | 701 | Chr06: 11182550-11187451 | AT5G24470 | PR |
| GmCCT22 | Glyma.06G196200 | REC-CCT | 559 | Chr06: 17608368-17614484 | AT5G61380 | TO |
| GmCCT23 | Glyma.07G049400 | REC-CCT | 723 | Chr07: 4201367-4206174 | AT5G24470 | PR |
| GmCCT24 | Glyma.07G091400 | BBOX-CCT | 428 | Chr07: 8539100-8549598 | AT1G25440 | |
| GmCCT25 | Glyma.07G182700 | TIFY-CCT-ZnF_GATA | 356 | Chr07: 35050481-35056486 | AT1G51600 | ZM |
| GmCCT26 | Glyma.08G016500 | CCT | 305 | Chr08: 1311495-1313676 | AT5G41380 | |
| GmCCT27 | Glyma.08G041100 | BBOX-CCT | 372 | Chr08: 3254251-3256417 | AT1G25440 | |
| GmCCT28 | Glyma.08G067200 | TIFY-CCT-ZnF_GATA | 359 | Chr08: 5163171-5168689 | AT3G21175 | ZML1 |
| GmCCT29 | Glyma.08G168900 | CCT | 285 | Chr08: 13433813-13436662 | AT3G12890 | ASML2 |
| GmCCT30 | Glyma.08G221800 | TIFY-CCT-ZnF_GATA | 335 | Chr08: 18006457-18012023 | AT1G51600 | ZML2 |
| GmCCT31 | Glyma.08G255200 | BBOX-BBOX-CCT | 349 | Chr08: 22599513-22602226 | AT3G02380 | COL2 |
| GmCCT32 | Glyma.10G021400 | BBOX-BBOX-CCT | 349 | Chr10: 1857312-1860298 | AT2G47890 | |
| GmCCT33 | Glyma.10G048100 | REC-CCT | 754 | Chr10: 4315721-4327545 | AT5G02810 | PRR7 |
| GmCCT34 | Glyma.10G134400 | CCT | 424 | Chr10: 36034798-36039695 | AT1G04500 | |
| GmCCT35 | Glyma.10G190300 | CCT | 420 | Chr10: 42314396-42318797 | AT1G04500 | |
| GmCCT36 | Glyma.10G274300 | BBOX-CCT | 420 | Chr10: 49716240-49718424 | AT1G25440 | |
| GmCCT37 | Glyma.11G022600 | CCT | 293 | Chr11: 1624015-1626279 | AT5G41380 | |
| GmCCT38 | Glyma.U033700 | CCT | 298 | scaffold_32: 104499-107620 | AT5G59990 | |
| [symbol] mCCT39 | Glyma.12G074400 | CCT | 280 | Chr12: 5601541-5604778 | AT5G59990 | |
| GmCCT40 | Glyma.12G196100 | BBOX-CCT | 424 | Chr12: 35736690-35741033 | AT3G21880 | |
| GmCCT41 | Glyma.13G100700 | CCT | 400 | Chr13: 21588820-21592548 | AT5G57180 | CIA |
| GmCCT42 | Glyma.13G093800 | BBOX-BBOX-CCT | 366 | Chr13: 20903248-20905141 | AT5G57660 | CO |
| GmCCT43 | Glyma.13G050300 | BBOX-BBOX-CCT | 362 | Chr13: 14630393-14632571 | AT3G02380 | CO |
| GmCCT44 | Glyma.13G009300 | BBOX-BBOX-CCT | 410 | Chr13: 2573536-2580122 | AT3G07650 | CO |
| GmCCT45 | Glyma.13G135900 | REC-CCT | 756 | Chr13: 24839736-24851002 | AT5G02810 | PRI |
| GmCCT46 | Glyma.09G184600 | BBOX-CCT | 481 | Chr09: 40986139-40988677 | AT1G25440 | |
| GmCCT47 | Glyma.13G306400 | BBOX-CCT | 424 | Chr13: 40256983-40261589 | AT3G21880 | |
| GmCCT48 | Glyma.13G331000 | CCT | 244 | Chr13: 42560606-42563362 | AT5G59990 | |
| GmCCT49 | Glyma.14G182800 | TIFY-CCT-ZnF_GATA | 308 | Chr14: 44629125-44634088 | AT4G24470 | ZI [symbol] |
| GmCCT50 | Glyma.14G190400 | BBOX-BBOX-CCT | 412 | Chr14: 45536718-45544087 | AT5G48250 | |
| GmCCT51 | Glyma.14G197900 | CCT | 221 | Chr14: 46327071-46328700 | AT1G07050 | |
| GmCCT52 | Glyma.15G219800 | CCT | 250 | Chr15: 38464689-38467299 | AT5G59990 | |
| GmCCT53 | Glyma.15G258200 | CCT | 279 | Chr15: 48862806-48865252 | AT3G12890 | ASI |
| GmCCT54 | Glyma.16G018000 | REC-CCT | 707 | Chr16: 1601747-1608339 | AT5G24470 | PRI |
| GmCCT55 | Glyma.16G050900 | BBOX-CCT | 382 | Chr16: 4874633-4878624 | AT1G28050 | |
| GmCCT56 | Glyma.16G067000 | CCT | 355 | Chr16: 6640310-6646267 | AT5G14370 | |
| GmCCT57 | Glyma.17G059100 | CCT | 399 | Chr17: 4486592-4490466 | AT5G57180 | CIA2 |
| GmCCT58 | Glyma.17G066600 | BBOX-BBOX-CCT | 375 | Chr17: 5146336-5148259 | AT5G57660 | COL5 |
| GmCCT59 | Glyma.17G102200 | REC-CCT | 580 | Chr17: 8018189-8023260 | AT5G61380 | TOC1 |
| GmCCT60 | Glyma.18G278100 | BBOX-BBOX-CCT | 353 | Chr18: 55964514-55967181 | AT3G02380 | COL2 |
| GmCCT61 | Glyma.19G039000 | BBOX-BBOX-CCT | 367 | Chr19: 5467685-5469895 | AT5G15840 | CO |
| GmCCT62 | Glyma.19G079600 | CCT | 355 | Chr19: 28916115-28930778 | AT5G14370 | |
| GmCCT63 | Glyma.19G099700 | BBOX-CCT | 386 | Chr19: 34682554-34686379 | AT2G33500 | |
| GmCCT64 | Glyma.19G207100 | BBOX-BBOX-CCT | 352 | Chr19: 46241151-46245817 | AT2G47890 | |
| GmCCT65 | Glyma.19G260400 | REC-CCT | 686 | Chr19: 50364717-50369677 | AT5G24470 | PRR5 |
| GmCCT66 | Glyma.20G060400 | BBOX-BBOX-CCT | 410 | Chr20: 18643038-18650833 | AT3G07650 | COL9 |
| GmCCT67 | Glyma.20G085100 | CCT | 444 | Chr20: 31774769-31779804 | AT1G04500 | |
| [symbol] mCCT68 | Glyma.20G115600 | BBOX-CCT | 419 | Chr20: 35746759-35748974 | AT1G68520 | |
| GmCCT69 | Glyma.20G200400 | CCT | 422 | Chr20: 43781164-43785175 | AT1G04500 | |

[symbol]

indicates data missing or illegible when filed

TABLE 2
List of all CCT-domain-containing proteins identified in this study

| Species | gene ID | domain1 | domain2 | domain3 |
|---|---|---|---|---|
| adzukibean | Vang0010ss00750.1 | | | CCT |
| adzukibean | Vang0010ss00800.1 | | | CCT |
| adzukibean | Vang0017s00340.1 | TIFY | CCT | ZnF_GATA |
| adzukibean | Vang0017s00360.1 | | | CCT |
| adzukibean | Vang0021ss01680.1 | | | CCT |
| adzukibean | Vang0022ss02670.1 | | | CCT |
| adzukibean | Vang0064ss00450.1 | TIFY | CCT | ZnF_GATA |
| adzukibean | Vang0070s00170.1 | BBOX | | CCT |
| adzukibean | Vang0084s00090.1 | REC | | CCT |
| adzukibean | Vang01g08240.1 | TIFY | CCT | ZnF_GATA |
| adzukibean | Vang01g09310.1 | TIFY | CCT | ZnF_GATA |
| adzukibean | Vang01g09960.1 | BBOX | BBOX | CCT |
| adzukibean | Vang01g10650.1 | | | CCT |
| adzukibean | Vang0238s00030.1 | REC | | CCT |
| adzukibean | Vang02g04910.1 | BBOX | | CCT |
| adzukibean | Vang02g13360.1 | BBOX | BBOX | CCT |
| adzukibean | Vang03g16900.1 | BBOX | BBOX | CCT |
| adzukibean | Vang04g02640.1 | | | CCT |
| adzukibean | Vang04g13110.1 | BBOX | | CCT |
| adzukibean | Vang04g15980.1 | BBOX | BBOX | CCT |
| adzukibean | Vang05g09750.1 | BBOX | BBOX | CCT |
| adzukibean | Vang06g00840.1 | | | CCT |
| adzukibean | Vang06g09620.1 | | | CCT |
| adzukibean | Vang06g17920.1 | DUF740 | DUF740 | CCT |
| adzukibean | Vang06g25290.1 | | | CCT |
| adzukibean | Vang09g02620.1 | REC | | CCT |
| adzukibean | Vang09g03140.1 | BBOX | | CCT |
| adzukibean | Vang10g01650.1 | BBOX | | CCT |
| adzukibean | Vang10g06540.1 | | | CCT |
| adzukibean | Vang11g11310.1 | REC | | CCT |
| adzukibean | Vang11g14640.1 | BBOX | | CCT |
| adzukibean | Vang11g15310.1 | | | CCT |
| adzukibean | Vang11g15630.1 | | TIFY | CCT |
| arabidopsis | AT1G04500.1 | | | CCT |
| arabidopsis | AT1G05290.1 | | | CCT |
| arabidopsis | AT1G07050.1 | | | CCT |
| arabidopsis | AT1G25440.1 | BBOX | | CCT |
| arabidopsis | AT1G28050.1 | BBOX | BBOX | CCT |
| arabidopsis | AT1G49130.1 | BBOX | | CCT |
| arabidopsis | AT1G51600.1 | TIFY | CCT | GATA |
| arabidopsis | AT1G63820.1 | | | CCT |
| arabidopsis | AT1G68520.1 | BBOX | | CCT |
| arabidopsis | AT1G73870.1 | BBOX | | CCT |
| arabidopsis | AT2G24790.1 | BBOX | BBOX | CCT |
| arabidopsis | AT2G32310.1 | | | CCT |
| arabidopsis | AT2G33350.1 | | | CCT |
| arabidopsis | AT2G33500.1 | BBOX | BBOX | CCT |
| arabidopsis | AT2G46670.1 | | | CCT |
| arabidopsis | AT2G46790.1 | REC | | CCT |
| arabidopsis | AT2G47890.1 | BBOX | BBOX | CCT |
| arabidopsis | AT3G02380.1 | BBOX | BBOX | CCT |
| arabidopsis | AT3G07650.1 | BBOX | BBOX | CCT |
| arabidopsis | AT3G12890.1 | | | CCT |
| arabidopsis | AT3G21175.1 | TIFY | CCT | GATA |
| arabidopsis | AT3G21880.1 | BBOX | | CCT |
| arabidopsis | AT4G15250.1 | BBOX | | CCT |
| arabidopsis | AT4G24470.3 | TIFY | CCT | ZnF_GATA |
| arabidopsis | AT4G25990.2 | | | CCT |
| arabidopsis | AT4G27900.1 | | | CCT |
| arabidopsis | AT5G02810.1 | REC | | CCT |
| arabidopsis | AT5G14370.1 | | | CCT |
| arabidopsis | AT5G15840.1 | BBOX | BBOX | CCT |
| arabidopsis | AT5G15850.1 | BBOX | BBOX | CCT |
| arabidopsis | AT5G24470.1 | | REC | CCT |
| arabidopsis | AT5G24930.1 | BBOX | BBOX | CCT |
| arabidopsis | AT5G41380.1 | | | CCT |
| arabidopsis | AT5G48250.1 | BBOX | BBOX | CCT |
| arabidopsis | AT5G53420.1 | | | CCT |
| arabidopsis | AT5G57180.2 | | | CCT |
| arabidopsis | AT5G57660.1 | BBOX | BBOX | CCT |
| arabidopsis | AT5G59990.1 | | | CCT |
| arabidopsis | AT5G60100.2 | REC | | CCT |
| arabidopsis | AT5G61380.1 | REC | | CCT |
| chickpeaCDC | Ca_00927.1 | BBOX | BBOX | CCT |
| chickpeaCDC | Ca_01359.1 | REC | | CCT |
| chickpeaCDC | Ca_01470.1 | TIFY | CCT | ZnF_GATA |
| chickpeaCDC | Ca_01471.1 | TIFY | CCT | ZnF_GATA |
| chickpeaCDC | Ca_01694.1 | BBOX | BBOX | CCT |
| chickpeaCDC | Ca_02437.1 | | | CCT |
| chickpeaCDC | Ca_02579.1 | | | CCT |
| chickpeaCDC | Ca_03219.1 | | | CCT |
| chickpeaCDC | Ca_03275.1 | BBOX | BBOX | CCT |
| chickpeaCDC | Ca_03974.1 | | | CCT |
| chickpeaCDC | Ca_04056.1 | REC | | CCT |
| chickpeaCDC | Ca_05292.1 | TIFY | CCT | ZnF_GATA |
| chickpeaCDC | Ca_05487.1 | BBOX | | CCT |
| chickpeaCDC | Ca_05769.1 | | | CCT |
| chickpeaCDC | Ca_05985.1 | BBOX | | CCT |
| chickpeaCDC | Ca_07641.1 | BBOX | | CCT |
| chickpeaCDC | Ca_09110.1 | BBOX | BBOX | CCT |
| chickpeaCDC | Ca_13165.1 | REC | | CCT |
| chickpeaCDC | Ca_13576.1 | | | CCT |
| chickpeaCDC | Ca_14041.1 | | | CCT |
| chickpeaCDC | Ca_14148.1 | | | CCT |
| chickpeaCDC | Ca_14236.1 | BBOX | BBOX | CCT |
| chickpeaCDC | Ca_14621.1 | REC | CCT | S_TKc |
| chickpeaCDC | Ca_14630.1 | | | CCT |
| chickpeaCDC | Ca_14968.1 | TIFY | CCT | ZnF_GATA |
| chickpeaCDC | Ca_16804.1 | TIFY | CCT | CCT |
| chickpeaCDC | Ca_17243.1 | | | CCT |
| chickpeaCDC | Ca_17522.1 | TIFY | CCT | GATA |
| chickpeaCDC | Ca_18243.1 | REC | | CCT |
| chickpeaCDC | Ca_20458.1 | CCT | | |
| chickpeaCDC | Ca_22148.1 | BBOX | BBOX | CCT |
| chickpeaCDC | Ca_22571.1 | | | CCT |
| chickpeaCDC | Ca_22631.1 | | | CCT |
| chickpeaCDC | Ca_22732.1 | BBOX | | CCT |
| chickpeaCDC | Ca_27452.1 | | | CCT |
| chickpeaCDC | Ca_28031.1 | | | CCT |
| commonbean | Phvul.001G074900.1 | | | CCT |
| commonbean | Phvul.001G087300.1 | BBOX | BBOX | CCT |
| commonbean | Phvul.001G137925.1 | | | CCT |
| commonbean | Phvul.001G203000.1 | BBOX | BBOX | CCT |
| commonbean | Phvul.002G158500.1 | | | CCT |
| commonbean | Phvul.002G213800.1 | TIFY | CCT | ZnF_GATA |
| commonbean | Phvul.002G291600.2 | | | CCT |
| commonbean | Phvul.002G316100.1 | BBOX | | CCT |
| commonbean | Phvul.003G036000.1 | BBOX | | CCT |
| commonbean | Phvul.003G125700.1 | | TIFY | CCT |
| commonbean | Phvul.003G141200.1 | | | CCT |
| commonbean | Phvul.003G149000.1 | BBOX | BBOX | CCT |
| commonbean | Phvul.003G188400.1 | REC | | CCT |
| commonbean | Phvul.004G046601.1 | | | CCT |
| commonbean | Phvul.004G157300.1 | BBOX | | CCT |
| commonbean | Phvul.005G049801.1 | | | CCT |
| commonbean | Phvul.005G113300.1 | BBOX | | CCT |
| commonbean | Phvul.006G005200.2 | BBOX | BBOX | CCT |
| commonbean | Phvul.007G027200.1 | BBOX | | CCT |
| commonbean | Phvul.007G162500.1 | REC | | CCT |
| commonbean | Phvul.007G166100.1 | BBOX | BBOX | CCT |
| commonbean | Phvul.007G270600.1 | | | CCT |
| commonbean | Phvul.008G022800.1 | BBOX | BBOX | CCT |
| commonbean | Phvul.008G022925.1 | | | CCT |
| commonbean | Phvul.008G147400.1 | | | CCT |
| commonbean | Phvul.008G182900.1 | TIFY | CCT | ZnF_GATA |
| commonbean | Phvul.008G190200.1 | BBOX | BBOX | CCT |
| commonbean | Phvul.008G254600.1 | TIFY | CCT | ZnF_GATA |
| commonbean | Phvul.008G257300.1 | | | CCT |
| commonbean | Phvul.009G035300.1 | TIFY | CCT | ZnF_GATA |
| commonbean | Phvul.009G035400.1 | TIFY | CCT | ZnF_GATA |
| commonbean | Phvul.009G045000.1 | REC | | CCT |
| commonbean | Phvul.009G053300.2 | | | CCT |
| commonbean | Phvul.009G085300.1 | BBOX | BBOX | CCT |
| commonbean | Phvul.009G118600.1 | | CCT | ZnF_GATA |
| commonbean | Phvul.009G185500.1 | REC | | CCT |
| commonbean | Phvul.009G258300.1 | REC | | CCT |
| commonbean | Phvul.010G119700.1 | REC | | CCT |
| commonbean | Phvul.011G069800.1 | | | CCT |
| commonbean | Phvul.011G070600.1 | | | CCT |
| commonbean | Phvul.L001944.1 | | | CCT |
| commonbean | Phvul.L002465.1 | | | CCT |
| cowpea | Vigun01g054600.1 | | | CCT |
| cowpea | Vigun01g065500.1 | | | CCT |
| cowpea | Vigun01g075800.1 | BBOX | | CCT |
| cowpea | Vigun01g187100.1 | BBOX | BBOX | CCT |
| cowpea | Vigun02g042100.1 | BBOX | | CCT |
| cowpea | Vigun02g177100.1 | | | CCT |
| cowpea | Vigun03g017700.1 | BBOX | | CCT |
| cowpea | Vigun03g042600.1 | | | CCT |
| cowpea | Vigun03g124600.1 | TIFY | CCT | ZnF_GATA |
| cowpea | Vigun03g337800.1 | REC | | CCT |
| cowpea | Vigun03g379600.1 | BBOX | BBOX | CCT |
| cowpea | Vigun03g387700.1 | | | CCT |
| cowpea | Vigun04g021700.1 | BBOX | | CCT |
| cowpea | Vigun04g068400.1 | BBOX | BBOX | CCT |
| cowpea | Vigun05g225000.1 | BBOX | | CCT |
| cowpea | Vigun05g253600.1 | | | CCT |
| cowpea | Vigun06g006800.1 | BBOX | | CCT |
| cowpea | Vigun07g011700.1 | | | CCT |
| cowpea | Vigun07g078600.1 | REC | | CCT |
| cowpea | Vigun07g116500.1 | BBOX | BBOX | CCT |
| cowpea | Vigun07g181300.1 | | | CCT |
| cowpea | Vigun07g270500.1 | BBOX | | CCT |
| cowpea | Vigun08g080300.1 | | | CCT |
| cowpea | Vigun08g109700.1 | TIFY | CCT | ZnF_GATA |
| cowpea | Vigun08g116900.1 | BBOX | BBOX | CCT |
| cowpea | Vigun08g124000.1 | | | CCT |
| cowpea | Vigun08g127200.1 | TIFY | CCT | ZnF_GATA |
| cowpea | Vigun09g004700.1 | REC | | CCT |
| cowpea | Vigun09g076300.1 | REC | | CCT |
| cowpea | Vigun09g140300.1 | REC | | CCT |
| cowpea | Vigun09g150200.1 | | | CCT |
| cowpea | Vigun09g180800.1 | TIFY | CCT | ZnF_GATA |
| cowpea | Vigun09g180900.1 | TIFY | CCT | ZnF_GATA |
| cowpea | Vigun09g217500.1 | BBOX | BBOX | CCT |
| cowpea | Vigun09g276400.1 | | CCT | ZnF_GATA |
| cowpea | Vigun10g152100.1 | REC | | CCT |
| cowpea | Vigun11g149100.1 | REC | | CCT |
| cowpea | Vigun11g150000.1 | | | CCT |
| maize | AC215811.3_FGP003 | | | CCT |
| maize | AC233888.1_FGP002 | BBOX | BBOX | CCT |
| maize | AC233946.1_FGP004 | | | CCT |
| maize | GRMZM2G001447_P01 | | | CCT |
| maize | GRMZM2G004483_P01 | | | CCT |
| maize | GRMZM2G005732_P03 | | | CCT |
| maize | GRMZM2G008482_P01 | | | CCT |
| maize | GRMZM2G012717_P01 | BBOX | | CCT |
| maize | GRMZM2G013398_P04 | BBOX | BBOX | CCT |
| maize | GRMZM2G013913_P01 | | | CCT |
| maize | GRMZM2G020081_P01 | REC | | CCT |
| maize | GRMZM2G021777_P01 | BBOX | BBOX | CCT |
| maize | GRMZM2G033962_P01 | REC | | CCT |
| maize | GRMZM2G038783_P01 | BBOX | BBOX | CCT |
| maize | GRMZM2G041991_P01 | BBOX | | CCT |
| maize | GRMZM2G042198_P01 | BBOX | BBOX | CCT |
| maize | GRMZM2G057529_P01 | | | CCT |
| maize | GRMZM2G058479_P01 | TIFY | CCT | ZnF_GATA |
| maize | GRMZM2G062218_P01 | | | CCT |
| maize | GRMZM2G062885_P01 | | | CCT |
| maize | GRMZM2G065896_P01 | TIFY | CCT | ZnF_GATA |
| maize | GRMZM2G075562_P01 | BBOX | BBOX | CCT |
| maize | GRMZM2G080509_P02 | TIFY | CCT | ZnF_GATA |
| maize | GRMZM2G092363_P01 | BBOX | | CCT |
| maize | GRMZM2G095598_P01 | BBOX | BBOX | CCT |
| maize | GRMZM2G095727_P05 | REC | | CCT |
| maize | GRMZM2G097454_P01 | | | CCT |
| maize | GRMZM2G106108_P01 | | | CCT |
| maize | GRMZM2G107595_P01 | | | CCT |
| maize | GRMZM2G107886_P01 | BBOX | | CCT |
| maize | GRMZM2G114137_P01 | BBOX | | CCT |
| maize | GRMZM2G123550_P01 | | | CCT |
| maize | GRMZM2G127426_P01 | | | CCT |
| maize | GRMZM2G133555_P01 | | | CCT |
| maize | GRMZM2G134066_P01 | | | CCT |
| maize | GRMZM2G134671_P01 | BBOX | | CCT |
| maize | GRMZM2G135446_P01 | | | CCT |
| maize | GRMZM2G144346_P01 | BBOX | | CCT |
| maize | GRMZM2G148453_P01 | | | CCT |
| maize | GRMZM2G148772_P01 | BBOX | BBOX | CCT |
| maize | GRMZM2G155370_P01 | | | CCT |
| maize | GRMZM2G159996_P01 | BBOX | | CCT |
| maize | GRMZM2G167560_P01 | BBOX | BBOX | CCT |
| maize | GRMZM2G176173_P01 | BBOX | | CCT |
| maize | GRMZM2G179024_P01 | REC | | CCT |
| maize | GRMZM2G367834_P01 | REC | | CCT |
| maize | GRMZM2G381691_P01 | | | CCT |
| maize | GRMZM2G405368_P01 | BBOX | BBOX | CCT |
| maize | GRMZM2G414423_P02 | | | CCT |
| maize | GRMZM2G474258_P01 | | | CCT |
| maize | GRMZM5G866699_P02 | CBFG-NFYB- | | CCT |
| | | HMF | | |
| maize | GRMZM5G868285_P02 | | | CCT |
| medicago | Medtr1g008220.1 | | | CCT |
| medicago | Medtr1g013450.1 | BBOX | BBOX | CCT |
| medicago | Medtr1g044785.1 | | | CCT |
| medicago | Medtr1g067110.1 | REC | | CCT |
| medicago | Medtr1g073350.1 | | | CCT |
| medicago | Medtr1g110870.1 | BBOX | | CCT |
| medicago | Medtr2g068730.1 | | | CCT |
| medicago | Medtr2g088900.1 | BBOX | | CCT |
| medicago | Medtr2g096080.1 | | | CCT |
| medicago | Medtr3g037390.1 | REC | | CCT |
| medicago | Medtr3g082630.1 | BBOX | BBOX | CCT |
| medicago | Medtr3g091340.1 | | | CCT |
| medicago | Medtr3g092780.1 | REC | | CCT |
| medicago | Medtr3g100040.1 | TIFY | CCT | ZnF_GATA |
| medicago | Medtr3g100050.1 | TIFY | CCT | ZnF_GATA |
| medicago | Medtr3g105710.1 | BBOX | BBOX | CCT |
| medicago | Medtr4g008090.1 | | | CCT |
| medicago | Medtr4g061360.1 | REC | | CCT |
| medicago | Medtr4g061823.1 | | | CCT |
| medicago | Medtr4g061910.1 | | | CCT |
| medicago | Medtr4g093730.1 | TIFY | CCT | ZnF_GATA |
| medicago | Medtr4g108880.1 | REC | | CCT |
| medicago | Medtr4g127420.1 | | | CCT |
| medicago | Medtr4g128930.1 | BBOX | BBOX | CCT |
| medicago | Medtr5g010120.1 | | | CCT |
| medicago | Medtr5g066510.1 | TIFY | CCT | ZnF_GATA |
| medicago | Medtr5g069480.1 | BBOX | BBOX | CCT |
| medicago | Medtr5g072780.1 | | | CCT |
| medicago | Medtr7g018170.1 | BBOX | BBOX | CCT |
| medicago | Medtr7g032240.1 | | | CCT |
| medicago | Medtr7g083540.1 | BBOX | | CCT |
| medicago | Medtr7g108150.1 | BBOX | BBOX | CCT |
| medicago | Medtr7g118260.1 | REC | | CCT |
| medicago | Medtr8g024260.1 | REC | | CCT |
| medicago | Medtr8g098725.1 | | | CCT |
| medicago | Medtr8g104190.1 | BBOX | | CCT |
| pea | Psat0s1849g0080.1 | BBOX | BBOX | CCT |
| pea | Psat0s3732g0120.1 | Adaptin_N | | CCT |
| pea | Psat0s3732g0240.1 | | | CCT |
| pea | Psat1g004200.1 | | | CCT |
| pea | Psat2g054040.1 | | | CCT |
| pea | Psat2g060680.1 | BBOX | BBOX | CCT |
| pea | Psat2g184400.1 | | | CCT |
| pea | Psat3g024360.1 | BBOX | BBOX | CCT |
| pea | Psat3g094880.1 | BBOX | | CCT |
| pea | Psat3g146040.1 | | CCT | H2A |
| pea | Psat3g193520.1 | BBOX | BBOX | CCT |
| pea | Psat4g013600.2 | | | CCT |
| pea | Psat4g049800.1 | REC | | CCT |
| pea | Psat4g159360.1 | | | CCT |
| pea | Psat5g032240.1 | BBOX | BBOX | CCT |
| pea | Psat5g047080.1 | TIFY | CCT | ZnF_GATA |
| pea | Psat5g071920.1 | REC | | CCT |
| pea | Psat5g077440.1 | | | CCT |
| pea | Psat5g105240.1 | BBOX | BBOX | CCT |
| pea | Psat5g199360.1 | REC | | CCT |
| pea | Psat5g200320.1 | REC | | CCT |
| pea | Psat5g265480.1 | BBOX | | CCT |
| pea | Psat5g282000.1 | | | CCT |
| pea | Psat6g004440.1 | | | CCT |
| pea | Psat6g025680.1 | BBOX | BBOX | CCT |
| pea | Psat6g107360.1 | | | CCT |
| pea | Psat6g124840.1 | REC | | CCT |
| pea | Psat6g131680.1 | | | CCT |
| pea | Psat6g228360.1 | BBOX | | CCT |
| pea | Psat7g011960.1 | BBOX | | CCT |
| pea | Psat7g031600.1 | | | CCT |
| pea | Psat7g037680.1 | | | CCT |
| pea | Psat7g101680.1 | TIFY | CCT | ZnF_GATA |
| pea | Psat7g221720.1 | REC | | CCT |
| pea | Psat7g260880.1 | TIFY | CCT | ZnF_GATA |
| peanut | arahy.Tifrunner.gnm1.ann1.0QJD35.1 | | | CCT |
| peanut | arahy.Tifrunner.gnm1.ann1.0U49HS.1 | TIFY | CCT | ZnF_GATA |
| peanut | arahy.Tifrunner.gnm1.ann1.129W39.1 | TIFY | CCT | ZnF_GATA |
| peanut | arahy.Tifrunner.gnm1.ann1.1K6ZHH.1 | REC | | CCT |
| peanut | arahy.Tifrunner.gnm1.ann1.22FLX8.1 | | | CCT |
| peanut | arahy.Tifrunner.gnm1.ann1.2F1UPX.2 | REC | | CCT |
| peanut | arahy.Tifrunner.gnm1.ann1.2L20XB.2 | TIFY | CCT | ZnF_GATA |
| peanut | arahy.Tifrunner.gnm1.ann1.3QBB6E.1 | | | CCT |
| peanut | arahy.Tifrunner.gnm1.ann1.5WR99K.1 | | | CCT |
| peanut | arahy.Tifrunner.gnm1.ann1.669CET.1 | BBOX | BBOX | CCT |
| peanut | arahy.Tifrunner.gnm1.ann1.6J3MKV.1 | BBOX | BBOX | CCT |
| peanut | arahy.Tifrunner.gnm1.ann1.7A5VUD.1 | BBOX | BBOX | CCT |
| peanut | arahy.Tifrunner.gnm1.ann1.857KFG.1 | | | CCT |
| peanut | arahy.Tifrunner.gnm1.ann1.86LJVS.1 | TIFY | CCT | ZnF_GATA |
| peanut | arahy.Tifrunner.gnm1.ann1.8MV22J.1 | BBOX | | CCT |
| peanut | arahy.Tifrunner.gnm1.ann1.9Y5DYJ.2 | | | CCT |
| peanut | arahy.Tifrunner.gnm1.ann1.A77B5H.1 | REC | | CCT |
| peanut | arahy.Tifrunner.gnm1.ann1.A9G6ZW.1 | REC | | CCT |
| peanut | arahy.Tifrunner.gnm1.ann1.AE0X9Y.1 | | | CCT |
| peanut | arahy.Tifrunner.gnm1.ann1.AGT1RS.1 | | | CCT |
| peanut | arahy.Tifrunner.gnm1.ann1.AY816Y.1 | BBOX | | CCT |
| peanut | arahy.Tifrunner.gnm1.ann1.B2EAID.1 | | | CCT |
| peanut | arahy.Tifrunner.gnm1.ann1.B9QMIW.1 | BBOX | BBOX | CCT |
| peanut | arahy.Tifrunner.gnm1.ann1.BQXP8E.2 | BBOX | | CCT |
| peanut | arahy.Tifrunner.gnm1.ann1.BU1BMX.1 | | | CCT |
| peanut | arahy.Tifrunner.gnm1.ann1.BVW291.1 | | | CCT |
| peanut | arahy.Tifrunner.gnm1.ann1.BXJN39.1 | | | CCT |
| peanut | arahy.Tifrunner.gnm1.ann1.D90B1M.1 | REC | | CCT |
| peanut | arahy.Tifrunner.gnm1.ann1.E88P3Q.1 | BBOX | BBOX | CCT |
| peanut | arahy.Tifrunner.gnm1.ann1.EF48U6.1 | | | CCT |
| peanut | arahy.Tifrunner.gnm1.ann1.EI5605.1 | TIFY | CCT | ZnF_GATA |
| peanut | arahy.Tifrunner.gnm1.ann1.FA5JII.1 | | | CCT |
| peanut | arahy.Tifrunner.gnm1.ann1.FCSG49.1 | | | CCT |
| peanut | arahy.Tifrunner.gnm1.ann1.FQS251.1 | | | CCT |
| peanut | arahy.Tifrunner.gnm1.ann1.GAW9KQ.2 | BBOX | | CCT |
| peanut | arahy.Tifrunner.gnm1.ann1.GMWG2V.1 | BBOX | BBOX | CCT |
| peanut | arahy.Tifrunner.gnm1.ann1.IR754M.1 | | | CCT |
| peanut | arahy.Tifrunner.gnm1.ann1.KU7VJQ.1 | | | CCT |
| peanut | arahy.Tifrunner.gnm1.ann1.LV381L.1 | BBOX | BBOX | CCT |
| peanut | arahy.Tifrunner.gnm1.ann1.N2A79V.1 | BBOX | BBOX | CCT |
| peanut | arahy.Tifrunner.gnm1.ann1.NK4UTA.3 | BBOX | BBOX | CCT |
| peanut | arahy.Tifrunner.gnm1.ann1.P8XRWE.1 | BBOX | BBOX | CCT |
| peanut | arahy.Tifrunner.gnm1.ann1.PDCB5X.1 | REC | | CCT |
| peanut | arahy.Tifrunner.gnm1.ann1.PEGORN.2 | REC | | CCT |
| peanut | arahy.Tifrunner.gnm1.ann1.QG9XFC.1 | TIFY | CCT | ZnF_GATA |
| peanut | arahy.Tifrunner.gnm1.ann1.S3IANQ.1 | BBOX | BBOX | CCT |
| peanut | arahy.Tifrunner.gnm1.ann1.TFJ3CW.1 | BBOX | BBOX | CCT |
| peanut | arahy.Tifrunner.gnm1.ann1.UNUG60.1 | | | CCT |
| peanut | arahy.Tifrunner.gnm1.ann1.V64N20.2 | REC | | CCT |
| peanut | arahy.Tifrunner.gnm1.ann1.V8BXHT.1 | | | CCT |
| peanut | arahy.Tifrunner.gnm1.ann1.V960AN.1 | REC | | CCT |
| peanut | arahy.Tifrunner.gnm1.ann1.WEZ503.1 | BBOX | | CCT |
| peanut | arahy.Tifrunner.gnm1.ann1.WVH2H8.1 | BBOX | | CCT |
| peanut | arahy.Tifrunner.gnm1.ann1.X5M96P.1 | | | CCT |
| peanut | arahy.Tifrunner.gnm1.ann1.XNQT3T.1 | | | CCT |
| peanut | arahy.Tifrunner.gnm1.ann1.Y6ILE8.1 | REC | | CCT |
| peanut | arahy.Tifrunner.gnm1.ann1.Y6Q3ES.1 | REC | | CCT |
| peanut | arahy.Tifrunner.gnm1.ann1.YRC4PH.1 | | | CCT |
| peanut | arahy.Tifrunner.gnm1.ann1.Z8TZMB.1 | REC | | CCT |
| peanut | arahy.Tifrunner.gnm1.ann1.ZRH25X.1 | | | CCT |
| peanut | arahy.Tifrunner.gnm1.ann1.ZS9VWH.1 | BBOX | BBOX | CCT |
| peanut | arahy.Tifrunner.gnm1.ann1.ZUL46H.1 | BBOX | | CCT |
| pigeon | C.cajan_00921.1 | | | CCT |
| pigeon | C.cajan_02757.1 | | | CCT |
| pigeon | C.cajan_03622.1 | BBOX | BBOXBBOX | CCT |
| pigeon | C.cajan_03709.1 | | | CCT |
| pigeon | C.cajan_04552.1 | | | CCT |
| pigeon | C.cajan_05381.1 | | | CCT |
| pigeon | C.cajan_08639.1 | BBOX | | CCT |
| pigeon | C.cajan_08715.1 | REC | | CCT |
| pigeon | C.cajan_08842.1 | | | CCT |
| pigeon | C.cajan_10458.1 | BBOX | BBOX | CCT |
| pigeon | C.cajan_10578.1 | BBOX | BBOX | CCT |
| pigeon | C.cajan_10962.1 | REC | | CCT |
| pigeon | C.cajan_11568.1 | | | CCT |
| pigeon | C.cajan_13216.1 | | | CCT |
| pigeon | C.cajan_13596.1 | | | CCT |
| pigeon | C.cajan_15626.1 | BBOX | | CCT |
| pigeon | C.cajan_17635.1 | BBOX | | CCT |
| pigeon | C.cajan_17916.1 | TIFY | CCT | ZnF_GATA |
| pigeon | C.cajan_17917.1 | | CCT | ZnF_GATA |
| pigeon | C.cajan_20131.1 | BBOX | BBOX | CCT |
| pigeon | C.cajan_21434.1 | | | CCT |
| pigeon | C.cajan_21442.1 | REC | | CCT |
| pigeon | C.cajan_22520.1 | | | CCT |
| pigeon | C.cajan_22947.1 | TIFY | CCT | ZnF_GATA |
| pigeon | C.cajan_24786.1 | | | CCT |
| pigeon | C.cajan_26721.1 | BBOX | BBOX | CCT |
| pigeon | C.cajan_29262.1 | BBOX | BBOX | CCT |
| pigeon | C.cajan_31872.1 | BBOX | | CCT |
| pigeon | C.cajan_32117.1 | TIFY | CCT | ZnF_GATA |
| pigeon | C.cajan_32168.1 | BBOX | BBOX | CCT |
| pigeon | C.cajan_36557.1 | BBOX | | CCT |
| pigeon | C.cajan_39146.1 | REC | | CCT |
| pigeon | C.cajan_39868.1 | REC | | CCT |
| rice | LOC_Os01g61900.1 | | | CCT |
| rice | LOC_Os02g01990.1 | | | CCT |
| rice | LOC_Os02g05470.1 | | | CCT |
| rice | LOC_Os02g05510.1 | TIFY | CCT | ZnF_GATA |
| rice | LOC_Os02g08150.1 | BBOX | BBOX | CCT |
| rice | LOC_Os02g39710.1 | BBOX | BBOX | CCT |
| rice | LOC_Os02g40510.1 | REC | | CCT |
| rice | LOC_Os02g49230.2 | BBOX | BBOX | CCT |
| rice | LOC_Os02g49880.1 | BBOX | | CCT |
| rice | LOC_Os03g04620.1 | | | CCT |
| rice | LOC_Os03g17570.3 | REC | | CCT |
| rice | LOC_Os03g22770.1 | BBOX | | CCT |
| rice | LOC_Os03g47970.1 | TIFY | CCT | ZnF_GATA |
| rice | LOC_Os03g50310.1 | BBOX | | CCT |
| rice | LOC_Os03g52450.1 | TIFY | CCT | ZnF_GATA |
| rice | LOC_Os04g42020.1 | BBOX | BBOX | CCT |
| rice | LOC_Os05g38990.1 | | | CCT |
| rice | LOC_Os05g51690.1 | | | CCT |
| rice | LOC_Os06g01340.1 | BBOX | | CCT |
| rice | LOC_Os06g15330.1 | BBOX | | CCT |
| rice | LOC_Os06g16370.1 | BBOX | BBOX | CCT |
| rice | LOC_Os06g19444.1 | BBOX | BBOX | CCT |
| rice | LOC_Os06g44450.1 | BBOX | BBOX | CCT |
| rice | LOC_Os06g48534.1 | TIFY | CCT | ZnF_GATA |
| rice | LOC_Os06g48610.1 | | | CCT |
| rice | LOC_Os07g15770.1 | | | CCT |
| rice | LOC_Os07g47140.1 | BBOX | | CCT |
| rice | LOC_Os07g49460.1 | REC | | CCT |
| rice | LOC_Os08g15050.1 | BBOX | | CCT |
| rice | LOC_Os08g42440.1 | BBOX | | CCT |
| rice | LOC_Os09g06464.1 | BBOX | | CCT |
| rice | LOC_Os09g33550.1 | BBOX | | CCT |
| rice | LOC_Os09g36220.1 | REC | | CCT |
| rice | LOC_Os10g32900.1 | | | CCT |
| rice | LOC_Os10g41100.1 | | | CCT |
| rice | LOC_Os11g01100.1 | | | CCT |
| rice | LOC_Os11g05930.1 | REC | | CCT |
| rice | LOC_Os12g01080.1 | | | CCT |
| rice | LOC_Os12g01100.1 | | | CCT |
| rice | LOC_Os12g16160.1 | | | CCT |
| soybean | Glyma.01G221100.1.p | | | CCT |
| soybean | Glyma.02G152900.1.p | BBOX | BBOX | CCT |
| soybean | Glyma.02G215600.1.p | TIFY | CCT | ZnF_GATA |
| soybean | Glyma.02G223700.1.p | BBOX | | CCT |
| soybean | Glyma.02G230800.1.p | | | CCT |
| soybean | Glyma.03G209800.1.p | BBOX | BBOX | CCT |
| soybean | Glyma.03G261300.1.p | REC | | CCT |
| soybean | Glyma.04G058900.1.p | BBOX | BBOX | CCT |
| soybean | Glyma.04G096200.1.p | TIFY | CCT | ZnF_GATA |
| soybean | Glyma.04G096300.1.p | TIFY | CCT | ZnF_GATA |
| soybean | Glyma.04G166300.1.p | REC | | CCT |
| soybean | Glyma.04G228300.1.p | REC | | CCT |
| soybean | Glyma.04G236400.1.p | | | CCT |
| soybean | Glyma.05G025000.1.p | REC | | CCT |
| soybean | Glyma.05G209800.1.p | | | CCT |
| soybean | Glyma.05G233700.1.p | BBOX | | CCT |
| soybean | Glyma.06G059600.1.p | BBOX | BBOX | CCT |
| soybean | Glyma.06G097900.1.p | TIFY | CCT | ZnF_GATA |
| soybean | Glyma.06G098000.1.p | TIFY | CCT | ZnF_GATA |
| soybean | Glyma.06G127900.1.p | | | CCT |
| soybean | Glyma.06G136600.1.p | REC | | CCT |
| soybean | Glyma.06G196200.1.p | REC | | CCT |
| soybean | Glyma.07G049400.1.p | REC | | CCT |
| soybean | Glyma.07G091400.1.p | BBOX | | CCT |
| soybean | Glyma.07G182700.1.p | TIFY | CCT | ZnF_GATA |
| soybean | Glyma.08G016500.1.p | | | CCT |
| soybean | Glyma.08G041100.1.p | BBOX | | CCT |
| soybean | Glyma.08G067200.1.p | TIFY | CCT | ZnF_GATA |
| soybean | Glyma.08G168900.1.p | | | CCT |
| soybean | Glyma.08G221800.1.p | TIFY | CCT | ZnF_GATA |
| soybean | Glyma.08G255200.1.p | BBOX | BBOX | CCT |
| soybean | Glyma.09G184600.1.p | BBOX | | CCT |
| soybean | Glyma.10G021400.1.p | BBOX | BBOX | CCT |
| soybean | Glyma.10G048100.1.p | REC | | CCT |
| soybean | Glyma.10G134400.1.p | | | CCT |
| soybean | Glyma.10G190300.1.p | | | CCT |
| soybean | Glyma.10G274300.1.p | BBOX | | CCT |
| soybean | Glyma.11G022600.1.p | | | CCT |
| soybean | Glyma.12G074400.1.p | | | CCT |
| soybean | Glyma.12G196100.1.p | BBOX | | CCT |
| soybean | Glyma.13G009300.1.p | BBOX | BBOX | CCT |
| soybean | Glyma.13G050300.1.p | BBOX | BBOX | CCT |
| soybean | Glyma.13G093800.1.p | BBOX | BBOX | CCT |
| soybean | Glyma.13G100700.1.p | | | CCT |
| soybean | Glyma.13G135900.1.p | REC | | CCT |
| soybean | Glyma.13G306400.1.p | BBOX | | CCT |
| soybean | Glyma.13G331000.1.p | | | CCT |
| soybean | Glyma.14G182800.1.p | TIFY | CCT | ZnF_GATA |
| soybean | Glyma.14G190400.1.p | BBOX | BBOX | CCT |
| soybean | Glyma.14G197900.1.p | | | CCT |
| soybean | Glyma.15G219800.1.p | | | CCT |
| soybean | Glyma.15G258200.1.p | | | CCT |
| soybean | Glyma.16G018000.1.p | REC | | CCT |
| soybean | Glyma.16G050900.1.p | BBOX | | CCT |
| soybean | Glyma.16G067000.1.p | | | CCT |
| soybean | Glyma.17G059100.1.p | | | CCT |
| soybean | Glyma.17G066600.1.p | BBOX | BBOX | CCT |
| soybean | Glyma.17G102200.1.p | REC | | CCT |
| soybean | Glyma.18G278100.1.p | BBOX | BBOX | CCT |
| soybean | Glyma.19G039000.1.p | BBOX | BBOX | CCT |
| soybean | Glyma.19G079600.1.p | | | CCT |
| soybean | Glyma.19G099700.1.p | BBOX | | CCT |
| soybean | Glyma.19G207100.1.p | BBOX | BBOX | CCT |
| soybean | Glyma.19G260400.1.p | REC | | CCT |
| soybean | Glyma.20G060400.1.p | BBOX | BBOX | CCT |
| soybean | Glyma.20G085100.1.p | | | CCT |
| soybean | Glyma.20G115600.1.p | BBOX | | CCT |
| soybean | Glyma.20G200400.1.p | | | CCT |
| soybean | Glyma.U033700.1.p | | | CCT |
| Creinhardtii | Cre06.g278200.t1.1 | BBOX | | CCT |
| Creinhardtii | Cre06.g278159.t1.1 | BBOX | BBOX | CCT |
| Creinhardtii | Cre02.g094150.t1.2 | REC | | CCT |
| Creinhardtii | Cre07.g351550.t1.1 | | | CCT |
| Creinhardtii | Cre15.g635100.t1.2 | | | CCT |
| Creinhardtii | Cre12.g534100.t1.1 | | | CCT |
| Creinhardtii | Cre16.g676421.t1.1 | REC | | CCT |
| Creinhardtii | Cre16.g675100.t1.1 | | | CCT |
| Csubellipsoidea | 64395 | BBOX | | CCT |
| Csubellipsoidea | 65695 | IF-2B | REC | CCT |
| Csubellipsoidea | 58803 | | | CCT |
| Csubellipsoidea | 55541 | | | CCT |
| Csubellipsoidea | 48136 | | | CCT |
| Dsalina | Dusal.0007s00046.1.p | BBOX | BBOX | CCT |
| Dsalina | Dusal.0068s00019.1.p | REC | | CCT |
| MpusillaCCMP1545 | 56149 | BBOX | | CCT |
| MpusillaCCMP1546 | 48139 | REC | | CCT |
| MpusillaCCMP1547 | 195113 | BBOX | | CCT |
| MspRCC299 | 101672 | BBOX | BBOX | CCT |
| MspRCC299 | 63995 | BBOX | | CCT |
| MspRCC299 | 59410 | PFAM | | CCT |
| MspRCC299 | 57487 | BBOX | BBOX | CCT |
| Olucimarinus | 37312 | BBOX | | CCT |
| Olucimarinus | 33434 | BBOX | | CCT |
| Olucimarinus | 24762 | | | CCT |
| Olucimarinus | 29755 | REC | | CCT |
| Olucimarinus | 41670 | REC | | CCT |

TABLE 3
Syntenic CCTs in legume genomes

| Soybean | adzukibean | chickpeaCDC | commonbean | cowpea | medicago |
|---|---|---|---|---|---|
| Glyma.01G221100.1 | Vang07g06400.1 | Ca_02437.1 | Phvul.002G158500.1 | Vigun02g177100.1 | Medtr5g010120.1 |
| Glyma.02G152900.1 | Vang06g17920.2 | . | Phvul.007G166100.1 | Vigun07g116500.2 | . |
| Glyma.02G215600.1 | Vang01g09310.1 | . | Phvul.008G182900.1 | Vigun08g109700.1 | Medtr5g066510.1 |
| Glyma.02G223700.1 | . | Ca_14236.1 | Phvul.008G190200.1 | . | Medtr5g069480.5 |
| Glyma.02G230800.1 | Vang01g08550.1 | Ca_17243.1 | Phvul.008G257300.1 | Vigun08g124000.1 | Medtr5g072780.1 |
| Glyma.03G209800.1 | Vang04g15980.1 | Ca_00927.1 | Phvul.001G203000.1 | Vigun01g187100.1 | Medtr7g108150.1 |
| Glyma.03G261300.1 | Vang09g08740.1 | Ca_01359.1 | Phvul.009G258300.1 | Vigun09g004700.1 | Medtr7g118260.2 |
| Glyma.04G058900.1 | Vang05g09750.1 | Ca_01694.1 | Phvul.009G085300.1 | Vigun09g217500.1 | Medtr3g105710.1 |
| Glyma.04G096200.1 | Vang0017s00340.2 | Ca_01471.1 | Phvul.009G035400.1 | Vigun09g180900.1 | Medtr3g100050.1 |
| Glyma.04G096300.1 | Vang0017s00360.1 | Ca_01470.1 | Phvul.009G035300.1 | Vigun09g180800.1 | Medtr3g100040.1 |
| Glyma.04G166300.1 | Vang09g02620.1 | Ca_18243.1 | Phvul.009G185500.1 | Vigun09g076300.1 | Medtr3g037390.1 |
| Glyma.04G228300.1 | Vang0084s00090.1 | Ca_04056.1 | Phvul.009G045000.1 | Vigun09g140300.1 | Medtr3g092780.2 |
| Glyma.04G236400.1 | Vang0090s00480.2 | Ca_03974.1 | Phvul.009G053300.2 | Vigun09g150200.1 | Medtr3g091340.1 |
| Glyma.05G025000.1 | Vang11g11310.1 | Ca_13165.1 | Phvul.003G188400.1 | Vigun03g337800.1 | Medtr4g108880.1 |
| Glyma.05G209800.1 | Vang10g06540.1 | Ca_05769.1 | Phvul.002G291600.2 | Vigun03g042600.1 | Medtr8g098725.1 |
| Glyma.05G233700.1 | Vang10g01650.1 | . | Phvul.002G316100.1 | Vigun03g017700.1 | Medtr8g104190.1 |
| Glyma.06G059600.1 | Vang05g09750.1 | Ca_01694.1 | Phvul.009G085300.1 | Vigun09g217500.1 | Medtr3g105710.1 |
| Glyma.06G097900.1 | Vang0017s00340.2 | Ca_01471.1 | Phvul.009G035400.1 | Vigun09g180900.1 | Medtr3g100050.1 |
| Glyma.06G098000.1 | Vang0017s00360.1 | Ca_01470.1 | Phvul.009G035300.1 | Vigun09g180800.1 | Medtr3g100040.1 |
| Glyma.06G127900.1 | Vang0090s00480.2 | Ca_03974.1 | Phvul.009G053300.2 | Vigun09g150200.1 | Medtr3g091340.1 |
| Glyma.06G136600.1 | Vang0084s00090.1 | Ca_04056.1 | Phvul.009G045000.1 | Vigun09g140300.1 | Medtr3g092780.2 |
| Glyma.06G196200.1 | Vang09g02620.1 | Ca_18243.1 | Phvul.009G185500.1 | Vigun09g076300.1 | Medtr3g037390.1 |
| Glyma.07G049400.1 | Vang0238s00030.1 | . | Phvul.010G119700.2 | Vigun10g152100.2 | Medtr8g024260.1 |
| Glyma.07G091400.1 | Vang02g04910.1 | . | Phvul.004G157300.1 | Vigun04g021700.1 | . |
| Glyma.07G182700.1 | Vang0064ss00450.1 | Ca_05292.1 | Phvul.002G213800.2 | Vigun03g124600.1 | Medtr4g093730.1 |
| Glyma.08G016500.1 | Vang10g06540.1 | Ca_05769.1 | Phvul.002G291600.2 | Vigun03g042600.1 | Medtr8g098725.1 |
| Glyma.08G041100.1 | Vang10g01650.1 | Ca_22732.1 | Phvul.002G316100.1 | Vigun03g017700.1 | Medtr8g104190.1 |
| Glyma.08G067200.1 | Vang0064ss00450.1 | Ca_05292.1 | Phvul.002G213800.2 | Vigun03g124600.1 | Medtr4g093730.1 |
| Glyma.08G168900.1 | . | Ca_13576.1 | . | . | Medtr2g068730.1 |
| Glyma.08G221800.1 | Vang01g08240.1 | Ca_17522.1 | Phvul.008G254600.1 | Vigun08g127200.1 | Medtr4g008090.1 |
| Glyma.08G255200.1 | Vang04g02640.1 | . | Phvul.008G022800.1 | Vigun05g024400.1 | Medtr7g018170.1 |
| Glyma.09G184600.1 | Vang02g04910.1 | . | Phvul.004G157300.1 | Vigun04g021700.1 | . |
| Glyma.10G021400.1 | Vang06g17920.2 | . | Phvul.007G166100.1 | Vigun07g116500.2 | . |
| Glyma.10G048100.1 | Vang0206s00130.1 | Ca_14041.1 | Phvul.007G162500.1 | Vigun07g078600.2 | Medtr1g067110.1 |
| Glyma.10G134400.1 | . | . | . | Vigun07g011700.1 | . |
| Glyma.10G190300.1 | Vang06g09620.1 | . | Phvul.L001944.1 | Vigun07g181300.1 | Medtr1g073350.1 |
| Glyma.10G274300.1 | Vang06g00840.1 | Ca_05487.1 | Phvul.007G027200.1 | Vigun07g270500.1 | Medtr1g110870.1 |
| Glyma.11G022600.1 | Vang07g06400.1 | Ca_02437.1 | Phvul.002G158500.1 | Vigun02g177100.1 | . |
| Glyma.12G074400.1 | Vang0010ss00750.1 | Ca_14630.1 | Phvul.011G069800.1 | Vigun11g150000.1 | Medtr4g061823.1 |
| Glyma.12G196100.1 | Vang09g03140.1 | Ca_14148.1 | Phvul.005G113300.1 | Vigun05g225000.4 | Medtr2g088900.1 |
| Glyma.13G009300.1 | Vang03g16900.1 | Ca_07641.1 | Phvul.006G005200.3 | . | Medtr3g082630.1 |
| Glyma.13G050300.1 | Vang02g13360.1 | . | Phvul.004G046601.1 | Vigun04g068400.1 | . |
| Glyma.13G093800.1 | Vang11g14640.1 | Ca_03275.1 | Phvul.003G149000.1 | Vigun03g379600.1 | Medtr4g128930.1 |
| Glyma.13G100700.1 | Vang11g15310.1 | Ca_03219.1 | Phvul.003G141200.1 | Vigun03g387700.1 | Medtr4g127420.1 |
| Glyma.13G135900.1 | Vang0206s00130.1 | Ca_14041.1 | Phvul.007G162500.1 | Vigun07g078600.1 | Medtr1g067110.1 |
| Glyma.13G306400.1 | Vang09g03140.1 | Ca_14148.1 | Phvul.005G113300.1 | Vigun05g225000.1 | Medtr2g088900.1 |
| Glyma.13G331000.1 | Vang0021ss01680.1 | Ca_02579.1 | Phvul.L002465.1 | Vigun05g253600.1 | Medtr2g096080.1 |
| Glyma.14G182800.1 | Vang01g09310.1 | Ca_14968.1 | Phvul.008G182900.1 | Vigun08g109700.1 | Medtr5g066510.1 |
| Glyma.14G190400.1 | Vang01g09960.2 | Ca_14236.1 | Phvul.008G190200.1 | Vigun08g116900.1 | Medtr5g069480.1 |
| Glyma.14G197900.1 | Vang01g08550.1 | Ca_17243.1 | . | . | Medtr5g072780.1 |
| Glyma.15G219800.1 | . | . | . | . | . |
| Glyma.15G258200.1 | Vang0022ss02670.1 | Ca_13576.1 | Phvul.005G049801.1 | Vigun01g054600.1 | Medtr2g068730.1 |
| Glyma.16G018000.1 | . | . | Phvul.010G119700.1 | Vigun10g152100.2 | Medtr8g024260.1 |
| Glyma.16G050900.1 | Vang04g13110.1 | Ca_05985.1 | Phvul.001G087300.1 | Vigun01g075800.1 | Medtr7g083540.1 |
| Glyma.16G067000.1 | . | . | . | Vigun01g065500.1 | Medtr7g032240.1 |
| Glyma.17G059100.1 | Vang11g15310.1 | Ca_03219.1 | Phvul.003G141200.2 | Vigun03g387700.1 | Medtr4g127420.1 |
| Glyma.17G066600.1 | Vang11g14640.1 | Ca_03275.1 | Phvul.003G149000.1 | Vigun03g379600.1 | Medtr4g128930.1 |
| Glyma.17G102200.1 | Vang11g11310.1 | Ca_13165.1 | Phvul.003G188400.1 | Vigun03g337800.1 | Medtr4g108880.1 |
| Glyma.18G278100.1 | Vang04g02640.1 | Ca_22148.1 | Phvul.008G022800.1 | Vigun05g024400.1 | Medtr7g018170.1 |
| Glyma.19G039000.1 | Vang02g13360.1 | . | Phvul.004G046601.1 | Vigun04g068400.1 | . |
| Glyma.19G079600.1 | Vang04g12460.1 | . | Phvul.008G147400.1 | Vigun01g065500.1 | . |
| Glyma.19G099700.1 | Vang04g13110.1 | Ca_05985.1 | Phvul.001G087300.1 | Vigun01g075800.1 | Medtr7g083540.1 |
| Glyma.19G207100.1 | Vang04g15980.1 | Ca_00927.1 | Phvul.001G203000.1 | Vigun01g187100.1 | Medtr7g108150.1 |
| Glyma.19G260400.1 | Vang09g08740.1 | Ca_01359.1 | Phvul.009G258300.1 | Vigun09g004700.1 | Medtr7g118260.1 |
| Glyma.20G060400.1 | . | Ca_07641.1 | . | . | Medtr3g082630.1 |
| Glyma.20G085100.1 | Vang06g25290.1 | Ca_20458.1 | Phvul.007G270600.1 | Vigun07g011700.1 | . |
| Glyma.20G115600.1 | Vang06g00840.1 | Ca_05487.1 | Phvul.007G027200.1 | Vigun07g270500.1 | Medtr1g110870.1 |
| Glyma.20G200400.1 | Vang06g09620.1 | . | Phvul.L001944.1 | Vigun07g181300.1 | Medtr1g073350.1 |
| Glyma.U033700.1 | Vang0010ss00750.1 | Ca_14630.1 | Phvul.011G069800.1 | Vigun11g150000.1 | Medtr4g061823.1 |

| | | | | soybean- | |
|---|---|---|---|---|---|
| Soybean | pea | peanut | pigeon | copy | arabidopsis |
| Glyma.01G221100.1 | Psat2g184400.1 | arahy.B2EAID.1 | C.cajan_13216.1 | Glyma.11G022600.1 | . |
| Glyma.02G152900.1 | . | . | C.cajan_10458.1 | Glyma.10G021400.1 | AT2G47890.1 |
| Glyma.02G215600.1 | . | . | . | Glyma.14G182800.1 | . |
| Glyma.02G223700.1 | . | arahy.TFJ3CW.1 | C.cajan_29262.1 | Glyma.14G190400.1 | . |
| Glyma.02G230800.1 | Psat2g054040.2 | arahy.XNQT3T.1 | C.cajan_05381.1 | Glyma.14G197900.1 | . |
| Glyma.03G209800.1 | Psat3g024360.1 | arahy.7A5VUD.1 | C.cajan_10578.1 | Glyma.19G207100.1 | . |
| Glyma.03G261300.1 | . | arahy.Y6ILE8.1 | C.cajan_10962.1 | Glyma.19G260400.1 | . |
| Glyma.04G058900.1 | . | arahy.ZS9VWH.1 | C.cajan_32168.1 | Glyma.06G059600.1 | . |
| Glyma.04G096200.1 | . | . | C.cajan_17917.1 | Glyma.06G097900.1 | . |
| Glyma.04G096300.1 | . | . | C.cajan_17916.1 | Glyma.06G098000.1 | . |
| Glyma.04G166300.1 | Psat5g199360.1 | . | . | Glyma.06G196200.1 | . |
| Glyma.04G228300.1 | Psat5g071920.1 | arahy.A9G6ZW.1 | . | Glyma.06G136600.1 | AT5G24470.1 |
| Glyma.04G236400.1 | Psat5g077440.1 | arahy.BVW291.1 | C.cajan_08842.1 | Glyma.06G127900.1 | AT5G53420.1 |
| Glyma.05G025000.1 | Psat4g049800.1 | arahy.2F1UPX.2 | . | Glyma.17G102200.1 | AT5G61380.1 |
| Glyma.05G209800.1 | Psat7g031600.1 | arahy.BU1BMX.1 | C.cajan_11568.1 | Glyma.08G016500.1 | AT5G41380.1 |
| Glyma.05G233700.1 | Psat7g011960.1 | . | C.cajan_15626.1 | Glyma.08G041100.1 | . |
| Glyma.06G059600.1 | . | . | C.cajan_32168.1 | Glyma.04G058900.1 | . |
| Glyma.06G097900.1 | . | arahy.0U49HS.1 | C.cajan_17917.1 | Glyma.04G096200.1 | . |
| Glyma.06G098000.1 | . | . | C.cajan_17916.1 | Glyma.04G096300.1 | . |
| Glyma.06G127900.1 | Psat5g077440.1 | arahy.5WR99K.1 | C.cajan_08842.1 | Glyma.04G236400.1 | AT5G3420. [symbol] |
| Glyma.06G136600.1 | Psat5g071920.1 | arahy.A9G6ZW.1 | . | Glyma.04G228300.1 | . |
| Glyma.06G196200.1 | Psat5g199360.1 | . | C.cajan_08715.1 | Glyma.04G166300.1 | . |
| Glyma.07G049400.1 | Psat4g164080.1 | . | C.cajan_39868.1 | Glyma.16G018000.1 | . |
| Glyma.07G091400.1 | . | arahy.QP34EV.1 | C.cajan_08639.1 | Glyma.09G184600.3 | . |
| Glyma.07G182700.1 | Psat7g101680.1 | . | C.cajan_22947.1 | Glyma.08G067200.1 | . |
| Glyma.08G016500.1 | Psat7g031600.1 | arahy.AGT1RS.1 | C.cajan_11568.1 | Glyma.05G209800.1 | . |
| Glyma.08G041100.1 | Psat7g011960.1 | arahy.8MV22J.1 | C.cajan_15626.1 | Glyma.05G233700.1 | . |
| Glyma.08G067200.1 | Psat7g101680.1 | . | C.cajan_22947.1 | Glyma.07G182700.1 | . |
| Glyma.08G168900.1 | . | . | . | Glyma.15G258200.1 | . |
| Glyma.08G221800.1 | Psat7g260880.1 | arahy.QG9XFC.1 | C.cajan_32117.1 | . | AT1G1600. [symbol] |
| Glyma.08G255200.1 | Psat3g193520.1 | arahy.S3IANQ.1 | C.cajan_20131.1 | Glyma.18G278100.1 | . |
| Glyma.09G184600.1 | . | arahy.WEZ503.1 | C.cajan_08639.1 | Glyma.07G091400.1 | . |
| Glyma.10G021400.1 | . | . | C.cajan_10458.1 | Glyma.02G152900.1 | . |
| Glyma.10G048100.1 | Psat6g124840.1 | . | C.cajan_41432.1 | Glyma.13G135900.1 | . |
| Glyma.10G134400.1 | . | arahy.22FLX8.1 | C.cajan_02757.1 | Glyma.10G134400.1 | . |
| Glyma.10G190300.1 | . | arahy.EF48U6.1 | C.cajan_22520.1 | Glyma.20G200400.2 | . |
| Glyma.10G274300.1 | Psat6g228360.1 | arahy.WVH2H8.1 | C.cajan_31872.1 | Glyma.20G115600.1 | AT1G68520.1 |
| Glyma.11G022600.1 | . | . | C.cajan_13216.1 | Glyma.01G221100.1 | . |
| Glyma.12G074400.1 | Psat7g221360.1 | arahy.IR754M.1 | C.cajan_21434.1 | Glyma.U033700.1 | . |
| Glyma.12G196100.1 | Psat5g265480.1 | . | C.cajan_36557.1 | Glyma.13G306400.4 | . |
| Glyma.13G009300.1 | Psat5g105240.1 | . | . | Glyma.13G009300.1 | . |
| Glyma.13G050300.1 | . | arahy.LV381L.1 | C.cajan_26721.1 | Glyma.19G039000.1 | . |
| Glyma.13G093800.1 | . | . | C.cajan_03622.1 | Glyma.17G066600.1 | AT5G [symbol] 7660. [symbol] |
| Glyma.13G100700.1 | Psat4g013600.2 | arahy.0QJD35.1 | C.cajan_03709.1 | Glyma.17G059100.1 | . |
| Glyma.13G135900.1 | Psat6g124840.1 | . | . | Glyma.10G048100.1 | AT5G [symbol] 2810. [symbol] |
| Glyma.13G306400.1 | Psat5g265480.1 | . | C.cajan_36557.1 | Glyma.12G196100.1 | . |
| Glyma.13G331000.1 | Psat5g282000.1 | arahy.KU7VJQ.1 | C.cajan_13596.1 | . | . |
| Glyma.14G182800.1 | . | . | . | Glyma.02G215600.1 | AT4G24470.1 |
| Glyma.14G190400.1 | Psat2g060680.2 | arahy.NK4UTA.1 | C.cajan_29262.1 | Glyma.02G223700.1 | . |
| Glyma.14G197900.1 | Psat2g054040.1 | arahy.XNQT3T.1 | C.cajan_05381.1 | Glyma.02G230800.2 | AT1G07050.1 |
| Glyma.15G219800.1 | . | . | . | Glyma.15G219800.1 | . |
| Glyma.15G258200.1 | . | arahy.9Y5DYJ.1 | . | Glyma.08G168900.1 | . |
| Glyma.16G018000.1 | . | arahy.PEG0RN.1 | . | Glyma.07G049400.1 | . |
| Glyma.16G050900.1 | Psat3g094880.1 | arahy.P8XRWE.1 | C.cajan_17635.1 | Glyma.19G099700.1 | . |
| Glyma.16G067000.1 | . | arahy.FCSG49.1 | C.cajan_00921.1 | . | AT5G [symbol] 4370. [symbol] |
| Glyma.17G059100.1 | . | arahy.0QJD35.1 | C.cajan_03709.1 | Glyma.13G100700.1 | AT5G [symbol] 7180. [symbol] |
| Glyma.17G066600.1 | . | arahy.6J3MKV.1 | C.cajan_03622.1 | Glyma.13G093800.1 | AT5G [symbol] 7660. [symbol] |
| Glyma.17G102200.1 | Psat4g049800.1 | arahy.D90B1M.1 | . | Glyma.05G025000.1 | AT5G61380.1 |
| Glyma.18G278100.1 | Psat3g193520.1 | arahy.669CET.1 | C.cajan_20131.1 | Glyma.08G255200.1 | . |
| Glyma.19G039000.1 | . | . | C.cajan_26721.1 | Glyma.13G050300.1 | . |
| Glyma.19G079600.1 | . | arahy.857KFG.1 | . | . | . |
| Glyma.19G099700.1 | . | arahy.P8XRWE.1 | C.cajan_17635.1 | Glyma.16G050900.1 | . |
| Glyma.19G207100.1 | Psat3g024360.1 | arahy.7A5VUD.1 | C.cajan_10578.1 | Glyma.03G209800.1 | AT2G47890.1 |
| Glyma.19G260400.1 | . | . | C.cajan_10962.1 | Glyma.03G261300.1 | AT2G [symbol] 6790. [symbol] |
| Glyma.20G060400.1 | Psat5g105240.1 | . | . | . | . |
| Glyma.20G085100.1 | . | . | C.cajan_02757.1 | Glyma.10G134400.1 | . |
| Glyma.20G115600.1 | Psat6g228360.1 | arahy.WVH2H8.1 | C.cajan_31872.1 | Glyma.10G274300.1 | . |
| Glyma.20G200400.1 | Psat6g131680.1 | arahy.EF48U6.1 | C.cajan_22520.1 | Glyma.10G190300.1 | AT2G [symbol] 3350. [symbol] |
| Glyma.U033700.1 | Psat7g221360.1 | arahy.IR754M.1 | C.cajan_21434.1 | Glyma.12G074400.1 | . |

[symbol]

indicates data missing or illegible when filed

TABLE 4
Single nucleotide polymorphism

| | non- | splice_{—} | | termination_{—} |
|---|---|---|---|---|
| Gene | Synonymous | site | INDEL | codon_snps |
| Glyma.01G221100 | 4 | 0 | 0 | 0 |
| Glyma.02G152900 | 6 | 0 | 0 | 0 |
| Glyma.02G215600 | 0 | 0 | 0 | 0 |
| Glyma.02G223700 | 2 | 0 | 0 | 0 |
| Glyma.02G230800 | 2 | 0 | 0 | 0 |
| Glyma.03G209800 | 0 | 0 | 0 | 0 |
| Glyma.03G261300 | 1 | 0 | 0 | 0 |
| Glyma.04G058900 | 2 | 0 | 0 | 0 |
| Glyma.04G096200 | 19 | 0 | 0 | 0 |
| Glyma.04G096300 | 3 | 0 | 0 | 0 |
| Glyma.04G166300 | 1 | 1 | 0 | 0 |
| Glyma.04G228300 | 1 | 0 | 0 | 0 |
| Glyma.04G236400 | 1 | 0 | 0 | 0 |
| Glyma.05G025000 | 2 | 0 | 0 | 0 |
| Glyma.05G209800 | 2 | 0 | 0 | 0 |
| Glyma.05G233700 | 2 | 0 | 1 | 0 |
| Glyma.06G059600 | 14 | 0 | 1 | 2 |
| Glyma.06G097900 | 4 | 0 | 1 | 0 |
| Glyma.06G098000 | 4 | 1 | 2 | 0 |
| Glyma.06G127900 | 0 | 0 | 0 | 0 |
| Glyma.06G136600 | 5 | 0 | 0 | 0 |
| Glyma.06G196200 | 4 | 0 | 1 | 0 |
| Glyma.07G049400 | 7 | 0 | 0 | 0 |
| Glyma.07G091400 | 7 | 0 | 0 | 0 |
| Glyma.07G182700 | 1 | 0 | 0 | 0 |
| Glyma.08G016500 | 4 | 0 | 0 | 0 |
| Glyma.08G041100 | 2 | 0 | 0 | 0 |
| Glyma.08G067200 | 1 | 0 | 0 | 0 |
| Glyma.08G168900 | 2 | 0 | 0 | 0 |
| Glyma.08G221800 | 2 | 0 | 0 | 0 |
| Glyma.08G255200 | 3 | 1 | 1 | 0 |
| Glyma.09G184600 | 2 | 1 | 2 | 0 |
| Glyma.10G021400 | 7 | 0 | 0 | 0 |
| Glyma.10G048100 | 9 | 0 | 0 | 0 |
| Glyma.10G134400 | 1 | 0 | 0 | 0 |
| Glyma.10G190300 | 6 | 0 | 0 | 0 |
| Glyma.10G274300 | 1 | 0 | 1 | 0 |
| Glyma.11G022600 | 1 | 0 | 0 | 0 |
| Glyma.12G074400 | 4 | 0 | 1 | 0 |
| Glyma.12G196100 | 3 | 0 | 0 | 0 |
| Glyma.13G009300 | 0 | 0 | 0 | 0 |
| Glyma.13G050300 | 1 | 0 | 0 | 0 |
| Glyma.13G093800 | 1 | 0 | 0 | 0 |
| Glyma.13G100700 | 5 | 0 | 0 | 0 |
| Glyma.13G135900 | 6 | 0 | 0 | 0 |
| Glyma.13G306400 | 4 | 0 | 0 | 0 |
| Glyma.13G331000 | 0 | 0 | 0 | 0 |
| Glyma.14G182800 | 0 | 0 | 0 | 0 |
| Glyma.14G190400 | 4 | 0 | 0 | 0 |
| Glyma.14G197900 | 0 | 0 | 0 | 0 |
| Glyma.15G219800 | 2 | 0 | 1 | 0 |
| Glyma.15G258200 | 0 | 0 | 0 | 0 |
| Glyma.16G018000 | 13 | 0 | 1 | 0 |
| Glyma.16G050900 | 4 | 0 | 0 | 0 |
| Glyma.16G067000 | 2 | 1 | 7 | 0 |
| Glyma.17G059100 | 0 | 0 | 0 | 0 |
| Glyma.17G066600 | 0 | 0 | 0 | 0 |
| Glyma.17G102200 | 3 | 0 | 0 | 0 |
| Glyma.18G278100 | 3 | 0 | 1 | 0 |
| Glyma.19G039000 | 2 | 0 | 0 | 0 |
| Glyma.19G079600 | 0 | 0 | 2 | 0 |
| Glyma.19G099700 | 5 | 0 | 1 | 0 |
| Glyma.19G207100 | 1 | 0 | 0 | 0 |
| Glyma.19G260400 | 8 | 0 | 0 | 0 |
| Glyma.20G060400 | 1 | 0 | 0 | 0 |
| Glyma.20G085100 | 1 | 0 | 3 | 0 |
| Glyma.20G115600 | 3 | 0 | 2 | 0 |
| Glyma.20G200400 | 3 | 0 | 0 | 0 |
| total | 214 | 5 | 29 | 2 |

TABLE 5
Agronomic trait being seed quality
and genes encoding CCT proteins

| gene | QTL TraitName |
|---|---|
| Glyma.01G221100 | Seed oil content |
| Glyma.01G221100 | Seed oil content |
| Glyma.01G221100 | Seed protein content |
| Glyma.01G221100 | Seed glycitein content |
| Glyma.01G221100 | Seed threonine content |
| Glyma.01G221100 | Seed glycitein content |
| Glyma.02G152900 | Seed length to width ratio |
| Glyma.02G215600 | Seed protein content |
| Glyma.02G215600 | Seed isoflavone content |
| Glyma.02G215600 | Seed oil content |
| Glyma.02G215600 | Seed sucrose content |
| Glyma.02G223700 | Seed glycitein content |
| Glyma.03G209800 | Seed linoleic acid content |
| Glyma.03G209800 | Seed oleic acid content |
| Glyma.03G209800 | Seed oil content |
| Glyma.03G209800 | Seed protein content |
| Glyma.03G209800 | Seed oil content |
| Glyma.03G209800 | Seed genistein content |
| Glyma.03G209800 | Seed oil content |
| Glyma.03G209800 | Seed protein content |
| Glyma.03G209800 | Seed stearic acid content |
| Glyma.03G209800 | Seed linolenic acid content |
| Glyma.03G209800 | Seed oil content |
| Glyma.04G058900 | Seed isoflavone content |
| Glyma.04G058900 | Seed genistein content |
| Glyma.04G058900 | Seed protein content |
| Glyma.04G228300 | Seed daidzein content |
| Glyma.04G228300 | Seed isoflavone content |
| Glyma.04G228300 | Seed protein content |
| Glyma.04G228300 | Seed oil content |
| Glyma.04G228300 | Seed protein content |
| Glyma.04G236400 | Seed protein content |
| Glyma.04G236400 | Seed protein content |
| Glyma.04G236400 | Flooding Tolerance |
| Glyma.05G025000 | Seed cysteine content |
| Glyma.05G025000 | Seed methionine content |
| Glyma.05G209800 | Seed oil content |
| Glyma.05G209800 | Seed oil content |
| Glyma.05G209800 | Seed oil content |
| Glyma.05G233700 | Pectin content |
| Glyma.05G209800 | Seed oil content |
| Glyma.05G209800 | Seed linoleic acid content |
| Glyma.05G233700 | Seed oil content |
| Glyma.05G233700 | Seed oleic acid content |
| Glyma.05G209800 | Seed arabinose-galactose content |
| Glyma.05G233700 | Seed oil content |
| Glyma.05G233700 | Seed daidzein content |
| Glyma.05G233700 | Seed isoflavone content |
| Glyma.05G233700 | Seed oil content |
| Glyma.05G233700 | Seed protein content |
| Glyma.05G233700 | Seed daidzein content |
| Glyma.05G233700 | Seed genistein content |
| Glyma.05G233700 | Seed isoflavone content |
| Glyma.05G233700 | Seed glycitein content |
| Glyma.05G233700 | Seed genistein content |
| Glyma.05G233700 | Seed genistein content |
| Glyma.05G233700 | Seed linoleic acid content |
| Glyma.05G233700 | Seed oil content |
| Glyma.05G233700 | Seed protein content |
| Glyma.05G233700 | Seed galactose content |
| Glyma.05G233700 | Seed oil content |
| Glyma.05G233700 | Seed oil content |
| Glyma.05G233700 | Seed stearic acid content |
| Glyma.05G233700 | Seed daidzein content |
| Glyma.05G233700 | Seed isoflavone content |
| Glyma.05G233700 | Seed daidzein content |
| Glyma.05G233700 | Seed genistein content |
| Glyma.05G233700 | Seed linoleic acid content |
| Glyma.05G233700 | Seed oleic acid content |
| Glyma.06G097900 | Seed oil content |
| Glyma.06G097900 | Seed isoflavone content |
| Glyma.06G098000 | Seed isoflavone content |
| Glyma.06G196200 | Seed glycitein content |
| Glyma.06G196200 | Seed glycinin and beta- |
| | conglycinin content |
| Glyma.06G196200 | Seed glycinin content |
| Glyma.06G196200 | Seed stearic acid content |
| Glyma.06G196200 | Seed oil plus protein content |
| Glyma.06G196200 | Seed methionine content |
| Glyma.06G196200 | Seed oil content |
| Glyma.06G196200 | Seed oil content |
| Glyma.06G196200 | Seed protein content |
| Glyma.06G196200 | Seed protein content |
| Glyma.07G049400 | Seed oil content |
| Glyma.07G049400 | Seed protein content |
| Glyma.07G049400 | Seed glycitein content |
| Glyma.07G091400 | Seed protein content |
| Glyma.07G091400 | Seed isoflavone content |
| Glyma.07G091400 | Seed daidzein content |
| Glyma.07G091400 | Seed isoflavone content |
| Glyma.07G091400 | Seed oil content |
| Glyma.07G091400 | Seed protein content |
| Glyma.07G091400 | Seed oil content |
| Glyma.08G168900 | Seed protein content |
| Glyma.08G168900 | Seed oil content |
| Glyma.08G168900 | Seed oil content |
| Glyma.08G168900 | Seed oil content |
| Glyma.08G221800 | Seed fucose content |
| Glyma.08G221800 | Seed palmitic acid content |
| Glyma.09G184600 | Seed oil content |
| Glyma.09G184600 | Seed palmitic acid content |
| Glyma.09G184600 | Seed linolenic acid content |
| Glyma.09G184600 | Seed palmitic acid content |
| Glyma.09G184600 | Seed daidzein content |
| Glyma.09G184600 | Seed genistein content |
| Glyma.09G184600 | Seed isoflavone content |
| Glyma.09G184600 | Seed stearic acid content |
| Glyma.09G184600 | Seed daidzein content |
| Glyma.09G184600 | Seed protein content |
| Glyma.09G184600 | Seed protein content |
| Glyma.10G021400 | Seed glycinin and beta- |
| | conglycinin content |
| Glyma.10G048100 | Seed oil content |
| Glyma.10G048100 | Seed protein content |
| Glyma.10G190300 | Seed protein content |
| Glyma.10G190300 | Seed protein content |
| Glyma.10G190300 | Seed glycitein content |
| Glyma.10G190300 | Seed tocopherol content |
| Glyma.10G274300 | Seed linoleic acid content |
| Glyma.10G274300 | Seed linolenic acid content |
| Glyma.10G274300 | Seed glucose content |
| Glyma.12G074400 | Seed protein content |
| Glyma.12G074400 | Seed daidzein content |
| Glyma.12G074400 | Seed genistein content |
| Glyma.12G074400 | Seed isoflavone content |
| Glyma.12G196100 | Seed oil content |
| Glyma.12G196100 | Seed protein content |
| Glyma.12G196100 | Seed oil content |
| Glyma.12G196100 | Seed oil content |
| Glyma.12G196100 | Seed protein content |
| Glyma.12G196100 | Seed protein content |
| Glyma.13G009300 | Seed protein content |
| Glyma.13G050300 | Seed isoflavone content |
| Glyma.13G093800 | Seed genistein content |
| Glyma.13G093800 | Seed linoleic acid content |
| Glyma.13G093800 | Seed oleic acid content |
| Glyma.13G135900 | Seed linoleic acid content |
| Glyma.13G135900 | Seed oleic acid content |
| Glyma.13G135900 | Seed glycitein content |
| Glyma.13G135900 | Seed oil content |
| Glyma.13G306400 | Seed isoflavone content |
| Glyma.13G331000 | Seed protein content |
| Glyma.13G331000 | Seed stearic acid content |
| Glyma.14G182800 | Seed isoflavone content |
| Glyma.14G182800 | Seed oil content |
| Glyma.14G190400 | Seed oil content |
| Glyma.14G197900 | Seed linoleic acid content |
| Glyma.14G197900 | Seed linoleic acid content |
| Glyma.14G197900 | Seed genistein content |
| Glyma.14G197900 | Seed linolenic acid content |
| Glyma.16G018000 | Seed isoflavone content |
| Glyma.16G018000 | Seed protein content |
| Glyma.16G018000 | Seed isoflavone content |
| Glyma.16G018000 | Seed sucrose content |
| Glyma.16G018000 | Seed soluble oligosaccharides |
| Glyma.16G050900 | Seed palmitic acid content |
| Glyma.16G050900 | Seed protein content |
| Glyma.16G050900 | Seed oil content |
| Glyma.16G050900 | Seed oil content |
| Glyma.16G067000 | Seed glycitein content |
| Glyma.16G067000 | Seed soluble oligosaccharides |
| Glyma.17G059100 | Seed linolenic acid content |
| Glyma.17G059100 | Seed genistein content |
| Glyma.17G059100 | Starch content |
| Glyma.17G066600 | Seed palmitic acid content |
| Glyma.17G102200 | Seed oil content |
| Glyma.17G102200 | Seed beta-conglycinin beta- |
| | subunit protein content |
| Glyma.17G102200 | Seed glycinin and beta- |
| | conglycinin content |
| Glyma.17G102200 | Seed isoflavone content |
| Glyma.17G102200 | Seed leucine content |
| Glyma.17G102200 | Seed methionine and |
| | cysteine content |
| Glyma.17G102200 | Seed palmitic + stearic |
| | acid content |
| Glyma.17G102200 | Seed glycitein content |
| Glyma.17G102200 | Seed palmitic acid content |
| Glyma.17G102200 | Seed linoleic acid content |
| Glyma.18G278100 | Seed stearic acid content |
| Glyma.19G039000 | Seed oil content |
| Glyma.19G039000 | Seed oil content |
| Glyma.19G039000 | Seed protein content |
| Glyma.19G039000 | Seed palmitic + stearic |
| | acid content |
| Glyma.19G039000 | Seed soluble |
| | oligosaccharides |
| Glyma.19G039000 | Seed sucrose content |
| Glyma.19G079600 | Seed oil content |
| Glyma.19G099700 | Seed oil content |
| Glyma.19G099700 | Seed sucrose content |
| Glyma.19G099700 | Seed isoleucine content |
| Glyma.19G099700 | Seed soluble oligosaccharides |
| Glyma.19G099700 | Seed tyrosine content |
| Glyma.19G099700 | Seed sucrose content |
| Glyma.19G099700 | Seed genistein content |
| Glyma.19G099700 | Seed genistein content |
| Glyma.19G099700 | Seed isoflavone content |
| Glyma.19G207100 | Seed palmitic acid content |
| Glyma.19G207100 | Seed daidzein content |
| Glyma.19G207100 | Seed genistein content |
| Glyma.19G207100 | Seed isoflavone content |
| Glyma.19G207100 | Seed oil content |
| Glyma.19G207100 | Seed protein content |
| Glyma.19G207100 | Seed hardness |
| Glyma.19G207100 | Seed oil content |
| Glyma.19G207100 | Seed oil content |
| Glyma.19G260400 | Seed daidzein content |
| Glyma.19G260400 | Seed protein content |
| Glyma.20G085100 | Seed lysine content |
| Glyma.20G085100 | Seed methionine content |
| Glyma.20G085100 | Seed protein content |
| Glyma.20G085100 | Seed threonine content |
| Glyma.20G085100 | Seed oil content |
| Glyma.20G085100 | Seed protein content |
| Glyma.20G085100 | Seed protein content |
| Glyma.20G085100 | Seed oil content |
| Glyma.20G085100 | Seed calcium content |
| Glyma.20G085100 | Seed oil content |
| Glyma.20G085100 | Seed protein content |
| Glyma.20G085100 | Seed linoleic acid content |
| Glyma.20G085100 | Seed oleic acid content |
| Glyma.20G085100 | Seed oil content |
| Glyma.20G085100 | Seed protein content |
| Glyma.20G115600 | Seed isoflavone content |
| Glyma.20G115600 | Seed delta-tocopherol content |
| Glyma.20G200400 | Seed genistein content |
| Glyma.20G200400 | Seed oleic acid content |

TABLE 6
Agronomic trait being seed set and genes encoding CCT proteins

| gene | QTL TraitName |
|---|---|
| Glyma.01G221100 | Seed weight |
| Glyma.01G221100 | Seed weight |
| Glyma.02G223700 | Seed Set |
| Glyma.03G209800 | Pod weight ratio |
| Glyma.03G209800 | Seed height |
| Glyma.03G209800 | Seed Number |
| Glyma.03G209800 | Seed weight |
| Glyma.03G209800 | Seed width |
| Glyma.04G058900 | Seed weight |
| Glyma.04G058900 | Seed height |
| Glyma.04G058900 | Seed size |
| Glyma.04G058900 | Seed width |
| Glyma.04G058900 | Seed Set |
| Glyma.04G058900 | Seed Set |
| Glyma.04G058900 | Seed weight |
| Glyma.04G058900 | Seed weight |
| Glyma.04G228300 | Seed height |
| Glyma.04G228300 | Seed weight |
| Glyma.04G228300 | Seed width |
| Glyma.04G228300 | Seed Set |
| Glyma.04G236400 | Seed Number |
| Glyma.04G236400 | Seed weight |
| Glyma.05G209800 | Seed height |
| Glyma.05G209800 | Seed size |
| Glyma.05G209800 | Seed weight |
| Glyma.05G233700 | Seed yield |
| Glyma.05G233700 | Seed weight |
| Glyma.06G059600 | Seed size |
| Glyma.06G059600 | Seed width |
| Glyma.06G059600 | Seed yield |
| Glyma.06G059600 | Seed weight |
| Glyma.06G097900 | Seed weight |
| Glyma.06G097900 | Seed weight |
| Glyma.06G097900 | Seed Number |
| Glyma.06G098000 | Seed weight |
| Glyma.06G097900 | Seed width |
| Glyma.06G097900 | Seed height |
| Glyma.06G097900 | Seed width |
| Glyma.06G196200 | Seed yield |
| Glyma.06G196200 | Seed Number |
| Glyma.06G196200 | Seed weight |
| Glyma.06G196200 | Pods Per Node |
| Glyma.06G196200 | Seed yield |
| Glyma.06G196200 | Seed Set |
| Glyma.06G196200 | Seed width |
| Glyma.06G196200 | Pods per plant |
| Glyma.06G196200 | Seed Set |
| Glyma.06G196200 | Seed weight |
| Glyma.06G196200 | Seed weight |
| Glyma.06G196200 | Seed weight |
| Glyma.06G196200 | Seed yield |
| Glyma.06G196200 | Seed yield |
| Glyma.06G196200 | Seed yield |
| Glyma.06G196200 | Seed yield |
| Glyma.06G196200 | Seed yield |
| Glyma.07G049400 | Seed width |
| Glyma.07G049400 | Seed height |
| Glyma.07G049400 | Seed size |
| Glyma.07G049400 | Seed width |
| Glyma.07G049400 | Seed yield |
| Glyma.07G049400 | Seed weight |
| Glyma.07G049400 | Seed yield |
| Glyma.07G091400 | Seed yield |
| Glyma.07G091400 | Seed height |
| Glyma.07G182700 | Seed weight |
| Glyma.08G016500 | Seed weight |
| Glyma.08G016500 | Seed weight |
| Glyma.08G067200 | Seed Set |
| Glyma.08G067200 | Seed width |
| Glyma.09G184600 | Seed Set |
| Glyma.09G184600 | Seed weight |
| Glyma.09G184600 | Seed weight |
| Glyma.09G184600 | Seed weight |
| Glyma.09G184600 | Seed weight |
| Glyma.09G184600 | Seeds per plant |
| Glyma.09G184600 | Seed yield |
| Glyma.10G134400 | Seed Set |
| Glyma.10G190300 | Seed weight |
| Glyma.10G190300 | Seed length to width ratio |
| Glyma.10G190300 | Seed width |
| Glyma.11G022600 | Seed Set |
| Glyma.11G022600 | Seed Number |
| Glyma.11G022600 | Seed weight |
| Glyma.11G022600 | Seed height |
| Glyma.11G022600 | Seed weight |
| Glyma.12G074400 | Seed weight |
| Glyma.12G196100 | Seed weight |
| Glyma.12G196100 | Seed weight |
| Glyma.12G196100 | Seed weight |
| Glyma.12G196100 | Seed yield |
| Glyma.13G093800 | Seed Set |
| Glyma.13G093800 | Seed weight |
| Glyma.13G135900 | Seed weight |
| Glyma.13G135900 | Seed weight |
| Glyma.14G182800 | Seed yield |
| Glyma.14G182800 | Seed yield |
| Glyma.15G219800 | Seed weight |
| Glyma.16G018000 | Seed yield |
| Glyma.16G018000 | Seed yield/Plant height |
| Glyma.16G050900 | Seed weight |
| Glyma.16G050900 | Seed weight |
| Glyma.16G050900 | Seed yield |
| Glyma.17G059100 | Seed Set |
| Glyma.17G059100 | Seed weight |
| Glyma.17G066600 | Seed weight |
| Glyma.17G066600 | Seed weight |
| Glyma.17G066600 | Seed weight |
| Glyma.17G102200 | Seed weight |
| Glyma.17G102200 | Seed yield |
| Glyma.17G102200 | Seed yield |
| Glyma.17G102200 | Seed weight |
| Glyma.17G102200 | Seed yield/Plant height |
| Glyma.17G102200 | Seeds per plant |
| Glyma.17G102200 | Seed weight |
| Glyma.17G102200 | Seed height |
| Glyma.18G278100 | Seed weight |
| Glyma.18G278100 | Seed yield |
| Glyma.19G039000 | Seed Set |
| Glyma.19G099700 | Seed Set |
| Glyma.19G099700 | Seed weight |
| Glyma.19G099700 | Seed yield |
| Glyma.19G207100 | Seed weight |
| Glyma.19G207100 | Seed yield/Plant height |
| Glyma.19G207100 | Seed Number |
| Glyma.19G207100 | Seed weight |
| Glyma.19G260400 | Seed yield/Plant height |
| Glyma.20G085100 | Seed weight |
| Glyma.20G085100 | Seed yield |
| Glyma.20G115600 | Seed Set |
| Glyma.20G200400 | Seed weight |
| Glyma.20G200400 | Seed weight |

TABLE 7
Agronomic traits being abiotic/biotic stress
tolerance and genes encoding CCT proteins

| gene | QTL TraitName |
|---|---|
| Glyma.01G221100 | Reaction to Heterodera glycines |
| Glyma.01G221100 | Reaction to Sclerotinia sclerotiorum infection |
| Glyma.02G152900 | Reaction to Phytophthora sojae infection |
| Glyma.03G209800 | Reaction to Bemisia tabaci |
| Glyma.04G058900 | Reaction to Heterodera glycines |
| Glyma.04G058900 | Reaction to Heterodera glycines |
| Glyma.04G096300 | Ozone sensitivity, leaflet |
| Glyma.04G228300 | Reaction to Fusarium solani f sp glycines infection |
| Glyma.04G228300 | Phosphorus content |
| Glyma.04G228300 | Phosphorus use efficiency |
| Glyma.04G228300 | Reaction to Soybean Mosaic Virus Infection (SMV) |
| Glyma.04G236400 | Flooding Tolerance |
| Glyma.04G236400 | Lodging |
| Glyma.05G025000 | Reaction to Heterodera glycines |
| Glyma.05G209800 | Reaction to Sclerotinia sclerotiorum infection |
| Glyma.05G209800 | Lodging |
| Glyma.05G233700 | Reaction to Bemisia tabaci |
| Glyma.06G059600 | Reaction to Fusarium solani f sp glycines infection |
| Glyma.06G059600 | Seed glycinin and beta-conglycinin content |
| Glyma.06G098000 | Manganese induced root necrosis |
| Glyma.06G098000 | Reaction to Fusarium solani f sp glycines infection |
| Glyma.06G127900 | Embryo Abortion |
| Glyma.06G136600 | Manganese induced root necrosis |
| Glyma.06G196200 | Reaction to Sclerotinia sclerotiorum infection |
| Glyma.06G196200 | R/V photothermal sensitivity |
| Glyma.06G196200 | Reaction to Aulacorthum solani, choice |
| Glyma.06G196200 | Reaction to Phytophthora sojae infection |
| Glyma.06G196200 | Lodging |
| Glyma.06G196200 | Lodging |
| Glyma.07G049400 | Reaction to Aulacorthum solani, choice |
| Glyma.07G091400 | Reaction to Spodoptera litura |
| Glyma.07G091400 | Reaction to Phytophthora sojae infection |
| Glyma.07G091400 | Row spacing response |
| Glyma.07G091400 | Reaction to Helicoverpa zea |
| Glyma.08G016500 | Reaction to Fusarium solani f sp glycines infection |
| Glyma.08G041100 | Reaction to Fusarium solani f sp glycines infection |
| Glyma.08G067200 | Reaction to Fusarium solani f sp glycines infection |
| Glyma.08G067200 | Reaction to Fusarium solani f sp glycines infection |
| Glyma.08G067200 | Reaction to Heterodera glycines |
| Glyma.08G067200 | Reaction to Heterodera glycines |
| Glyma.08G168900 | Acid phosphatase activity |
| Glyma.08G168900 | Phosphorus content |
| Glyma.08G168900 | Phosphorus use efficiency |
| Glyma.08G255200 | Reaction to Heterodera glycines |
| Glyma.08G221800 | Reaction to Sclerotinia sclerotiorum infection |
| Glyma.08G221800 | Lodging |
| Glyma.08G255200 | Reaction to Sclerotinia sclerotiorum infection |
| Glyma.08G221800 | Reaction to Phytophthora sojae infection |
| Glyma.09G184600 | Flooding Tolerance |
| Glyma.10G021400 | Reaction to Fusarium solani f sp glycines infection |
| Glyma.10G021400 | Reaction to Meloidogyne incognita |
| Glyma.10G048100 | Reaction to Fusarium solani f sp glycines infection |
| Glyma.10G048100 | Reaction to Meloidogyne incognita |
| Glyma.10G048100 | Reaction to Meloidogyne incognita |
| Glyma.10G134400 | Reaction to Heterodera glycines |
| Glyma.10G190300 | Drought Tolerance |
| Glyma.10G190300 | Reaction to Lamprosema indicata |
| Glyma.10G190300 | Reaction to Sclerotinia sclerotiorum infection |
| Glyma.10G190300 | Probability an electron reduces an end acceptor at |
| | PSI electron acceptor side |
| Glyma.10G190300 | Reaction to Heterodera glycines |
| Glyma.11G022600 | Reaction to Fusarium solani f sp glycines infection |
| Glyma.12G074400 | Reaction to Phytophthora sojae infection |
| Glyma.12G074400 | Reaction to Heterodera glycines |
| Glyma.12G196100 | Reaction to Helicoverpa zea |
| Glyma.12G196100 | Drought Tolerance |
| Glyma.12G196100 | Drought Susceptibility Index |
| Glyma.13G009300 | Reaction to Phytophthora sojae infection |
| Glyma.13G050300 | Reaction to Aphis glycines |
| Glyma.13G050300 | Reaction to Phytophthora sojae infection |
| Glyma.13G093800 | Reaction to Fusarium solani f sp glycines infection |
| Glyma.13G093800 | Flooding Tolerance |
| Glyma.13G093800 | Reaction to Heterodera glycines |
| Glyma.13G093800 | Reaction to Meloidogyne javanica |
| Glyma.13G093800 | Reaction to Phytophthora sojae infection |
| Glyma.13G135900 | Reaction to Phytophthora sojae infection |
| Glyma.13G331000 | Reaction to Helicoverpa zea |
| Glyma.14G190400 | Iron Efficiency |
| Glyma.14G197900 | Iron Efficiency |
| Glyma.15G219800 | Reaction to Heterodera glycines |
| Glyma.15G258200 | Lodging |
| Glyma.16G018000 | Drought Susceptibility Index |
| Glyma.16G018000 | Reaction to Helicoverpa zea |
| Glyma.16G067000 | Reaction to Soybean Mosaic Virus Infection (SMV) |
| Glyma.16G067000 | Reaction to Bemisia tabaci |
| Glyma.17G066600 | Phosphorus content |
| Glyma.17G066600 | Reaction to Heterodera glycines |
| Glyma.17G059100 | Reaction to Sclerotinia sclerotiorum infection |
| Glyma.17G066600 | Reaction to Sclerotinia sclerotiorum infection |
| Glyma.17G066600 | Root phosphorus content |
| Glyma.17G066600 | Shoot phosphorus content |
| Glyma.17G102200 | Drought Susceptibility Index |
| Glyma.17G102200 | Manganese induced leaflet necrosis |
| Glyma.17G102200 | Reaction to Sclerotinia sclerotiorum infection |
| Glyma.17G102200 | Reaction to Sclerotinia sclerotiorum infection |
| Glyma.18G278100 | Reaction to Aulacorthum solani, choice |
| Glyma.18G278100 | Reaction to Heterodera glycines |
| Glyma.18G278100 | Reaction to Phytophthora sojae infection |
| Glyma.18G278100 | Reaction to Helicoverpa zea |
| Glyma.18G278100 | Reaction to Heterodera glycines |
| Glyma.19G039000 | Reaction to Sclerotinia sclerotiorum infection |
| Glyma.19G039000 | UV-B induced leaflet shape response |
| Glyma.19G039000 | Chlorimuron Ethyl Sensitivity |
| Glyma.19G099700 | Chlorimuron Ethyl Sensitivity |
| Glyma.19G207100 | Reaction to Fusarium solani f sp glycines infection |
| Glyma.19G207100 | Lodging |
| Glyma.19G207100 | Lodging |
| Glyma.19G207100 | Reaction to Fusarium solani f sp glycines infection |
| Glyma.19G207100 | Flooding Tolerance |
| Glyma.19G207100 | Reaction to Fusarium infection |
| Glyma.20G085100 | Reaction to Heterodera glycines |
| Glyma.20G085100 | Ozone sensitivity, leaflet |
| Glyma.20G115600 | Reaction to Phytophthora sojae infection |
| Glyma.20G115600 | Reaction to Fusarium solani f sp glycines infection |
| Glyma.20G200400 | Reaction to Fusarium solani f sp glycines infection |
| Glyma.20G200400 | Reaction to Popilliae japnoica |
| Glyma.20G200400 | Reaction to Heterodera glycines |
| Glyma.20G200400 | Reaction to Heterodera glycines |
| Glyma.20G200400 | Reaction to Heterodera glycines |
| Glyma.20G200400 | Reaction to Heterodera glycines |

TABLE 8
Agronomic trait being flowering time
and genes encoding CCT protein

| gene | QTL TraitName |
|---|---|
| Glyma.02G152900 | First flower |
| Glyma.02G215600 | First flower |
| Glyma.02G223700 | First flower |
| Glyma.04G166300 | Reproductive stage length |
| Glyma.04G228300 | R8 Full Maturity |
| Glyma.04G228300 | R8 Full Maturity |
| Glyma.04G236400 | First flower |
| Glyma.04G236400 | R8 Full Maturity |
| Glyma.06G059600 | R8 Full Maturity |
| Glyma.06G097900 | R8 Full Maturity |
| Glyma.06G196200 | First flower |
| Glyma.06G196200 | Photoperiod sensitivity |
| Glyma.06G196200 | R3 Beginning pod |
| Glyma.06G196200 | R7 Beginning Maturity |
| Glyma.06G196200 | First flower |
| Glyma.06G196200 | First flower |
| Glyma.06G196200 | R5 Beginning Seed |
| Glyma.06G196200 | R8 Full Maturity |
| Glyma.06G196200 | R8 Full Maturity |
| Glyma.06G196200 | First flower |
| Glyma.06G196200 | First flower |
| Glyma.07G049400 | R8 Full Maturity |
| Glyma.07G091400 | First flower |
| Glyma.07G091400 | R8 Full Maturity |
| Glyma.07G091400 | Seed filling period |
| Glyma.10G190300 | Reproductive stage length |
| Glyma.10G190300 | R8 Full Maturity |
| Glyma.12G074400 | First flower |
| Glyma.12G074400 | R8 Full Maturity |
| Glyma.12G196100 | R8 Full Maturity |
| Glyma.13G009300 | R8 Full Maturity |
| Glyma.13G050300 | R8 Full Maturity |
| Glyma.13G135900 | Photoperiod sensitivity |
| Glyma.13G135900 | Reproductive stage length |
| Glyma.13G135900 | R8 Full Maturity |
| Glyma.14G182800 | Reproductive period |
| Glyma.16G018000 | R8 Full Maturity |
| Glyma.16G018000 | R8 Full Maturity |
| Glyma.16G018000 | Reproductive stage length |
| Glyma.18G278100 | R8 Full Maturity |
| Glyma.18G278100 | R8 Full Maturity |
| Glyma.19G207100 | First flower |
| Glyma.19G207100 | R8 Full Maturity |
| Glyma.19G207100 | First flower |
| Glyma.19G260400 | First flower |
| Glyma.19G207100 | Flower form |
| Glyma.19G260400 | First flower |
| Glyma.19G260400 | First flower |
| Glyma.19G260400 | R8 Full Maturity |
| Glyma.19G260400 | Reproductive stage length |
| Glyma.20G115600 | R8 Full Maturity |

TABLE 9
Agronomic trait being development-related
traits and genes encoding CCT proteins

| gene | QTL TraitName |
|---|---|
| Glyma.01G221100 | Pubescence Density |
| Glyma.02G223700 | Canopy wilt |
| Glyma.02G215600 | Shattering |
| Glyma.02G230800 | Internode length |
| Glyma.02G215600 | Node Number |
| Glyma.02G215600 | Canopy wilt |
| Glyma.03G209800 | Leaflet chlorophyll content |
| Glyma.03G209800 | Leaflet Width |
| Glyma.03G209800 | Petiole color |
| Glyma.03G209800 | UV-B induced leaflet shape response |
| Glyma.03G209800 | UV-B induced plant damage |
| Glyma.03G209800 | Plant Height |
| Glyma.04G058900 | Canopy wilt |
| Glyma.04G058900 | Branching |
| Glyma.04G228300 | Plant Height |
| Glyma.04G228300 | Seed coat cracking |
| Glyma.04G228300 | Lodging |
| Glyma.04G228300 | Plant Height |
| Glyma.04G228300 | Root weight, dry |
| Glyma.04G236400 | Leaflet shape |
| Glyma.04G236400 | Leaflet Width |
| Glyma.05G233700 | Plant Height |
| Glyma.05G209800 | Plant Height |
| Glyma.05G209800 | Cell wall polysaccharide composition |
| Glyma.05G209800 | Root Nodule Size |
| Glyma.05G233700 | Leaflet Length |
| Glyma.05G233700 | Leaflet Width |
| Glyma.06G097900 | shoot weight, fresh |
| Glyma.06G059600 | Flower form |
| Glyma.06G098000 | Plant weight |
| Glyma.06G098000 | shoot weight, dry |
| Glyma.06G097900 | Root nodule number |
| Glyma.06G097900 | Root nodule weight |
| Glyma.06G097900 | shoot weight, dry |
| Glyma.06G136600 | Node Number |
| Glyma.06G196200 | Plant Height |
| Glyma.06G196200 | Root length, coarse |
| Glyma.06G196200 | Root volume |
| Glyma.06G196200 | Plant Height |
| Glyma.07G049400 | Plant Height |
| Glyma.07G049400 | Seed height |
| Glyma.07G091400 | Seed height |
| Glyma.07G091400 | Canopy Height |
| Glyma.07G091400 | Leaflet area |
| Glyma.07G091400 | Root nodule weight, dry |
| Glyma.07G091400 | Leaflet chlorophyll content |
| Glyma.07G091400 | Internode length |
| Glyma.07G091400 | Pods Per Node |
| Glyma.07G182700 | Plant Height |
| Glyma.08G016500 | Seed delta-tocopherol content |
| Glyma.08G016500 | Seed gamma-tocopherol content |
| Glyma.08G016500 | Pods per plant |
| Glyma.08G041100 | Stem strength |
| Glyma.08G067200 | Canopy wilt |
| Glyma.08G067200 | Pubescence Density |
| Glyma.08G067200 | Pubescence Density |
| Glyma.08G067200 | Seed coat cracking |
| Glyma.08G067200 | Leaflet area |
| Glyma.08G067200 | Canopy wilt |
| Glyma.09G184600 | Pods per plant |
| Glyma.10G021400 | Root dry weight to shoot dry weight ratio |
| Glyma.10G048100 | Row spacing response |
| Glyma.10G048100 | Seed length to height ratio |
| Glyma.10G048100 | Seed length to height ratio |
| Glyma.10G134400 | Net photosynthetic rate |
| Glyma.10G190300 | Pods per plant |
| Glyma.10G190300 | Total growth duration |
| Glyma.10G190300 | Root surface area, medium |
| Glyma.10G190300 | Root volume |
| Glyma.10G190300 | Root weight, dry |
| Glyma.10G190300 | Branching |
| Glyma.10G190300 | Leaflet shape |
| Glyma.10G190300 | Leaflet Width |
| Glyma.10G190300 | Plant Height |
| Glyma.11G022600 | Hypocotyl Length |
| Glyma.12G074400 | Leaflet area |
| Glyma.12G074400 | Plant Height |
| Glyma.12G074400 | Total growth duration |
| Glyma.12G074400 | Branching |
| Glyma.12G196100 | Neutral detergent fiber content |
| Glyma.12G196100 | Plant Height |
| Glyma.12G196100 | Pubescence Density |
| Glyma.12G196100 | Plant Height |
| Glyma.12G196100 | Canopy wilt |
| Glyma.13G009300 | Leaf phosphorus content |
| Glyma.13G050300 | Node Number |
| Glyma.13G050300 | Leaflet chlorophyll content |
| Glyma.13G093800 | Seed coat cracking |
| Glyma.13G093800 | Canopy Height |
| Glyma.13G093800 | Embryo Abortion |
| Glyma.13G093800 | Leaflet Width |
| Glyma.13G093800 | Hypocotyl Length |
| Glyma.13G093800 | Plant Height |
| Glyma.13G306400 | Plant Height |
| Glyma.13G306400 | Neutral detergent fiber content |
| Glyma.13G306400 | Plant Height |
| Glyma.13G331000 | Acid detergent fiber content |
| Glyma.13G331000 | Plant Height |
| Glyma.14G197900 | Leaflet Length |
| Glyma.14G197900 | Somatic embryos per explant |
| Glyma.16G018000 | Plant Height |
| Glyma.16G018000 | Leaflet area |
| Glyma.16G018000 | Shattering |
| Glyma.16G050900 | Leaflet shape |
| Glyma.16G050900 | Plant Height |
| Glyma.16G050900 | Shattering |
| Glyma.16G050900 | shoot weight, dry |
| Glyma.16G050900 | Leaflet trigonelline content |
| Glyma.16G050900 | Seed viability |
| Glyma.17G066600 | Plant weight |
| Glyma.17G066600 | Plant weight, dry |
| Glyma.17G059100 | Canopy wilt |
| Glyma.17G066600 | Root Weight |
| Glyma.17G066600 | Root weight, dry |
| Glyma.17G066600 | shoot weight, dry |
| Glyma.17G102200 | Node Number |
| Glyma.17G102200 | Canopy wilt |
| Glyma.17G102200 | Canopy wilt |
| Glyma.17G102200 | Canopy wilt |
| Glyma.17G102200 | Canopy wilt |
| Glyma.17G102200 | Canopy wilt |
| Glyma.18G278100 | Plant Height |
| Glyma.18G278100 | Plant Height |
| Glyma.18G278100 | Plant Height |
| Glyma.19G039000 | Shattering |
| Glyma.19G039000 | Leaflet Length |
| Glyma.19G039000 | Leaflet shape |
| Glyma.19G039000 | Leaflet Width |
| Glyma.19G099700 | Plant Height |
| Glyma.19G099700 | Seed length to width ratio |
| Glyma.19G099700 | Plant Height |
| Glyma.19G099700 | Leaflet Length |
| Glyma.19G099700 | Leaflet shape |
| Glyma.19G099700 | Leaflet Length |
| Glyma.19G099700 | Leaflet Width |
| Glyma.19G207100 | Pods per plant |
| Glyma.19G207100 | Canopy wilt |
| Glyma.19G207100 | Canopy wilt |
| Glyma.19G207100 | Plant Height |
| Glyma.19G207100 | Height/Lodging |
| Glyma.19G207100 | Leaflet Length |
| Glyma.19G207100 | Plant Height |
| Glyma.19G207100 | Branching |
| Glyma.19G207100 | Canopy wilt |
| Glyma.19G207100 | Leaflet shape |
| Glyma.19G207100 | Leaflet Width |
| Glyma.19G207100 | Seed Coat Hardness |
| Glyma.19G260400 | Leaflet Length |
| Glyma.19G260400 | Leaflet shape |
| Glyma.19G260400 | Leaflet Width |
| Glyma.19G260400 | Plant Height |
| Glyma.19G260400 | Plant Height |
| Glyma.20G085100 | Internode length |
| Glyma.20G115600 | Pods per plant |
| Glyma.20G115600 | Leaflet Width |
| Glyma.20G115600 | Specific leaflet weight |
| Glyma.20G200400 | Canopy Width |
| Glyma.20G200400 | Root nodule number |

TABLE 10
SNPs significantly associated with seed protein and oil content and 100-seed weight. Significant variants

| | | | | | | | SNP | SNP |
|---|---|---|---|---|---|---|---|---|
| | | | | | | | Distance | Distance |
| | | | | | Seed | | to 5′ | to 3′ |
| Trait/ | | | Oil | Protein | weigth | SNP/gene | flanking | flanking |
| gene model | Chr | Position | p-value | p-value | p-value | Annotation | gene Ihpl | gene Ihpl |
| Glyma.20G084400 | 20 | 31,650,003 start | | | | N/A | | |
| | | 31,650,419 stop | | | | | | |
| SNPI | 20 | 31,656,273 | | 4.91E−08 | | intergenic | 5,854 | 9,917 |
| SNP2 | 20 | 31,658,904 | 2.41E−22 | 7.25E−08 | | intergenic | 8,485 | 7,286 |
| SNP3 | 20 | 31,658,920 | | 1.82E−07 | | intergenic | 8,501 | 7,270 |
| SNP4 | 20 | 31,661,646 | | 1.98E−07 | | intergenic | 11,227 | 4,544 |
| SNP5 | 20 | 31,664,872 | 4.91E−22 | 6.00E−08 | 4.38NE−14 | intergenic | 14,453 | 1,318 |
| Glyma.20G084500 | 20 | 31,666,190 stop | | | | MOS4- | | |
| | | 31,674,959 start | | | | associated | | |
| | | | | | | complex 3B | | |
| Glyma.20G084600 | 20 | 31,668,842 start | | | | N/A | | |
| | | 31,669,146 stop | | | | | | |
| SNP6 | 20 | 31,680,622 | 2.56E−21 | 8.96E−08 | | intergenic | 5,663 | 6,191 |
| SNP7 | 20 | 31,682,844 | | 4.60E−07 | | intergenic | 7,885 | 3,969 |
| SNP8 | 20 | 31,685,116 | | 2.95E−07 | | intergenic | 10,157 | 1,697 |
| Glyma.20G084700 | 20 | 31,686,813 stop | | | | unknown | | |
| | | 31,687,483 start | | | | protein | | |
| Glyma.20G084800 | 20 | 31,689,766 start | | | | N/A | | |
| | | 31,690,181 stop | | | | | | |
| SNP9 | 20 | 31,691,428 | 2.72E−22 | 2.88E−07 | | intergenic | 1,247 | 15,919 |
| SNP10 | 20 | 31,692,813 | 3.96E−20 | 6.63E−08 | | intergenic | 2,632 | 14,534 |
| SNP11 | 20 | 31,693,770 | | 5.05E−08 | | intergenic | 3,589 | 13,577 |
| SNP12 | 20 | 31,696,275 | 1.86E−19 | 8.61E−08 | 1.03E−12 | intergenic | 6,094 | 11,072 |
| Glyma.20G084900 | 20 | 31,707,347 stop | | | | hAT | | |
| | | 31,709,780 start | | | | dimerisation | | |
| | | | | | | domain- | | |
| | | | | | | containing | | |
| | | | | | | protein | | |
| SNP13 | 20 | 31,713,462 | 8.25E−20 | 3.45E−07 | | intergenic | 3,682 | 32,024 |
| SNP14 | 20 | 31,718,263 | 6.74E−19 | 3.26E−08 | | intergenic | 8,483 | 27,223 |
| SNP15 | 20 | 31,718,476 | | 1.45E−07 | | intergenic | 8,696 | 27,010 |
| SNP16 | 20 | 31,724,545 | 4.71E−20 | | | intergenic | 14,765 | 20,941 |
| SNP17 | 20 | 31,727,573 | 1.40E−20 | 4.49E−08 | | intergenic | 17,793 | 17,913 |
| SNP18 | 20 | 31,730,753 | 4.78E−21 | 3.24E−08 | | intergenic | 20,973 | 14,733 |
| SNP19 | 20 | 31,731,003 | 1.56E−20 | 6.28E−08 | | intergenic | 21,223 | 14,483 |
| SNP20 | 20 | 31,733,134 | | 2.79E−07 | | intergenic | 23,354 | 12,352 |
| SNP21 | 20 | 31,737,391 | 1.37E−22 | 1.84E−07 | | intergenic | 27,611 | 8,095 |
| SNP22 | 20 | 31,738,091 | | | | intergenic | 28,311 | 7,395 |
| Glyma.20G085000 | 20 | 31,745,486 stop | | | | Conversed | | |
| | | | | | | oligomeric | | |
| | | | | | | complex | | |
| | | | | | | COG6 | | |
| InDell | 20 | 31,746,064 | 4.25E−21 | 4.87E−12 | 4.87E−12 | intron 2 | N/A | |
| Glyma20G085000 | 20 | 31,748,473 start | | | | intergenic | 3,087 | 23,210 |
| SNP23 | 20 | 31,751,560 2-bp ins | 2.253E−23 | | | intergenic | 6,565 | 19,732 |
| SNP24 | 20 | 31,755,038 | 2.78E−18 | | | intergenic | 10,686 | 15,611 |
| SNP25 | 20 | 31,759,159 | 4.68E−20 | | | intergenic | 11,820 | 14,477 |
| SNP26 | 20 | 31,760,293 | 1.40E−18 | | | intergenic | 3,087 | 23,210 |
| SNP27 | 20 | 31,761,452 | 1.71E−18 | 2.59E−07 | | intergenic | 12,979 | 13,318 |
| Glyma.20G085100 | 20 | 31,774,770 start | | | | CCT motif | | |
| | | | | | | family | | |
| | | | | | | protein | | |
| InDel2 | 20 | 31,778,798- | 6.17E−24 | 2.69E−08 | 2.90E−1 | exon 4 | N/A | N/A |
| | | 31,779,118 | | | | | | |
| Glyma.20G085100 | 20 | 31.779,804 stop | | | | | | |
| Glyma.20G085200 | 20 | 31,784,964 stop | | | | GATA type | | |
| | | 31,786,969 start | | | | zinc finger | | |
| | | | | | | transcription | | |
| | | | | | | factor family | | |
| | | | | | | protein | | |
| SNP28 | 20 | 31,791,856 | 7.92E−22 | | IP | intergenic | 4,887 | 31,317 |
| SNP29 | 20 | 31,793,219 | 3.45E−21 | 6.44E−08 | | intergenio | 6,250 | 29,954 |
| SNP30 | 20 | 31,795,724 | 1.59E−18 | | | intergenic | 8,755 | 27,449 |
| SNP31 | 20 | 31,801,624 | 8.03E−20 | 1.16E−07 | 126E−12 | intergenic | 14,655 | 21,549 |
| SNP32 | 20 | 31,812,853 | 7.85E−19 | | | intergenic | 25,884 | 10,320 |
| Glyma.20G085200 | 20 | 31,823,173 start | | | | protein of | | |
| | | 31,827,117 stop | | | | unknown | | |
| | | | | | | function | | |
| | | | | | | (DUF3527) | | |

TABLE 11
Genotyping analysis of the 321-bp InDel
in 30 RILs and their parental lines.

| Common | TE | Oil | Protein |
|---|---|---|---|
| Name | insertion | content [%] | content 1% 1 |
| P1479752 | no | 9.6 | 44.4 |
| I1376 | no | 11.3 | 46.0 |
| I628 | no | 11.5 | 44.2 |
| I976 | no | 11.5 | 48.0 |
| I695 | no | 11.9 | 46.5 |
| I1338 | no | 12.0 | 46.9 |
| I828 | no | 12.1 | 46.8 |
| I668 | no | 12.2 | 44.8 |
| I1375 | no | 12.3 | 46.7 |
| I680 | no | 12.3 | 44.2 |
| I1530 | no | 12.5 | 45.6 |
| I144 | no | 13.2 | 46.4 |
| I1593 | no | 13.6 | 46.2 |
| I665 | no | 13.6 | 46.1 |
| I1630 | no | 13.7 | 46.4 |
| I200 | no | 15.3 | 38.8 |
| I306 | yes | 15.9 | 39.0 |
| I646 | yes | 16.1 | 38.4 |
| I1652 | yes | 16.3 | 38.8 |
| I1342 | yes | 16.7 | 38.9 |
| I1490 | yes | 17.1 | 38.7 |
| I684 | yes | 17.5 | 41.2 |
| I477 | yes | 17.6 | 39.9 |
| I32 | yes | 17.6 | 40.6 |
| I1234 | yes | 17.6 | 39.0 |
| I347 | yes | 17.7 | 41.3 |
| I333 | yes | 17.8 | 38.6 |
| I1596 | yes | 18.2 | 37.9 |
| I259 | yes | 18.3 | 39.4 |
| I113 | yes | 18.3 | 39.7 |
| I581 | yes | 18.6 | 37.6 |
| Williams82 | yes | 20.5 | 39.3 |
| Average | no | 12.4 | 45.5 |
| Average | Yes | 17.6 | 39.3 |
| Difference between | — | 5.2 | 6.2 |
| average contents | — | 4.00E−13 | 3.53E−10 |
| p-value if-test) | | | |

TABLE 12
TE genotypes of accessions used for biparental QTL mapping experiments.

| high oil/low protein parent | | | | | | low oil/high protein parent | | | | | | Chr20 | |
|---|---|---|---|---|---|---|---|---|---|---|---|---|---|
| | | | TE | Oil | Protein | | | | TE | Oil | Protein | oil and | |
| | | | inser- | Content | Content | | | | inser- | Content | Content | protein | |
| P1 | Name | Species | tion | [1%] | [1%] | PI | Name | Species | tion | [1%] | [1%] | QTL | Reference |
| P1518671 | Williams | Glycine | yes | 20.3 | 41.5 | P1479752 | GD 50388-2 | Glycine | no | 9.4 | 46.4 | yes | this study |
| | 82 | max | | | | | | soja | | | | | |
| P1562374 | Parker | Glycine | yes | 22.2 | 42.6 | P1468916 | | Glycine | no | 10.1 | 44 | yes | Sebolt |
| | | max | | | | | | soja | | | | | et al. |
| | | | | | | | | | | | | | (2000) |
| P1518671 | Williams | Glycine | yes | 20.3 | 41 5 | PI483460B | | Glycine | no | 8.9 | 48 | yes | Patil |
| | 82 | max | | | | | | soja | | | | (protein) | et al. |
| | | | | | | | | | | | | | 2018 |
| | R05-638 | Glycine | yes | 23^{a} | 40.8^{a} | P1659697 | R05-1415 | Glycine | no | 18.4^{a} | 48.3^{a} | yes | Wang |
| | | max | | | | | | max | | | | | et al. |
| | | | | | | | | | | | | | (2015a), |
| | | | | | | | | | | | | | Wang |
| | | | | | | | | | | | | | et al. |
| | | | | | | | | | | | | | (2015b) |
| | R05.4256 | Glycine | yes | 23^{a} | 40.8^{a} | | V97-1346 | MIX | no | 18.4^{a} | 48.3^{a} | yes | Wang |
| | | max | | | | | | | | | | | et [symbol] |
| | | | | | | | | | | | | | (2015a), |
| | | | | | | | | | | | | | Wang |
| | | | | | | | | | | | | | et [symbol] |
| | | | | | | | | | | | | | (2015b) |
| P1595645 | Benning | Glycine | yes | 18.4 | 42^{b} | P1619083 | Danbaekkong | Glycine | no | N/A | 51^{b} | yes | Warringt [symbol] |
| | | max | | | | | | max | | | | (protein) | (2015) |
| P1556814 | A3733 | Glycine | yes | 20.5^{c.} | 42^{c} | PI437088A | DV-147 | Glycine | no | 16^{c} | 48^{c.} | yes | Chung |
| | | max | | | | | | max | | | | | e [symbol] |
| | | | | | | | | | | | | | (2003) |
| P1556536 | Coker 237 | Glycine | yes | 20^{d} | 42.6^{d} | P197100 | 1249 | Glycine | yes | 18.6^{d} | 45.1^{d} | no | Fasoula |
| | | max | | | | | | max | | | | | [symbol] |
| | | | | | | | | | | | | | (2004), |
| | | | | | | | | | | | | | (Lee et al. |
| | | | | | | | | | | | | | (1996)) |
| P1595362 | Magellan | Glycine | yes | 20.2^{c} | 38^{c} | PI567516C | Ba yue zha | Glycine | yes | 17.8 | 42.6 | no | Pathan |
| | | max | | | | | | | | | | | et al. |
| | | | | | | | | | | | | | (2013) |
| P1508266 | Young | Glycine | yes | 19.4 | 44.2 | P1416937 | Houjaku | Glycine | yes | 15.6 | 47.1 | no | Fasoula |
| | | max | | | | | Kuwazu | max | | | | | et al. |
| | | | | | | | | | | | | | (2004) |
| | | | | | | | | | | | | | (Lee et al. |
| | | | | | | | | | | | | | (1996)) |
| P1548631 | Williams | Glycine | yes | 19.8 | 45.6 | P1548667 | Essex | Glycine | yes | 20 | 43.9 | no | Hyten |
| | | max | | | | | | max | | | | | et al. |
| | | | | | | | | | | | | | (2004) |
| P1548389 | Minsoy | Glycine | yes | 19.7 | 39.4 | PI290136 | Noir1 | Glycine | yes | 19.8 | 42.9 | no | Orf et al. |
| | | max | | | | | | max | | | | | (1999), |
| | | | | | | | | | | | | | (Mansu [symbol] |
| | | | | | | | | | | | | | (1993), |
| | | | | | | | | | | | | | Mansur [symbol] |
| | | | | | | | | | | | | | (1996), |
| | | | | | | | | | | | | | Specht e [symbol] |
| | | | | | | | | | | | | | (2001)) |
| P1546487 | Archer | Glycine | yes | 21 | 43.3 | P1290136 | Noir 1 | Glycine | yes | 19.8 | 42.9 | no | Orf et al. |
| | | max | | | | | | max | | | | | (1999) |
| P1546487 | Archer | Glycine | yes | 21 | 43.3 | P1548389 | Minsoy | Glycine | yes | 19.7 | 39.4 | no | Orf et al. |
| | | max | | | | | | max | | | | | (1999), |
| P1548667 | Essex | Glycine | yes | 20 | 43.9 | P1548402 | Peking | Glycine | yes | 18.5 | 38.2 | no | Qiu et al. |
| | | max | | | | | | max | | | | | (1999), |

For oil and protein content:
^{a}from Wang et al. 2015a and b, converted from 13% moisture content to dry weight;
^{b}from Warrington et al. 2015
^{c}from Chung et al. 2003
^{d}from Lee et al. 1996
^{e}from Pathan et al. 2013
all other phenotype values from GRIN. [symbol]
indicates data missing or illegible when filed

TABLE 13
Information of accessions used for RNA-Seq and POWR1 expression levels.

| | Common | Species | TE | | | |
|---|---|---|---|---|---|---|
| P1 | Name | Subspecies | insertion | FPICM | AvarageFPKM | Stdev |
| P1518671 | Williams 82 | Glycine max | yes | 1.010692 | 0.6679555 | 0.18984621 |
| P1540666 | Jack | Glycine max | yes | 0.724321 | | |
| P1548348 | [symbol] | Glycine max | yes | 0.508646 | | |
| P154863 1 | Williams | Glycine max | yes | 0.673904 | | |
| P1548668 | Lee | Glycine max | yes | 0.589683 | | |
| P1598124 | Maverick | Glycine max | yes | 0.502487 | | |
| P1647962 | R95-1705 | Glycine max | no | 0.847271 | 0.9701485 | 0.28815252 |
| . | JL275 | Glycine max | no | 0.632052 | | |
| . | LLL05-14 | Glycine max | no | 1.282608 | | |
| . | S09-2902-145 | Glycine max | no | 1.118663 | | |

[symbol]

indicates data missing or illegible when filed

TABLE 14
Agronomic traits that can be improved using compositions

QTL TraitName
Acid detergent fiber content
Acid phosphatase activity
Branching
Canopy Height
Canopy Width
Canopy wilt
Cell wall polysaccharide
composition
Chlorimuron Ethyl Sensitivity
Drought Susceptibility Index
Drought Tolerance
Embryo Abortion
First flower
Flooding Tolerance
Flower form
Height/Lodging
Hypocotyl Length
Internode length
Iron Efficiency
Leaf phosphorus content
Leaflet area
Leaflet chlorophyll content
Leaflet Length
Leaflet shape
Leaflet trigonelline content
Leaflet Width
Lodging
Manganese induced leaflet
necrosis
Manganese induced root necrosis
Net photosynthetic rate
Neutral detergent fiber content
Node Number
Ozone sensitivity, leaflet
Pectin content
Petiole color
Phosphorus content
Phosphorus use efficiency
Photoperiod sensitivity
Plant Height
Plant weight
Plant weight, dry
Pod weight ratio
Pods Per Node
Pods per plant
Probability an electron reduces
an end acceptor at PSI electron
acceptor side
Pubescence Density
R/V photothermal sensitivity
R3 Beginning pod
R5 Beginning Seed
R7 Beginning Maturity
R8 Full Maturity
Reaction to Aphis glycines
Reaction to Aulacorthum solani,
choice
Reaction to Aulacorthum solani,
no choice
Reaction to Bemisia tabaci
Reaction to Fusarium infection
Reaction to Fusarium solani f sp
glycines infection
Reaction to Helicoverpa zea
Reaction to Heterodera glycines
Reaction to Lamprosema indicata
Reaction to Meloidogyne incognita
Reaction to Meloidogyne javanica
Reaction to Phytophthora sojae
infection
Reaction to Popilliae japnoica
Reaction to Sclerotinia
sclerotiorum infection
Reaction to Soybean Mosaic Virus
Infection (SMV)
Reaction to Spodoptera litura
Reproductive period
Reproductive stage length
Root dry weight to shoot dry
weight ratio
Root length, coarse
Root length, fine
Root length, medium
Root nodule number
Root Nodule Size
Root nodule weight
Root nodule weight, dry
Root phosphorus content
Root Surface Area
Root surface area, coarse
Root surface area, fine
Root surface area, medium
Root volume
Root volume, coarse
Root volume, fine
Root volume, medium
Root Weight
Root weight, dry
Row spacing response
Seed arabinose-galactose content
Seed beta-conglycinin beta-
subunit protein content
Seed calcium content
Seed coat cracking
Seed Coat Hardness
Seed cysteine content
Seed daidzein content
Seed delta-tocopherol content
Seed filling period
Seed fucose content
Seed galactose content
Seed gamma-tocopherol
content
Seed genistein content
Seed glucose content
Seed glycinin and beta-
conglycinin content
Seed glycinin content
Seed hardness
Seed height
Seed isoflavone content
Seed isoleucine content
Seed length to height ratio
Seed leucine content
Seed linoleic acid content
Seed linolenic acid content
Seed lysine content
Seed methionine and
cysteine content
Seed methionine content
Seed Number
Seed oil content
Seed oil plus protein content
Seed oleic acid content
Seed palmitic acid content
Seed palmitic + stearic
acid content
Seed protein content
Seed Set
Seed size
Seed soluble
oligosaccharides
Seed stearic acid content
Seed sucrose content
Seed threonine content
Seed tyrosine content
Seed viability
Seed weight
Seed width
Seed yield
Seed yield/Plant height
Seeds per plant
Shattering
Shoot phosphorus content
shoot weight, dry
shoot weight, fresh
Somatic embryos per
explant
Specific leaflet weight
Starch content
Stem strength
Total growth duration
UV-B induced leaflet shape
response
UV-B induced plant damage

TABLE 15
List of QTLs co-located with GmCCTs and Diversity Pi values for GmCCTs

| Gene | | | | QTL | | | | |
|---|---|---|---|---|---|---|---|---|
| Name | ID | gene | locus | chr | QTL start | QTL stop | interval | QTL Trait Name |
| POWR2 | GmCCT34 | Glyma.10G134400 | Chr10: 36034798- | Chr10 | 33,311,029 | 38,033,182 | 4,722,153 | Net |
| | | | 36039695 | | | | | photosynthetic rate |
| | | | | Chr10 | 29,480,120 | 36,986,811 | 7,506,691 | Reaction to |
| | | | | | | | | Heterodera glycines |
| | | | | Chr10 | 29,427,575 | 38,574,461 | 9,146,886 | Seed Set |
| | | | | | | | 0 | |
| POWR3 | GmCCT35 | Glyma.10G190300 | Chr10: 42314396- | Chr10 | 41,204,461 | 44,713,925 | 3,509,464 | Branching |
| | | | 42318797 | | | | | |
| | | | | Chr10 | 37,300,089 | 43,004,879 | 5,704,790 | Drought |
| | | | | | | | | Tolerance |
| | | | | Chr10 | 42,295,191 | 44,713,925 | 2,418,734 | Leaflet shape |
| | | | | Chr10 | 42,295,191 | 44,713,925 | 2,418,734 | Leaflet Width |
| | | | | Chr10 | 42,295,191 | 44,713,925 | 2,418,734 | Plant Height |
| | | | | Chr10 | 36,986,811 | 44,713,925 | 7,727,114 | Pods per plant |
| | | | | Chr10 | 41,204,461 | 43,788,256 | 2,583,795 | Probability an |
| | | | | | | | | electron reduces |
| | | | | | | | | an end acceptor |
| | | | | | | | | at PSI electron |
| | | | | | | | | acceptor side |
| | | | | Chr10 | 42,295,191 | 44,713,925 | 2,418,734 | R8 Full Maturity |
| | | | | Chr10 | 42,295,191 | 46,492,285 | 4,197,094 | Reaction to |
| | | | | | | | | Heterodera glycines |
| | | | | Chr10 | 38,957,017 | 43,004,879 | 4,047,862 | Reaction to |
| | | | | | | | | Lamprosema |
| | | | | | | | | indicata |
| | | | | Chr10 | 40,306,793 | 43,004,879 | 2,698,086 | Reaction to |
| | | | | | | | | Sclerotinia |
| | | | | | | | | sclerotiorum |
| | | | | | | | | infection |
| | | | | Chr10 | 41,204,461 | 44,713,925 | 3,509,464 | Reproductive |
| | | | | | | | | stage length |
| | | | | Chr10 | 40,306,793 | 48,423,831 | 8,117,038 | Root surface |
| | | | | | | | | area, medium |
| | | | | Chr10 | 40,306,793 | 48,423,831 | 8,117,038 | Root volume |
| | | | | Chr10 | 40,306,793 | 48,423,831 | 8,117,038 | Root volume, |
| | | | | | | | | medium |
| | | | | Chr10 | 40,306,793 | 48,423,831 | 8,117,038 | Root weight, dry |
| | | | | Chr10 | 42,295,191 | 44,713,925 | 2,418,734 | Seed glycitein |
| | | | | | | | | content |
| | | | | Chr10 | 41,204,461 | 43,509,732 | 2,305,271 | Seed length to |
| | | | | | | | | width ratio |
| | | | | Chr10 | 36,986,811 | 43,004,879 | 6,018,068 | Seed protein |
| | | | | | | | | content |
| | | | | Chr10 | 36,986,811 | 44,713,925 | 7,727,114 | Seed protein |
| | | | | | | | | content |
| | | | | Chr10 | 42,295,191 | 44,713,925 | 2,418,734 | Seed tocopherol |
| | | | | | | | | content |
| | | | | Chr10 | 36,986,811 | 43,004,879 | 6,018,068 | Seed weight |
| | | | | Chr10 | 41,204,461 | 43,509,732 | 2,305,271 | Seed width |
| | | | | Chr10 | 39,108,281 | 43,004,879 | 3,896,598 | Total growth |
| | | | | | | | | duration |
| | | | | | | | 0 | |
| POWR1 | GmCCT67 | Glyma.20G085100 | Chr20: 31774769- | Chr20 | 25,275,083 | 34,052,339 | 8,777,256 | Internode length |
| | | | 31779804 | | | | | |
| | | | | Chr20 | 25,693,363 | 34,302,228 | 8,608,865 | Ozone sensitivity, |
| | | | | | | | | leaflet |
| | | | | Chr20 | 25,275,083 | 34,302,228 | 9,027,145 | Reaction to |
| | | | | | | | | Heterodera glycines |
| | | | | Chr20 | 25,693,363 | 34,052,339 | 8,358,976 | Seed calcium |
| | | | | | | | | content |
| | | | | Chr20 | 25,727,028 | 35,362,576 | 9,635,548 | Seed linoleic |
| | | | | | | | | acid content |
| | | | | Chr20 | 25,275,083 | 34,052,339 | 8,777,256 | Seed lysine |
| | | | | | | | | content |
| | | | | Chr20 | 25,275,083 | 34,052,339 | 8,777,256 | Seed methionine |
| | | | | | | | | content |
| | | | | Chr20 | 27,664,504 | 33,590,931 | 5,926,427 | Seed oil content |
| | | | | Chr20 | 25,727,028 | 34,302,228 | 8,575,200 | Seed oil content |
| | | | | Chr20 | 25,275,083 | 34,302,228 | 9,027,145 | Seed oil content |
| | | | | Chr20 | 25,674,167 | 35,362,576 | 9,688,409 | Seed oil content |
| | | | | Chr20 | 25,674,167 | 35,362,576 | 9,688,409 | Seed oil content |
| | | | | Chr20 | 25,727,028 | 35,362,576 | 9,635,548 | Seed oleic acid |
| | | | | | | | | content |
| | | | | Chr20 | 27,664,504 | 33,590,931 | 5,926,427 | Seed protein |
| | | | | | | | | content |
| | | | | Chr20 | 25,727,028 | 34,302,228 | 8,575,200 | Seed protein |
| | | | | | | | | content |
| | | | | Chr20 | 25,275,083 | 34,052,339 | 8,777,256 | Seed protein |
| | | | | | | | | content |
| | | | | Chr20 | 25,275,083 | 34,302,228 | 9,027,145 | Seed protein |
| | | | | | | | | content |
| | | | | Chr20 | 25,498,552 | 35,362,576 | 9,864,024 | Seed protein |
| | | | | | | | | content |
| | | | | Chr20 | 25,275,083 | 34,052,339 | 8,777,256 | Seed threonine |
| | | | | | | | | content |
| | | | | Chr20 | 25,727,028 | 34,052,339 | 8,325,311 | Seed weight |
| | | | | Chr20 | 27,664,504 | 33,590,931 | 5,926,427 | Seed yield |
| | | | | | | | 0 | |
| POWR4 | GmCCT69 | Glyma.20G200400 | Chr20: 43781164- | Chr20 | 43,382,152 | 45,170,799 | 1,788,647 | Canopy Width |
| | | | 43785175 | | | | | |
| | | | | Chr20 | 43,634,534 | 44,710,552 | 1,076,018 | Reaction to |
| | | | | | | | | Heterodera glycines |
| | | | | Chr20 | 42,426,240 | 45,134,969 | 2,708,729 | Reaction to |
| | | | | | | | | Heterodera glycines |
| | | | | Chr20 | 43,198,876 | 47,757,190 | 4,558,314 | Reaction to |
| | | | | | | | | Heterodera glycines |
| | | | | Chr20 | 41,415,974 | 46,787,225 | 5,371,251 | Reaction to |
| | | | | | | | | Heterodera glycines |
| | | | | Chr20 | 40,623,814 | 46,787,225 | 6,163,411 | Reaction to |
| | | | | | | | | Popilliae japnoica |
| | | | | Chr20 | 43,382,152 | 45,170,799 | 1,788,647 | Root nodule |
| | | | | | | | | number |
| | | | | Chr20 | 42,798,354 | 44,710,552 | 1,912,198 | Seed genistein |
| | | | | | | | | content |
| | | | | Chr20 | 42,953,527 | 44,710,552 | 1,757,025 | Seed oleic acid |
| | | | | | | | | content |
| | | | | Chr20 | 40,623,814 | 44,710,552 | 4,086,738 | Seed weight |
| | | | | Chr20 | 42,165,892 | 46,678,762 | 4,512,870 | Seed weight |
| | | | | Chr20 | 38,172,219 | 46,678,762 | 8,506,543 | Reaction to |
| | | | | | | | | Fusarium solani |
| | | | | | | | | f sp glycines |
| | | | | | | | | infection |

SEQUENCES

| | | Sequence | |
|---|---|---|---|
| | | type | |
| | Organism | DNA; | |
| SEQ | (Latin | Protein; | |
| ID | name) OR | RNA or | |
| NO. | Synthetic | DNA/RNA | Sequence |
| 1 | WT >POWR1- | Protein | MLQDVVHPSTPAEQLPIDEISGPISARIFELCDPDFFPHTLQNSEV |
| | TE PROTEIN | | TSSSNCCHEEKSSYATTISPPLDVVDNNKFNINSNSSNIVTTTSSS |
| | SEQ | | TTTTSTTTNNNNNATNGNNLSIFFDTQDEIDNDISASIDFSSSPSF |
| | | | VVPPLLPISTQQDQFDFPSAQPQVQLSTAAGSILTGLSHYPTDPVI |
| | | | APLIGAPLPSVFDDDCISSIPSYVPLNPSSPSCSYLSPGIGVYMPP |
| | | | PGSLNTALSADSSGLFGGNILLGSELQAHELDYQGENGGMYCTDSI |
| | | | QRVFNSPDLQALGNESQKLVAGAGSSATLAPEISHLEDSTLKVGKL |
| | | | SVEQRKEKIHRYMKKRNERNFSKKIKYACRKTLADSRPRVRGRFAK |
| | | | NDDFGESHKQGSSNHEDDDEEIIVKEDDDMVDSSDIFAHISGVNSF |
| | | | KCNYSIQSLI |
| 2 | WT >POWR1- | DNA | ATGTTGCAGGATGTTGTCCACCCCTCAACACCGGCTGAGCAACTCC |
| | TE | | CCATTGATGAGATTTCAGGCCCGATTAGTGCTCGAATTTTCGAACT |
| | NUCLEOTIDE | | TTGCGACCCCGATTTCTTCCCACACACACTGCAAAATTCTGAGGTT |
| | ACID SEQ | | ACCTCCAGCTCAAATTGTTGCCATGAAGAGAAGTCCTCATATGCCA |
| | | | CAACCATATCTCCACCTTTAGATGTAGTAGACAACAATAAGTTCAA |
| | | | TATCAATAGCAATAGCAGCAACATAGTCACCACTACCTCATCTAGC |
| | | | ACTACCACAACCAGCACCACAACCAACAACAACAACAACGCAACGA |
| | | | ACGGCAATAATCTTTCTATCTTCTTTGACACTCAAGATGAAATTGA |
| | | | CAATGACATCTCAGCCTCCATAGACTTCTCATCATCTCCATCTTTT |
| | | | GTCGTTCCACCACTTCTCCCAATCTCAACTCAGCAGGATCAGTTTG |
| | | | ATTTCCCTTCAGCTCAGCCACAGGTGCAACTATCAACAGCAGCAGG |
| | | | TTCAATTTTGACGGGCCTCTCTCACTACCCTACAGATCCTGTGATT |
| | | | GCACCCCTTATTGGAGCTCCGTTACCATCTGTTTTTGATGATGATT |
| | | | GCATATCTTCCATCCCTTCTTATGTGCCTCTCAACCCTTCATCACC |
| | | | CTCTTGCTCTTATCTCAGTCCTGGCATAGGAGTGTACATGCCACCT |
| | | | CCTGGTTCCCTTAACACTGCCTTATCTGCTGACAGTTCTGGATTGT |
| | | | TTGGTGGGAACATTCTACTGGGGTCTGAACTGCAGGCACATGAATT |
| | | | GGACTACCAGGGAGAAAATGGTGGAATGTATTGTACAGATTCAATT |
| | | | CAAAGGGTGTTTAACTCCCCAGACCTTCAGGCACTTGGTAATGAGA |
| | | | GTCAGAAACTTGTAGCTGGGGCTGGAAGCTCTGCCACTTTGGCACC |
| | | | AGAAATCTCACACTTGGAGGACTCTACCTTGAAGGTTGGAAAACTC |
| | | | TCTGTTGAGCAGAGGAAGGAAAAGATTCATAGATACATGAAGAAGA |
| | | | GAAACGAAAGAAATTTCAGCAAGAAAATCAAGTATGCTTGCCGCAA |
| | | | AACTTTGGCGGATAGCCGGCCCCGGGTTAGAGGAAGGTTTGCAAAG |
| | | | AATGATGACTTTGGAGAGAGCCATAAACAAGGAAGTAGCAATCATG |
| | | | AAGATGATGATGAAGAGATAATTGTGAAAGAAGATGATGATATGGT |
| | | | TGATTCCTCAGATATCTTTGCACATATCAGCGGAGTGAACTCTTTC |
| | | | AAATGCAACTATTCCATCCAGTCCTTGATTTGA |
| 3 | Mutant | Dna | Chr20:31774769..31779804-Forward: |
| | >POWR1_w_ | | key: 5′UTR CDS 3′UTR |
| | TE_truncated | | CATCATCTTAGCTAACACAACACAGCATACACAACTTCTCTCTCTC |
| | CCT_CDS_ | | TACTACTTTCTTTTTATGTCACCTTCTTGATGTCTTTAATTTGTTT |
| | CCT67 | | TCTTTTTTAGAAAAAAATTAATCCTTTTTAAGGCTTTAATTTCCTA |
| | >POWR1_{+TE} | | AGCATGCAATATTATTGTTTTTAGTCACCTTCAAGTTGAGGAACAT |
| | (Glyma. | | ATACACGTTTGTGACCACACCAAATTCCACTTCTTTCTAAGTGTGT |
| | 20G085100) | | GTACATAGATTTTGTTTTATATATATTTGAGGTTCACCCTTCATTG |
| | Genomic | | CCTCCTTAATTCTGTGCAAAAGAGGATTCATCACCACCATGTTGCA |
| | DNA | | GGATGTTGTCCACCCCTCAACACCGGCTGAGCAACTCCCCATTGTA |
| | (nucleotide) | | ATCACCTCAAGCTTGTTACATTTCATATCTCGATCATGTATATTTA |
| | sequence: | | GTGTTAATGCATGTATTGGAACTAAGCTATATATGTATGTGTGTAT |
| | 17 bp | | GTATCTTTGTGTCATGCAATATTTATGACCACAGCTAAATGGTTTC |
| | duplications | | CTTCTGGGTTTTGTGATCGTGTGTAACTTGACAAGTTTGTTTGGAT |
| | are | | CAAGGGCTTGTTTTTTTCTTTCAAAATAATGATTTCACACTCCAAG |
| | bolded and | | AGTGTGTAAACCTTGGGAGGGAAGAGAAAGACAGGAAAGAGACCCC |
| | underlined | | AGAAAAGAACAAAAGAAGTCTAAAATTGATGTACGGATTAATAGCG |
| | | | TGTTTGAAAACCCATTGAGAAGTAAGAAAATCTCATCTGAGATATG |
| | | | AAAGTTATGACTATTAACTTCCTAATAAACTCAAGAAATCACATCT |
| | | | GAGATGTGAAACCAAACATGCTCTTACGAATTGTAGAAATTTTGAT |
| | | | GGTTTTTCTCTGGGGTACTCTTTCTTATATTTTGAGGCCAATTATT |
| | | | ACTAATTTCTCTAGGAATTATTCGATCAATTGCTCCATTCCTGCAT |
| | | | TTTAATTCTTTCCATGCATGAATCACGAATATGGTTTTGTTAATGT |
| | | | TTGTTGGCATGTTAATTAATTTCATCCTAATTAGTTCTCTGAGAAA |
| | | | CCTGAACAATATTTCCCTTATTTGGTTATATTCCTGGAGCTAAGTG |
| | | | GAAGCTGCTAGTGCTATTAGCCATCTTAGAAAACCCACAAGCCATG |
| | | | CTCACTATTTGTGGGACGGAACGTTTTCTTTCCTCTATTTATATGG |
| | | | TTGGAGAGCATAACGTAATTTTATTGACCGAACAATAGGAAGGAAT |
| | | | CAAAGACTCCGTGACCGTGCTGTCAACTCTTTTGATATTTATTATC |
| | | | ATCCAATGGGCCCTACCACCAAAATGTTTAATGTCATTTTCCAAGG |
| | | | GGCAAAGTTGTAAAAATGATGTAACATTTCCGTGATAGTAATTTAG |
| | | | ACATAGCTTTCGGTTCTTGTAGAATATATATATCTCTTTGGGGGCT |
| | | | TGTTGTGTAAGCTTTGCTAATTTTTGATATTCTTTATTCAGTTAGA |
| | | | TAGGGAGAAGAGGTCTTAGTGACAAACAATTCATATGGATAGCATA |
| | | | TATAGCATTATGGGGTTCTCTTCAAGTGGCTTTCAATTCCCATTTA |
| | | | AGTTCAAATTTTCCACTTAAAATGAACAAAAATAACATAGCATGAG |
| | | | GTTCCTTGTTTGTTTTTGCATACTTGTATTTCATACTAACATTGGC |
| | | | TACCTTGGCTCCTACCTGTCACATGAAGGCATAGATGCACATCACT |
| | | | TTCACCAATAAATAACCCAACAATGCAAACCCTTTAGGAAAATTTC |
| | | | TCTTCAGTCTATTACCAAATATGGACAAAACTGACTAGACTATGCA |
| | | | ACCAACCACAGTCATATGTAGAATTCTTCGTTGGGGCCTTTGCACA |
| | | | TCGATTTATATTGTTTTCCTCTTGATAAAGAAGGAAGGGGGTGTTG |
| | | | TGGCAGATACTATTATATCATTTGGCATGTCTTTCACTTTTGAATG |
| | | | CCCACTACATCTACAAGCCAAAGCTTCAATTGATTAGAGAATATTA |
| | | | GCTTTTGAATTTCTTTTCTGTTCTAAGTGATCAGATCAGACTCATG |
| | | | TCTATCAACAAAAAGAGGAATTGGATTCAGATTTCAAACCCATCAT |
| | | | AATGAAGAAAAAAATAAAAAAAATAAACTATAGAGAATCACGTTTC |
| | | | CTAGATTGTTTATAAAGAAGAGGCTTTGTTAAGACTATTCTCAGGC |
| | | | TGTGTTTTAGTAGTTTATGGTAAACATGTTGTTCACAATGCTTATA |
| | | | TGTCACCACTCATTGCAGGATGAGATTTCAGGCCCGATTAGTGCTC |
| | | | GAATTTTCGAACTTTGCGACCCCGATTTCTTCCCACACACACTGCA |
| | | | AAATTCTGAGGTTACCTCCAGCTCAAATTGTTGCCATGAAGAGAAG |
| | | | TCCTCATATGCCACAACCATATCTCCACCTTTAGATGTAGTAGACA |
| | | | ACAATAAGTTCAATATCAATAGCAATAGCAGCAACATAGTCACCAC |
| | | | TACCTCATCTAGCACTACCACAACCAGCACCACAACCAACAACAAC |
| | | | AACAACGCAACGAACGGCAATAATCTTTCTATCTTCTTTGACACTC |
| | | | AAGATGAAATTGACAATGACATCTCAGCCTCCATAGACTTCTCATC |
| | | | ATCTCCATCTTTTGTCGTTCCACCACTTCTCCCAATCTCAACTCAG |
| | | | CAGGATCAGTTTGATTTCCCTTCAGCTCAGCCACAGGTGCAACTAT |
| | | | CAACAGCAGCAGGTTCAATTTTGACGGGCCTCTCTCACTACCCTAC |
| | | | AGATCCTGTGATTGCACCCCTTATTGGAGCTCCGTTACCATCTGTT |
| | | | TTTGATGATGATTGCATATCTTCCATCCCTTCTTATGTGCCTCTCA |
| | | | ACCCTTCATCACCCTCTTGCTCTTATCTCAGTCCTGGCATAGGAGT |
| | | | GTACATGCCACCTCCTGGTTCCCTTAACACTGCCTTATCTGCTGAC |
| | | | AGTTCTGGATTGTTTGGTGGGAACATTCTACTGGGGTCTGAACTGC |
| | | | AGGCACATGAATTGGACTACCAGGGAGAAAATGGTGGAATGTATTG |
| | | | TACAGATTCAATTCAAAGGGTGTTTAACTCCCCAGACCTTCAGGTA |
| | | | TGTGCAATTTCGCAAGCCAATTAGAGTTTAATAGACATTCATTGTC |
| | | | TGGTATAAAAGTTTTTACATTATCAATCAATCAGATACCATTGTTG |
| | | | ATATAAATTTTAAAATAATTGTTATAATAATTAATAATTTAATGTA |
| | | | CTTGATAATTTGTGATTTGATAATAATATAAAAAAAATTTACACTG |
| | | | CATTATTATTTATTTTTCCTGTCGACTGTCAATAAACTAAATGAAA |
| | | | ATTTTCAGTTCCAATGTTCAATGTGTTCAGAATAAGGAAAAAGAAG |
| | | | TTTAATAATGCTGCAAAGGTTACTATACCTTGCAGCAGTGAAGTTT |
| | | | TTATTTTAAAATAGAAGAGGCTTTATCAGAGGTGGACTTTTGGGGG |
| | | | AAAGCTCAGGGTCCACAAATCTCTAAACTATAAACTCATAGGTGCC |
| | | | CCATGACCATCAAATAGTAGGTAGCACAAGATATGAGTCCATTTAT |
| | | | AAAGTCACATGCATTAAAAAATACTATAAATTTGGCCTAGCAAGAA |
| | | | GGAAGAACCACTTTCATCCAAAAGAAAAATAGAAAAAAGGATAATA |
| | | | AACTGTAGCATCATTAGATAGAAAGACCCACTTCAAGGGTGGCAGT |
| | | | GTTATATCTCTTTCTACAGTCTATAAAGTTAATGTGCAGTTTTTAT |
| | | | TGAATAAGTAAGAAATTGATCTTTAATTATAATTTCTCTCTCAGGC |
| | | | ACTTGGTAATGAGAGTCAGAAACTTGTAGCTGGGGCTGGAAGCTCT |
| | | | GCCACTTTGGCACCAGAAATCTCACACTTGGAGGACTCTACCTTGA |
| | | | AGGTTGGAAAACTCTCTGTTGAGCAGAGGAAGGAAAAGATTCATAG |
| | | | ATACATGAAGAAGAGAAACGAAAGAAATTTCAGCAAGAAAATCAAG |
| | | | GTACTACATCTGAACACCAACATTAACAAACAAATTTCAAATCTTA |
| | | | TACTGTTTTACATGATTTCCAATCTACTGCATCAACCAAGCCTTAT |
| | | | GCATATTTTCAAAATTCAACTAATGATGCAATTTTTTTTATATAAA |
| | | | AAAAATGCAGTATGCTTGCCGCAAAATTAGAGAGAGGGTTGGAGTA |
| | | | GCGCCTATTGTAGAGAAGATGGTGGAAAATAGACTTAGGTGGTTTG |
| | | | GGCATGTAGAGAGAAGACCGGTAGACTCTGTAGTGAGGAGAGTAGA |
| | | | CCAGATGGAGAGAAGACAAACAATTCGAGGCAGAGGAAGACCCAAA |
| | | | AAGACTATAAGAGAGGTTATAAAAAAGGATCTCGAAATTAATGGTT |
| | | | TGGATAGAAGTATGGTACTTGATAGAACATTATGGCGGAAGTTGAT |
| | | | CCATGTAGCCGACCCCACCTAGTGGGATAAGGCGTTGTTGTTGTTG |
| | | | TTGTTGTTGTATGCTTGCCGCAAAACTTTGGCGGATAGCCGGCCCC |
| | | | GGGTTAGAGGAAGGTTTGCAAAGAATGATGACTTTGGAGAGAGCCA |
| | | | TAAACAAGGAAGTAGCAATCATGAAGATGATGATGAAGAGGTAAGA |
| | | | TTCCCTTAATCGGATACTGTTGTTCAACTTGCCTTAGTCTAAAAAT |
| | | | TAAAATACAAAAAAATTCCCGATCACTTTTACCTTTTCAATTATTT |
| | | | GATGGCATAATTCCTTGATGTTATATTCCTTCCATTTTTTGTACTT |
| | | | GCAGATAATTGTGAAAGAAGATGATGATATGGTTGATTCCTCAGAT |
| | | | ATCTTTGCACATATCAGCGGAGTGAACTCTTTCAAATGCAACTATT |
| | | | CCATCCAGTCCTTGATTTGAATTAAATTATTAGTTTGACTAGTGAA |
| | | | AGCTTATTTATATAATTAGCTTCTGTAGATTAATTTTGGTAGGACA |
| | | | CTTTTCCCATCCCGGTTCTCTAAAATCCGGGTTTAGTGGTTTGAGT |
| | | | AAACTGAATAAATGGGGTCAAAATAAATATACCAATAAGTTAAGTG |
| | | | AGTTAGAAACGTACAGAAATTGGAAACTGTATACATTTTTGCAGAT |
| | | | ATATATTATCTTTTTCATTAAGTTGTACCAGAACATGGAGTTGTGT |
| | | | TAACCAAGAAAATTTCCAGTTACCCCCATCCAAGACTGATGTAACC |
| | | | AATTGATGTAGCTTCTTTTAT |
| 5 | WT >POWR2_ | Protein | MLQDVIHPSTPAEQLPIDEISSPISARIFELCEPDFFPDTLQNSDV |
| | CCT34_Glyma. | | TSSSNCCHEEKSSYATTISPPLDLVDNKININNNSNIVTTTSSSTT |
| | 10G134400_ | | TTSTTTNNNNNNTTNSNNLSILFDTQDEIDNDISASIDFSSCRSLV |
| | protein | | VPPLLSISTQQDQFDFSSAQPQVQLSAAAGSVLKGLSHYPTDHVIA |
| | | | PLIGSPLPSVFDEDCISSIPSYVPLNPSSPSCSYLSPGIGVYMPPP |
| | | | GSLNTALSADSSGLFGGNILLGSELQAHELDYQGENGGIFCTDSIQ |
| | | | RVFNPPDLQALGTETQKLVAGAGSSATLTPEISHLEDSNLKVGKLS |
| | | | VEQRKEKIHRYMKKRNERNFSKKIKYACRKTLADSRPRVRGRFAKN |
| | | | DEFGESHRQGSSNHEEDDEEIIVKEDDDMVDSSDIFAHISGVNSFK |
| | | | CNYSIQSLI |
| 6 | WT | DNA | ATGTTGCAGGATGTTATCCACCCCTCAACACCGGCTGAGCAACTCC |
| | >POWR2_CCT34_ | | CCATTGATGAGATTTCAAGCCCGATTAGTGCTCGAATTTTCGAACT |
| | Glyma. | | TTGCGAGCCTGATTTCTTCCCAGACACACTGCAAAATTCAGATGTT |
| | 10G134400_ | | ACTTCCAGCTCAAATTGTTGCCATGAAGAGAAGTCCTCATATGCCA |
| | CDS | | CAACCATATCTCCACCTTTAGATTTAGTAGACAACAAGATCAATAT |
| | | | CAATAACAATAGCAACATAGTCACTACTACCTCATCTAGCACTACC |
| | | | ACAACCAGCACCACAACCAACAACAACAACAACAACACAACGAACA |
| | | | GCAATAACCTGTCCATCCTCTTTGACACTCAAGATGAAATTGACAA |
| | | | TGACATCTCAGCCTCCATAGACTTCTCATCATGTCGATCTTTAGTT |
| | | | GTTCCACCACTTCTCTCAATCTCAACTCAGCAGGATCAGTTTGATT |
| | | | TCTCTTCAGCTCAGCCACAGGTGCAACTATCAGCAGCAGCAGGTTC |
| | | | AGTTTTGAAGGGCCTCTCTCACTACCCTACAGATCATGTGATTGCA |
| | | | CCCCTTATTGGATCTCCGTTACCATCTGTTTTTGATGAAGATTGCA |
| | | | TATCTTCCATCCCTTCTTATGTGCCTCTCAACCCATCATCACCCTC |
| | | | TTGCTCTTATCTCAGTCCTGGCATAGGAGTGTACATGCCTCCTCCT |
| | | | GGTTCCCTTAACACTGCCTTATCTGCTGACAGTTCTGGATTGTTTG |
| | | | GTGGGAACATTCTACTGGGGTCTGAACTGCAGGCACATGAATTGGA |
| | | | CTATCAGGGAGAAAATGGTGGAATATTTTGCACAGATTCAATTCAG |
| | | | AGGGTGTTTAACCCCCCAGATCTTCAGGCACTTGGTACTGAGACTC |
| | | | AGAAACTTGTAGCTGGGGCTGGAAGTTCTGCCACTTTGACACCAGA |
| | | | AATCTCACACTTGGAGGACTCTAACTTGAAAGTTGGAAAACTCTCT |
| | | | GTTGAGCAGAGGAAGGAAAAGATTCATAGATACATGAAGAAGAGAA |
| | | | ATGAAAGAAATTTCAGCAAGAAAATCAAGTATGCTTGCCGCAAAAC |
| | | | TTTAGCAGATAGCCGGCCCCGGGTTAGAGGAAGGTTTGCAAAGAAT |
| | | | GATGAGTTTGGAGAGAGCCATAGACAAGGAAGTAGCAATCATGAAG |
| | | | AAGATGATGAAGAAATAATTGTGAAAGAAGATGATGATATGGTTGA |
| | | | TTCCTCAGATATCTTTGCACATATCAGTGGAGTGAACTCTTTCAAA |
| | | | TGCAACTATTCCATCCAGTCCTTGATTTGA |
| 8 | cct34-2-2 | DNA | ATGTTGCAGGATGTTATCCACCCACACCGGCTGAGC |
| | Shown in | | |
| | FIG. 12D | | |
| 9 | cct34-2-2 | DNA | ATCATGTGATTGCACCCCTTATCTCCGTTACC |
| | Shown in | | |
| | FIG. 12D | | |
| 10 | cct34-2-2 | DNA | CTCAGTCCTGGCATAGGAGTGTACATGCCTTCCTGGTTCCCTTAAC |
| | Shown in | | ACTG |
| | FIG. 12D | | |
| 11 | cct34-4-5 | DNA | ATGTTGCAGGATGTTATCCACCCCACCGGCTGAGC |
| | Shown in | | |
| | FIG. 12D | | |
| 12 | cct34-4-5 | DNA | ATCATGTGATTGCACCCCTTATCTCCGTTACC |
| | Shown in | | |
| | FIG. 12D | | |
| 13 | cct34-4-5 | DNA | CTCAGATGTTACTTCCTGGTTCCCTTAACACTG |
| | Shown in | | |
| | FIG. 12D | | |
| 14 | cct34-4-7 | DNA | ATGTTGCAGGATGTTATCCACCCTCAACACCGGCTGAGC |
| | Shown in | | |
| | FIG. 12D | | |
| 15 | cct34-4-7 | DNA | ATCATGTGATTGCACCCCTTATCTCCGTTACC |
| | Shown in | | |
| | FIG. 12D | | |
| 16 | cct34-4-7 | DNA | CTCAGTCCTGGCATAGGAGTGTACATGTCCTGGTTCCCTTAACACT |
| | Shown in | | G |
| | FIG. 12D | | |
| 17 | gRNA1 | DNA | CCACCCCTCAACACCGGCTGAGC |
| 18 | gRNA2 | DNA | CCCCTTATTGGATCTCCGTTACC |
| 19 | gRNA3 | DNA | GGAGTGTACATGCCTCCTCCTGG |
| 20 | PCR primer | DNA | TATCCATATGACGTTCCAGATTACGCC |
| 21 | PCR primer | DNA | ACCTCAGAATTTTGCAGTGTGTGTG |
| 22 | PCR primer | DNA | CTGCTGTTGATGGAGGACTT |
| 23 | PCR primer | DNA | CTCCTGGAGAAGCAGAAGTT |
| 24 | A 17-bp | DNA | GTATGCTTGCCGCAAAA |
| | target | | |
| | site | | |
| | duplication | | |
| | upon | | |
| | insertion | | |
| | of LINE | | |
| | transposon | | |
| | (in | | |
| | POWR1+TE | | |
| 25 | WT Protein | Protein | MLQDVFDQPLPPKAEPQQQHPIWLQDEISSPLSARIFELCNTDFFP |
| | sequence | | ETLPNSEVTSSSNCCYEENSSYATTNISVTVDVDNKLNSNSNTVTT |
| | of POWR3 | | PTSTTTTNTNNNTTNSSNLSIIFDSQEEIDNDISASIDFSLSPSFN |
| | >G.maxWm82. | | VPPFLPATSQQEQFDFSSVQHQVQLAACSVVEGFSQYPNDSVAPPF |
| | a2.v1\| | | MGAPLPSVFEEDCISSVPSYMALNPSSPCTYLSPGMPPYMPHGPLT |
| | Glyma. | | TALSTGSSGIFGGNILLGSELQAQELEYQGENGRMYCTDSIQRVFN |
| | 10G190300. | | PPDLQALGTESQQLVPGAGSSASLPEISNLEDSSFKVGKLSVEQRK |
| | 1.p | | EKINRYMKKRNERNFSKKIKYACRKTLADSRPRVRGRFAKNDDFGE |
| | | | TNRTTSSNHEEDDEEEVVVKDEDDMVDSSDIFAHISGVNSFKCNYS |
| | | | FQSWI* |
| 26 | WT | DNA | GTCAACCCAAAATCCAACTCAAGACAAGGAGAGTGGCATTGGCAGA |
| | >G.maxWm82. | | TGGCACCCGAGAGCCAAGAGGGTTTGCAGTGAATGAATGGCCCACC |
| | a2.v1\| | | TTATGTCTCGCTTTGACGCCAGCTTGACGCCTTTAGGGAGAACCCC |
| | Glyma. | | TTTGCCCTATATATGAAACATAACTAATTCTTTGAGTCTTTGACAT |
| | 10G190300 | | CCTCATACTCTCCATCATCCTAGCTAACTACTCATCATCATATTCA |
| | Chr10: | | TAACTTTCCCCCCTCTTTCTCTCTCTTTTTCCCTATGGAGTGTCCT |
| | 42314396.. | | TGAGCACATGTTTTAAGTCACCAACACACGTGTGAATTTCCCACAT |
| | 42318797 | | TTTGCACTCCTATATAGTACTCTCTTTCTTTCTAGATATAGCTTTA |
| | reverse | | ATTTTGTGTACATTTTTTTTGTATTAAAGTTTCTGGGGTGCCTTGA |
| | key: 5′UTR | | TTGCCTCGTGCAAAGGGAAACATACATACATACACCATGTTGCAGG |
| | CDS | | ATGTTTTTGATCAGCCACTGCCACCAAAGGCCGAACCACAACAGCA |
| | 3′UTR | | ACATCCCATTGTAATCACCTCATCTTGCCAGCAGTCTCAATATTCA |
| | CDS of | | TCTATTATGTTAATGCATACATGCATGTGTTCTTCCTACCTTAATG |
| | POWR3 | | ACATAGTGTAATCCTAACTCAATATTAATTTCATGAAAACTTTACA |
| | | | AGTTTGTTAATTGTTTCCATTTTGGAACGAATCTCTTTCTCATATG |
| | | | TTTGAATATATTCAAATCTAAGGCTAAGTTGTTTGTTTACAGTTGT |
| | | | CTCGTGATATTGGCCCACTTGTGATTCTCTTCTTTTTTTCATGCAT |
| | | | GCATGCGTGAATGATATATATATATATATATATATATATATATATT |
| | | | ATTGGTTTGTTTTTATGTTTGTTGCAAGTTTGATGTATTATTTGCC |
| | | | AGTGCTAATAGAATTAAAGCCATTCTCAAATTCTTTTCCAGGCTTC |
| | | | CCTGTCAATTTGCATGGAAGAACGGGTTCTTTTCTCTCAATTTATA |
| | | | TAGTTGGGGAGCATAATGCTTATTGACTAGCAATAAGAAAGAACGT |
| | | | ATTAGAGATTCCGCAATAACTATTAGACTTTGTCCTTTTTTATATA |
| | | | TTATCCTATCCTAACCAAGACAATGGCATTCACCACAATGTCATTT |
| | | | TCTGAGGGCAACATTGTAAACTGCAAAGTGATATCAATTTCTACAT |
| | | | GATTTCCAGTGTGATAGCTAGCCATTTAAACGTAGCTTTCGATTAT |
| | | | TGTGGAATATATATCTTTCTGTGTTTGTCTATTTGCCAATTTTGAT |
| | | | ATTCTTATTCATTTAGAGAGGGGAGAGGAGACTTGAGGAACACTTC |
| | | | ACATGTACAGCATTATGTGTTCTTTTAAGTGCCTTTTAATTCCTAT |
| | | | TAAATCAGCTTCCATACACTGAACAAACACAACGTAGCATGGCATG |
| | | | AGATTCCTTCTTTGTTACAAACATACAAGTAACATTGTCACATTAA |
| | | | GGCATAAATGCCCATCTTTTTCAATTTCCTAAAAAAGCCAACCCTC |
| | | | TTGTTGAATTTGTTATCTGTACATGACAAAATATAAACACAACTGA |
| | | | CTAGAGGACACAGCCAACCAAATATGTATTTGAAGTTCCTCATGAT |
| | | | GGGGTATTCTACATTGATTTATTTCGTTGATTTCTAGACAAAGGGT |
| | | | TTGTGTAGTGATGAACATAACTGATGCTTATAGGCGTGTTACTTAC |
| | | | TCACTTTAGAATGCTCACTATTTGATGAAGACATGCAATAAACCAA |
| | | | AGTTTAGACTGATAATAACAAGAGAATATTATCTTTTGAAAGTTTA |
| | | | GACTCTGATCATTATAGGCATGGTCCCAATTTCTATTTTGCTGATA |
| | | | GAAACGAGTCTGATAAGATCACATGGCCATCCCATAAGGGAAAATA |
| | | | AATGGTGTAGAATCAGTCTTCTACCTGTTTATAAAGGGTCTATCAA |
| | | | AATTCAAAATGTTATTATCAATTTGTTTAGTAGTTATTTTTGGTAG |
| | | | TCATGATGCTTACATTGTTTTTATATCAGTGGCTGCAGGATGAAAT |
| | | | TTCAAGCCCTCTTAGTGCTCGAATTTTCGAACTTTGTAACACTGAT |
| | | | TTTTTCCCAGAGACTTTGCCAAATTCTGAGGTTACTTCCAGCTCAA |
| | | | ACTGTTGTTATGAGGAGAACTCCTCATATGCCACAACAAATATATC |
| | | | TGTAACTGTAGATGTAGACAATAAGTTGAATAGCAATAGCAACACA |
| | | | GTCACCACCCCAACTAGCACTACCACCACCAACACCAACAACAACA |
| | | | CAACCAACAGTAGTAACCTGTCTATTATCTTTGACTCCCAAGAAGA |
| | | | AATTGACAATGACATCTCTGCCTCCATAGACTTCTCACTATCTCCA |
| | | | TCTTTCAATGTTCCCCCATTTCTCCCAGCCACATCCCAGCAAGAAC |
| | | | AGTTTGATTTCTCTTCTGTGCAGCATCAGGTCCAACTAGCAGCATG |
| | | | TTCAGTTGTGGAGGGCTTCTCTCAGTATCCCAATGACTCTGTTGCA |
| | | | CCTCCTTTTATGGGGGCTCCATTACCTTCTGTTTTTGAAGAGGACT |
| | | | GCATATCTTCTGTCCCTTCTTATATGGCTCTTAACCCCTCATCTCC |
| | | | TTGCACTTATCTCAGTCCTGGCATGCCACCATACATGCCTCATGGC |
| | | | CCCCTTACTACTGCCTTATCAACTGGTAGTTCTGGAATATTTGGTG |
| | | | GGAACATTCTTCTTGGTTCTGAACTTCAGGCACAAGAATTGGAATA |
| | | | CCAAGGAGAAAATGGAAGAATGTATTGTACAGATTCAATTCAGCGG |
| | | | GTGTTTAACCCTCCAGACCTTCAGGTATGGATGATTTTCAAACTAT |
| | | | TTTGTTAAGTCATCATTGCCTTTTGTAGTTTTGTTAACAATACCAT |
| | | | CAAAATGAAGTGTTCAGTTCAAATGTTCAATATGCTCAGTAATGAG |
| | | | AGAAAAAGAAGTCCATTGTCCAAGGATTTTTTTATTTCTAATGATG |
| | | | CTAAGCTGTTGTTGTAAGCTTGCTCTTTCAGGTTTCTAGTCTGAAA |
| | | | GGTTGCAGCAGTAAGGTTCTTGACAATAAAGAGTGACTTTACAAGA |
| | | | GGTGAAAAGTTTGCAAAAGATAACGGCCCACAAATTTCTAAGAAGT |
| | | | TTATATGCCCCATAGAATATATGCCCATATCCATCAAACCAGTAGC |
| | | | TAGCATATCAACCCCTTTTATAATCTCATTCTCACCTGTAAAAAGT |
| | | | AGCATAAAATCTGAGCTAGCTAGTAGCAGGAAGGAATCACAACTTT |
| | | | CCACAAAAATTGATAACAATCATCATCAGAAAAGACCCACTTCATG |
| | | | AGTGGTTCTTTTTGCATATCGCTTTCCGGAACCTCTAACTAAAGCT |
| | | | AATGACAGTTTGTTATGACCAATTTTCCTTTGCGTAGCAAGAAAGT |
| | | | AACTGATATTGTTTGTGATGTCTTTCCTAATTCCTAGGCTCTTGGC |
| | | | ACTGAGAGTCAGCAACTAGTACCAGGGGCTGGAAGTTCTGCCAGTC |
| | | | TACCAGAAATCTCAAACTTAGAGGACTCCTCCTTCAAGGTTGGAAA |
| | | | ACTTTCTGTTGAGCAAAGGAAAGAAAAGATCAATAGATACATGAAG |
| | | | AAAAGAAATGAAAGAAACTTCAGCAAGAAAATTAAGGTACACTCAA |
| | | | GCTTCTTCCCATCTTCAAAACAGCCATATCATGTTACATATGCATA |
| | | | TGCTTATAAACAACCACTTTTTATTGCATACTTTCAAGTTCAACTA |
| | | | ATGATGAAACCAAATATGCAGTATGCCTGCCGCAAAACTCTGGCAG |
| | | | ATAGCCGACCTCGAGTCAGAGGAAGGTTTGCAAAGAATGATGACTT |
| | | | TGGGGAAACCAATAGAACTACAAGTAGCAATCATGAAGAAGATGAT |
| | | | GAAGAAGAAGTAAGATTTCCAGGGCTCATTCATTTATTAAACACTT |
| | | | AACAGGACATTTTTTATTCAACTTGCCCAAGTTAAAAAAAAAAATC |
| | | | CCTATCACTTGTACCTTGTCAATTATTTGCTGATATAGTTTTTTAA |
| | | | TCCTGCAGGTAGTTGTGAAAGATGAAGATGATATGGTTGATTCCTC |
| | | | AGATATCTTTGCTCATATCAGTGGGGTGAACTCCTTCAAATGCAAC |
| | | | TATTCCTTCCAGTCCTGGATTTGACTTAATAGTTTGATCAGTATAC |
| | | | CTACATTTAGCTTGTGTAGATTTTTTGCATGGCCATAGACCACCCC |
| | | | AGTTTTCTCATATCTGGTTTTGATGGATGAGGAAACTGAATAATTA |
| | | | GGGTCCTAATACTATACAATAAGTGAATGAAGAACATTTAGAAAGA |
| | | | AACTGTATACATTTTTTGCAGACACTATTTCTTACTACTTAAATGT |
| | | | ATTAAGAATCACATGTTGTATTAACTTTGTCTCCATGTACTAGTGG |
| | | | AGCTCATTCATGTACTCTCTTTTATAATATAGTGATGAGGAACTAG |
| | | | TTTTGGTTACATTCAAGTGCTTAAAGCTTAAAGCCCAAGGTTTAAA |
| | | | ATATTTTTACTGGCTTTGAAACTTTTTTTTT |
| 27 | Mutant | DNA | ATGTTGCAGGATGTTTTTGATCAGCCACTGCCACCAAAGGCCGAAC |
| | POWR3- | | CACAACAGCAACATCCCATTTGGCTGCAGGATGAAATTTCAAGCCC |
| | Glyma. | | TCTTAGTGCTCGAATTTTCGAACTTTGTAACACTGATTTTTTCCCA |
| | 10G190300- | | GAGACTTTGCCAAATTCTGAGGTTACTTCCAGCTCAAACTGTTGTT |
| | CDS sequence | | ATGAGGAGAACTCCTCATATGCCACAACAAATATATCTGTAACTGT |
| | Mutant | | AGATGTAGACAATAAGTTGAATAGCAATAGCAACACAGTCACCACC |
| | comprising | | CCAACTAGCACTACCACCACCAACACCAACAACAACACAACCAACA |
| | a 4 bp | | GTAGTAACCTGTCTATTATCTTTGACTCCCAAGAAGAAATTGACAA |
| | (GGGC) | | TGACATCTCTGCCTCCATAGACTTCTCACTATCTCCATCTTTCAAT |
| | deletion | | GTTCCCCCATTTCTCCCAGCCACATCCCAGCAAGAACAGTTTGATT |
| | on exon 3 | | TCTCTTCTGTGCAGCATCAGGTCCAACTAGCAGCATGTTCAGTTGT |
| | | | GGAGGGCTTCTCTCAGTATCCCAATGACTCTGTTGCACCTCCTTTT |
| | | | ATGGGGGCTCCATTACCTTCTGTTTTTGAAGAGGACTGCATATCTT |
| | | | CTGTCCCTTCTTATATGGCTCTTAACCCCTCATCTCCTTGCACTTA |
| | | | TCTCAGTCCTGGCATGCCACCATACATGCCTCATGGCCCCCTTACT |
| | | | ACTGCCTTATCAACTGGTAGTTCTGGAATATTTGGTGGGAACATTC |
| | | | TTCTTGGTTCTGAACTTCAGGCACAAGAATTGGAATACCAAGGAGA |
| | | | AAATGGAAGAATGTATTGTACAGATTCAATTCAGCGGGTGTTTAAC |
| | | | CCTCCAGACCTTCAGGCTCTTGGCACTGAGAGTCAGCAACTAGTAC |
| | | | CAGGGGCTGGAAGTTCTGCCAGTCTACCAGAAATCTCAAACTTAGA |
| | | | GGACTCCTCCTTCAAGGTTGGAAAACTTTCTGTTGAGCAAAGGAAA |
| | | | GAAAAGATCAATAGATACATGAAGAAAAGAAATGAAAGAAACTTCA |
| | | | GCAAGAAAATTAAGTATGCCTGCCGCAAAACTCTGGCAGATAGCCG |
| | | | ACCTCGAGTCAGAGGAAGGTTTGCAAAGAATGATGACTTTGGGGAA |
| | | | ACCAATAGAACTACAAGTAGCAATCATGAAGAAGATGATGAAGAAG |
| | | | AAGTAGTTGTGAAAGATGAAGATGATATGGTTGATTCCTCAGATAT |
| | | | CTTTGCTCATATCAGTGGGGTGAACTCCTTCAAATGCAACTATTCC |
| | | | TTCCAGTCCTGGATTTGA |
| 28 | WT Protein | Protein | MIPISLLLIETSLIRSHSHPRKGKQMDEISSPLSARIFELCNTDFF |
| | sequence | | PEALQNSEVTSSSNCCYEENSSYATTNISVTVDVDNKLNSNSNTVT |
| | of POWR4- | | TPTSTTTTNTNNNTTNSSNLSIFFDSQEEIDNDISASIDFSLSPSF |
| | Glyma. | | NVPPFLPVSSQQEQFDFSSVQHQVQLAACSVVEGFTQYPTDAVAPP |
| | 20G200400 | | FMGAPLPSVFEEDCISSVPSYMPLNPSSPCTYLSPGMPPYMPHGPL |
| | >G.maxWm82. | | TTALSTDSSGIFGGNILLGSELQTQELEYQGENGRMYCTDSIQRVF |
| | a2.v1\| | | NPPDLQALGTESQQLVPGAGGSGTLTPEISNLEDSSFKVGKLSVEQ |
| | Glyma. | | RKEKINRYMKKRNERNFSKKIKYACRKTLADSRPRVRGRFAKNDDF |
| | 20G200400. | | GETNRTTSSNHEEDDEEEVVMKDEDDMVDSSDIFAHISGLNSFKCN |
| | 1.p | | YSIQSWI* |
| 29 | WT CDS of | DNA | ACACCCTCATACTCTCCATCATCCTAGCTAACTCATCATCATATTC |
| | POWR4 | | ATAACTCCCCCCCCTCTCTCTCTCTCTCCCTCCCTCCTTCCCTATG |
| | >G.maxWm82. | | GAGTGCCCTTGAGCACATGTTTTTAGTCACCAACACACGTGTGAAT |
| | a2.v1\| | | TTTCCCACATTTTGCACTTCCATATAGCTCTTTTCTGGGGTGCCTT |
| | Glyma. | | GATTGCCTCGTGCAAGGGGATACATACACACACACACACACCATGT |
| | 20G200400\| | | TGCAGGATGTTTTTGATCAGCCACTGCCACCAAAGGCCGAACCACA |
| | Chr20: | | ACAGCAACTTCCCCTTGTAATAATCTCCAGCCAGCGGTCTCATTCA |
| | 43781164.. | | TATCTTCCATGTTAATGCATGCATGTGTTCTGCCTACCTTAATTAA |
| | 43785175 | | TTAGTTAATTTGTGTCATGACTCATGACATGGTGTGATCTTAACCC |
| | forward | | AAAATTCATGAAACTTTACAAGTTTGTTTGTTTCCATTTTGGAATC |
| | key: 5′UTR | | TCTTTCTCAGGAATATATTCAAGTCTAAGGCTAAGTTGTTTACAAT |
| | CDS 3′UTR | | TGTCTCGTGATATTGGACCACTTGTGATTCCATTTTTTCATGCATG |
| | | | CATGAATGATATATATGGTTGTTTTATTTTTATGTTTTTTTGCAAG |
| | | | TTTGATATATTATTTCCCAGTGCTAATAGAATTAAAGCCATTCTCA |
| | | | AATTCTTTTCCAGGCTTCTCTGGCAATTTGCAGGGAGGAACGGGTT |
| | | | ATTTTCTCTCAATTTATAGAGTTGGGGGGGGGAGGGGGGGGGGGCA |
| | | | TAATGCTTATTGACTAGCAATAAGAAAGAACGTATTAGAGATTCCG |
| | | | CAATAATTATTAGTCTTTGTCCTTTTTTTTCTTCTATATTATCCTA |
| | | | TCCAATCCAAGACAATGCCATTTACCAAAGATGTCATTTTCTGAGG |
| | | | GCAACATTGTAAACTGCAAGTGATATCAATTTCTACATGATTTTCA |
| | | | GTGTGATAGCTAGCCATTTAAACATAGCTTTCGATTGTTGTGGAAT |
| | | | ATATATCTTTCTGTGGTTGCCTATTTGCCAATTTTGATATTCTTAT |
| | | | TCATTTAGAGAGGGGAGAGGAGACTTGAGGAACACTTCACATATAT |
| | | | AGCATTATGTGTTCTTTTAAGTGCCTTTTAATTTAATTCCTATTAA |
| | | | ATCAGTTTCCATACACTAAACAAACACAACGTAGCATGGCATGAGG |
| | | | TTCCTTCTTTGTTACAAACATACAAGTAACATTGTCACGCTAAGGC |
| | | | ATAAATGCCCATCTTTTTCAATTTCCTAAAAAAACCAACCCTCTTG |
| | | | TAGAATTTATCTGTACATGACCAAATATAAGCACAACTGATAGAGG |
| | | | ACACAACCAACCTAATCTGTATTTGGAGTTCCTCATGATGGGGTAT |
| | | | TGTACATTGATTTATTTCGTTGATTTCTAGACAAAGGGCTTGTAGT |
| | | | GATGAACATAACTGATGCTTATAGGCGTGTTACTCACTATTTGATG |
| | | | AAGACACGCAATAAACCAAAGTTTAGACTGATAATAACAAGAGAAT |
| | | | ATTATTATCTTTTGAAAGTTTAGACTCTGATCATTATAGGCATGAT |
| | | | CCCAATTTCTCTTTTGCTGATAGAAACGAGTCTGATAAGATCACAC |
| | | | AGCCATCCCCGTAAGGGAAAACAAATGGTGTAGAATCAGTCTTCTA |
| | | | CCTGTTTATAAAGGGTCTATCAAAATGTTATCAATATGTGTCTAAT |
| | | | AGTTTTTTTGGTAGTCATGATGCTTACATTGTTTTTCATATCACTG |
| | | | GCTGCAGGATGAAATTTCAAGCCCTCTTAGTGCTCGAATCTTCGAA |
| | | | CTTTGTAACACTGATTTTTTTCCAGAGGCTTTGCAGAATTCTGAGG |
| | | | TTACTTCCAGCTCAAACTGTTGTTACGAGGAGAACTCCTCATATGC |
| | | | CACAACAAATATATCTGTAACTGTAGATGTAGACAATAAGTTGAAT |
| | | | AGCAATAGCAACACAGTCACCACCCCAACTAGCACTACCACCACCA |
| | | | ACACCAACAACAACACAACCAACAGTAGTAACCTGTCTATTTTCTT |
| | | | TGACTCCCAAGAAGAGATTGACAATGACATTTCTGCCTCCATAGAC |
| | | | TTCTCACTCTCTCCATCTTTCAATGTTCCCCCATTTCTCCCAGTCT |
| | | | CATCCCAGCAAGAACAGTTTGATTTCTCTTCTGTGCAGCATCAGGT |
| | | | CCAACTAGCAGCATGTTCGGTTGTGGAGGGCTTCACTCAGTATCCC |
| | | | ACTGACGCTGTTGCACCTCCTTTTATGGGGGCTCCATTACCTTCTG |
| | | | TTTTTGAAGAGGACTGCATATCTTCAGTCCCTTCTTATATGCCTCT |
| | | | TAACCCCTCATCTCCTTGCACTTATCTCAGTCCTGGCATGCCACCA |
| | | | TACATGCCTCATGGCCCCCTTACTACTGCCTTATCAACTGATAGTT |
| | | | CTGGAATATTTGGTGGGAACATTCTTCTTGGTTCTGAACTTCAGAC |
| | | | ACAAGAATTGGAATACCAAGGAGAAAATGGAAGAATGTATTGTACA |
| | | | GATTCAATTCAGCGGGTGTTTAACCCTCCAGACCTTCAGGTATGAA |
| | | | TGATTTTCAAACTATTTAGTTAAGTTCTCCATCATTGCCTTTGTTA |
| | | | ACAATACCATCCTAATGAAAAGTGTTCGGTTCGTTCAATATGCTCA |
| | | | GTAACGAGAGAAAAAGAAGTCCTTGTCCAAGGATTTTTTTTTTTTA |
| | | | TTTCTTATGATGCTAAGCTGTTGTTGTTAGCTTGCTCTTTCAGGTT |
| | | | TCTAGTCTGAAAGGTTGCAGCAGTAAGGTTCTTGACTATAAAGAGT |
| | | | GGCTTTACAAGAGGTGAAAAATCTGCAAAAGATAACGGTCCACAAA |
| | | | TTTCTAAGAAGCTGATATGCCCCATAGAATATATGCCCATATCCAT |
| | | | CCATACAGTAGCTAGCATATCAACCCCTTTTATAATCTCATTCTCA |
| | | | CCTGTAAAAAGTAGCATAAAATCTGAGCTAGCTAGTAGCAGGAAGG |
| | | | AATCACAACATTCCACAAAAATTGATAACGAACAACATCATCATCA |
| | | | GAAAAGACCCACTTCATGACAGTTTGTGATGATCAATTTTCCTTTG |
| | | | CGTAGCAAGAAAGTAACTGATTTTGTTTGCAATTTCTTTCCTAGGC |
| | | | ACTTGGTACTGAGAGCCAGCAACTAGTACCCGGGGCTGGTGGTTCT |
| | | | GGCACTCTAACACCAGAAATCTCAAACTTAGAGGACTCCTCCTTCA |
| | | | AGGTTGGAAAACTTTCTGTTGAGCAAAGGAAAGAAAAGATCAATAG |
| | | | ATACATGAAGAAAAGAAATGAAAGAAACTTCAGCAAGAAAATTAAG |
| | | | GTACAGTCAAGCTCCTTCCCATCTTCAAAACAGCCACATCATCAAA |
| | | | CAAAATTGAAATCTTATCATGTTACATATGCATATGCAAGGTATTC |
| | | | CTTGCTTATAAACAACCACTTGTTATTGCATACTTTCAAGTTCAAC |
| | | | TAATGATGCAACCAAATATGCAGTATGCCTGCCGCAAAACTCTGGC |
| | | | AGATAGCCGACCTCGAGTTAGAGGAAGGTTTGCAAAGAATGATGAC |
| | | | TTTGGAGAAACCAATAGAACTACTAGTAGCAATCATGAAGAAGATG |
| | | | ATGAAGAAGAAGTAAGATTTCCAGGGTTCATTCATTTATTAAACAC |
| | | | TTAACAGGACGCTTTTTATTCAACTATTTGTTGATATAGTTTTTTA |
| | | | ACCCTGCAGGTAGTTATGAAAGATGAAGATGATATGGTTGATTCCT |
| | | | CAGATATCTTTGCTCATATCAGTGGGCTGAACTCCTTCAAATGCAA |
| | | | CTATTCCATCCAGTCCTGGATTTGACTTAATAGTTTGATCAGTATA |
| | | | CCTACATTTAGCCTCTGTAGATTTTTTGCATGGCCATATACCACCC |
| | | | CAGATTTCTCCTATCTGGTTTTGATGGATGAGGAAACTGAATAATT |
| | | | GGGGTCCTAATACTATACAATAAGTGAATGAAAAATATATAGAAAA |
| | | | AAAACTGTATACATTTTTTGCAGACACTACCTTTCTTACTACTTGT |
| | | | ATGTACTAAGAATCACATGTTGTATTAACTCAAACGATTTCATTTG |
| | | | TCTCCATGTACTAGTGGAGCTCATTCATGTACTCTCTTTTATAATA |
| | | | TAGTGATAA |
| 30 | Mutant CDS | DNA | ATGATCCCAATTTCTCTTTTGCTGATAGAAACGAGTCTGATAAGAT |
| | sequence | | CACACAGCCATCCCCGTAAGGGAAAACAAATGGATGAAATTTCAAG |
| | of POWR4 | | CCCTCTTAGTGCTCGAATCTTCGAACTTTGTAACACTGATTTTTTT |
| | protein | | CCAGAGGCTTTGCAGAATTCTGAGGTTACTTCCAGCTCAAACTGTT |
| | | | GTTACGAGGAGAACTCCTCATATGCCACAACAAATATATCTGTAAC |
| | | | TGTAGATGTAGACAATAAGTTGAATAGCAATAGCAACACAGTCACC |
| | | | ACCCCAACTAGCACTACCACCACCAACACCAACAACAACACAACCA |
| | | | ACAGTAGTAACCTGTCTATTTTCTTTGACTCCCAAGAAGAGATTGA |
| | | | CAATGACATTTCTGCCTCCATAGACTTCTCACTCTCTCCATCTTTC |
| | | | AATGTTCCCCCATTTCTCCCAGTCTCATCCCAGCAAGAACAGTTTG |
| | | | ATTTCTCTTCTGTGCAGCATCAGGTCCAACTAGCAGCATGTTCGGT |
| | | | TGTGGAGGGCTTCACTCAGTATCCCACTGACGCTGTTGCACCTCCT |
| | | | TTTATGGGGGCTCCATTACCTTCTGTTTTTGAAGAGGACTGCATAT |
| | | | CTTCAGTCCCTTCTTATATGCCTCTTAACCCCTCATCTCCTTGCAC |
| | | | TTATCTCAGTCCTGGCATGCCACCATACATGCCTCATGGCCCCCTT |
| | | | ACTACTGCCTTATCAACTGATAGTTCTGGAATATTTGGTGGGAACA |
| | | | TTCTTCTTGGTTCTGAACTTCAGACACAAGAATTGGAATACCAAGG |
| | | | AGAAAATGGAAGAATGTATTGTACAGATTCAATTCAGCGGGTGTTT |
| | | | AACCCTCCAGACCTTCAGGCACTTGGTACTGAGAGCCAGCAACTAG |
| | | | TACCCGGGGCTGGTGGTTCTGGCACTCTAACACCAGAAATCTCAAA |
| | | | CTTAGAGGACTCCTCCTTCAAGGTTGGAAAACTTTCTGTTGAGCAA |
| | | | AGGAAAGAAAAGATCAATAGATACATGAAGAAAAGAAATGAAAGAA |
| | | | ACTTCAGCAAGAAAATTAAGTATGCCTGCCGCAAAACTCTGGCAGA |
| | | | TAGCCGACCTCGAGTTAGAGGAAGGTTTGCAAAGAATGATGACTTT |
| | | | GGAGAAACCAATAGAACTACTAGTAGCAATCATGAAGAAGATGATG |
| | | | AAGAAGAAGTAGTTATGAAAGATGAAGATGATATGGTTGATTCCTC |
| | | | AGATATCTTTGCTCATATCAGTGGGCTGAACTCCTTCAAATGCAAC |
| | | | TATTCCATCCAGTCCTGGATTTGA |
| 31 | AtPOWR1 | DNA | TATAAATAAATTGGTTACGATAACTTCCATAACCTTCATTAGTTTC |
| | full- | | TCCAGAAGAAACCTGTAAGAAATTTCTTTCTCTTCTCTGTCTTTAA |
| | length | | ACAGATTTTTATTCTGTTCTGTTTGATTAATTATTATCTCTTTTGT |
| | genomic | | TCCAGTATTTTAATTCTTTAGATTTTCTTGTTCAGACATCACTAGC |
| | DNA | | TCCACTACCACATTCAAGAAGCCTCCTTCAATAATCTTTCCCTTTT |
| | sequence | | CCTGGGGAATTTTCTCTCTGGATTCATTCCCGGAAAATCATTCACA |
| | | | ATTTGGAGACCATAAAGAACACATAAACATGTCGCAGGACGTAATT |
| | | | TCGTCTCATGAGCAGCTCTCCATGGTACGTAATAATCCGTGCAATG |
| | | | TTTATTGTTTGTCCTCGAATCTGTTTTTATGCCCTTGAATGGTTTC |
| | | | TCTTCTTTGGGTTTCCTCTTTTCTATCATACATTGCTTAAATGATT |
| | | | AAAAGCATGAACCTTTTTTTCCTGAAAATTTTGCATGTATATATCT |
| | | | ATGATTTATTCGCAAGGAATAAAATTATTTTCTGAAATCTTTGTAT |
| | | | TTTGTAGGTAACATCGTTTTCTTAATTTTCTTAGTTATTCGATTTT |
| | | | ATCTTTTGTCAAATTGCTTCATTCCTTTTACTTTCTTTTCTGTTTC |
| | | | TGGTTATTCTAGATACAATTCAGTTCATATTCCAAAAGTCTGAAAC |
| | | | GATAATTAGTAAAGCAAACTTTTTAGTGCTAACTCTTTTGTACGGT |
| | | | GTTCATACGTAGTTACGTACCTTCTCGCTAGCCAACCACAAATCGT |
| | | | CACAATACTTAATCAGATAGAATAGAACAGTTTATCAGAATTCATA |
| | | | AGATGCTACATCTCTTCTTTTAGTAATTCCAGTAATTAAAATTTGA |
| | | | ACTTTTATTTATACTTTCGAATAGAATCACACGAATTTTTATTAGA |
| | | | AATATATAATGAGTGTATCAAAAGCAATGATTCTTGCTCTTACCTC |
| | | | GGCCTTTATTTGTTGTTAAAAAATCAGATGTTAGTTTAAATTAGGG |
| | | | AACGTTTTGGTTCTAAAGAAACGCAAGAATCCAAAAACTTTCCTGT |
| | | | TATGGAAACTCTCCCGCCTTATCGTTAATTTCTTCTCTAGATTCCC |
| | | | TCTTGATGAAAACCACATAGAAGGAACAAGATCTAATCGTATAAAT |
| | | | ACATAGTTCTCTCTAAACGACTGTGTTAAGTTAAAACTAACCTTAG |
| | | | GTTTATTCAAAGTTATTGTTTCTTGTTTCACAAGAAAATCCCACCA |
| | | | ATTAGCTTTTTATGCAAGCCTTCAACGTATGTCAATAAACAAACAA |
| | | | TTAGATGCATATATTTACTTGTTCTATTTTCTGCTAATTTGATCTT |
| | | | TCTCCTGCTGTTGTTATTTTGTAGGATGAGATCACAAGTCCTCTCA |
| | | | CCGCACAGATCTTCGATTTCTGCGATTCACAGTTATTTCAAGAAAC |
| | | | CTTCAATCAAACTTCTGAAGTAACCTCTGCTTCAAACGGTTGTGGC |
| | | | TATGTCGAAAACAACAACACCAACAACTTTCCAGACAAATCCAACA |
| | | | GTGGTTCGAATCAAGATCATGAAGACAACAACGACAACGCTGATTT |
| | | | ATCAATCATATTCGATTCACAAGATGATTTCGATAACGATATCACA |
| | | | GCTTCTATCGATTTCTCTTCATCCATCCAGTTCCCAGCCTCCGATC |
| | | | AACTCCAAGAGCAGTTTGATTTCACCGGAATTCAACTTCACCAGCC |
| | | | TCCGAACACTCTCTACTCTTCCTCCTCTGGTGACCTTCTGCCTCCG |
| | | | CCTTTATCGGTTTTCGAAGAAGATTGTCTTTCTTCTGTGCCAAGTT |
| | | | ACAATCTTGGTTCCATAAACCCTTCTTCTCCTTCTTGCTCTTTTTT |
| | | | GGGTAATACCGGTTTACCGACTTACATGACCGTAACCGGGAATATG |
| | | | ATGAACACCGGTTTAGGATCTGGTTTTTACTCCGGGAATATCCATC |
| | | | TCGGTTCTGATTTCAAACCATCACATGATCAATTGATGGAAATTCA |
| | | | AGCTGATAATGGTGGATTGTTCTGTCCAGATCCGATTAAACCCATC |
| | | | TTTAATCCAGGAGATCATCATCTCCAGGTATATATTCCAAACCTTA |
| | | | TATATAAATAATGAAAGGTATTGCTGGCAAACAAATTGTGTAATGA |
| | | | AACCAGATTAGTAAACCGGTCTGATACTAACCGGTTATGGGGTTTG |
| | | | TTTTCTTAATAGGGACTTGACGGTGTCGAGAACCAGAACCACATGG |
| | | | TGGCACAGCCGGTTCTCCCGCAGTTAGGGACGGAGATAACCGGTTT |
| | | | AGACGACCCGAGTTTCAACAAAGTCGGAAAACTCTCGGCAGAGCAG |
| | | | AGAAAAGAGAAGATTCATAGGTATATGAAGAAGAGGAACGAGAGGA |
| | | | ATTTCAGCAAGAAAATCAAGGTCTTTGTGCTTAACCATCATAACCG |
| | | | GATTAAACCAGTGGTTATCCATATACCGATTTGTTCACATGTTTTT |
| | | | TTTGTTATATTTTGTTGTAGTATGCATGTAGGAAGACGTTGGCAGA |
| | | | TAGTCGTCCAAGAGTTAGAGGAAGATTTGCAAAGAATGATGAATTT |
| | | | GGTGAACCAAATAGACAAGCCTGTTCAAGCCATCATGAAGATGATG |
| | | | ATGATGATGTAAGATTAATCTTGTTTGATTACAATTAGAAAACAGA |
| | | | TTAGTTTTTGTTTGATATAATAAAGCGTTTCTAAACGGGGGTGTGG |
| | | | TTTGCAGGTGGGGGTAAAGGAAGAAGAACAGTTAGTTGATTCTTCG |
| | | | GACATATTTTCACACATAAGTGGTGTGAACTCTTTCAAATGCAATT |
| | | | ATCCAATCCAGTCTTGGATTTGAGTTGGATTACTTACTTGGTCTTA |
| | | | TGATTTACAAAAATAAAACAAAAATAGTATTGTTATAGAGGTCTGT |
| | | | AATTTGTATATTTATTAGTACATTTTTTGGCCATGACTCATAGTTT |
| | | | GTCGAATAAGTAGAACATAAGAAAAGAGAGAGATGAATAATGGGTG |
| | | | TAGGCAGGTAATCATGAATAAAATTGTAGTGAGTGAGAGGGGTTGT |
| | | | TTATGTTACATAGAACATTACAAAATTTTGTAGATATATATTATAT |
| | | | GGTATGATAATTATGATGTCTATTAATGTATTTGATGGGTGTTGTA |
| | | | TAAATAATAATCATTTGAACATTATAAATAAGCAGTTGTGTTCAAG |
| | | | T |
| 32 | The | DNA | ATGTCGCAGGACGTAATTTCGTCTCATGAGCAGCTCTCCATGGATG |
| | AtPOWR1 | | AGATCACAAGTCCTCTCACCGCACAGATCTTCGATTTCTGCGATTC |
| | full- | | ACAGTTATTTCAAGAAACCTTCAATCAAACTTCTGAAGTAACCTCT |
| | length | | GCTTCAAACGGTTGTGGCTATGTCGAAAACAACAACACCAACAACT |
| | cDNA | | TTCCAGACAAATCCAACAGTGGTTCGAATCAAGATCATGAAGACAA |
| | sequence | | CAACGACAACGCTGATTTATCAATCATATTCGATTCACAAGATGAT |
| | | | TTCGATAACGATATCACAGCTTCTATCGATTTCTCTTCATCCATCC |
| | | | AGTTCCCAGCCTCCGATCAACTCCAAGAGCAGTTTGATTTCACCGG |
| | | | AATTCAACTTCACCAGCCTCCGAACACTCTCTACTCTTCCTCCTCT |
| | | | GGTGACCTTCTGCCTCCGCCTTTATCGGTTTTCGAAGAAGATTGTC |
| | | | TTTCTTCTGTGCCAAGTTACAATCTTGGTTCCATAAACCCTTCTTC |
| | | | TCCTTCTTGCTCTTTTTTGGGTAATACCGGTTTACCGACTTACATG |
| | | | ACCGTAACCGGGAATATGATGAACACCGGTTTAGGATCTGGTTTTT |
| | | | ACTCCGGGAATATCCATCTCGGTTCTGATTTCAAACCATCACATGA |
| | | | TCAATTGATGGAAATTCAAGCTGATAATGGTGGATTGTTCTGTCCA |
| | | | GATCCGATTAAACCCATCTTTAATCCAGGAGATCATCATCTCCAGG |
| | | | GACTTGACGGTGTCGAGAACCAGAACCACATGGTGGCACAGCCGGT |
| | | | TCTCCCGCAGTTAGGGACGGAGATAACCGGTTTAGACGACCCGAGT |
| | | | TTCAACAAAGTCGGAAAACTCTCGGCAGAGCAGAGAAAAGAGAAGA |
| | | | TTCATAGGTATATGAAGAAGAGGAACGAGAGGAATTTCAGCAAGAA |
| | | | AATCAAGTATGCATGTAGGAAGACGTTGGCAGATAGTCGTCCAAGA |
| | | | GTTAGAGGAAGATTTGCAAAGAATGATGAATTTGGTGAACCAAATA |
| | | | GACAAGCCTGTTCAAGCCATCATGAAGATGATGATGATGATGTGGG |
| | | | GGTAAAGGAAGAAGAACAGTTAGTTGATTCTTCGGACATATTTTCA |
| | | | CACATAAGTGGTGTGAACTCTTTCAAATGCAATTATCCAATCCAGT |
| | | | CTTGGATTTGA |
| 33 | The | protein | MSQDVISSHEQLSMDEITSPLTAQIFDFCDSQLFQETFNQTSEVTS |
| | AtPOWR1 | | ASNGCGYVENNNTNNFPDKSNSGSNQDHEDNNDNADLSIIFDSQDD |
| | full- | | FDNDITASIDFSSSIQFPASDQLQEQFDFTGIQLHQPPNTLYSSSS |
| | length | | GDLLPPPLSVFEEDCLSSVPSYNLGSINPSSPSCSFLGNTGLPTYM |
| | protein | | TVTGNMMNTGLGSGFYSGNIHLGSDFKPSHDQLMEIQADNGGLFCP |
| | sequence | | DPIKPIFNPGDHHLQGLDGVENQNHMVAQPVLPQLGTEITGLDDPS |
| | | | FNKVGKLSAEQRKEKIHRYMKKRNERNFSKKIKYACRKTLADSRPR |
| | | | VRGRFAKNDEFGEPNRQACSSHHEDDDDDVGVKEEEQLVDSSDIFS |
| | | | HISGVNSFKCNYPIQSWI |
| 34 | Primer | | ctggcagaacttccagcccc |
| 35 | Primer | | ccaggactgagataagtgca |
| 36 | Primer | | ccaggactgagataagtgca |
| 37 | Primer | | ATTTATCCCGAATGGTAACGC |
| 38 | Primer | | TACCGGAGATGTGTTTGAAGG |
| 39 | Primer | | AACGTGAAACGGATTTGTCAG |
| 40 | | | AAATTTGGTGAAACCAAGACC |
| 41 | | | ATTTTGCCGATTTCGGAAC |
| 42 | | | ATTTATCCCGAATGGTAACGC |
| 43 | | | TACCGGAGATGTGTTTGAAGG |
| 44 | | | AACGTGAAACGGATTTGTCAG |
| 45 | | | AAATTTGGTGAAACCAAGACC |
| 46 | | | ATTTTGCCGATTTCGGAAC |

(DNA Synthetic)
SEQ ID NO: 4

| | |
|---|---|
| 1 | gtgccgaatt cggatccagc gatctggatt ttagtactgg attttggttt taggaattag |
| 61 | aaattttatt gatagaagta ttttacaaat acaaatacat actaagggtt tcttatatgc |
| 121 | tcaacacatg agcgaaaccc tataggaacc ctaattccct tatctgggaa ctactcacac |
| 181 | attattatgg agaaactcga gcttgtcgat cgactctagc tagagaagct cactttccaa |
| 241 | caaccttagt aatttctccc ttaacataat gaacaaattc ttcaagttga tcagctctag |
| 301 | aagcaagtct atcttcttct tgtccaagat aagcttgtct agcttcaaga ataactggtt |
| 361 | gatattgagc tggaagtctt tccatagccc aatcagcagc aacatccttt ggagcaatct |
| 421 | ttccagtaac agcagaatac caaattctag aaagagtaag aacaacattt ctttcatctc |
| 481 | cagcccaatc tggtggagaa ttccaaagag taagagtttc attaagagct tcaaaaagat |
| 541 | cttgttctgg aactggatca aaaagttctt cagcagctgg tccaacaaga gcaacagaat |
| 601 | gttctctagc cttagtaaga agaatagcaa gatcaatatc aatagtagct ggttcaaaaa |
| 661 | ttccagcaag aatatcattt ctttgccatt ctccaaattg aagttctctc ttagctggat |
| 721 | atctccatgg aataatatca tcatgaacaa caatagtaac ttcaacagct ctaagaattt |
| 781 | cagattctcc tggagaagca gaagtttcaa gaagatcatt aataagagct cttctagtag |
| 841 | tttcatcaag tctaacagta acagtaacaa gaagatcaat atcagaatgt ggcttaagtc |
| 901 | ctccatcaac agcagatcca taaagatgaa cagcaagaag agtaggttca agatgtcttt |
| 961 | caataactcc aacaacttca gaaagttgag tagaaacttc agcaataaca gcttctctca |
| 1021 | tgcaagcagt agcaacagaa gcagaaattc tgaaagaaca gaacttttgc atgaaaatag |
| 1081 | aatccttctt aagaacaagc atagaattag cagagttctt aagcttctta gatccgaaaa |
| 1141 | caagaaaaga agatgactta ggaacttgtg gcttatggaa attagagtta ggattaagag |
| 1201 | tttgaattcc ttgagccata ttgttaattt gagccattgt atcgataatt gtaaatgtaa |
| 1261 | ttgtaatgtt gtttgttgtt tgttgttgtt ggtaattgtt gtaaaaatga gctcttatac |
| 1321 | tcgagcgtgt cctctccaaa tgaaatgaac ttccttatat agaggaaggg tcttgcgaag |
| 1381 | gatagtggga ttgtgcgtca tcccttacgt cagtggagat gtcacatcaa tccacttgct |
| 1441 | ttgtagacgt ggttggaacc tcttcttttt ccacgatgct cctcgtgggt gggggtccat |
| 1501 | ctttgggacc actgtcggca gagagatctt gaatgatagc ctttccttta tcgcaatgat |
| 1561 | ggcatttgta ggagccacct tccttttcta ctgtcctttc gatgaagtga cagatagctg |
| 1621 | ggcaatggaa tccgaggagg tttcccgaaa ttatcctttg ttgaaaagtc tcaatagccc |
| 1681 | tttgatcttc tgagactgta tctttgacat ttttggagta gaccagagtg tcgtgctcca |
| 1741 | ccatgttatc acatcaatcc acttgctttg tagacgtggt tggaacctct tctttttcca |
| 1801 | cgatgctcct cgtgggtggg ggtccatctt tgggaccact gtcggcagag agatcttgaa |
| 1861 | tgatagcctt tcctttatcg caatgatggc atttgtagga gccaccttcc ttttctactg |
| 1921 | tcctttcgat gaagtgacag atagctgggc aatggaatcc gaggaggttt cccgaaatta |
| 1981 | tcctttgttg aaaagtctca atagcccttt gatcttctga gactgtatct ttgacatttt |
| 2041 | tggagtagac cagagtgtcg tgctccacca tgttgacctc cgcaagaatt caagcttagc |
| 2101 | gttcctaaca cctggagaac cttttatgta cttcacaacc ctcaatgctg cttccaggtg |
| 2161 | tgactgtttg ggttgctgca tgaattgact cagcacctgc actgcaaagc tgatatctgg |
| 2221 | ccttgtgatt gtcaagtaga gaagctttcc caccagtctc tgataggatc ccacatcctc |
| 2281 | taactctgca tcatcagtct ttcctaagtg cttgtcatat tctacagtag tcagcctttg |
| 2341 | gttctgttcc attggggttg acactggtct gcagcctccc agaccaacac ctgatatcag |
| 2401 | ttccagtgca tacttcctct ggttcagtag gattcccttt tctgatctca gcacctcaat |
| 2461 | gcctaagaag tattttagtt ctcccaaatc tttcatcttg aaatgctgat gcagggttgc |
| 2521 | ctttgcttct gaaatcaaaa cattgctgct gcctgttatt aacagatcat ccacataaat |
| 2581 | caggattatg acaaggtcag tcccctctct tttggtaaac aaggagtgat cataagcact |
| 2641 | ttgcataaaa ccagcctgca taaggacagt ggtaagcttg atgttccact gccttgatgc |
| 2701 | ttgctttaaa ccatagaggg attcaacagc ctgcacactt tggtttcccc ttggctgtga |
| 2761 | aaaccctgag gcagagacat ataaacttct tccatgaggt caccttgtag aaaagcattg |
| 2821 | ttgacatcca tctggaaaag gaaccagccc ttggaagcag caacagatat gacagctttt |
| 2881 | acagtgacca ttttggccac tggagaaaaa gtttcatggt agtcaaggcc ttcttgctga |
| 2941 | gtgtatccct tggccactag ccttgcctta aacctgtcaa cttcaccatt agctttgtat |
| 3001 | ttaattttgt acacccattt ggaccctata ggctgtttac cagggggtaa agggacaatc |
| 3061 | tcccaggtgt tattatcctc aagagcctgt atctcaaggg acatggcctc catccatttc |
| 3121 | tcatcttgag ctgcttcttt gaaggattta ggttcagtat aagtggagaa agcactcaaa |
| 3181 | taagcttgat agtgaacagg caagtgatca taggaaacat agttggctat agggtatgga |
| 3241 | acatccctag agcctttgtt cagtgtcaca aagtccttga gccagatggg aggacctgca |
| 3301 | ttgcgtttag gtctagtgtg caggtttgct ggaactagag agggatcagc tacagcagta |
| 3361 | tggtgctcaa attcagcatt tgctaggtca ggctcagctg actcaactga ctctgcagga |
| 3421 | gcatgcaggt ggcctgaagg tgcagcatca gctgaagtga tatgattttg ggtagaatga |
| 3481 | gtccccaaag tgatatcctc attggcctct acaatgtcag gaataagaac tggagtatca |
| 3541 | tcatcatcag tatgaaagct ggaaggaaaa atatcattat acactagctg caaagctgta |
| 3601 | tcttcagagc taagtgaacc tgctcgagtg aacatatcag gttcatggga gatggactct |
| 3661 | ttgaaaggga actgaaactc tctgaaaact acatccctgc tcactatgat caccttatta |
| 3721 | tccaagtcat acaacctgta acccttttga gtctcagaat aaccaatgaa gatggttcgc |
| 3781 | ttggctctag gtgcaagttt gtcacctttg ggcagtgttg ctgcaaaagc aagacaccca |
| 3841 | aacactctca agtgatcaag cttggcatgt ttctgataga gtagttcata tggacatttg |
| 3901 | ccttgtaaga ttggagtagg gagtctattg atcaggtata caacagtctt gacacagtct |
| 3961 | ccccaaaacc tggtaggtac accactctga aacttaagtg cccttgccat ctcaaggatg |
| 4021 | tgtctgtgct ttctctccac aacaccattc tgttgtggtg tgtagggaca gctactttga |
| 4081 | tgaacaatcc caagagaggc cagcaactca ttacaacttc tccactagaa ttcgagctcg |
| 4141 | gagctgtagg ccggcctagc ttattctcac ccctaattaa gacaagctgt aggtcggccc |
| 4201 | ctgttcatgt tgttcatcaa ttattgttta accgagttaa aggagatggt caattttaca |
| 4261 | gtagtaaata tgacaagaaa tacacgaaat ttgtgtttaa gtattgaagg acttccattt |
| 4321 | ttttgaatcc accatcctaa aactaatagt gtctaatttt cccgtaacaa tttcttttgt |
| 4381 | ataacattca aataaatgtg gttgacgata tatatttagc ttgcaaattt cctaaaaatt |
| 4441 | attgctccaa acttagtatg cacttaggat tggattgtgt cattattgta cttggcaagg |
| 4501 | gtacatgtgt taattatatg cacttaagcg tgtcattgta cttggctact taaaattgtt |
| 4561 | ttttgttcct cggatgatgt tgatgagtta taaaaaagaa atatatgcct tgaaggaaag |
| 4621 | aaagtttatt tttacggttt atgttaaaat acaatacttt caactatgta gattgtggag |
| 4681 | caaacaagct ttcagtttct ctatcgtgtt ccgatgacac gttaataatg aacttttcct |
| 4741 | ataaacatca attagaagat gttgattttg ttggtcaatt ggtgttattg ttgtgactac |
| 4801 | tttatatttt cgttagattc caagcattat ttccgtctat agggtttgct cttaactgat |
| 4861 | tgttttgagt tattaattat tcaagttgat ttgattggaa tagtaaatct ggatctatta |
| 4921 | tgtaatatat tagggacgaa atcttgcaca ttgagaatga tgtagtctcc tcaaaatttt |
| 4981 | gataaccatt gttttcgagt gatagatgtt gtctatgtca aggttgtcga tgaaattagc |
| 5041 | aatgatggac agactttgtc gaacaagttt tgtaatgcca tcacagaaaa gcatctagaa |
| 5101 | gtactgaaaa acattttttt taacatatac ttttacaaaa tcaagagaat gaataattaa |
| 5161 | atatgaaata agaattacaa aaattttata tttttttaat aaatttcaaa tcaataataa |
| 5221 | gaaagaatgg gataaaatta aagaatatgt tggaagatgt gttattagta tttctgtgca |
| 5281 | taagaatgct tccaaaatta aactctttcc ccaaaattgt atgagtaaaa tttcaaatgt |
| 5341 | ctcgttttaa atataacata agctaaatta aacccagctt agtaaaaaaa atgcatttcc |
| 5401 | gtaaatttcg atgtccaaac aattgtaatg acttaaactc agaacaaaac ctatttttat |
| 5461 | accatgtaaa ttcttacaca ttataatcgt atgtttatcc atccatgcac cccaacaaat |
| 5521 | gaagaaaagg aataaaaaaa aaggtgaatt tgccaaaaac aaattaatgg gatgctaaca |
| 5581 | ttaacataag gcggcatagg gcatcattta attgtgaata aaaaggcttg gtcaacccaa |
| 5641 | tttcttctcc tagttactag ttattaacca tgtcatttgg catatatata ataatccata |
| 5701 | tccatataaa aatacatgtc ccaaaaattg aattccacta caccccacca aagtggtgaa |
| 5761 | ttaatggcca aacgccactc tcgcgttgac acccgcgagt gccaggcttg acgactctac |
| 5821 | gtacaacctc atcaattcct ctatatatgc acataactaa ctttctctgt ttttgacacc |
| 5881 | ctcatacacc ccatcatctt agctaacaca acacagcata cacaacttct ctctctctac |
| 5941 | tactttcttt ttatgtcacc ttcttgatgt ctttaatttg ttttcttttt tagaaaaaaa |
| 6001 | ttaatccttt ttaaggcttt aatttcctaa gcatgcaata ttattgtttt tagtcacctt |
| 6061 | caagttgagg aacatataca cgtttgtgac cacaccaaat tccacttctt tctaagtgtg |
| 6121 | tgtacataga ttttgtttta tatatatttg aggttcaccc ttcattgcct ccttaattct |
| 6181 | gtgcaaaaga ggattcatca ccaccccatg tacccatacg atgttcctga ctatgcgggc |
| 6241 | tatccctatg acgtcccgga ctatgcagga ttgtatccat atgacgttcc agattacgcc |
| 6301 | actagagctg cttacccata cgatgttcct gactatgcgg gctatcccta tgacgtcccg |
| 6361 | gactatgcag gattgtatcc atatgacgtt ccagattacg caatgttgca ggatgttgtc |
| 6421 | cacccctcaa caccggctga gcaactcccc attgatgaga tttcaggccc gattagtgct |
| 6481 | cgaattttcg aactttgcga ccccgatttc ttcccacaca cactgcaaaa ttctgaggtt |
| 6541 | acctccagct caaattgttg ccatgaagag aagtcctcat atgccacaac catatctcca |
| 6601 | cctttagatg tagtagacaa caataagttc aatatcaata gcaatagcag caacatagtc |
| 6661 | accactacct catctagcac taccacaacc agcaccacaa ccaacaacaa caacaacgca |
| 6721 | acgaacggca ataatctttc tatcttcttt gacactcaag atgaaattga caatgacatc |
| 6781 | tcagcctcca tagacttctc atcatctcca tcttttgtcg ttccaccact tctcccaatc |
| 6841 | tcaactcagc aggatcagtt tgatttccct tcagctcagc cacaggtgca actatcaaca |
| 6901 | gcagcaggtt caattttgac gggcctctct cactacccta cagatcctgt gattgcaccc |
| 6961 | cttattggag ctccgttacc atctgttttt gatgatgatt gcatatcttc catcccttct |
| 7021 | tatgtgcctc tcaacccttc atcaccctct tgctcttatc tcagtcctgg cataggagtg |
| 7081 | tacatgccac ctcctggttc ccttaacact gccttatctg ctgacagttc tggattgttt |
| 7141 | ggtgggaaca ttctactggg gtctgaactg caggcacatg aattggacta ccagggagaa |
| 7201 | aatggtggaa tgtattgtac agattcaatt caaagggtgt ttaactcccc agaccttcag |
| 7261 | gcacttggta atgagagtca gaaacttgta gctggggctg gaagctctgc cactttggca |
| 7321 | ccagaaatct cacacttgga ggactctacc ttgaaggttg gaaaactctc tgttgagcag |
| 7381 | aggaaggaaa agattcatag atacatgaag aagagaaacg aaagaaattt cagcaagaaa |
| 7441 | atcaagtatg cttgccgcaa aactttggcg gatagccggc cccgggttag aggaaggttt |
| 7501 | gcaaagaatg atgactttgg agagagccat aaacaaggaa gtagcaatca tgaagatgat |
| 7561 | gatgaagaga taattgtgaa agaagatgat gatatggttg attcctcaga tatctttgca |
| 7621 | catatcagcg gagtgaactc tttcaaatgc aactattcca tccagtcctt gatttgagct |
| 7681 | tattaaatta ttagtttgac tagtgaaagc ttatttatat aattagcttc tgtagattaa |
| 7741 | ttttggtagg acacttttcc catcccggtt ctctaaaatc cgggtttagt ggtttgagta |
| 7801 | aactgaataa atggggtcaa aataaatata ccaataagtt aagtgagtta gaaacgtaca |
| 7861 | gaaattggaa actgtataca tttttgcaga tatatattat ctttttcatt aagttgtacc |
| 7921 | agaacatgga gttgtgttaa ccaagaaaat ttccagttac ccccatccaa gactgatgta |
| 7981 | accaattgat gtagcttctt ttatggtagt caagcagatc gttcaaacat ttggcaataa |
| 8041 | agtttcttaa gattgaatcc tgttgccggt cttgcgatga ttatcatata atttctgttg |
| 8101 | aattacgtta agcatgtaat aattaacatg taatgcatga cgttatttat gagatgggtt |
| 8161 | tttatgatta gagtcccgca attatacatt taatacgcga tagaaaacaa aatatagcgc |
| 8221 | gcaaactagg ataaattatc gcgcgcggtg tcatctatgt tactagatcg acgctttacg |
| 8281 | aggatgcaca tgtgaccgag ggacacgaag tgatccgttt aaactatcag tgtttgacag |
| 8341 | gatatattgg cgggtaaacc taagagaaaa gagcgtttat tagaataatc ggatatttaa |
| 8401 | aagggcgtga aaaggtttat ccgttcgtcc atttgtatgt gccagccgcc tttgcgacgc |
| 8461 | tcaccgggct ggttgccctc gccgctgggc tggcggccgt ctatggccct gcaaacgcgc |
| 8521 | cagaaacgcc gtcgaagccg tgtgcgagac accgcggccg ccggcgttgt ggatacctcg |
| 8581 | cggaaaactt ggccctcact gacagatgag gggcggacgt tgacacttga ggggccgact |
| 8641 | cacccggcgc ggcgttgaca gatgaggggc aggctcgatt tcggccggcg acgtggagct |
| 8701 | ggccagcctc gcaaatcggc gaaaacgcct gattttacgc gagtttccca cagatgatgt |
| 8761 | ggacaagcct ggggataagt gccctgcggt attgacactt gaggggcgcg actactgaca |
| 8821 | gatgaggggc gcgatccttg acacttgagg ggcagagtgc tgacagatga ggggcgcacc |
| 8881 | tattgacatt tgaggggctg tccacaggca gaaaatccag catttgcaag ggtttccgcc |
| 8941 | cgtttttcgg ccaccgctaa cctgtctttt aacctgcttt taaaccaata tttataaacc |
| 9001 | ttgtttttaa ccagggctgc gccctgtgcg cgtgaccgcg cacgccgaag gggggtgccc |
| 9061 | ccccttctcg aaccctcccg gcccgctaac gcgggcctcc catcccccca ggggctgcgc |
| 9121 | ccctcggccg cgaacggcct caccccaaaa atggcagcgc tggccaattc ccgagtgcgc |
| 9181 | ggaaccccta tttgtttatt tttctaaata cattcaaata tgtatccgct catgagacaa |
| 9241 | taaccctgat aaatgcttca ataatattga aaaaggaaga gtatggctaa aatgagaata |
| 9301 | tcaccggaat tgaaaaaact gatcgaaaaa taccgctgcg taaaagatac ggaaggaatg |
| 9361 | tctcctgcta aggtatataa gctggtggga gaaaatgaaa acctatattt aaaaatgacg |
| 9421 | gacagccggt ataaagggac cacctatgat gtggaacggg aaaaggacat gatgctatgg |
| 9481 | ctggaaggaa agctgcctgt tccaaaggtc ctgcactttg aacggcatga tggctggagc |
| 9541 | aatctgctca tgagtgaggc cgatggcgtc ctttgctcgg aagagtatga agatgaacaa |
| 9601 | agccctgaaa agattatcga gctgtatgcg gagtgcatca ggctctttca ctccatcgac |
| 9661 | atatcggatt gtccctatac gaatagctta gacagccgct tagccgaatt ggattactta |
| 9721 | ctgaataacg atctggccga tgtggattgc gaaaactggg aagaggacac tccatttaaa |
| 9781 | gatccgcgcg agctgtatga ttttttaaag acggaaaagc ccgaagagga acttgtcttt |
| 9841 | tcccacggcg acctgggaga cagcaacatc tttgtgaaag atggcaaagt aagtggcttt |
| 9901 | attgatcttg ggagaagcgg cagggcggac aagtggtatg acattgcctt ctgcgtccgg |
| 9961 | tcgctcaggg aggatatcgg ggaagaacag tatgtcgagc tattttttga cttactgggg |
| 10021 | atcaagcctg attgggagaa aataaaatat tatattttac tggatgaatt gttttagctg |
| 10081 | tcagaccaag tttactcata tatactttag attgatttaa aacttcattt ttaatttaaa |
| 10141 | aggatctagg tgaagatcct ttttgataat ctcatgacca aaatccctta acgtgagttt |
| 10201 | tcgttccact gagcgtcaga ccccgtagaa aagatcaaag gatcttcttg agatcctttt |
| 10261 | tttctgcgcg taatctgctg cttgcaaaca aaaaaaccac cgctaccagc ggtggtttgt |
| 10321 | ttgccggatc aagagctacc aactcttttt ccgaaggtaa ctggcttcag cagagcgcag |
| 10381 | ataccaaata ctgtccttct agtgtagccg tagttaggcc accacttcaa gaactctgta |
| 10441 | gcaccgccta catacctcgc tctgctaatc ctgttaccag tggctgctgc cagtggcgat |
| 10501 | aagtcgtgtc ttaccgggtt ggactcaaga cgatagttac cggataaggc gcagcggtcg |
| 10561 | ggctgaacgg ggggttcgtg cacacagccc agcttggagc gaacgaccta caccgaactg |
| 10621 | agatacctac agcgtgagct atgagaaagc gccacgcttc ccgaagggag aaaggcggac |
| 10681 | aggtatccgg taagcggcag ggtcggaaca ggagagcgca cgagggagct tccaggggga |
| 10741 | aacgcctggt atctttatag tcctgtcggg tttcgccacc tctgacttga gcgtcgattt |
| 10801 | ttgtgatgct cgtcaggggg gcggagccta tggaaaaacg ccagcaacgc ggccttttta |
| 10861 | cggttcctgg cagatcctag atgtggcgca acgatgccgg cgacaagcag gagcgcaccg |
| 10921 | acttcttccg catcaagtgt tttggctctc aggccgaggc ccacggcaag tatttgggca |
| 10981 | aggggtcgct ggtattcgtg cagggcaaga ttcggaatac caagtacgag aaggacggcc |
| 11041 | agacggtcta cgggaccgac ttcattgccg ataaggtgga ttatctggac accaaggcac |
| 11101 | caggcgggtc aaatcaggaa taagggcaca ttgccccggc gtgagtcggg gcaatcccgc |
| 11161 | aaggagggtg aatgaatcgg acgtttgacc ggaaggcata caggcaagaa ctgatcgacg |
| 11221 | cggggttttc cgccgaggat gccgaaacca tcgcaagccg caccgtcatg cgtgcgcccc |
| 11281 | gcgaaacctt ccagtccgtc ggctcgatgg tccagcaagc tacggccaag atcgagcgcg |
| 11341 | acagcgtgca actggctccc cctgccctgc ccgcgccatc ggccgccgtg gagcgttcgc |
| 11401 | gtcgtcttga acaggaggcg gcaggtttgg cgaagtcgat gaccatcgac acgcgaggaa |
| 11461 | ctatgacgac caagaagcga aaaaccgccg gcgaggacct ggcaaaacag gtcagcgagg |
| 11521 | ccaagcaggc cgcgttgctg aaacacacga agcagcagat caaggaaatg cagctttcct |
| 11581 | tgttcgatat tgcgccgtgg ccggacacga tgcgagcgat gccaaacgac acggcccgct |
| 11641 | ctgccctgtt caccacgcgc aacaagaaaa tcccgcgcga ggcgctgcaa aacaaggtca |
| 11701 | ttttccacgt caacaaggac gtgaagatca cctacaccgg cgtcgagctg cgggccgacg |
| 11761 | atgacgaact ggtgtggcag caggtgttgg agtacgcgaa gcgcacccct atcggcgagc |
| 11821 | cgatcacctt cacgttctac gagctttgcc aggacctggg ctggtcgatc aatggccggt |
| 11881 | attacacgaa ggccgaggaa tgcctgtcgc gcctacaggc gacggcgatg ggcttcacgt |
| 11941 | ccgaccgcgt tgggcacctg gaatcggtgt cgctgctgca ccgcttccgc gtcctggacc |
| 12001 | gtggcaagaa aacgtcccgt tgccaggtcc tgatcgacga ggaaatcgtc gtgctgtttg |
| 12061 | ctggcgacca ctacacgaaa ttcatatggg agaagtaccg caagctgtcg ccgacggccc |
| 12121 | gacggatgtt cgactatttc agctcgcacc gggagccgta cccgctcaag ctggaaacct |
| 12181 | tccgcctcat gtgcggatcg gattccaccc gcgtgaagaa gtggcgcgag caggtcggcg |
| 12241 | aagcctgcga agagttgcga ggcagcggcc tggtggaaca cgcctgggtc aatgatgacc |
| 12301 | tggtgcattg caaacgctag ggccttgtgg ggtcagttcc ggctgggggt tcagcagccc |
| 12361 | ctgctcggat ctgttggacc ggacagtagt catggttgat gggctgcctg tatcgagtgg |
| 12421 | tgattttgtg ccgagctgcc ggtcggggag ctgttggctg gctggtggca ggatatattg |
| 12481 | tggtgtaaac aaattgacgc ttagacaact taataacaca ttgcggacgt ttttaatgta |
| 12541 | ctggggttga acactct |

(DNA Synthetic)
SEQ ID NO: 7

| | |
|---|---|
| 1 | gtgccgaatt cggatccagc gatctggatt ttagtactgg attttggttt taggaattag |
| 61 | aaattttatt gatagaagta ttttacaaat acaaatacat actaagggtt tcttatatgc |
| 121 | tcaacacatg agcgaaaccc tataggaacc ctaattccct tatctgggaa ctactcacac |
| 181 | attattatgg agaaactcga gcttgtcgat cgactctagc tagagaagct cactttccaa |
| 241 | caaccttagt aatttctccc ttaacataat gaacaaattc ttcaagttga tcagctctag |
| 301 | aagcaagtct atcttcttct tgtccaagat aagcttgtct agcttcaaga ataactggtt |
| 361 | gatattgagc tggaagtctt tccatagccc aatcagcagc aacatccttt ggagcaatct |
| 421 | ttccagtaac agcagaatac caaattctag aaagagtaag aacaacattt ctttcatctc |
| 481 | cagcccaatc tggtggagaa ttccaaagag taagagtttc attaagagct tcaaaaagat |
| 541 | cttgttctgg aactggatca aaaagttctt cagcagctgg tccaacaaga gcaacagaat |
| 601 | gttctctagc cttagtaaga agaatagcaa gatcaatatc aatagtagct ggttcaaaaa |
| 661 | ttccagcaag aatatcattt ctttgccatt ctccaaattg aagttctctc ttagctggat |
| 721 | atctccatgg aataatatca tcatgaacaa caatagtaac ttcaacagct ctaagaattt |
| 781 | cagattctcc tggagaagca gaagtttcaa gaagatcatt aataagagct cttctagtag |
| 841 | tttcatcaag tctaacagta acagtaacaa gaagatcaat atcagaatgt ggcttaagtc |
| 901 | ctccatcaac agcagatcca taaagatgaa cagcaagaag agtaggttca agatgtcttt |
| 961 | caataactcc aacaacttca gaaagttgag tagaaacttc agcaataaca gcttctctca |
| 1021 | tgcaagcagt agcaacagaa gcagaaattc tgaaagaaca gaacttttgc atgaaaatag |
| 1081 | aatccttctt aagaacaagc atagaattag cagagttctt aagcttctta gatccgaaaa |
| 1141 | caagaaaaga agatgactta ggaacttgtg gcttatggaa attagagtta ggattaagag |
| 1201 | tttgaattcc ttgagccata ttgttaattt gagccattgt atcgataatt gtaaatgtaa |
| 1261 | ttgtaatgtt gtttgttgtt tgttgttgtt ggtaattgtt gtaaaaatga gctcttatac |
| 1321 | tcgagcgtgt cctctccaaa tgaaatgaac ttccttatat agaggaaggg tcttgcgaag |
| 1381 | gatagtggga ttgtgcgtca tcccttacgt cagtggagat gtcacatcaa tccacttgct |
| 1441 | ttgtagacgt ggttggaacc tcttcttttt ccacgatgct cctcgtgggt gggggtccat |
| 1501 | ctttgggacc actgtcggca gagagatctt gaatgatagc ctttccttta tcgcaatgat |
| 1561 | ggcatttgta ggagccacct tccttttcta ctgtcctttc gatgaagtga cagatagctg |
| 1621 | ggcaatggaa tccgaggagg tttcccgaaa ttatcctttg ttgaaaagtc tcaatagccc |
| 1681 | tttgatcttc tgagactgta tctttgacat ttttggagta gaccagagtg tcgtgctcca |
| 1741 | ccatgttatc acatcaatcc acttgctttg tagacgtggt tggaacctct tctttttcca |
| 1801 | cgatgctcct cgtgggtggg ggtccatctt tgggaccact gtcggcagag agatcttgaa |
| 1861 | tgatagcctt tcctttatcg caatgatggc atttgtagga gccaccttcc ttttctactg |
| 1921 | tcctttcgat gaagtgacag atagctgggc aatggaatcc gaggaggttt cccgaaatta |
| 1981 | tcctttgttg aaaagtctca atagcccttt gatcttctga gactgtatct ttgacatttt |
| 2041 | tggagtagac cagagtgtcg tgctccacca tgttgacctc cgcaagaatt caagcttagc |
| 2101 | gcctgtgtgg atcatctaaa tgtgcattcc gcaagccaat taatgttcca actgaagcag |
| 2161 | attgcttaca taatttatat acgataccaa atatgcttca cgagcataat ttttaagttg |
| 2221 | ctacctcttg gtaaggggca gctaattgaa actagggatc atgatctccg tttgttcaaa |
| 2281 | aatcctttgg tgtgttttca aaatatcact ttagaatcta atccaagagt tctccctacc |
| 2341 | taataaatat ctttaaccta taataccaaa atctaataat tcgtgggatg atcaccatcc |
| 2401 | ttcacacaac tcatccctgc ttttcctccg tgataagcat gaagaataaa aaacccacta |
| 2461 | atatacgcca attcactgga ttgagaacaa aattcaaaca caatatgcca gcatacagtt |
| 2521 | gataattata tatcagctcc acaaattgat aacaaacagt agtaacatta atgggacaat |
| 2581 | agagttctac acaaaccaaa cttattttca aaggtcatta cagacccatt acacatcaca |
| 2641 | tttcttctta aaatgtttgg gaaaatccta catcgaacat cacacagaaa tcagctccag |
| 2701 | caagctcatc ctcccacctt tctcttcttc tttggttgca gatctcctcc cagttgggac |
| 2761 | agatcgattc tagtctcgta cagtccagtg atggattggt ggatcagagt agcatccagc |
| 2821 | acctccttag tggaagtgta tctctttcta tcgatagtag tatcgaagta cttgaaagca |
| 2881 | gctggagctc ccaggttagt cagagtgaac aggtggatga tgttctcagc ttgctctctg |
| 2941 | attggcttat ctctgtgctt gttgtaagcg gacagcacct tatccaggtt agcatcagcc |
| 3001 | aggatcactc tcttggagaa ctcggagatt tgctcgatga tctcatccag gtagtgcttg |
| 3061 | tgttgctcca cgaacagttg cttttgctcg ttatcctctg gggatccctt cagcttctcg |
| 3121 | tagtgggaag ccaggtacag gaagttcacg tacttggatg gcagagccag ctcgtttccc |
| 3181 | ttttgcagct ctccagcgga agccagcatt ctctttcttc cgttctccag ctcgaacagg |
| 3241 | gagtactttg gcagcttgat gatcagatcc ttcttcacct ccttgtatcc cttagcctcc |
| 3301 | aggaaatcga ttgggttctt ctcgaaggag gatctctcca tgatagtgat tcccagcagc |
| 3361 | tccttcacgg acttcagctt cttggacttt cccttctcca ccttagccac caccagcacg |
| 3421 | gagtaagcca cagttgggga atcgaatcct ccgtacttct ttggatccca atccttcttt |
| 3481 | ctagcgatca gcttatcgga gtttctcttt ggcaggatgg actccttgga gaatcctcca |
| 3541 | gtttgcacct cagtcttttt cacgatgttc acttgtggca tggacagcac ctttctcaca |
| 3601 | gtagcgaaat ctcttccctt atcccacacg atctctccag tctctccgtt agtctcgatc |
| 3661 | agtggtcttt ttctgatctc tccgttagcc agagtgatct cagtcttgaa gaagttcatg |
| 3721 | atgttggagt agaagaagta cttagcagta gcctttccga tctcttgctc ggacttagcg |
| 3781 | atcatctttc tcacatcgta caccttgtaa tctccgtaca cgaactcgga ctccagcttt |
| 3841 | gggtacttct tgatcagagc agttcccacc acagcgttca ggtaagcatc gtgagcgtgg |
| 3901 | tggtagttgt tgatctctct caccttgtag aattggaaat cctttctgaa atcggacacc |
| 3961 | agcttggact tcagagtgat caccttcacc tctctgatca gcttatcgtt ctcatcgtac |
| 4021 | ttagtgttca ttctggaatc caggatttga gccacgtgct tagtgatttg tctagtctcc |
| 4081 | accagttgtc tcttgatgaa tccagcctta tccagctcgg acagtcctcc tctctcagcc |
| 4141 | ttagtcaggt tatcgaactt tctttgagtg atcagcttag cgttcagcag ttgtctccag |
| 4201 | tagttcttca tcttcttcac cacctcctcg gatggcacgt tatcggactt tcctctgttc |
| 4261 | ttatcggatc tagtcagcac cttgttatcg atggaatcat ccttcaggaa ggattgtggc |
| 4321 | acgatgtgat ccacatcgta atcggacagt ctgttgatat ccagctcttg atccacgtac |
| 4381 | atatctcttc cgttttgcag gtagtacagg tacagcttct cgttttgcag ttgagtgttc |
| 4441 | tccactgggt gctccttcag gatttgggat cccagctcct tgattccctc ctcgattctc |
| 4501 | ttcattctct ctctggagtt cttttgtccc ttttgagtag tttggttctc tctagccatc |
| 4561 | tcgatcacga tgttctctgg cttgtgtcta cccatcacct tcaccagctc atccaccacc |
| 4621 | ttcacagttt gcaggattcc cttcttgata gctggggatc cagccaggtt agcgatgtgc |
| 4681 | tcgtgcaggg aatctccttg tccggacact tgagcctttt ggatatcctc cttgaaagtc |
| 4741 | agggaatcat cgtggatcag ttgcatgaag tttctgttag cgaatccatc ggacttcagg |
| 4801 | aaatccagga tagtctttcc ggattgctta tctctgattc cgttgatcag ctttctggac |
| 4861 | agtctacccc atccagtgta tcttcttctc ttcagttgct tcatcacctt atcatcgaac |
| 4921 | aggtgagcgt aagtcttaag tctctcctcg atcatctctc tatcctcgaa cagagtcaga |
| 4981 | gtcagcacga tatcctccag gatatcctcg ttctcctcgt tatccaggaa atccttatcc |
| 5041 | ttgatgatct tcagcagatc gtggtaagtt cccagggaag cgttgaatct atcctccact |
| 5101 | ccggagatct ccacggaatc gaagcactcg atcttcttga agtaatcctc cttcagttgc |
| 5161 | ttcacagtca cctttctgtt agtcttgaac agcagatcca cgatagcctt cttttgctct |
| 5221 | ccggacagga aagctggctt tctcattccc tcagtcacgt acttcacctt agtcagctcg |
| 5281 | ttgtacacag tgaagtactc gtacagcagg gagtgctttg gcagcacctt ctcgtttggc |
| 5341 | aggttcttat cgaagttagt cattctctcg atgaaggatt gagcggaagc tcccttatcc |
| 5401 | accacctgca catcaacaaa ttttggtcat atattagaaa agttataaat taaaatatac |
| 5461 | acacttataa actacagaaa agcaattgct atatactaca ttcttttatt ttgaaaaaaa |
| 5521 | tatttgaaat attatattac tactaattaa tgataattat tatatatata tcaaaggtag |
| 5581 | aagcagaaac ttacctcctc gaagttccat ggagtgatag tctcctcgga ctttctagtc |
| 5641 | atccaagcga atctggagtt tcctctagcc agtggtccca cgtagtatgg gattctgaaa |
| 5701 | gtcaggatct tctcgatctt ctctctgtta tccttcagga atgggtagaa atcctcttgt |
| 5761 | ctcctcagga tagcgtgcag ctctcccagg tggatttggt gtgggatgga tccgttatcg |
| 5821 | aaagttcttt gctttctcag cagatcctct ctgttcagct tcaccagcag ctcctcagtt |
| 5881 | ccatccatct tctccaggat tggcttgatg aacttgtaga actcctcttg ggaagctcct |
| 5941 | ccatcgatgt atccagcgta tccgttcttg gattgatcga agaagatctc cttgtacttc |
| 6001 | tctggcagtt gttgtctcac cagagccttc agcagagtca gatcttggtg gtgctcatcg |
| 6061 | tatctcttga tcatggaagc ggacagtgga gccttagtga tctcagtgtt cactctcagg |
| 6121 | atatcggaca gcaggatagc atcggacagg ttcttagcag ccaggaacag atcagcgtat |
| 6181 | tgatctccga tttgagccag caggttatcc agatcatcat cgtaagtatc cttggacagt |
| 6241 | tgcagcttag catcctcagc cagatcgaag ttggacttga agtttggagt cagtcccagg |
| 6301 | gacagagcga tcaggtttcc gaacagtccg ttcttcttct ctcctggcag ttgagcgatc |
| 6361 | aggttctcca gtctcctgga cttggacagt ctagcggaca ggatagcctt agcatccact |
| 6421 | ccggaagcgt tgattgggtt ctcctcgaac agttggttgt aagtttgcac cagttggatg |
| 6481 | aacagcttat ccacatcgga gttatctggg ttcagatctc cctcgatcag gaagtgtcct |
| 6541 | ctgaacttga tcatgtgagc cagagccagg tagatcagtc tcagatcagc cttatcagtg |
| 6601 | gaatccacca gcttctttct caggtggtag atagttgggt acttctcgtg gtaagccacc |
| 6661 | tcatccacga tgtttccgaa gattgggtgt ctctcgtgct tcttatcctc ctccaccagg |
| 6721 | aaggactcct ccagtctgtg gaagaaggaa tcatccacct tagccatctc gttggagaag |
| 6781 | atctcttgca ggtagcagat tctgttcttt cttctagtgt atcttcttct agcagttctc |
| 6841 | ttcagtctag tagcctcagc agtctctccg gaatcgaaca gcagagctcc gatcaggttc |
| 6901 | ttcttgatgg agtgtctatc agtgtttccc agcaccttga acttcttgga tggcaccttg |
| 6961 | tactcatcag tgatcacagc ccatcccacg gagttagttc cgatatccag tccgatggag |
| 7021 | tacttcttat ccattctgtc gagtcaacaa tcacagataa atcagaatca aaatccatta |
| 7081 | cgtaatctta agtacggatc tgaagcaatc tcgtgaaaaa aaaccctaat ccacggcaat |
| 7141 | agaatcaaca aacaaggcag ttgctaacag atctgagcaa tatctctcga aaaacgatat |
| 7201 | agaatcaacg aataaggctt tttgtcaacg gatctgaaca atttcttgaa aaaccacaga |
| 7261 | agtataaaaa ataaggctgt tgtttaacgg atctgaagta attcatgaaa aagccctgat |
| 7321 | ctatggttac gtttaacgga tctgaagtaa tcaatgaaaa aagccctaat ctacgattaa |
| 7381 | tgaatcaaca attaaggctt gttgtaaagg gatctgacat gaaaaagctc taattagagc |
| 7441 | aaatctacga ttaaagaatc atcaaattaa ggacgttgtt gtaaagataa taaaagctga |
| 7501 | atctgattat gaaaattgaa gatcgatgaa accaaacaaa acacaatcaa atcaacatcg |
| 7561 | tattgatcaa agaacataca caattcatgg aaagaacgaa gaagaaaaac agaagaaatc |
| 7621 | gactaacctt gaagggagag gtagggaaat acacagagag aatggaatat attggaattg |
| 7681 | aactgcgaag gaaggaggag gggtgtgggg cgttttatag cataaggtgt ggaacccttc |
| 7741 | tccagtcata ttgtgacgcg tgttcatgta tggtgtcact tggtgcctat gacacgggta |
| 7801 | atatttggta ttggatgcat tttcggagag tccttctagg aaattgagaa ttgcaatttg |
| 7861 | cgtggtagtt ggcggtttcg taatcttgac ggagtcacat gcaccttcaa gcttcgcttt |
| 7921 | atctgttacg acgtcgttgt tatattgggc ccaccccgtt gcaagtggag cgtccgaagt |
| 7981 | tgatacgggc cccactatcc attgcctgaa gtcggctgct tttacattat taaaaatcta |
| 8041 | tggaaaagct gtcatgctag atttttttta ttatggacca gaaaagaata attgaaaagt |
| 8101 | tttcttatta tggagcaaaa aaatctttaa taattatttt ttaaataaaa tcataattat |
| 8161 | tccactaaat tctttcttat ttaatttcac cttgtaagat atattttcag tagatttttc |
| 8221 | aagtattttt ttaacataca ttacttgtat acatttagtt acagtttata taaaattatt |
| 8281 | catttaaaaa atatttagat attttttaaa aaataattca aaatattttt tttaatgaac |
| 8341 | tttccttttt cctaattttt ctttcttatt tcttattaga tgtttttact accaatcgtt |
| 8401 | tagggaagaa tttttttctc taatgaggag atgtgttaaa tgatgcaact taaggacaga |
| 8461 | ttcaccaata acataagact aaaagatagc tcattgacaa aaacaataat gatgttaaag |
| 8521 | aaaacgaatc catttccatt cggataagcc acaccattta cgaacaagtt gcctactaat |
| 8581 | aatatatggt gtcagttttt tttttttttg ggctgaattg gaggggaaat tatccccaaa |
| 8641 | gtttgattaa aaatcacgcg acaaagtaac aaaactttta ttccgctaaa aaaattagtc |
| 8701 | aaagtcaatt aaatctggta ttagaattac ttttccgcta atgatatatc cacaataaca |
| 8761 | atgtattaat attgaaaaaa ataaagttta tagagtgtga ctttttcttt catttttacg |
| 8821 | taaattgaaa aaaaaatcaa gcctaatata taaactttca ttgatcctca cctaaatcct |
| 8881 | aaagttcttg agcccaatta aaaaacttac tggaggataa aggcccaaac ttcttttttt |
| 8941 | ttttaggaac agagcatgta atgggtaatg aggcagccta agcccataac catgttagcc |
| 9001 | cgtatatatt tttgttaatt agatttccgg tttcttattt ttttttagat ttcagttttt |
| 9061 | gtttctcaaa aaaataaaat aacttttagt gctgtattaa ctttctgtaa ataattttag |
| 9121 | tctcaataaa gattaattct gaataataaa aataaacgag gacaattttt ttcacaactt |
| 9181 | ttaattcttt atttattttt tacacttaat tttgggaaac atggacctag tgtcaaagaa |
| 9241 | ctcaaactca acattacaaa aacttactac atattcaagg atcatggtct gaactaatct |
| 9301 | ccacataaca tataatcaca tttttcaatt aaaaagaaga attaataatg tattaaatga |
| 9361 | tgatcgacaa gatcacatga tttgagattt ttttttatga tgggaaaaat atgatttgag |
| 9421 | attaggtaac aatctttcca ttttagatag acttatgtct ccactagaat tcgagctcgg |
| 9481 | aggacgtcgt tgtggttggt gctttcctta cattctgagc ctctttcctt ctaatccact |
| 9541 | catctgcatc ttcttgtgtc cttactaata cctcattggt tccaaattcc ctccctttaa |
| 9601 | gcaccagctc gtttctgttc ttccacagcc tcccaagtat ccaagggact aaagcctcca |
| 9661 | cattcttcag atcaggatat tcttgtttaa gatgttgaac tctatggagg tttgtatgaa |
| 9721 | ctgatgatct aggaccggat aagttccctt cttcatagcg aacttattca aagaatgttt |
| 9781 | tgtgtatcat tcttgttaca ttgttattaa tgaaaaaata ttattggtca ttggactgaa |
| 9841 | cacgagtgtt aaatatggac caggccccaa ataagatcca ttgatatatg aattaaataa |
| 9901 | caagaataaa tcgagtcacc aaaccacttg ccttttttaa cgagacttgt tcaccaactt |
| 9961 | gatacaaaag tcattatcct atgcaaatca ataatcatac aaaaatatcc aataacacta |
| 10021 | aaaaaattaa aagaaatgga taatttcaca atatgttata cgataaagaa gttacttttc |
| 10081 | caagaaattc actgatttta taagcccact tgcattagat aaatggcaaa aaaaaacaaa |
| 10141 | aaggaaaaga aataaagcac gaagaattct agaaaatacg aaatacgctt caatgcagtg |
| 10201 | ggacccacgg ttcaattatt gccaattttc agctccaccg tatatttaaa aaataaaacg |
| 10261 | ataatgctaa aaaaatataa atcgtaacga tcgttaaatc tcaacggctg gatcttatga |
| 10321 | cgaccgttaa ggaaattgtg gttgtcggac gaagtccagt aataaacggc gtcaaagtgg |
| 10381 | ttgcagccgg cacacacgag tcgtgtttat caactcaaag cacaaatact tttcctcaac |
| 10441 | ctaaaaataa ggcaattagc caaaaacaac tttgcgtgta aacaacgctc aatacacgtg |
| 10501 | tcattttatt attagctatt gcttcaccgc cttagctttc tcgtgaccta gtcgtcctcg |
| 10561 | tcttttcttc ttcttcttct ataaaacaat acccaaagag ctcttcttct tcacaattca |
| 10621 | gatttcaatt tctcaaaatc ttaaaaactt tctctcaatt ctctctaccg tgatcaaggt |
| 10681 | aaatttctgt gttccttatt ctctcaaaat cttcgatttt gttttcgttc gatcccaatt |
| 10741 | tcgtatatgt tctttggttt agattctgtt aatcttagat cgtagacgat tttctgggtt |
| 10801 | tgatcgttag atatcatctt aattctcgat tagggtttca tagatatcat ccgatttgtt |
| 10861 | caaataattt gagttttgtc gaataattac tcttcgattt gtgatttcta tctagatctg |
| 10921 | gtgttagttt ctagtttgtg cgatcgaatt tgtcgattaa tctgagtttt tctgattaac |
| 10981 | agatgcagat cttaatgaca aagcaccagt ggtctagtgg tagaatagta ccctgccacg |
| 11041 | gtacagaccc gggttcgatt cccggctggt gcagctcagc cggtgttgag ggggttttag |
| 11101 | agctagaaat agcaagttaa aataaggcta gtccgttatc aacttgaaaa agtggcaccg |
| 11161 | agtcggtgca acaaagcacc agtggtctag tggtagaata gtaccctgcc acggtacaga |
| 11221 | cccgggttcg attcccggct ggtgcaggta acggagatcc aataaggttt tagagctaga |
| 11281 | aatagcaagt taaaataagg ctagtccgtt atcaacttga aaaagtggca ccgagtcggt |
| 11341 | gcaacaaagc accagtggtc tagtggtaga atagtaccct gccacggtac agacccgggt |
| 11401 | tcgattcccg gctggtgcag gagtgtacat gcctcctccg ttttagagct agaaatagca |
| 11461 | agttaaaata aggctagtcc gttatcaact tgaaaaagtg gcaccgagtc ggtgccaaca |
| 11521 | aagcaccagt ggtctagtgg tagaatagta ccctgccacg gtacagaccc gggttcgatt |
| 11581 | cccggctggt gcagcttctc tagctagagt cgatcgacaa gctcgagttt ctccataata |
| 11641 | atgtgtgagt agttcccaga taagggaatt agggttccta tagggtttcg ctcatgtgtt |
| 11701 | gagcatataa gaaaccctta gtatgtattt gtatttgtaa aatacttcta tcaataaaat |
| 11761 | ttctaattcc taaaaccaaa atccagtact aaaatccaga tcgctttacg aattcccatg |
| 11821 | gggaggtcaa catggtggag cacgacactc tggtctactc caaaaatgtc aaagatacag |
| 11881 | tctcagaaga tcaaagggct attgagactt ttcaacaaag gataatttcg ggaaacctcc |
| 11941 | tcggattcca ttgcccagct atctgtcact tcatcgaaag gacagtagaa aaggaaggtg |
| 12001 | gctcctacaa atgccatcat tgcgataaag gaaaggctat cattcaagat ctctctgccg |
| 12061 | acagtggtcc caaagatgga cccccaccca cgaggagcat cgtggaaaaa gaagaggttc |
| 12121 | caaccacgtc tacaaagcaa gtggattgat gtgataacat ggtggagcac gacactctgg |
| 12181 | tctactccaa aaatgtcaaa gatacagtct cagaagatca aagggctatt gagacttttc |
| 12241 | aacaaaggat aatttcggga aacctcctcg gattccattg cccagctatc tgtcacttca |
| 12301 | tcgaaaggac agtagaaaag gaaggtggct cctacaaatg ccatcattgc gataaaggaa |
| 12361 | aggctatcat tcaagatctc tctgccgaca gtggtcccaa agatggaccc ccacccacga |
| 12421 | ggagcatcgt ggaaaaagaa gaggttccaa ccacgtctac aaagcaagtg gattgatgtg |
| 12481 | acatctccac tgacgtaagg gatgacgcac aatcccacta tccttcgcaa gacccttcct |
| 12541 | ctatataagg aagttcattt catttggaga ggacacgctc gagtataaga gctcattttt |
| 12601 | acaacaatta ccaacaacaa caaacaacaa acaacattac aattacattt acaattatcg |
| 12661 | atacaatggt caagactaat cttttccttt tcttcatctt ctcgcttctc ctctcacttt |
| 12721 | ccagcgcgga catggtgtcc aagggcgaag aggtgattaa agagttcatg aggttcaaag |
| 12781 | tgagaatgga gggcagtatg aacggccatg agttcgagat cgaaggtgaa ggcgaaggta |
| 12841 | gaccatacga gggcacccag acggcgaaac tgaaagtgac gaagggcggc ccactgccct |
| 12901 | tcgcctggga cattctgagt ccacaattta tgtacggttc caaggcatac gttaagcacc |
| 12961 | ccgccgacat ccctgattac aaaaagctgt cgttcccgga ggggtttaaa tgggaaagag |
| 13021 | tgatgaattt tgaggacgga gggctcgtca ctgtgactca agactcctca cttcaagacg |
| 13081 | gcaccctaat ttacaaggtg aagatgaggg gaacaaattt cccccctgat ggccctgtta |
| 13141 | tgcagaaaaa aacaatggga tgggaggcat ccaccgagag actttacccg cgcgacggag |
| 13201 | tgctcaaagg cgaaatccat caggcactga aactaaagga cggcggccac tacctggttg |
| 13261 | agttcaaaac tatatatatg gccaagaaac ccgtccagtt gccgggatat tactatgtgg |
| 13321 | acacaaaact ggatataacg tcccacaacg aagattacac catagtggaa cagtatgagc |
| 13381 | gcagtgaggg tcgtcaccat ctattcctcg gacatggcac gggttccacg ggctcgggtt |
| 13441 | ccagcggcac ggcaagctcg gaggacaaca acatggcggt gatcaaagag ttcatgcggt |
| 13501 | ttaaagtccg tatggagggc agcatgaatg ggcacgaatt tgaaattgag ggagaaggcg |
| 13561 | agggccgacc atatgagggg acacaaaccg cgaaactaaa agtcaccaaa ggtgggccgc |
| 13621 | ttccttttgc ctgggacatt ctctctcctc agttcatgta tggctccaag gcctacgtta |
| 13681 | aacatccggc tgatatcccc gattataaga aattgtcttt cccggagggg tttaaatggg |
| 13741 | agcgggttat gaatttcgag gatggtggac tggttactgt tacacaagat tcgagtctgc |
| 13801 | aggatggcac attgatatac aaagtcaaaa tgaggggtac gaattttccc ccggacggcc |
| 13861 | cggtgatgca gaaaaaaact atgggctggg aggccagcac ggagaggctg tacccgaggg |
| 13921 | acggggtttt gaaaggggag atacaccagg cgcttaaact gaaggacggg gggcactatc |
| 13981 | tggtggaatt taagacgata tatatggcga aaaagcccgt gcagctacct ggctactact |
| 14041 | acgtcgatac caaactcgat attacctctc ataacgagga ctacactatc gtggagcaat |
| 14101 | atgaaagatc agaaggacgt caccaccttt ttctatacgg aatggacgag ctttacaaaa |
| 14161 | aggacgagtt gtaagcttgt cctgctttaa tgagatatgc gagaagccta tgatcgcatg |
| 14221 | atatttgctt tcaattctgt tgtgcacgtt gtaaaaaacc tgagcatgtg tagctcagat |
| 14281 | ccttaccgcc ggtttcggtt cattctaatg aatatatcac ccgttactat cgtattttta |
| 14341 | tgaataatat tctccgttca atttactgat tgtaccctac tacttatatg tacaatatta |
| 14401 | aaatgaaaac aatatattgt gctgaatagg tttatagcga catctatgat agagcgccac |
| 14461 | aataacaaac aattgcgttt tattattaca aatccaattt taaaaaaagc ggcagaaccg |
| 14521 | gtcaaaccta aaagactgat tacataaatc ttattcaaat ttcaaaagtg ccccaggggc |
| 14581 | tagtatctac gacacaccga gcggcgaact aataacgctc actgaaggga actccggttc |
| 14641 | cccgccggcg cgcatgggtg agattccttg aagttgagta ttggccgtcc gctctaccga |
| 14701 | aagttacggg caccattcaa cccggtccag cacggcggcc gggtaaccga cttgctgccc |
| 14761 | cgagaattat gcagcatttt tttggtgtat gtgggcccca aatgaagtgc aggtcaaacc |
| 14821 | ttgacagtga cgacaaatcg ttgggcgggt ccagggcgaa ttttgcgaca acatgtcgag |
| 14881 | gctcagcagg accgctcaga gaggatgcac atgtgaccga gggacacgaa gtgatccgtt |
| 14941 | taaactatca gtgtttgaca ggatatattg gcgggtaaac ctaagagaaa agagcgttta |
| 15001 | ttagaataat cggatattta aaagggcgtg aaaaggttta tccgttcgtc catttgtatg |
| 15061 | tgccagccgc ctttgcgacg ctcaccgggc tggttgccct cgccgctggg ctggcggccg |
| 15121 | tctatggccc tgcaaacgcg ccagaaacgc cgtcgaagcc gtgtgcgaga caccgcggcc |
| 15181 | gccggcgttg tggatacctc gcggaaaact tggccctcac tgacagatga ggggcggacg |
| 15241 | ttgacacttg aggggccgac tcacccggcg cggcgttgac agatgagggg caggctcgat |
| 15301 | ttcggccggc gacgtggagc tggccagcct cgcaaatcgg cgaaaacgcc tgattttacg |
| 15361 | cgagtttccc acagatgatg tggacaagcc tggggataag tgccctgcgg tattgacact |
| 15421 | tgaggggcgc gactactgac agatgagggg cgcgatcctt gacacttgag gggcagagtg |
| 15481 | ctgacagatg aggggcgcac ctattgacat ttgaggggct gtccacaggc agaaaatcca |
| 15541 | gcatttgcaa gggtttccgc ccgtttttcg gccaccgcta acctgtcttt taacctgctt |
| 15601 | ttaaaccaat atttataaac cttgttttta accagggctg cgccctgtgc gcgtgaccgc |
| 15661 | gcacgccgaa ggggggtgcc cccccttctc gaaccctccc ggcccgctaa cgcgggcctc |
| 15721 | ccatcccccc aggggctgcg cccctcggcc gcgaacggcc tcaccccaaa aatggcagcg |
| 15781 | ctggccaatt cccgagtgcg cggaacccct atttgtttat ttttctaaat acattcaaat |
| 15841 | atgtatccgc tcatgagaca ataaccctga taaatgcttc aataatattg aaaaaggaag |
| 15901 | agtatggcta aaatgagaat atcaccggaa ttgaaaaaac tgatcgaaaa ataccgctgc |
| 15961 | gtaaaagata cggaaggaat gtctcctgct aaggtatata agctggtggg agaaaatgaa |
| 16021 | aacctatatt taaaaatgac ggacagccgg tataaaggga ccacctatga tgtggaacgg |
| 16081 | gaaaaggaca tgatgctatg gctggaagga aagctgcctg ttccaaaggt cctgcacttt |
| 16141 | gaacggcatg atggctggag caatctgctc atgagtgagg ccgatggcgt cctttgctcg |
| 16201 | gaagagtatg aagatgaaca aagccctgaa aagattatcg agctgtatgc ggagtgcatc |
| 16261 | aggctctttc actccatcga catatcggat tgtccctata cgaatagctt agacagccgc |
| 16321 | ttagccgaat tggattactt actgaataac gatctggccg atgtggattg cgaaaactgg |
| 16381 | gaagaggaca ctccatttaa agatccgcgc gagctgtatg attttttaaa gacggaaaag |
| 16441 | cccgaagagg aacttgtctt ttcccacggc gacctgggag acagcaacat ctttgtgaaa |
| 16501 | gatggcaaag taagtggctt tattgatctt gggagaagcg gcagggcgga caagtggtat |
| 16561 | gacattgcct tctgcgtccg gtcgctcagg gaggatatcg gggaagaaca gtatgtcgag |
| 16621 | ctattttttg acttactggg gatcaagcct gattgggaga aaataaaata ttatatttta |
| 16681 | ctggatgaat tgttttagct gtcagaccaa gtttactcat atatacttta gattgattta |
| 16741 | aaacttcatt tttaatttaa aaggatctag gtgaagatcc tttttgataa tctcatgacc |
| 16801 | aaaatccctt aacgtgagtt ttcgttccac tgagcgtcag accccgtaga aaagatcaaa |
| 16861 | ggatcttctt gagatccttt ttttctgcgc gtaatctgct gcttgcaaac aaaaaaacca |
| 16921 | ccgctaccag cggtggtttg tttgccggat caagagctac caactctttt tccgaaggta |
| 16981 | actggcttca gcagagcgca gataccaaat actgtccttc tagtgtagcc gtagttaggc |
| 17041 | caccacttca agaactctgt agcaccgcct acatacctcg ctctgctaat cctgttacca |
| 17101 | gtggctgctg ccagtggcga taagtcgtgt cttaccgggt tggactcaag acgatagtta |
| 17161 | ccggataagg cgcagcggtc gggctgaacg gggggttcgt gcacacagcc cagcttggag |
| 17221 | cgaacgacct acaccgaact gagataccta cagcgtgagc tatgagaaag cgccacgctt |
| 17281 | cccgaaggga gaaaggcgga caggtatccg gtaagcggca gggtcggaac aggagagcgc |
| 17341 | acgagggagc ttccaggggg aaacgcctgg tatctttata gtcctgtcgg gtttcgccac |
| 17401 | ctctgacttg agcgtcgatt tttgtgatgc tcgtcagggg ggcggagcct atggaaaaac |
| 17461 | gccagcaacg cggccttttt acggttcctg gcagatccta gatgtggcgc aacgatgccg |
| 17521 | gcgacaagca ggagcgcacc gacttcttcc gcatcaagtg ttttggctct caggccgagg |
| 17581 | cccacggcaa gtatttgggc aaggggtcgc tggtattcgt gcagggcaag attcggaata |
| 17641 | ccaagtacga gaaggacggc cagacggtct acgggaccga cttcattgcc gataaggtgg |
| 17701 | attatctgga caccaaggca ccaggcgggt caaatcagga ataagggcac attgccccgg |
| 17761 | cgtgagtcgg ggcaatcccg caaggagggt gaatgaatcg gacgtttgac cggaaggcat |
| 17821 | acaggcaaga actgatcgac gcggggtttt ccgccgagga tgccgaaacc atcgcaagcc |
| 17881 | gcaccgtcat gcgtgcgccc cgcgaaacct tccagtccgt cggctcgatg gtccagcaag |
| 17941 | ctacggccaa gatcgagcgc gacagcgtgc aactggctcc ccctgccctg cccgcgccat |
| 18001 | cggccgccgt ggagcgttcg cgtcgtcttg aacaggaggc ggcaggtttg gcgaagtcga |
| 18061 | tgaccatcga cacgcgagga actatgacga ccaagaagcg aaaaaccgcc ggcgaggacc |
| 18121 | tggcaaaaca ggtcagcgag gccaagcagg ccgcgttgct gaaacacacg aagcagcaga |
| 18181 | tcaaggaaat gcagctttcc ttgttcgata ttgcgccgtg gccggacacg atgcgagcga |
| 18241 | tgccaaacga cacggcccgc tctgccctgt tcaccacgcg caacaagaaa atcccgcgcg |
| 18301 | aggcgctgca aaacaaggtc attttccacg tcaacaagga cgtgaagatc acctacaccg |
| 18361 | gcgtcgagct gcgggccgac gatgacgaac tggtgtggca gcaggtgttg gagtacgcga |
| 18421 | agcgcacccc tatcggcgag ccgatcacct tcacgttcta cgagctttgc caggacctgg |
| 18481 | gctggtcgat caatggccgg tattacacga aggccgagga atgcctgtcg cgcctacagg |
| 18541 | cgacggcgat gggcttcacg tccgaccgcg ttgggcacct ggaatcggtg tcgctgctgc |
| 18601 | accgcttccg cgtcctggac cgtggcaaga aaacgtcccg ttgccaggtc ctgatcgacg |
| 18661 | aggaaatcgt cgtgctgttt gctggcgacc actacacgaa attcatatgg gagaagtacc |
| 18721 | gcaagctgtc gccgacggcc cgacggatgt tcgactattt cagctcgcac cgggagccgt |
| 18781 | acccgctcaa gctggaaacc ttccgcctca tgtgcggatc ggattccacc cgcgtgaaga |
| 18841 | agtggcgcga gcaggtcggc gaagcctgcg aagagttgcg aggcagcggc ctggtggaac |
| 18901 | acgcctgggt caatgatgac ctggtgcatt gcaaacgcta gggccttgtg gggtcagttc |
| 18961 | cggctggggg ttcagcagcc cctgctcgga tctgttggac cggacagtag tcatggttga |
| 19021 | tgggctgcct gtatcgagtg gtgattttgt gccgagctgc cggtcgggga gctgttggct |
| 19081 | ggctggtggc aggatatatt gtggtgtaaa caaattgacg cttagacaac ttaataacac |
| 19141 | attgcggacg tttttaatgt actggggttg aacactct |

1. A genetically modified legume (Fabaceae) plant having an improved agronomic trait, the plant comprising a nucleic acid sequence encoding a CCT motif-containing protein (CCT protein), wherein the nucleic acid sequence encoding the CCT protein comprises a nucleic acid modification and wherein the nucleic acid modification modifies the expression of the CCT protein in the plant thereby improving the agronomic trait of the plant, wherein the CCT protein is Seed Protein-Oil-Weight-Regulator 1 (POWR1) protein, POWR2 protein, or both, and wherein the improved agronomic trait is improved protein, improved oil accumulation in seeds and improved seed yield.

2-6. (canceled)

7. The genetically modified plant of claim 1, wherein the legume is common bean, cowpea, soybean, chickpea, pea, or Medicago.

8. The genetically modified plant of claim 1, wherein the legume is a soybean species (Glycine max, hispida).

9. (canceled)

10. The genetically modified plant of claim 8, wherein the CCT protein is a GmCCT67 (POWR1) protein and wherein the GmCCT67 protein is encoded by a nucleic acid sequence comprising at least about 75% or more, at least about 85% or more, at least about 95% or more, or 100% sequence identity with the nucleic acid sequence of SEQ ID NO: 2.

11. The genetically modified plant of claim 10, wherein the nucleic acid modification reduces the expression of the GmCCT67 protein in the plant thereby increasing oil content of seeds and reducing protein content of seeds.

12. (canceled)

13. The genetically modified plant of claim 10, wherein the nucleic acid modification increases the expression of the GmCCT67 protein in the plant thereby reducing oil content of seeds and increasing protein content of seeds.

14-16. (canceled)

17. The genetically modified plant of claim 11, wherein the nucleic acid modification in the nucleic acid sequence encoding the GmCCT67 protein comprises a transposable element (TE) insertion, and wherein the nucleic acid sequence comprising the TE insertion comprises at least about 75% or more, at least about 85% or more, at least about 95% or more, or 100% sequence identity with the nucleic acid sequence of SEQ ID NO: 3.

18. (canceled)

19. The genetically modified plant of claim 13, wherein the nucleic acid modification in the nucleic acid sequence encoding the GmCCT67 protein comprises an expression construct for expression of the GmCCT67 protein, wherein the expression construct comprises a nucleic acid sequence encoding the GmCCT67 protein operably linked to a ubiquitin promoter or a native promoter and wherein the expression construct comprises a nucleic acid sequence comprising at least about 75% or more, at least about 85% or more, at least about 95% or more, or 100% sequence identity with the nucleic acid sequence of SEQ ID NO: 4.

20. (canceled)

21. The genetically modified plant of claim 8, wherein the CCT protein is a GmCCT34 (POWR2) protein and wherein the GmCCT34 protein is encoded by a nucleic acid sequence comprising at least about 75% or more, at least about 85% or more, at least about 95% or more, or 100% sequence identity with the nucleic acid sequence of SEQ ID NO: 6.

22. The genetically modified plant of claim 21, wherein the nucleic acid modification reduces the expression of GmCCT34 (POWR2) in the plant, thereby increasing oil content of seeds and reducing protein content of seeds.

23. (canceled)

24. The genetically modified plant of claim 21, wherein the nucleic acid modification increases the expression of GmCCT34 (POWR2) in the plant, thereby reducing oil content of seeds and increasing protein content of seeds.

25-27. (canceled)

28. The genetically modified plant of claim 21, wherein the nucleic acid modification in the nucleic acid sequence encoding the GmCCT34 protein comprises an expression construct for expression of the GmCCT34 protein, wherein the expression construct comprises a nucleic acid sequence encoding the GmCCT34 protein operably linked to a promoter and wherein the expression construct for expression of GmCCT34 (POWR2) comprises a nucleic acid sequence comprising at least about 75% or more, at least about 85% or more, at least about 95% or more, or 100% sequence identity with the nucleic acid sequence of SEQ ID NO: 7.

29. (canceled)

30. The genetically modified plant of claim 21, wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein.

31. (canceled)

32. The genetically modified plant of claim 21, wherein the nucleic acid modification in the nucleic acid sequence encoding the GmCCT34 protein comprises a nucleic acid sequence of SEQ ID NO: 8 to 10 or any combination thereof.

33. The genetically modified plant of claim 21, wherein the nucleic acid modification in the nucleic acid sequence encoding the GmCCT34 protein comprises a nucleic acid sequence of SEQ ID NO: 11 to 13 or any combination thereof.

34. The genetically modified plant of claim 21, wherein the nucleic acid modification in the nucleic acid sequence encoding the GmCCT34 protein comprises a nucleic acid sequence of SEQ ID NO: 14 to 16 or any combination thereof.

35-50. (canceled)

51. The genetically modified plant of claim 1, wherein the plant is a soybean species (Glycine max, hispida), wherein
a. the CCT protein is GmCCT67 (POWR1) and the nucleic acid modification in the nucleic acid sequence encoding the GmCCT67 protein comprises a nucleic acid sequence comprising at least about 75% or more, at least about 85% or more, at least about 95% or more, or 100% sequence identity with the nucleic acid sequence of SEQ ID NO: 3; and
b. the CCT protein is GmCCT34 (POWR2), wherein the nucleic acid modification in the nucleic acid sequence encoding the GmCCT34 protein comprises a genomic deletion of a 1.3-Mb region of the soybean genome (Chr10: 35253890-36584337) comprising a nucleic acid sequence encoding the GmCCT34 CCT protein, wherein the nucleic acid modification in the nucleic acid sequence encoding the GmCCT34 protein comprises a nucleic acid sequence of SEQ ID NO: 8 to 10, wherein the nucleic acid modification in the nucleic acid sequence encoding the GmCCT34 protein comprises a nucleic acid sequence of SEQ ID NO: 11 to 13, wherein the nucleic acid modification in the nucleic acid sequence encoding the GmCCT34 protein comprises a nucleic acid sequence of SEQ ID NO: 14 to 16, or any combination thereof.

52-88. (canceled)

89. A method of improving an agronomic trait of a legume plant, the method comprising: introducing a nucleic acid modification into a nucleic acid sequence encoding a CCT motif-containing protein (CCT protein), wherein the nucleic acid modification modifies the expression of the CCT protein in the plant thereby improving the agronomic trait of the plant, wherein the CCT protein is Seed Protein-Oil-Weight-Regulator 1 (POWR1) protein, POWR2 protein, or both, and wherein the improved agronomic trait is improved protein and oil accumulation in seeds.

90. A kit for improving an agronomic trait of a plant, the kit comprising:
one or more genetically modified plant having an improved agronomic trait of claim 1.
